(19) United States
(12) Patent Application Publication
(10) Pub. No.: US 2024/0066135 A1
(43) Pub. Date: Feb. 29, 2024

(54) DRUG CONJUGATES AND USES THEREOF

(71) Applicant: SUPERTRANS MEDICAL LTD, Tel Aviv (IL)

(72) Inventors: Jacob T. RENDELL, Mevaseret Zion (IL); Lewis F. NEVILLE, Rehovot (IL); Joseph P. VACCA, Telford, PA (US)

(21) Appl. No.: 18/044,757

(22) PCT Filed: Sep. 9, 2021
(86) PCT No.: PCT/IL2021/051098

§ 371 Date: Mar. 9, 2023

Related U.S. Application Data
(60) Provisional application No. 63/077,286, filed on Sep. 11, 2020.

(51) Int. Cl. A61K 47/58; A61K 31/5377; A61K 38/14; A61P 31/04

(52) U.S. Cl. CPC A61K 47/58; A61K 31/5377; A61K 38/14; A61P 31/04

(57) ABSTRACT

The invention generally relates to the design and production of drug conjugates, and specifically conjugates of antibiotic agents to cationic and non-cationic molecular transporters.

### TECHNOLOGICAL FIELD

The invention generally relates to the design and production of drug conjugates, and specifically conjugates of antibiotic agents to cationic and non-cationic molecular transporters.

### BACKGROUND

The design of novel compounds with antibacterial activity is one of the most acute issues of modern chemical biology, biotechnology, and medicine. Despite a broad range of available antibiotics, there are significant problems of drug resistance and side effects which remain unsolved until now.

Bacteria are exceptionally adaptable organisms and have repeatedly proven their ability to resist novel antibiotic agents. Many current antibiotics exhibit undesirable properties such as systemic toxicity, short half-life, and increased susceptibility to bacterial resistance. Some of them further exhibit exceptionally poor oral bioavailability and are therefore administered systemically through an intravenous (IV) route. All these call for the development of more efficient delivery systems to achieve a precision antibacterial efficacy in consort with a precision organ-targeted therapy.

One promising strategy to overcoming these problems is by chemical conjugation of antibiotics to various types of transporter compounds. Use of specific subgroups facilitates the enhancement of specific properties of a given antibiotic (polymers, lipids, nanoparticles, or therapeutic molecules hybrids) or specific targeting of the antibiotic to bacteria (steroids, glycosyl receptors, siderophores, peptides, or antibodies). These conjugated drugs can also display controlled, sustained release and may vary in antibiotic class type, synthetic methods, compositions, varying drug-conjugate bond stabilities, and antibacterial activity.

The need for preventive solutions is also related to the intrinsic and acquired antibiotic resistance. With the increasingly rapid appearance and global spread of antibiotic-resistant bacteria, prevention of infections with appropriately targeted drugs assume greater urgency and importance.

The rise of multidrug resistance in Gram-negative bacteria (“urgent-threat pathogens”/“priority 1 pathogens”) has become a particularly serious challenge. Gram-negative bacteria (GNB) differ from Gram-positive bacteria (GPB) in the structure of the outer envelope, and as a result, in the penetration and retention of chemical agents. The GNB outer envelope consists of three principal layers: (1) the outer membrane, containing the lipopolysaccharide, (2) the peptidoglycan cell wall with partially cross-linked peptide chains, and (3) the cytoplasmic or inner membrane. GPB generally lack the outer membrane, which serves as a permeability barrier that excludes certain drugs and antibiotics from penetrating into the cell wall. This feature is one of the main factors contributing to the intrinsic antibiotic resistances observed in GNB.

Although recent global attention has focused on the issue of multi-drug resistance (MDR) in GNB, the antibiotic resistance in GPB is also a serious concern.

GPB (e.g., staphylococci, streptococci and enterococci) are among the most common bacterial causes of clinical infection and are associated with a diverse spectrum of pathologies, ranging from mild skin and soft tissue infections to life-threatening systemic sepsis and meningitis.

Methicillin-resistant Staphylococcus aureus (MRSA) is perhaps the paradigm of GPB antibiotic resistance. MRSA is a pathogen of concern due to its inherent resistance to almost all β-lactam antibiotics (penicillins, cephalosporins, carbapenems), apart from the novel cephalosporins, yet is almost entirely susceptible to vancomycin.

Nevertheless, resistance of MRSA to such antibiotics is particularly problematic in the context of severe infections, where use of second-line agents confers a proven loss of survival-benefit. Similarly, glycopeptide-resistant enterococci (GRE) are recognized as emerging pathogens, particularly in immunocompromised or hospitalized patients, and have been associated with outbreaks in healthcare facilities globally.

Therefore, despite the apparent availability of many new antibiotics, there is a pressing need for antibiotics with novel spectra of activities and appropriate pharmacokinetic (PK) profiles, and the potential to combat the continuing emergence of antibiotic resistance.

Among strategies for improving the therapeutic potential of known antibiotics, drug conjugates are on the top of the list. One promising approach is to use molecular transporters (MoTr)—a class of peptides and nonpeptidic agents that enable or enhance the delivery of a variety of cargos, small molecules, metals, imaging agents, iron particles, and peptides, through biological membranes. In the context of mammalian cells, MoTr-drug conjugates have advanced to clinical trials for various indications, including stroke, psoriasis, and ischemic damage. Despite this progress, little is known about the ability of MoTrs to enter nonmammalian cells, especially organisms that possess a cell wall.

Certain conjugates of MoTr and marketed antibiotics were previously described, predominantly those using vancomycin in GPB but with very limited data on GNB [1-3]. Vancomycin-lipopeptide conjugates were found effective in vancomycin-resistant GPB Enterococci strain [4]. Other types of vancomycin derivatives were reported to inactivate carbapenem-resistant GNB Acinetobacter baumannii [5] and are now considered in the development of therapeutics for other carbapenem-resistant GNB strains [6-11]. A particular problem to be considered is the inherent nephrotoxicity of many antibiotics, amongst them vancomycin, especially when given alone [11].

### REFERENCES

1. WO2019/165051;
2. Antonoplis et al, ‘Vancomycin-arginine conjugate inhibits growth of carbapenem-resistant E. coli and targets cell-wall synthesis’, ACS Chem Biol 2019, 14(9):2065-2070;
3. Wu et al, ‘Vancomycin C-terminus guanidine modifications and further insights into an added mechanism of action imparted by a peripheral structural modification’, ACS Infect Dis 2020 Jul. 14;
4. Muhlberg et al, ‘Vancomycin-lipopeptide conjugates with high antimicrobial activity on vancomycin-resistant Enterococci’, Pharmaceuticals 2020, 13(6):110;
5. Sarkar et al, ‘Vancomycin derivative inactivates carbapenem-resistant Acinetobacter baumannii and induces autophagy. ACS Chem Biol 2020, 15(4):884-889;
6. Ma et al, ‘Design and synthesis of new vancomycin derivatives’ Chemistry Select 2020, 5(22):6670-6673;
7. Brennan-Krohn et al, ‘New strategies and structural considerations in development of therapeutics for carbapenem-resistant Enterobacteriaceae’, Translational Res 2020, 220:14-32;
8. U.S. Pat. No. 10,081,1655;
9. U.S. Pat. No. 10,626,148;
10. Haldar et al, J. Med. Chem. 2019, 62, 7, 3184-3205;
11. Haldar et al, ACS Infect. Dis. 2016, 2, 2, 132-139.

### GENERAL DESCRIPTION

The primary focus of the invention has been to find new ways to improve the therapeutic potential of antibiotics. The clinical shortcoming of both existing and emerging antibiotic therapy has been related to difficulties in identifying novel bacterial targets, poor pharmacokinetic profiles of hits with associated toxicity issues, lack of targeting/precision treatment, and often, an increasing prevalence of antibiotic-resistant bacteria, typically originating in hospital settings and resulting from prolonged exposure of patients in such settings. Despite the significant progress in the prevention of infections and deaths from resistant bacteria and fungi in recent years, antibiotic-resistance remains one of the leading causes of deaths, and worldwide, 10 million patients might succumb to MDR infections, a number expected to surpass the mortality rate of cancer by 2050.

On the toxicity issue, since antibiotics, for the most part, are short- and fast-acting, they are usually administered in high and multiple daily doses to maintain therapeutic concentrations, which in turn induces damage to the commensal human microbiota. Among the most common side effects related to the use of antibiotics are gastrointestinal symptoms such as vomiting, abdominal pain, diarrhea, decreased appetite together with organ-related injuries (e.g. nephrotoxicity). Indeed, some drugs, e.g., vancomycin, require monitoring of blood concentrations during therapy to ensure the attainment of therapeutic AUC/MIC as well as to mitigate toxic side effects, especially related to the kidney. This is apart from the fact that prolonged duration of antibiotic therapy may select for resistant or dormant bacterial survivors and promote bacterial resistance.

Thus, strategies to overcoming these deficiencies and the efforts to limit the dosage and improve the efficacy and precision of antibiotics are of great interest. Antibiotic conjugation, and the use of molecular transporters (MoTr) in particular, has provided a new set of tools to improve properties of antibiotics, either by impacting PK, or by conferring new antibacterial spectrum of activities not associated with the parental antibiotic. The use of MoTr further significantly shortens the development time and de-risking the therapeutic development. They are further optimally adapted for developing innovative drug repurposing solutions using computational approaches, data mining and integrative analysis over heterogeneous biomedical databases.

As used herein, the “molecular transporter” or MoTr is a chemical moiety that is selected and structured to chemically associate to a chemical entity (herein a cargo moiety) which the transporter is aimed to deliver into a target. The MoTr is generally selected not to impose a direct therapeutic effect but rather assist in the transport of the cargo moiety. The MoTr generally cover many classes of molecules that exhibit cell-penetrating behavior or capabilities, beyond the cell-penetrating peptides. The inventors of the technology disclosed herein have demonstrated that many non-peptidic MoTrs could function in a similar fashion or in a superior fashion to cell-penetrating peptides. It has been known that for a given MoTr, the transport into the cell is a function of arginine content rather than its peptide backbone, and more specifically, is dependent on the number and spatial arrangement of guanidinium groups.

The inventors have significantly improved this framework to design a highly versatile and highly adaptable MoTr platform with specific combinations of closely located cationic and lipophilic moieties. Thus, the MoTr moiety comprises a cationic moiety or group and a lipophilic moiety or group, both being chemically associated directly and indirectly to the cargo moiety, as shown herein. The MoTr platform of the invention can be conjugated to various diagnostic and therapeutic types of cargo moieties. In the example of an antibiotic cargo, the MoTr platform of the invention has proved to be highly advantageous in improving PK, minimum inhibitory concentration (MIC), in vitro toxicity/in vivo tolerability and therapeutic safety/efficacy balance overall. In other words, the use of the MoTr platform of the invention permitted to achieve an inhibition of bacterial burden at a lower dose of the antibiotic through precision targeting of the bacteria in consort with precision organ targeting as compared to the unconjugated forms. Furthermore, with GPB antibiotic such as vancomycin, the use of the MoTr platform of the invention enabled to extend the applicability of the conjugated antibiotic to a number of GNB strains (including strains with multiple resistance mechanisms), whereas the corresponding unconjugated antibiotic was deemed highly ineffective. Moreover, the use of the MoTr platform of the invention also proved to be effective with other types of administration routes, which in the example of the vancomycin-MoTr conjugate was highly advantageous in providing the option to a less invasive subcutaneous administration instead of the conventional IV route.

These findings were highly surprising and far-reaching in terms of their potential to impact conventional health care procedures and methods of application of antibiotics, and provides an opportunity for wide-ranging applicability in acute and outpatient care in home and hospital settings.

More specifically, the invention describes new active agents generated through covalent conjugation of aryl or heteroaryl subunits, or straight and branched alkyls, cyclic alkyls, heteroalkyls and heterocyclyls carrying cationic nitrogen-containing functional groups, comprising primary, secondary, tertiary or quaternary amines, amidines, guanidines, imidazolines, ureas and others.

The cationic and lipophilic segments together making up the MoTr platform may be associated to each other directly or via linking moieties or may be separately associated to different moieties on the cargo moiety. Thus, each compound of the invention comprises a cargo moiety, at times a therapeutically active moiety (e.g., an antibiotic), a lipophilic moiety and a cationic moiety, wherein both the lipophilic moiety and the cationic moiety are directly or indirectly associated to the cargo moiety. In other words, compounds of the invention may be generally represented by one of the structured depicted herein. Compounds of the invention are also structured to optionally permit hydrogen bonding with the target site. The bonding may be through hydrogen atoms present on the target site or on compounds of the invention.

In the broadest sense, the compounds or conjugates of the invention are of the general Formula A-MoTr (referred to herein as a compound of Formula (X)), wherein A is a medicinal or diagnostic cargo moiety, such as a drug moiety, and MoTr is a molecular transporter moiety, as defined. The transporter moiety may be a single moiety comprising the lipophilic and cationic segments (and optionally a linker moiety) or comprise multiple moieties, one of which containing the lipophilic segment and another comprising the cationic segment.

As disclosed herein, compounds or conjugates or the invention may be provided in a free acid or free base form, in anionic or cationic form (namely in a salt form), in an enantiomerically pure form, as a mixture of isomers, as hydrates, in crystalline, co-crystalline forms or amorphic forms.

The “medicinal or diagnostic cargo moiety”, designated “A”, is the diagnostic or therapeutic or otherwise the active to be delivered. Putting it differently, the cargo moiety A is the substance which the transporter carries to the target. The cargo moiety may be any such compound known to have a diagnostic or therapeutic utility and which is effective for achieving an intended purpose (diagnosis or therapeutic) following administration to a subject's body. Thus, cargo moieties used in the present compositions or in accordance with methods and uses disclosed herein, may be any of contrasting agents, fluorescent agents, radioactive agents, or optical imaging agents used in diagnosis or any therapeutic agents, optionally selected from small molecule drugs (having molecular weights smaller than about 2 KDa), antibiotics, antiviral agents, chemotherapeutic agents, anticancer drugs, nucleosides, polynucleotides, proteins, peptides/proteins/enzymes, nucleic acids, metal-based materials, catalysts, site-specific cellular targeting agents and others, when associated to an MoTr moiety as defined. As shown herein, the association between the MoTr and a particular cargo moiety, namely a diagnostic agent or a therapeutic agent, improves deliverability of the cargo moiety, increases its targeted effect, in some cases reduces the amount of the diagnostic or therapeutic agent that is required to achieve the effect, reduces toxicity, enables delivery of the diagnostic or therapeutic agent in a way previously impossible, etc., as compared to the diagnostic or therapeutic agent when administrated not associated to the MoTr.

As used herein, the term “moiety”, when used in reference to any component of the general structures or specific compounds disclosed herein, designates a group or a functionality having connectivity to another group or functionality in the A-MoTr compound of the invention. For example, the cargo moiety designated A has a connectivity, e.g., via chemical covalent association, to the MoTr moiety. The MoTr is similarly constructed or a linker moiety, a cationic moiety and a lipophilic moiety, each defining a chemical group or functionality. The term “moiety” is used interchangeably with “group”, “functionality” and “feature”.

In some embodiments, of a compound A-MoTr of the invention, the cargo moiety A is a therapeutic agent or a drug. In some embodiments, A is an antibiotic. In some embodiments, the antibiotic is as defined herein, e.g., vancomycin.

In some embodiments, the MoTr moiety is the group -L-Hp-X(v) and the compound may be represented by Formula (I):

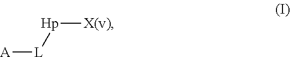

wherein
A is a cargo moiety, the cargo moiety being as defined herein, in some embodiments, an antibiotic moiety,
L is a linker moiety, which may be absent,
Hp is a lipophilic moiety;
v is 1 or 2, and
X is an N-containing cationic group.
   The invention also provides a compound of Formula (I), as shown above, wherein
A is a cargo moiety,
L is a linker moiety, which may be absent,
Hp is a lipophilic moiety selected from C1-C5alkylenes (which may be linear or branched), C6-C12arylene, C3-C10heteroarylene, C10-C20aralkylene, C6-C16heteroaralkylene, C5-C10carbocyclylene and C3-C10heterocarbocyclylene,
v is 1 or 2, and
X is an N-containing cationic group.

In some embodiments, in a compound of Formula (I), A is an antibiotic.

In some embodiments, compounds of Formula (I) may be represented by Formula (Ia) or (Ib):

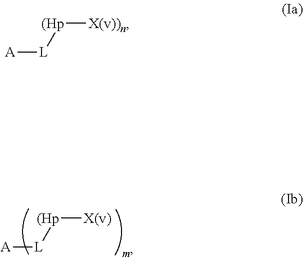

wherein each of A, L, Hp, X and v are as defined above; and wherein each of n and m, independently of the other, is an integer designating the number of repeating moieties, each integer being between 1 and 10.

As shown, integer “n” is the number of times moiety -(Hp-X) is linearly repeated. For example, where n is 1, the compound of Formula (Ia) is

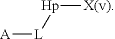

In cases where n is 2, the compound of Formula (Ia) is

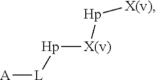

etc. Thus, in cases where n is greater than 1, each repeating unit is associated in a linear fashion in the direction shown in the Formula.

Similarly, integer “m” is the number of times moiety -(L-Hp-X) is linearly repeated. Where m=1, the compound of Formula (Ib) is

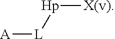

In cases where m=2, the compound of Formula (Ib) is

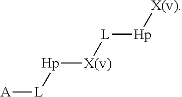

In each of the repeated units, the variables multiplied may be the same or different. For example, in the compound having the structure

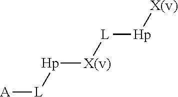

each of the X moieties may be the same or different. Similarly, each of the L or Hp moieties, independently, may be the same or different.

In each of the aforementioned structural Formulae, Hp and X may be associated to each other directly or may be associated via a linker -Lh-, as defined herein. In some embodiments, linker -Lₕ- is present and compounds of the invention may be represented by Formula (II):

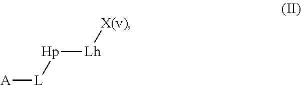

wherein each of A, L, Hp, v and X is as disclosed herein and wherein Lh is a linker moiety associating Hp to X. In such embodiments, the moiety -(Hp-Lh-X) may appear n times, wherein n is between 1 and 10 and the moiety -(L-Hp-Lh-X) may be repeated m times, wherein m (independently from n) is an integer between 1 and 10. The repetition of the groups may be as depicted for structures of Formulae (Ia) and (Ib) above.

In some embodiments, the lipophilic moiety Hp may be associated directly on the cargo moiety or may be a pendant group extending from the linker moiety L such that the lipophilic group Hp is not associated to X. Such compounds may be of Formulae (III), (IV), (V), (VI) and (VII):

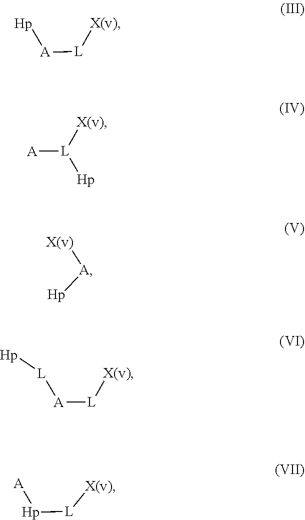

wherein
A is a cargo moiety, being in some embodiments an antibiotic moiety,
L is a linker moiety which may be absent,
Hp is a lipophilic unit,
v is 1 or 2; and
X is an N-containing cationic group.

A compound of Formula (III) may be of Formula (IIIa):

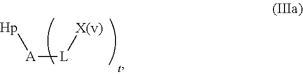

wherein t is an integer between 1 and 10.

In some embodiments, where t=1, the compound may be of the structure

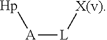

In cases t=2, the compound may be of the structure

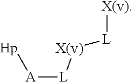

Thus, in cases where t is greater than 1, each repeating unit is associated in a linear fashion in the direction shown in the Formula.

Compounds of the invention may alternatively be represented by Formula (VIII) and (IX):

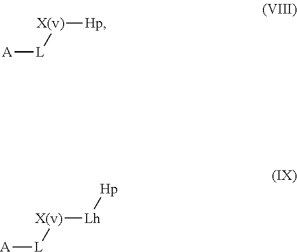

wherein each of A, L, X, v and Hp are as defined herein, and wherein Hp may be associated with X directly or via linker moiety -Lₕ-.

In some embodiments, compounds of Formula (VIII) may be represented by Formula (VIIIa) or (VIIIb):

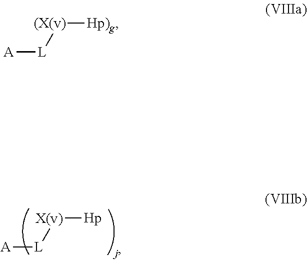

wherein each of A, L, X, v and Hp are as defined herein; and wherein each of g and j, independently of the other, is an integer designating the number of repeating moieties, the integer being between 1 and 10.

As shown, integer “g” is the number of times moiety -(X-Hp) is linearly repeated and integer “j” is the number of times moiety -(L-X-Hp) is linearly repeated.

Linear repetition is as demonstrated hereinabove.

In each of the general compounds of the invention (I) through (XI), where one or more of the moieties or groups appears two or more times, each appearance is selected independently by the other appearance. For example, in a structure of Formula (VI), each linker moiety L, appearing twice in the structure, may be selected independently.

In some cases one L may be present and the other absent; or one may be selected to be a specific functional group while the other L may be the same or different group.

In other aspects of the invention, there is provided a compound of any one of Formulae (I), (Ia), (Ib), (II), (III), (IV), (V), (VI), (VII), (VIII) and (IX), wherein A is an antibiotic moiety.

In other aspects of the invention, any of the structures (I), (Ia), (Ib), (II), (III), (IV), (V), (VI), (VII), (VIII) and (IX) disclosed herein may be presented by MoTr, being Formula (X). As defined herein, in a compound of Formula (X), A is as defined herein and MoTr is the sequence derived from structure (I), namely -L-Hp-X(v) or -L-Hp-Lₕ-X(v), wherein each of L, Hp, Lₕ, X and v are as defined herein.

In some embodiments, MoTr comprises a phenyl or a biphenyl moiety and an N-containing moiety is amine or guanidinyl. In some embodiments, the MoTr is selected from:

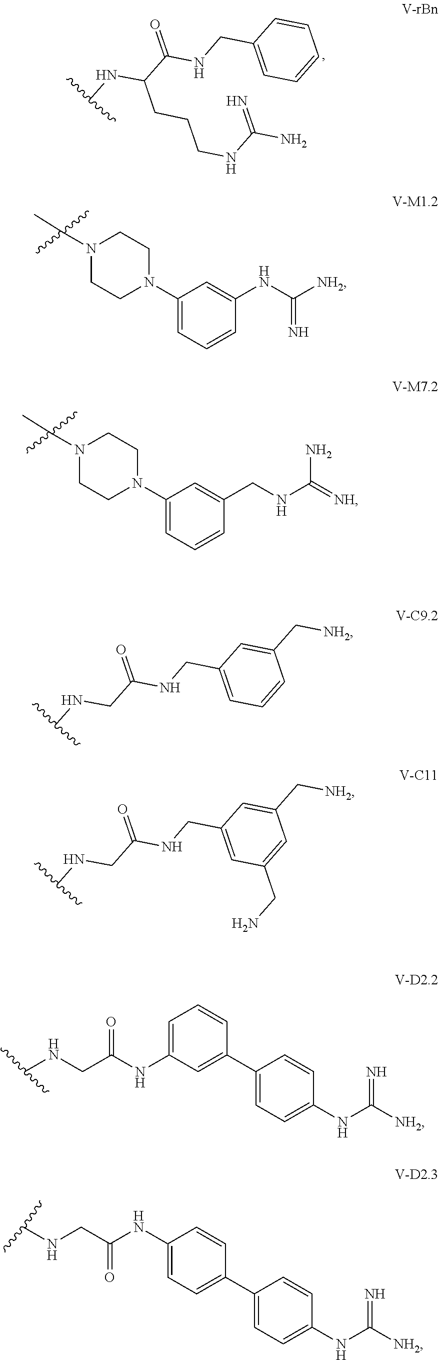

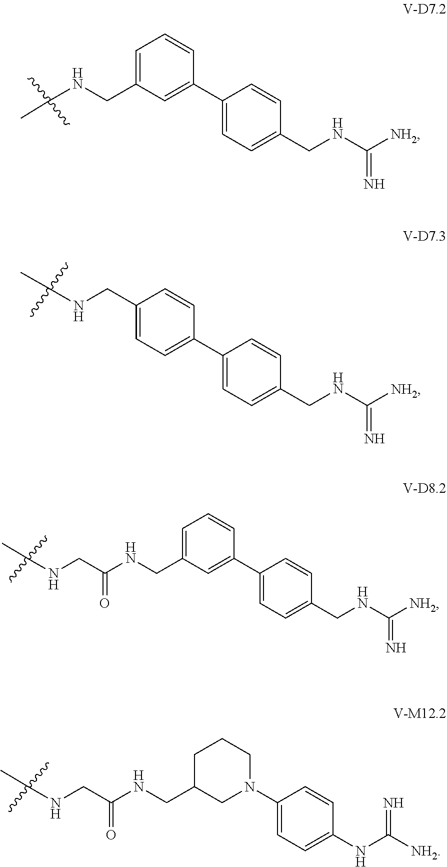 [SMILES: *NCc1cccc(-c2ccc(CNC(=N)N)cc2)c1 ; *NCc1ccc(-c2ccc(CNC(=N)N)cc2)cc1 ; *NCC(=O)NCc1cccc(-c2ccc(CNC(=N)N)cc2)c1 ; *NCC(=O)NCC1CCCN(c2ccc(NC(=N)N)cc2)C1]

In some embodiments, MoTr is selected from

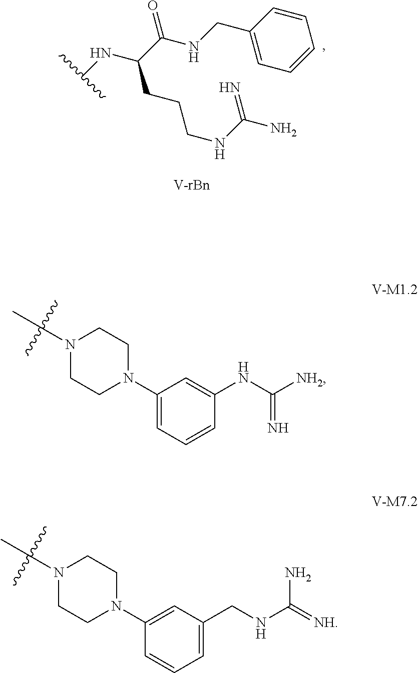

In some embodiments, MoTr is selected from (or is any one of):

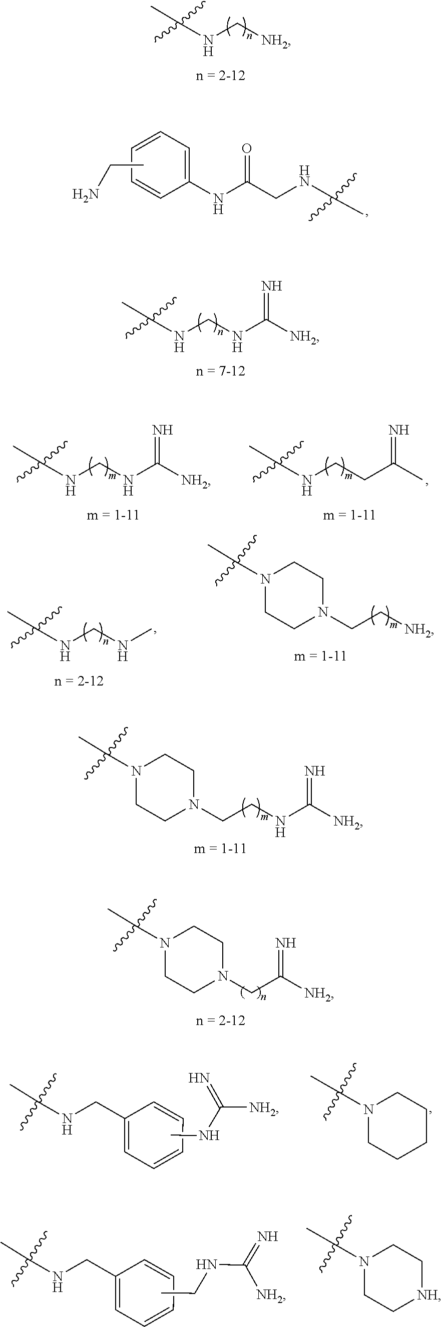

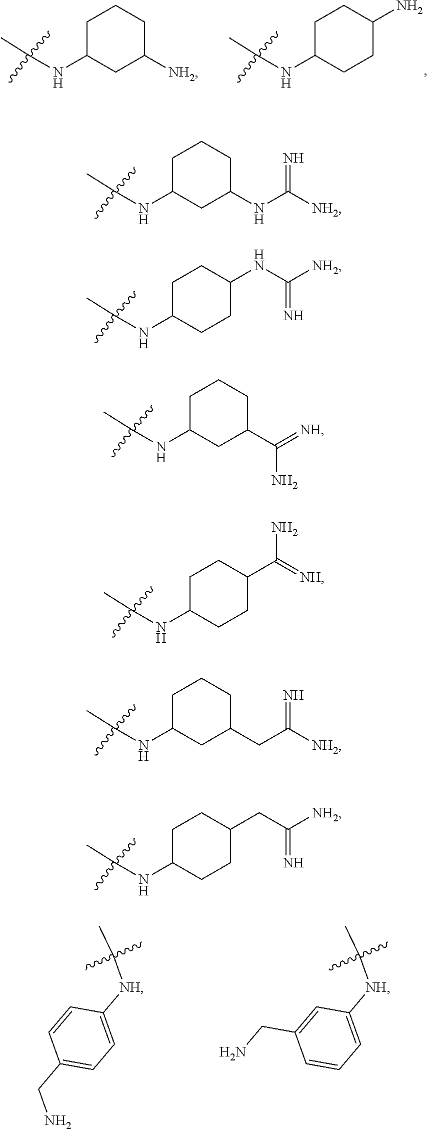

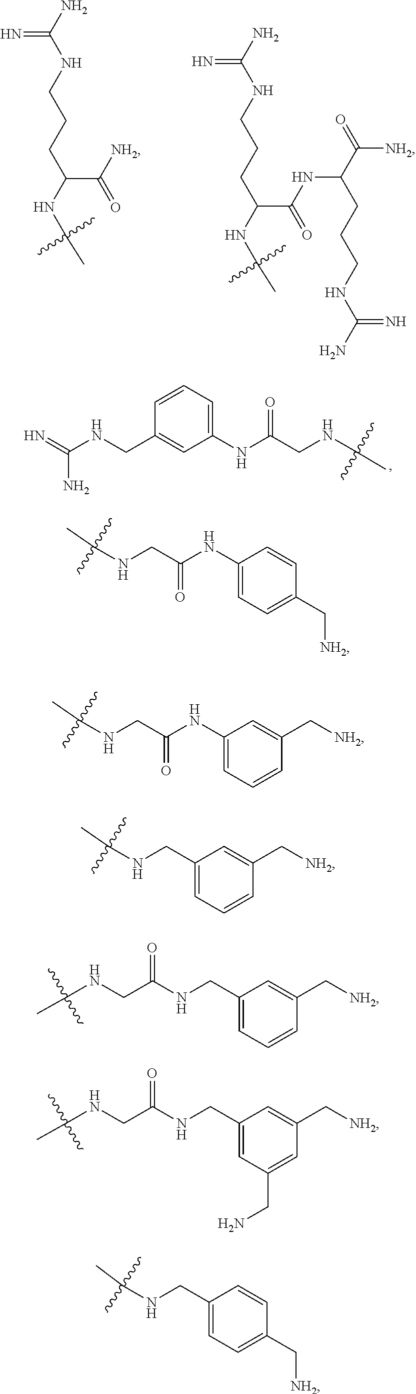

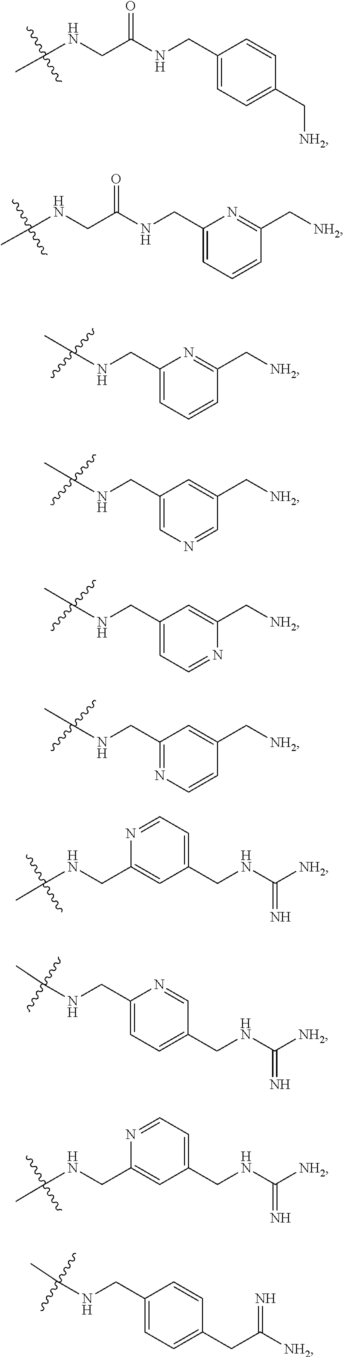

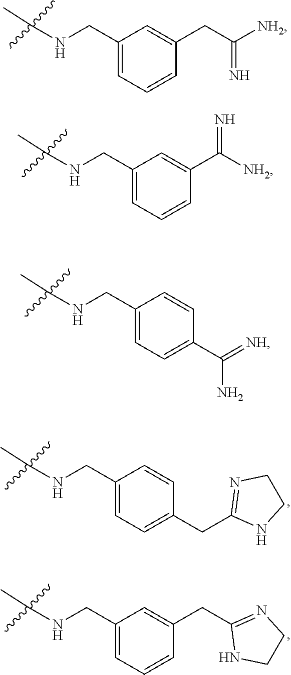

Hp is as defined herein

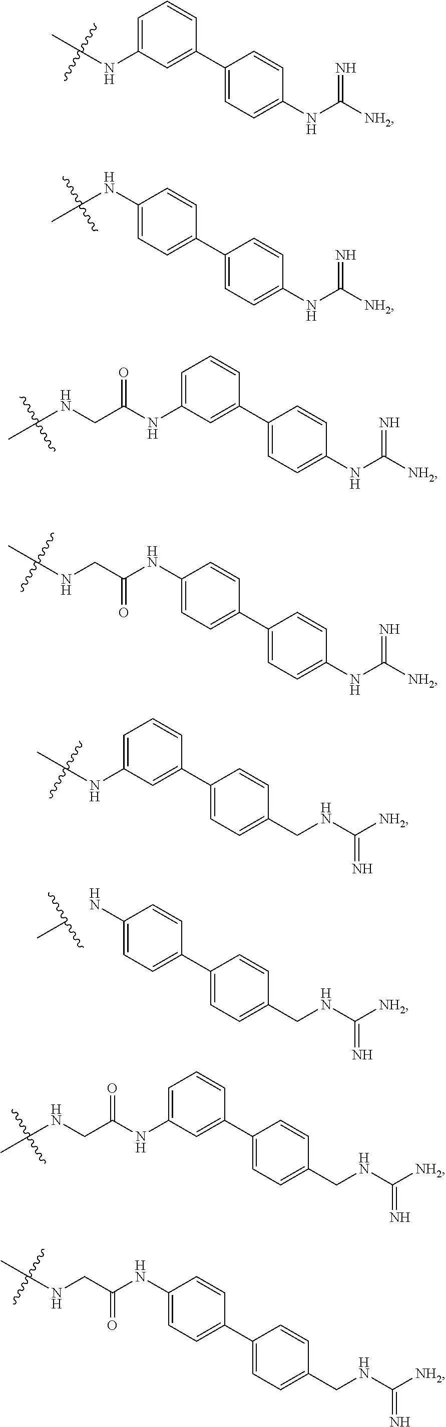

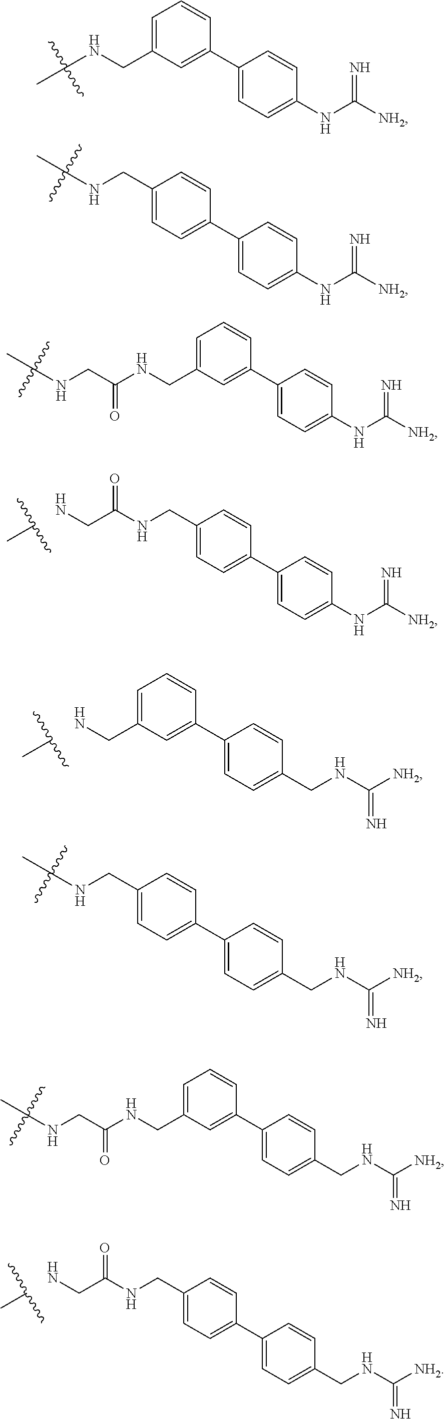

Any MoTr structure provided above constitutes a separate embodiment of the invention.

In some embodiments, MoTr is selected from (or is any one of):

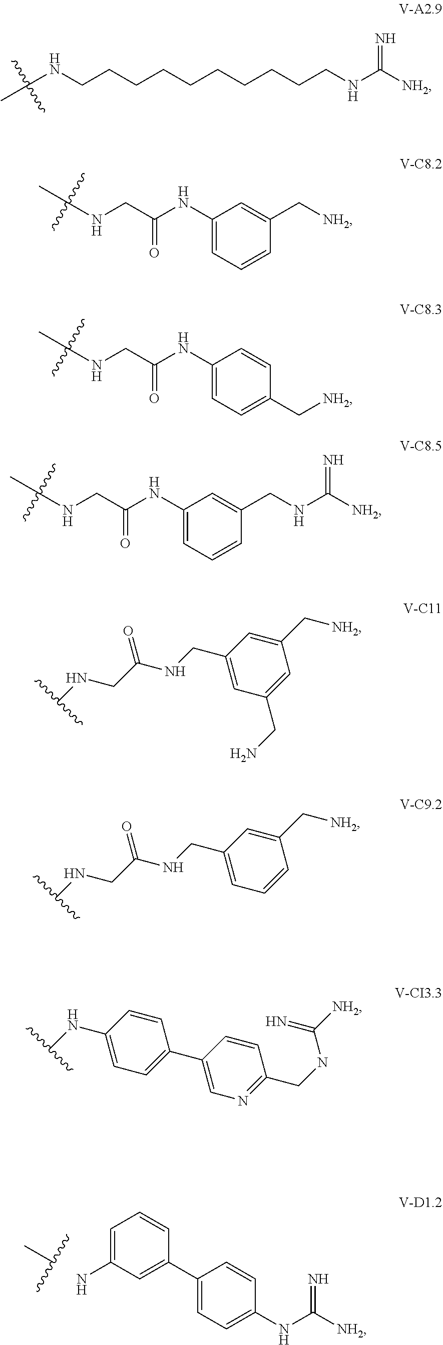

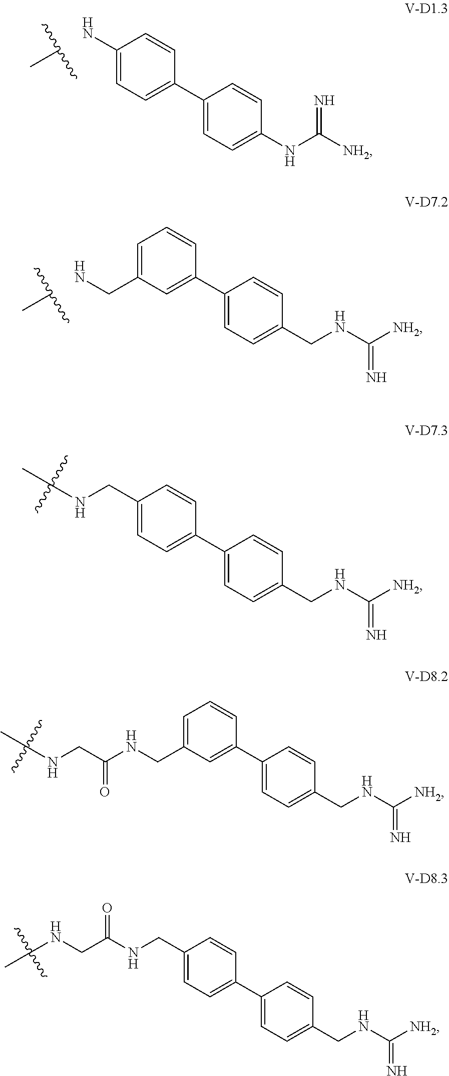

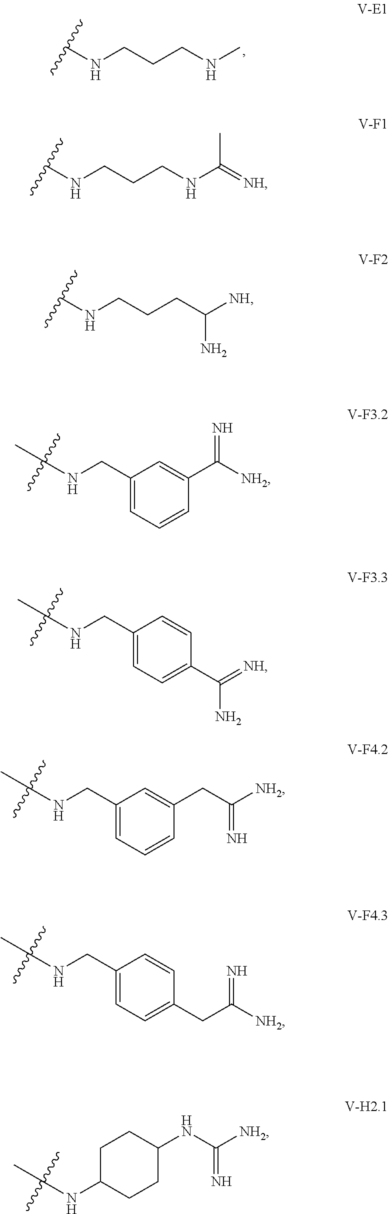

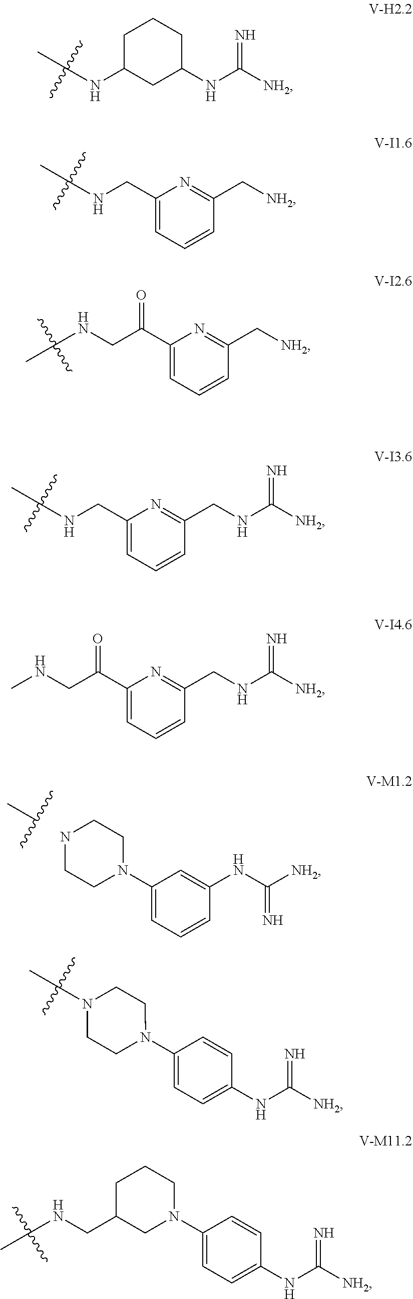

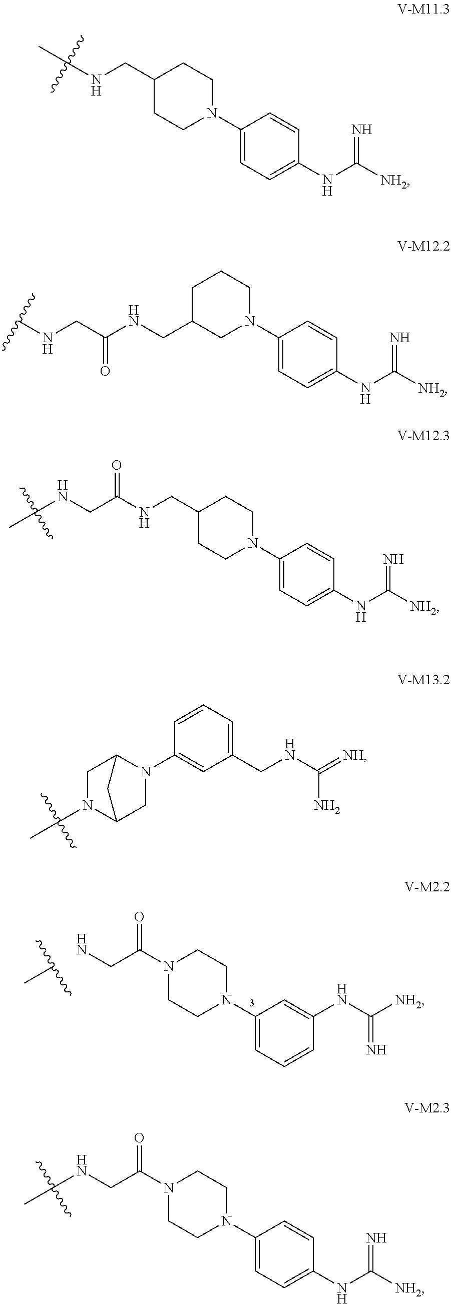

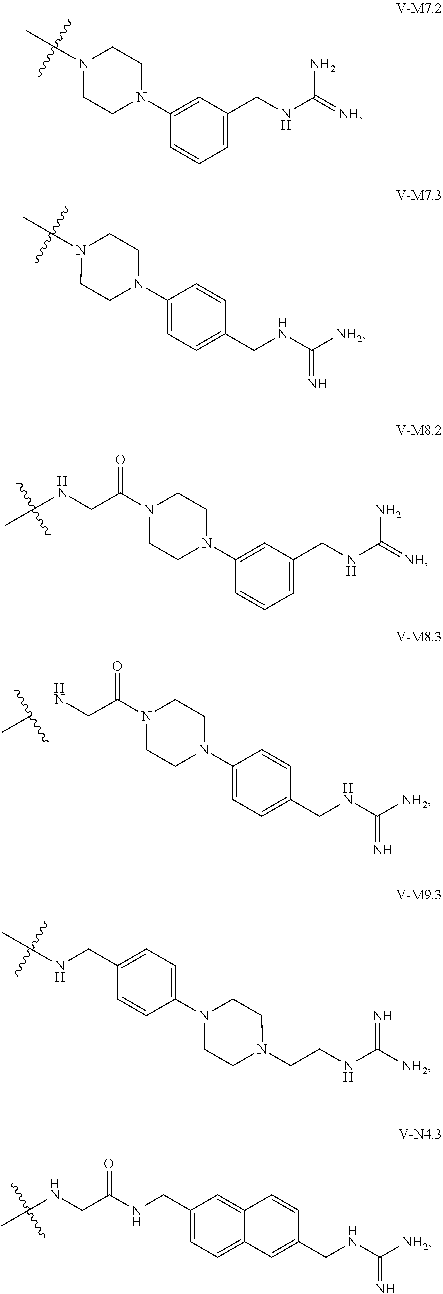

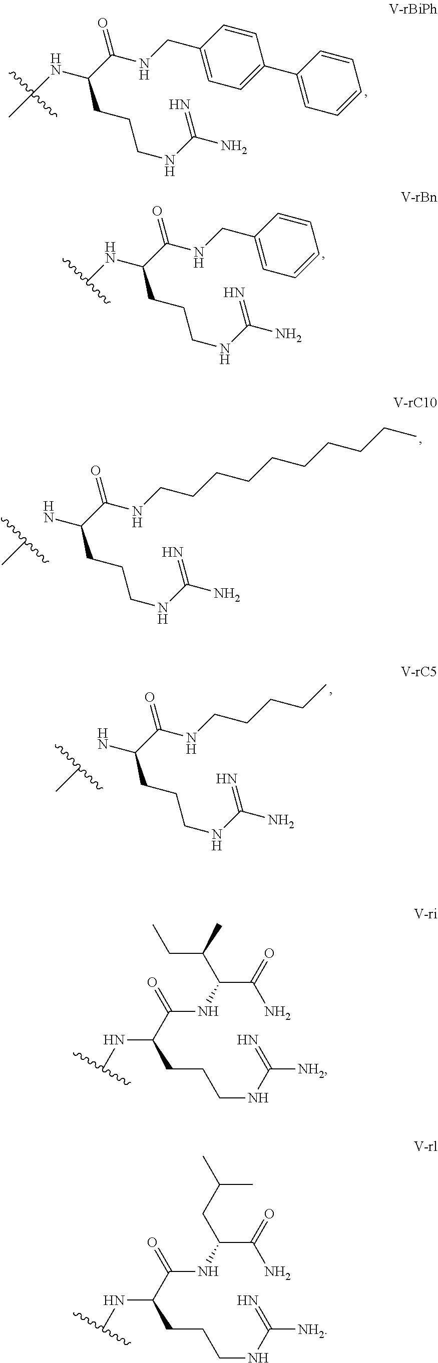

Any MoTr structure provided above constitutes a separate embodiment of the invention.

In some embodiments, the MoTr is any one of:

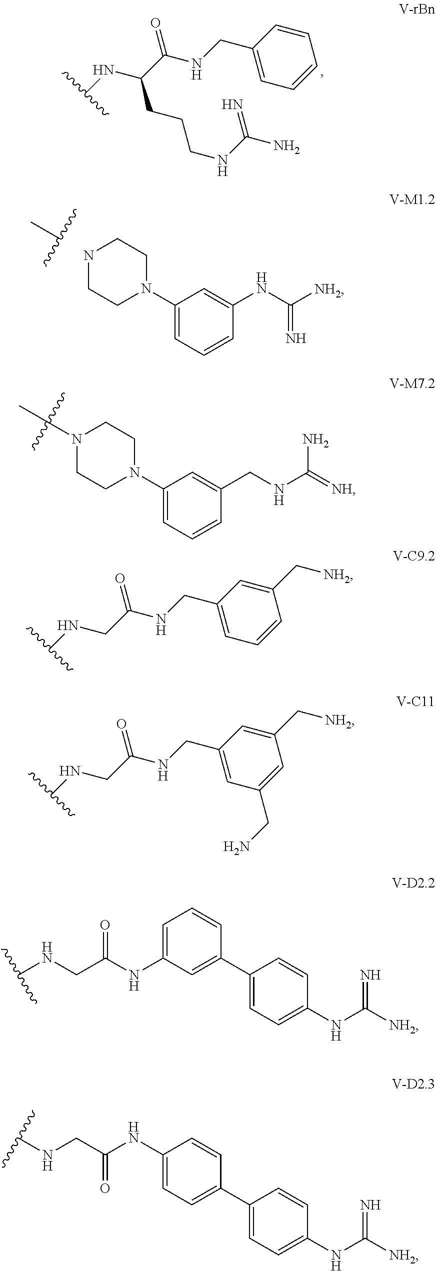

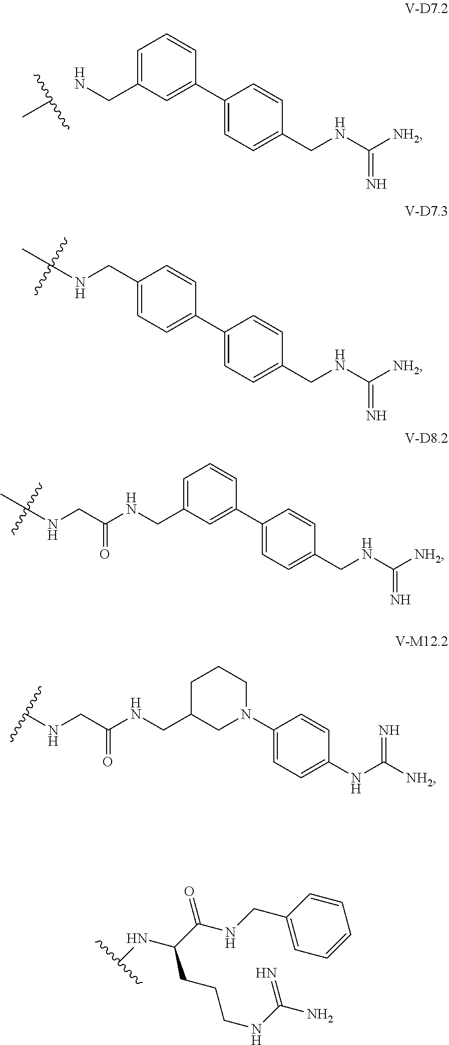

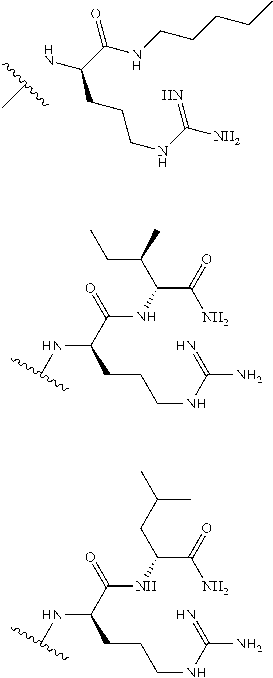

In each of the MoTr structures provided herein, the symbol [symbol] designates a point of connectivity to the cargo moiety, e.g., antibiotic, labeled A.

In some embodiments, the MoTr is aryl-based. In some embodiments, the MoTr is alkyl-based. As used herein, “aryl-based” or “alkyl-based” refers to presence or absence of an aromatic or a heteroaromatic ring structure in the MoTr structure. Where such a group is present, the MoTr is regarded as aryl-based, and where absent—it is alkyl-based. Thus, in some embodiments, the aryl-based MoTr are one or more of

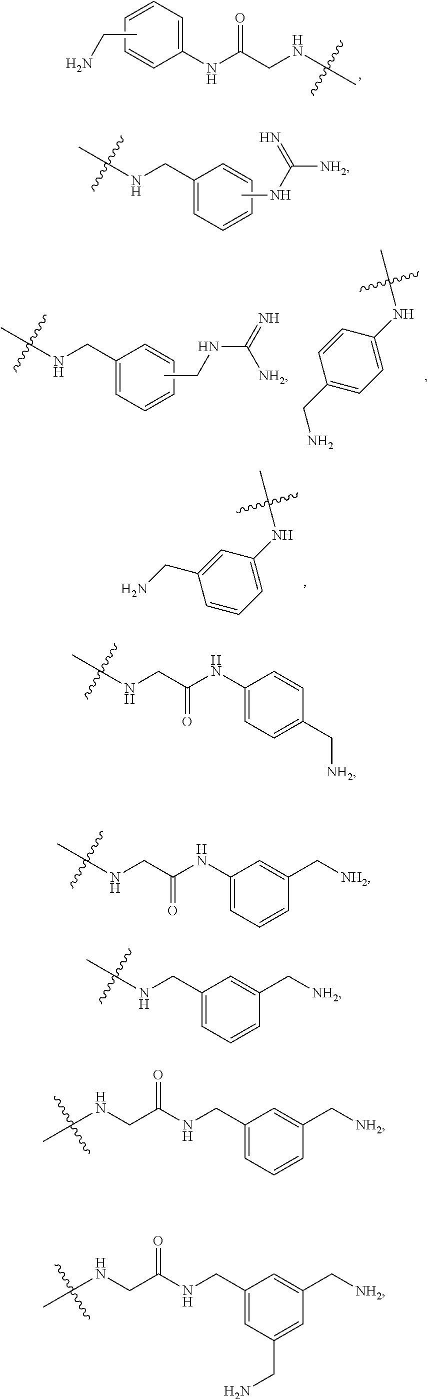

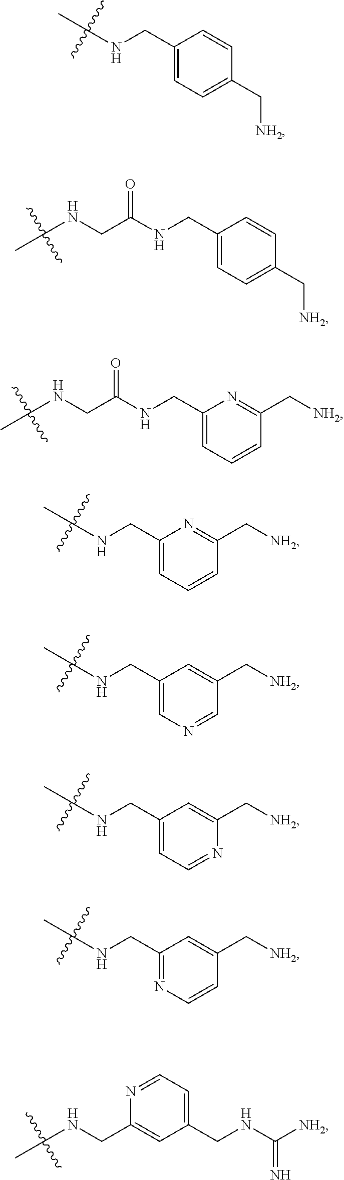

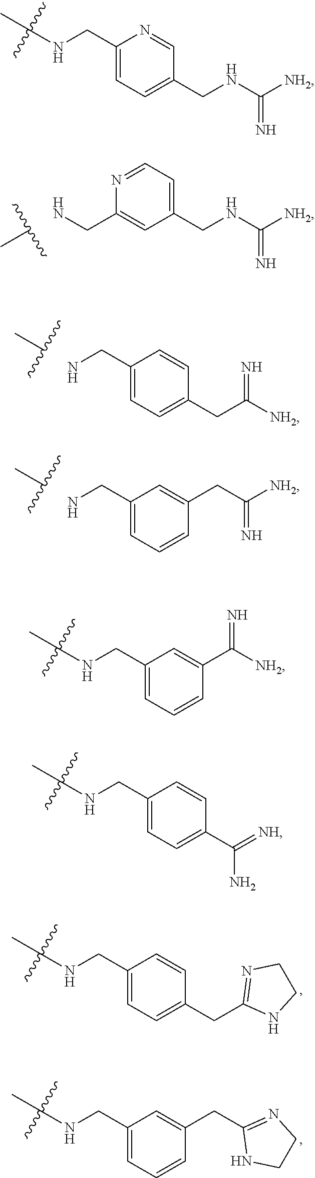

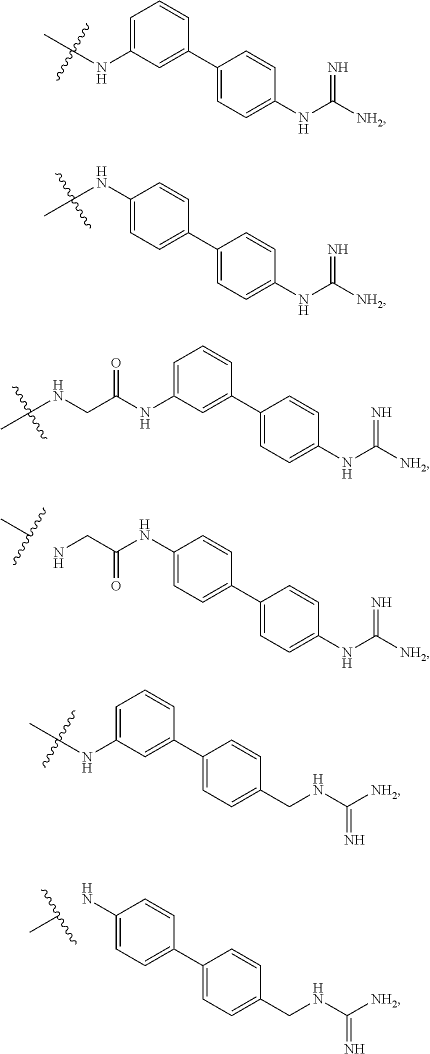

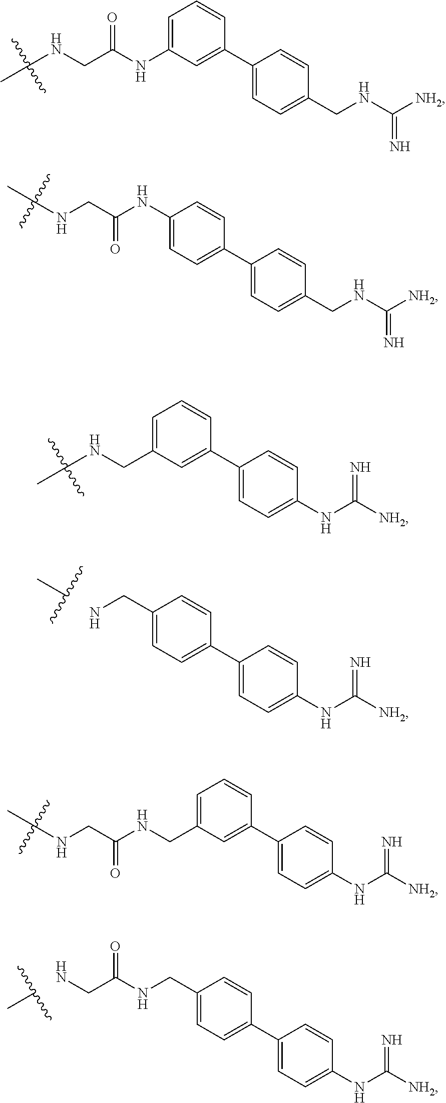

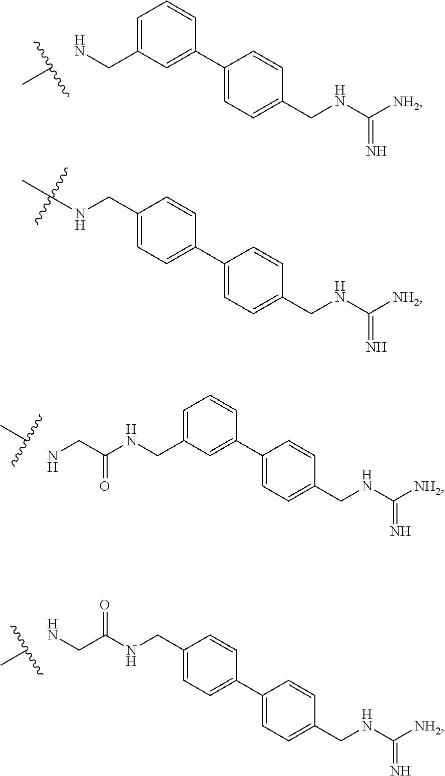

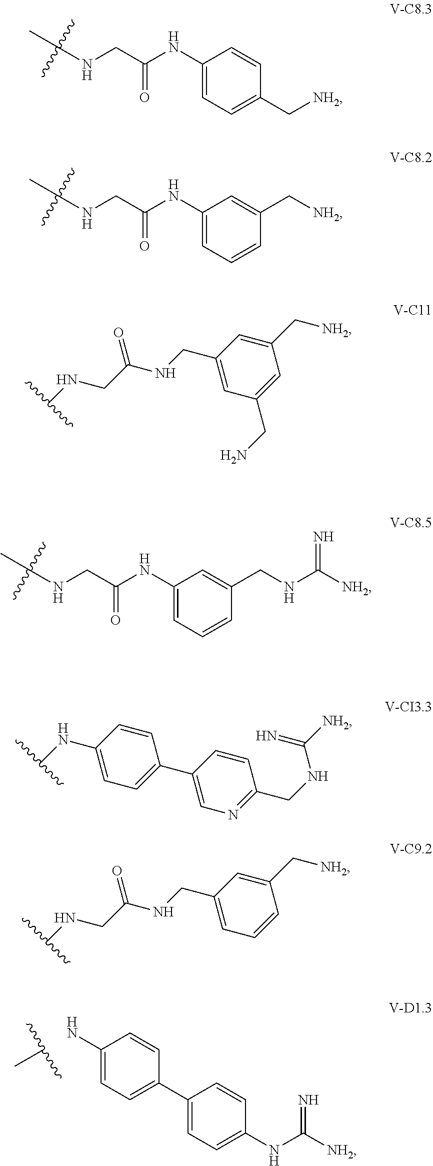

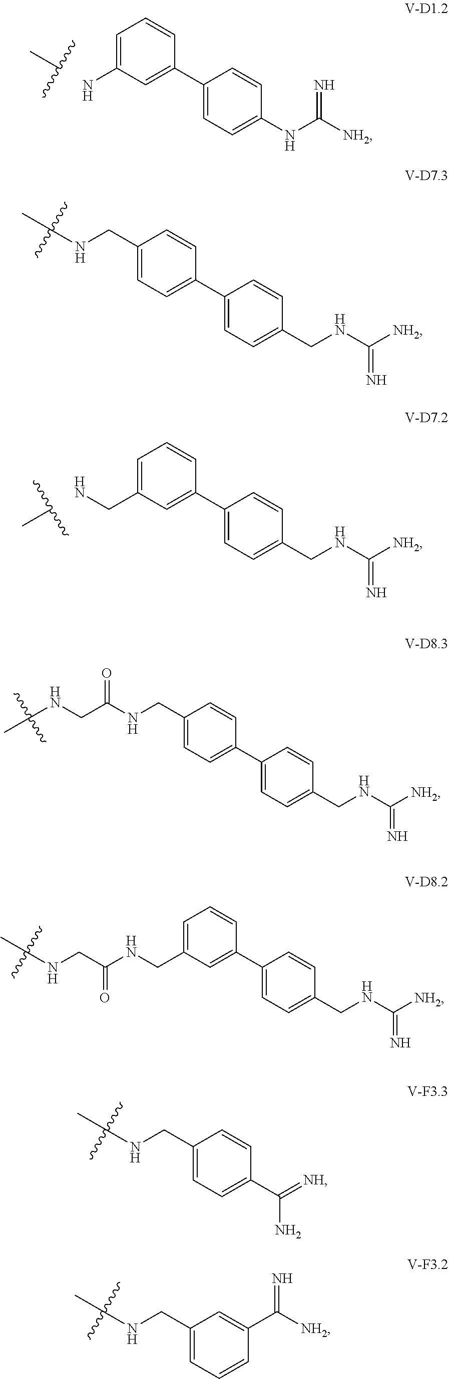

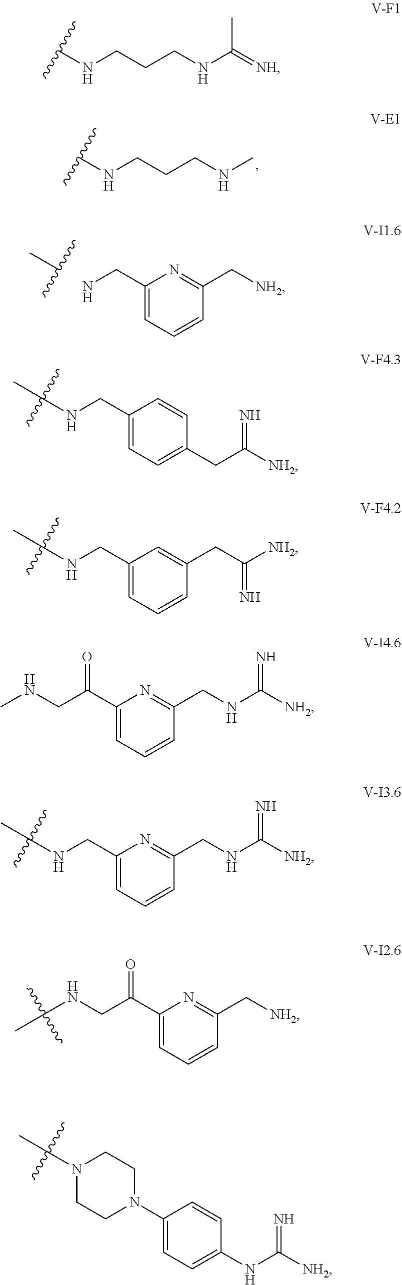

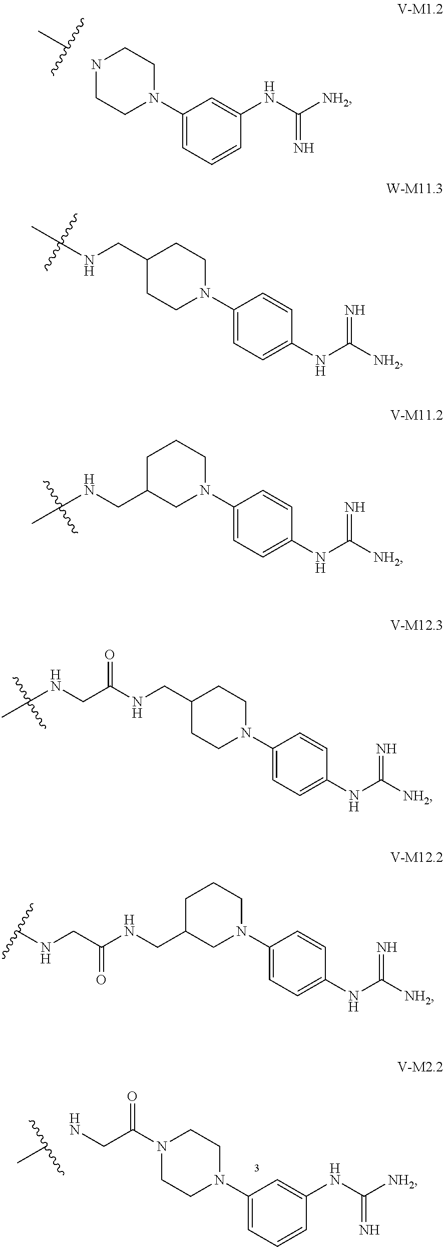

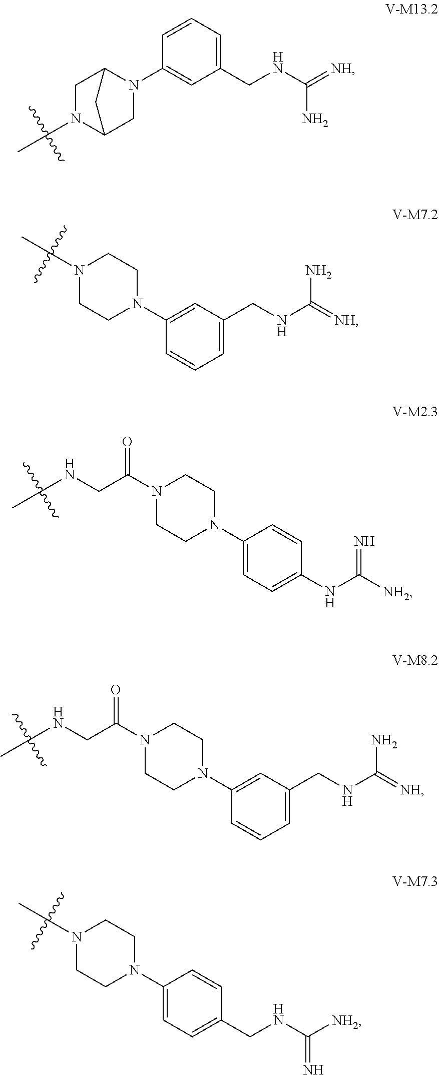 [SMILES: *N1CC2CC1CN2c1cccc(CNC(=N)N)c1 ; *N1CCN(c2cccc(CNC(=N)N)c2)CC1 ; *NCC(=O)N1CCN(c2ccc(NC(=N)N)cc2)CC1 ; *NCC(=O)N1CCN(c2cccc(CNC(=N)N)c2)CC1 ; *N1CCN(c2ccc(CNC(=N)N)cc2)CC1]

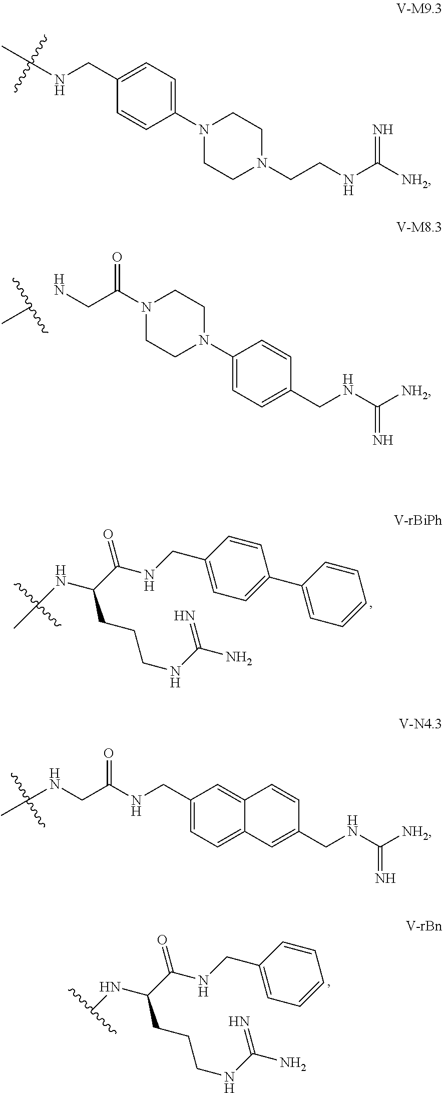

wherein each of the MoTr above constitutes a separate embodiment of the invention.

In some embodiments, the MoTr is alkyl-based and is selected from

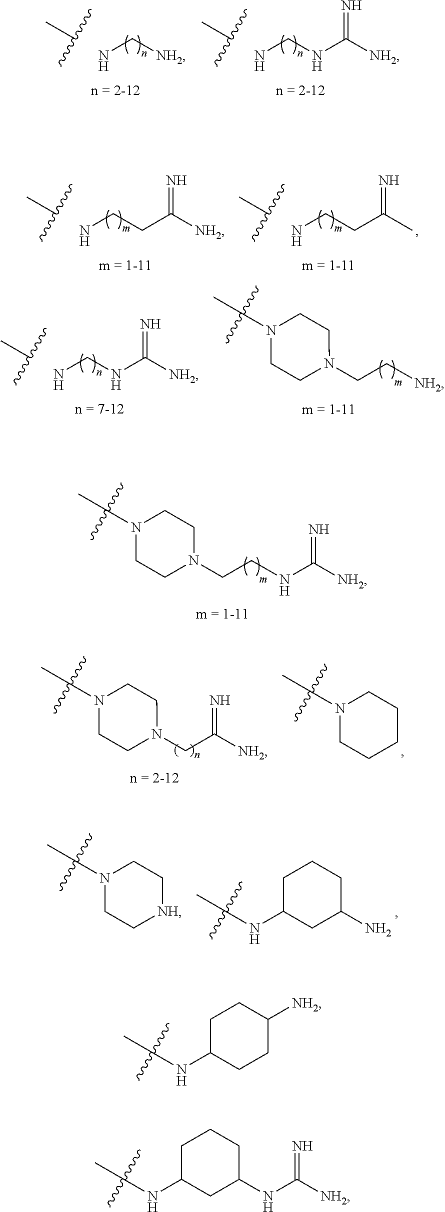

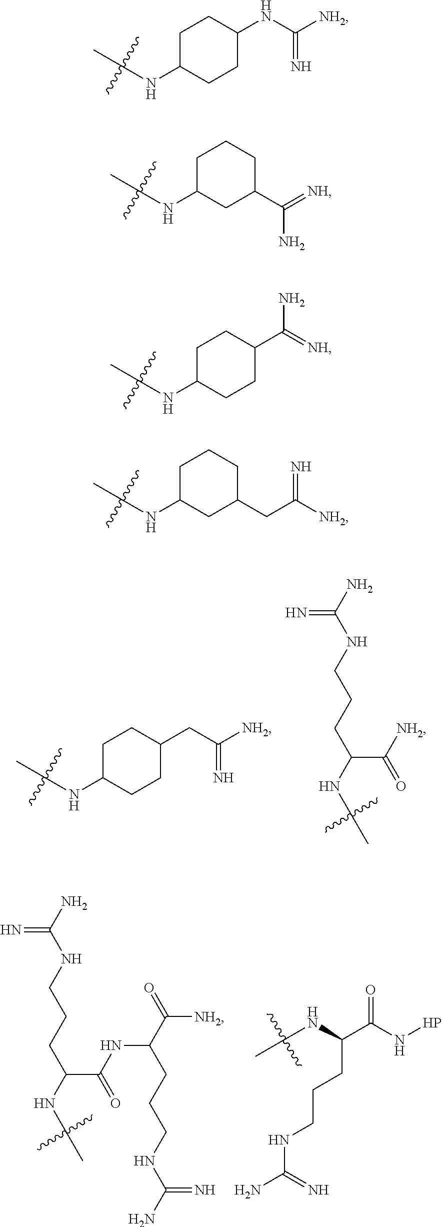

Hp is as defined herein

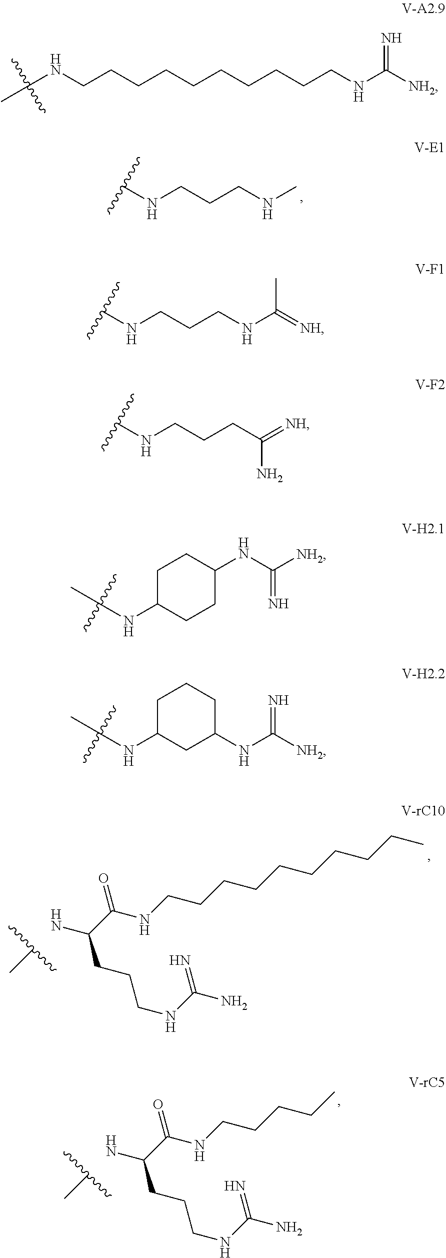

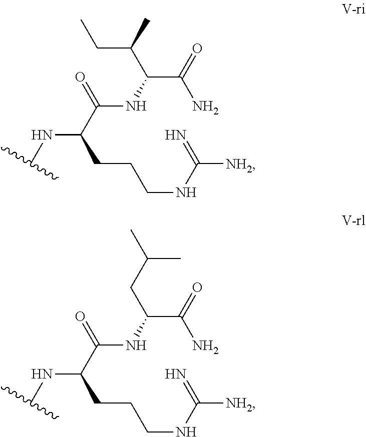

wherein each of the above MoTr structure constitutes a separate embodiment of the invention.

In some embodiments, the MoTr comprises a short alkyl or alkylene group having no more than 5-carbon-atom in a straight or branched carbon chain.

The invention also provides a compound of the structure A-MoTr, wherein A is an antibiotic as defined and MoTr is any one structure of the above aryl-based and alkyl-based structures, or any of the MoTr structures generically or specifically disclosed herein. In some embodiments, the MoTr is an aryl-based structure.

In some embodiments, the antibiotic is vancomycin and the MoTr is an aryl-based or an alkyl-based MoTr structure.

In some embodiments, A is an antibiotic and MoTr is selected from

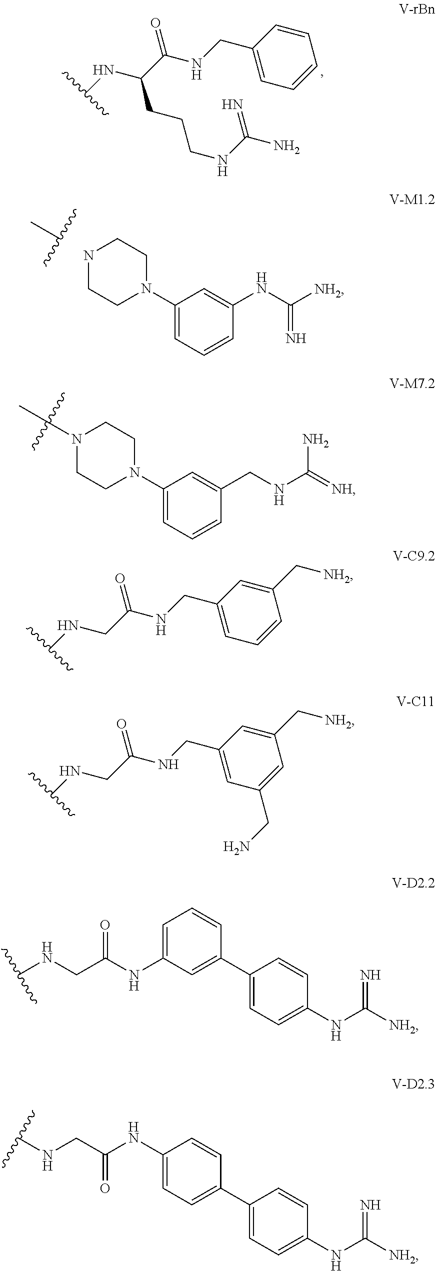

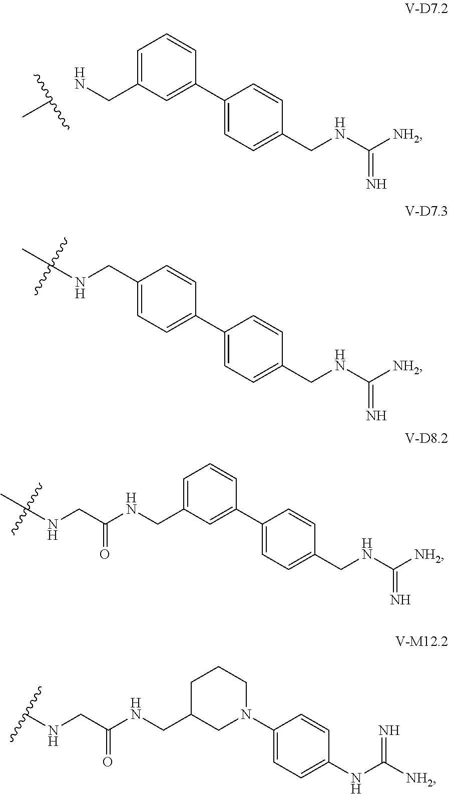

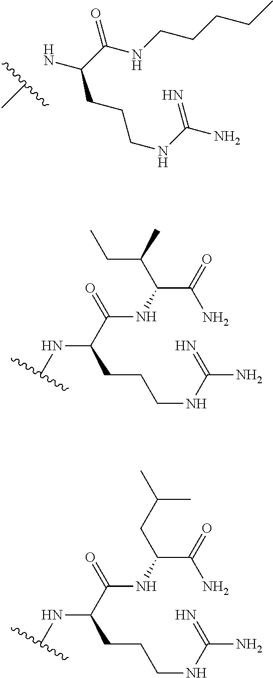

wherein each of the above MoTr constitutes an independent embodiment.

In some embodiments, the antibiotic is vancomycin and the MoTr is any one of

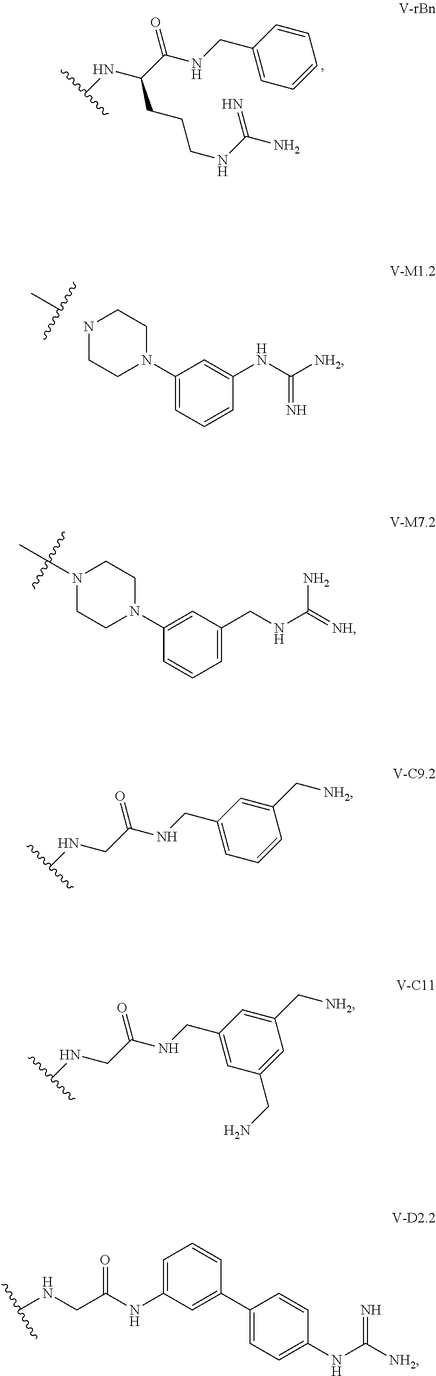

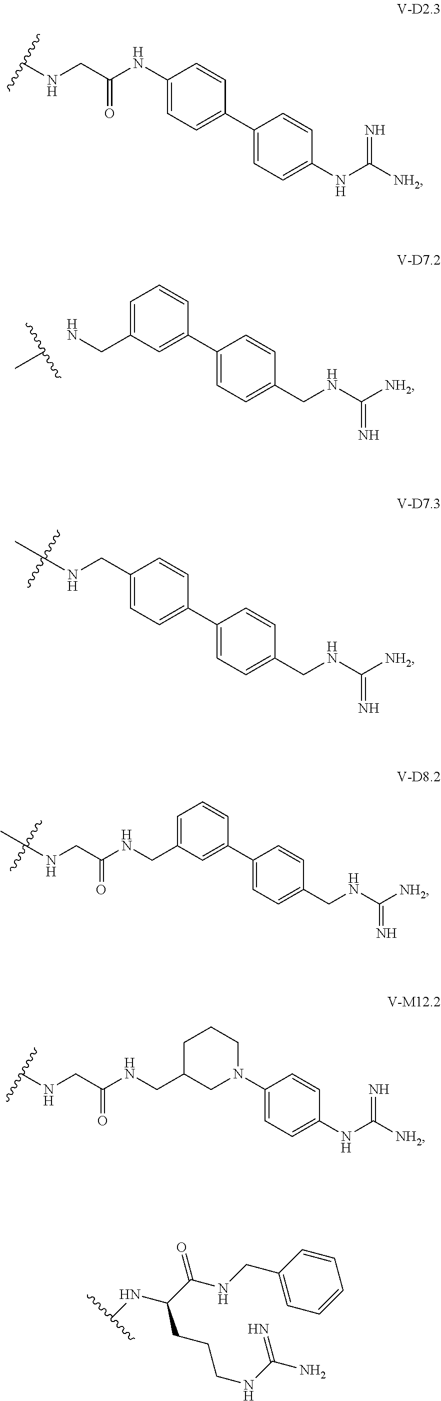

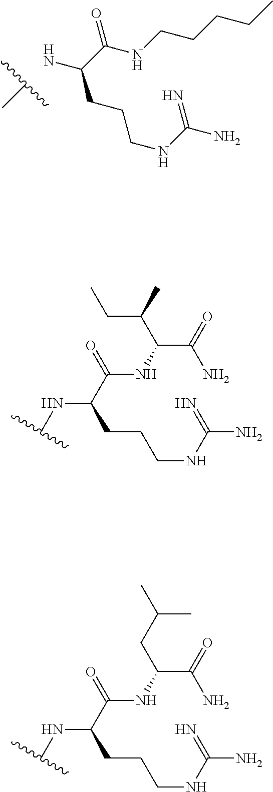

wherein each of the above MoTr constitutes an independent embodiment.

In some embodiments, the MoTr is

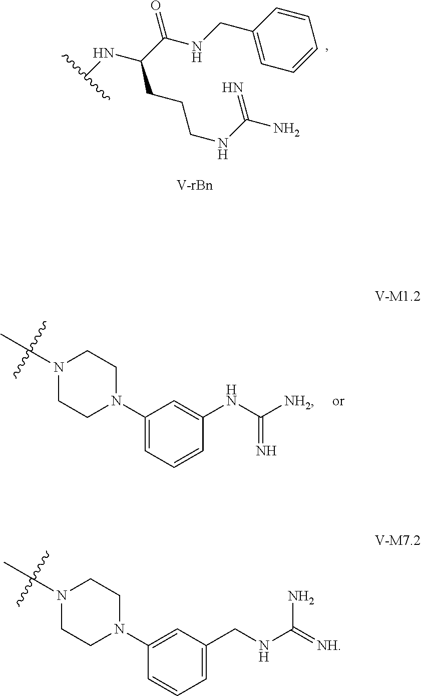

In any of the structures of the invention, integer v designates the number of X groups on the substituted group (may be L group, A group or any other group as defined). Where v is greater than 1, each X may be associated to the other or to the substituted group, or as stated herein to a lipophilic group optionally via a ligand moiety. In some embodiments, in all compounds of the invention, v is 1. In other embodiments, in all compounds of the invention, v is 2.

Preparation of certain antibiotic cationic MoTr conjugates of the invention is presently demonstrated (see EXAMPLES 1-2).

The unprecedented properties of the compounds of the invention have been revealed in the example of vancomycin as an optional cargo. Vancomycin is a standard of care glycopeptide antibiotic commonly used for the treatment of GPB infections. It is generally considered ineffective for the treatment of GNB because of its inability to breach the outer membrane and reach cell-wall targets at therapeutically relevant concentrations. The inventors have found that amino-, amidino-, guanidino- and imidazolino-containing conjugates of, e.g., vancomycin or linezolid and other antibiotics as defined herein, transformed the antibiotic to be effective against GNB such as Enterobacteriaceae that are considered intrinsically resistant to these drugs. Moreover, the cationic vancomycin-conjugates reported herein were found effective against various strains of GNB, including multi-drug resistant E. coli (carbapenem-R, 3^{rd}-gen ceph-R (ESBL+)), Acinetobacter baumanii and, importantly, Klebsiella pneumoniae (see EXAMPLE 3).

Thus, compounds of the invention of the general Formula (X), wherein A is an antibiotic such as vancomycin and MoTr is any of the structures depicted herein are used for the preparation of compositions for preventing and treating infections associated with GNB, including multi-drug resistant pathogens and may generally be useful against various strains of GNB.

Further, in the example of the cationic vancomycin-D-arginine conjugate (V-r), the inventors have succeeded to demonstrate that the spectrum of activity can be further expanded to include GNB and GPB strains with multiple resistance mechanisms. The microbial susceptibility of V-r (Minimum Inhibitory Concentration, MIC) towards E. coli including β-lactamase expressing Ambler classes A, B, and D significantly lower than the free vancomycin. Of particular importance is antimicrobial activity against metallo-betalactamases (e.g. NDM-1 E. coli), a major global pathogen which is resistant to existing therapies including carbapenem antibiotics. Moreover, the addition of 8×MIC V-r to E. coli was acutely bactericidal and was further associated with a low frequency of detectable mutants, frequency-of-resistance (FoR) of <2.3×10⁻¹⁰. Remarkably, V-r remained effective, and in many cases even more effective, against its natural GPB targets and antibiotic resistant GPB strains such as S. aureus, S. pneumoniae and others. In vivo, V-r markedly reduced E. coli burden by >7 log₁₀ CFU/g in a thigh muscle model (see EXAMPLE 4).

These findings establish a precedent for a novel class GNB antibiotics produced by transforming the commonly used and selective GPB antibiotics to include cationic features through a simple and scalable synthesis protocol. Such an approach, in consort with effective in silico predictions, might expedite antibiotic development and increase the overall probability-of-success of drug candidates. Most importantly, this would help to arrest the insidious pandemic of difficult-to-treat bacterial infections.

More specifically, the compounds of the invention represent a new class of antibiotics with improved antibacterial activity in GPB and an extended spectrum of antibacterial activities to include GNB, with examples of E coli (including multi-drug resistant strains), Acinetobacter baumanii, Klebsiella, and other GNB pathogens.

In other words, the present compounds and the ensuing compositions and methods provide the advantages of more effective, broader spectrum, less toxic and safe treatment of bacterial infections with GPB and/or GNB, including GPB and GNP antibiotic-resistant strains, bacteria at a stationary-phase, persistent bacteria, and bacterial films. The present compounds can be effective against common GPB infections, such as with members of the genera Staphylococci, Streptococci, Enterococci, Bacilli, Corynebacteria, Clostridia, e.g., Corynebacterium diphtheriae, Bacillus anthracis, Clostridioides difficile. They can be further effective against difficult-to-treat GPB infections, such as with methicillin-resistant Staphylococcus aureus (S. aureus, MRSA), multidrug-resistant Staphylococcus epidermidis (MRSE), and further, in vancomycin-resistant strains, such as vancomycin-resistant enterococci (VRE), e.g., enterococcusfaecium and Enterococcus faecalis, prevalent in hospitals and health care facilities. They can be further effective against GNB infections and GNB infections with inherent or acquired multiple drug resistance such as GNB related pneumonia, bloodstream infections, wound or surgical site infections, and meningitis. Specific examples are outbreaks of multidrug-resistant-Acinetobacter, highly resistant Burkholderia cepacia, Enterobacter cloacae, Enterococcus spp., Klebsiella pneumoniae, Proteus mirabilis, Pseudomonas aeruginosa and Staphylococcus saprophyticus in hospitals and immunocompromised patients, and widespread infections of Neisseria gonorrhoeae, Vibrio Cholerae, Bacteroides fragilis and others.

Additional advantages of the compounds of the invention have been revealed in studies showing the feasibility of reducing the therapeutic dose and using alternative more accessible modes of administration. Using the example of the V-r conjugate in recognized models of complicated Urinary Tract Infection (cUTI) driven by GNB pathogens, the inventors have found that V-r allows for a much lower effective dose in the treatment of cUTIs, thereby significantly mitigating potential toxicities. Furthermore, the inventors have shown that compared to currently used IV antibiotics (e.g., Piperacillin-Tazobactam, Ceftazidime/Avibactam, Ertapenem, Cefiderocol), the total dose of V-r is expected to be substantially less, ranging from approximately 0.96-13% of the total doses of comparator drugs. Still further, V-r was amenable for administration via IV and sub cutaneous (SC) routes (see EXAMPLE 5). This latter is critically important for hospitalized cUTI patients (and other infections), whereby the SC route might offer a ‘go-home sooner’ and an early discharge option, and further, an option of a continued low dose V-r therapy from outpatient departments or within the community clinical settings. Such a low dose therapy would rule out the need for measuring blood concentrations of the drug. All these features would eventually contribute to a decrease in further resistance breakouts in a hospital setting, as well as reducing health care costs associated with lengthy hospital stays.

Ultimately, compounds, compositions and methods of the invention can be applied in effective dosage forms, internally, topically, or subcutaneously as an active or a preventive treatment for a wide range of GPB and GNB infections. In view of their relatively high solubility and apparent wide-range applicability, it seems feasible that they can be produced in non-conventional forms such as premade ready-to-use bags for IV infusion, for example, or dermal or transdermal patches. Another attractive application would be to adapt them into medical devices (including wearable devices) for subcutaneous (SC) injection, alone or coupled with diagnostic tools for point-of-care (POC) testing for rapid triage and treatment decisions. Owing to their relatively straightforward method of production (starting from commercially available clinical grade FDA-approved drugs) and remarkable biological efficiency, they can be further applied to biological and non-biological surfaces or medical devices in vivo or in vitro for eradication of bacterial biofilms, and especially device-related MRSA infections—the most difficult to treat infection imposing a significant threat to the health care systems, world-wide.

Any compound specifically disclosed or encompassed within general formulae is a compound of the invention and constitutes an independent and separate embodiment of the invention. Any compound encompassed by the expression “selected from” constitutes an independent embodiment and may be regarded as a singular selection.

Any compound disclosed herein with reference to a specific use or composition is a compound disclosed per se as a novel compound irrespective of its stated use.

Excluded from compounds of the invention are any of the compounds disclosed in the publications [1-11] above.

### BRIEF DESCRIPTION OF THE DRAWINGS

FIGS. 1A-1F provide a summary of antimicrobial susceptibility profiles (Minimum Inhibitory Concentration, MIC) of selected compounds against a selection of GNB pathogens.

FIG. 2 shows antimicrobial susceptibility profiles of vancomycin-arginine (V-r) and vancomycin against selected GNB and GPB pathogens, including multi-drug resistant strains.

FIG. 3 illustrates the efficacy of the vancomycin-D-arginine conjugate (V-r) compared to vancomycin against E. coli uropathogens UTI89 and NCTC-13441 and the resulting change in bacterial load over a 24 h time period (time-kill). V-r, but not vancomycin, demonstrated rapid bactericidal activity to limits of detection (LOD, 100 CFU/ml) within 1 or 4 h of exposure and maintained up to 24 h.

FIG. 4 illustrates the efficacy of V-r in reducing E. coli UTI89 bacterial burden in a 24 h thigh muscle infection model in neutropenic CD-1 mice. These bactericidal effects of V-r were significantly superior to vancomycin and ciprofloxacin with a log₁₀ 1.4 reduction from stasis.

FIG. 5 illustrates the efficacy of V-r in abrogating bacterial burden (E. coli CTX-M-15) in a complicated urinary tract infection (cUTI) model in mice. ED₅₀ (mg/kg) values are represented. The ED₅₀ (mg/kg) was calculated by regression analysis and determined to be 1.8-8.9 mg/kg for bladder, urine, and kidney.

FIG. 6 illustrates the effect of V-r in the urine in the cUTI model, with maximal inhibition of bacterial burden at a dose of V-r at 25 mg/kg given q12h for 3 days.

FIG. 7 illustrates the effect of V-r in the bladder in the cUTI model, with maximal inhibition of bacterial burden at a dose of V-r at 25 mg/kg given q12h for 3 days.

FIG. 8 illustrates the effect of V-r in the kidney in the cUTI model, with maximal inhibition of bacterial burden at a dose of V-r at 50 mg/kg given q12h for 3 days.

FIG. 9 illustrates the PK characteristics of V-r in mice following a single IV administration (1-50 mg/kg, identical to the cUTI model) and the resulting ratio of drug:MIC in the urine. The V-r PK profile was linear, with target AUCs at the effective 25 and 50 mg/kg doses of 43 and 104 mg·h/l, respectively. The ratio of V-r:MIC was 70 and 119, which is in excess to drive the desired antibacterial effect.

FIG. 10 illustrates the feasibility of a low dose V-r therapy. Figure shows PK profile of V-r (25 mg/kg) in a beagle dog following a 60 min IV infusion, yielding an AUC of 580 mg·h/l. Under various assumptions, the required allometric human q12h doses of V-r to elicit such AUCs might be expected to be 0.94-2.35 mg/kg or 1.31-3.29 g for a 10 day therapy in a 70 kg patient, which is significantly lower than the vancomycin dose to treat GPB infections (15 mg/kg q12h for 10 days=20 g total amount required for complete treatment regimen).

FIG. 11 illustrates the feasibility of adaptation of V-r as a low-dose therapy to treat E. coli-associated cUTIs via sub-cutaneous administration. Figure shows comparison of the PK profiles of V-r in mice following IV (10 mg/kg) and sub-cutaneous (SC) administration (20 mg/kg). The exposure of V-r following sub-cutaneous administration is essentially equivalent to the exposure following IV administration of V-r. These data suggest that V-r is amenable for administration via both the IV and SC routes.

### DETAILED DESCRIPTION OF EMBODIMENTS

It should be appreciated that the invention is not limited to specific methods, and experimental conditions described herein, and that the terminology used herein for the purpose of describing specific embodiments is not intended to be limiting.

The increasing prevalence of bacterial resistance to antibiotics is a worldwide, critical public health concern and associated with significant morbidity and mortality. Despite the emergence of multi-drug resistant bacterial pathogens, the available antibiotic arsenal is dwindling with a sparse pipeline of drugs currently in pre-clinical and clinical development. Consequently, novel antibiotic discovery and development strategies are urgently required to combat such threats in consort with unique approaches aimed to accelerate compound development towards clinical testing and commercialization.

In its main aspect the invention pertains to compounds, e.g., antibiotic conjugates comprising lipophilic moieties, which facilitate enhanced intermolecular interaction/uptake through the target outer membrane; and (one or several) N-containing cationic group(s) that can be either directly attached to the antibiotic cargo or via one or more linkers.

Compounds of the invention may be represented by a structure of any of Formulae (I) through (IX), each being an embodiment of a compound of the form A-MoTr (herein designated Formula (X)). In the compounds below, the cargo moiety may be an antibiotic moiety.

In some embodiments, compounds of the invention may be represented by Formula (I):

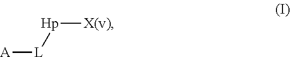

as defined above.

In some embodiments, in a compound of Formula (I):
A is a cargo moiety, the cargo moiety being as defined herein, being in some embodiments, an antibiotic moiety,
L is a linker moiety, which may be absent,
Hp is a lipophilic unit directly associated with X or via linker moiety -Lₕ-(namely -Lₕ- may or may not be present), wherein optionally Hp is selected from C1-C5alkylene, C1-C5heteroalkylenes, C5-C10cycloalkyls, or alkylenes and C5-C10heterocycloalkylenes, C6-C12arylene, C3-C10heteroarylene, C10-C20aralkyene, C6-C16heteroaralkylene, C5-C10carbocyclylene and C3-C10heterocarbocyclylene; in some embodiments, where -Lₕ- is present to link Hp to X, -Lₕ- is selected as defined herein;
v is 1 or 2, and
X is an N-containing cationic group.

In some embodiments, compounds of the invention may be represented by Formula (Ia) or (Ib):

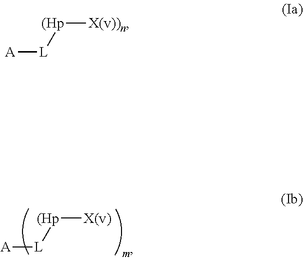

wherein each of A, L, Hp, X and v are as defined above, and wherein Hp and X may or may not be associated via a linker -Lₕ-; and each of n and m, independently of the other, is an integer designating the number of repeating moieties, the integer being between 1 and 10.

As shown, integer “n” is the number of times moiety -(Hp-X) is linearly repeated. For example, where n is 1, the compound of Formula (Ia) is

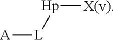

In cases where n is 2, the compound of Formula (Ia) is

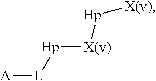

etc. Thus, in cases where n is greater than 1, each repeating unit is associated in a linear fashion in the direction shown in the Formula.

Similarly, integer “m” is the number of times moiety -(L-Hp-X) is linearly repeated. Where m=1, the compound of Formula (Ib) is

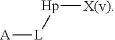

In cases where m=2, the compound of Formula (Ib) is

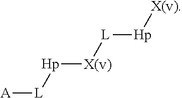

In each of the aforementioned embodiments, Hp and X may or may not be associated via a linker -Lₕ-, as defined. In some embodiments, linker -Lₕ- is present and compounds of the invention may be represented by Formula (TI):

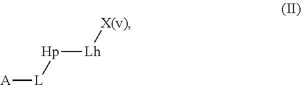

wherein each of A, L, Hp, v and X is as disclosed herein and wherein Lₕ is a linker moiety associating Hp to X. In such embodiments, the moiety -(Hp-Lₕ-X) may appear n times, wherein n is between 1 and 10 and the moiety -(L-Hp-Lₕ-X) may be repeated m times, wherein m (independently from n) is an integer between 1 and 10. The repetition of the groups may be as depicted for structures of Formulae (Ia) and (Ib) above.

In some embodiments, the lipophilic moiety Hp may be associated directly on the antibiotic moiety or may be a pendant group extending from the linker moiety L such that the lipophilic group Hp is not (directly) associated to X. Such compounds may be of the structures (III), (IV), (V), (VI) and (VII):

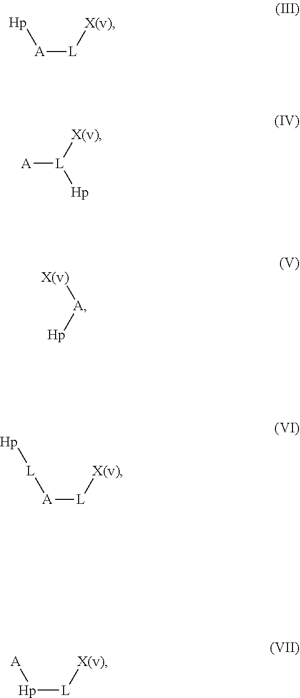

wherein
A is a cargo moiety, being in some embodiments an antibiotic moiety,
L is a linker moiety which may be absent (in each of structures (III) and (IV), thus yielding structure (V)),
Hp is a lipophilic unit associated with the antibiotic moiety directly or indirectly via a linker moiety, as defined and depicted,
v is 1 or 2; and
X is an N-containing cationic group.

A compound of Formula (III) may be of Formula (IIIa):

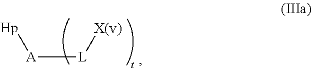

wherein t is an integer between 1 and 10.

In some embodiments, where t=1, the compound may be of the structure

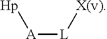

In cases t=2, the compound may be of the structure

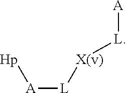

Thus, in cases where n is greater than 1, each repeating unit is associated in a linear fashion in the direction shown in the Formula.

Compounds of the invention may alternatively be represented by Formula (VIII) and (IX):

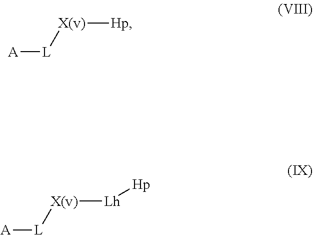

wherein each of A, L, X, v and Hp are as defined herein, and wherein Hp may be associated with X directly or via linker moiety -Lₕ-.

In some embodiments, compounds of the invention may be represented by Formula (VIIIa) or (VIIIb):

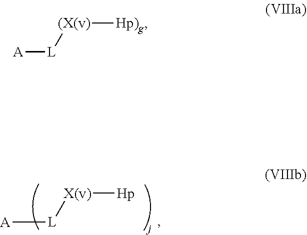

wherein each of A, L, X, v and Hp are as defined herein; and
each of g and j, independently of the other, is an integer designating the number of repeating moieties, the integer being between 1 and 10.

As shown, integer “g” is the number of times moiety -(X-Hp) is linearly repeated and integer “j” is the number of times moiety -(L-X-Hp) is linearly repeated.

Linear repetition is as demonstrated hereinabove.

In other aspects of the invention, there is provided a compound of any one of structures (I), (Ia), (Ib), (II), (III), (IV), (V), (VI), (VII), (VIII) and (IX) wherein A is an antibiotic moiety.

In other aspects of the invention, any of the structures (I), (Ia), (Ib), (II), (III), (IV), (V), (VI), (VII), (VIII) and (IX) disclosed herein may be presented as structure (X):

A-MoTr (X)
wherein A is as defined herein and MoTr is the sequence derived from structure (I), namely -L-Hp-X(v) or -L-Hp-Lₕ-X(v), wherein each of L, Hp, Lₕ, X and v are as defined herein.

In some embodiments, MoTr comprises a phenyl or a biphenyl moiety and an N-containing moiety that is an amine or guanidinyl. Such compounds of the invention include

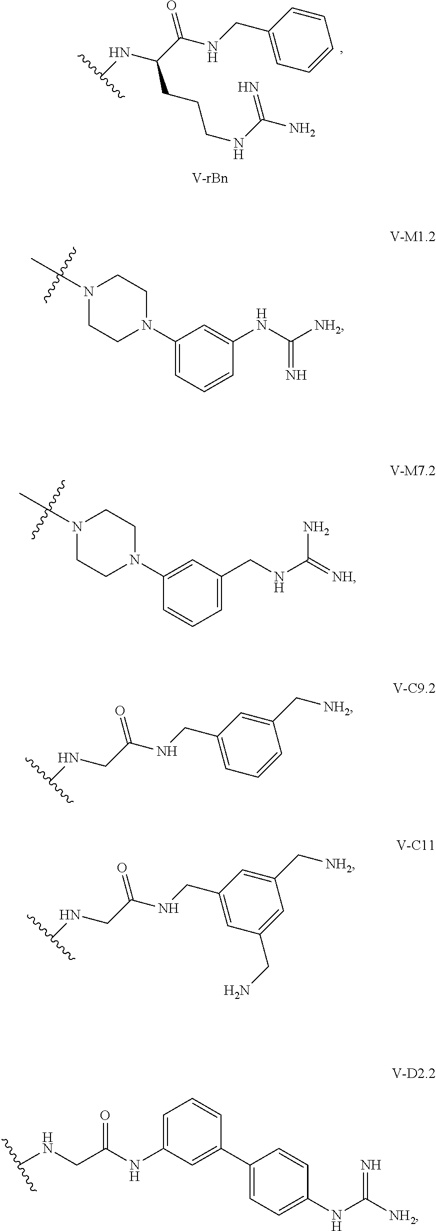

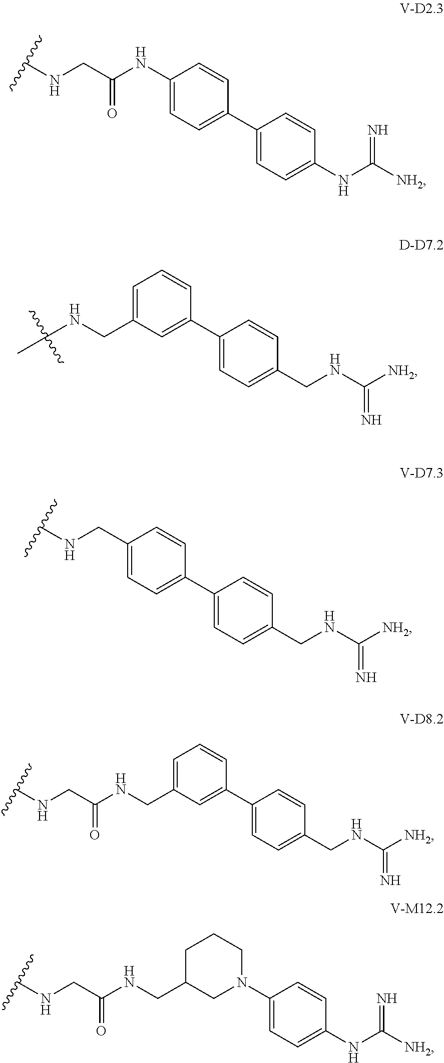 [SMILES: *NCC(=O)Nc1ccc(-c2ccc(NC(=N)N)cc2)cc1 ; *NCc1cccc(-c2ccc(CNC(=N)N)cc2)c1 ; *NCc1ccc(-c2ccc(CNC(=N)N)cc2)cc1 ; *NCC(=O)NCc1cccc(-c2ccc(CNC(=N)N)cc2)c1 ; *NCC(=O)NCC1CCCN(c2ccc(NC(=N)N)cc2)C1]

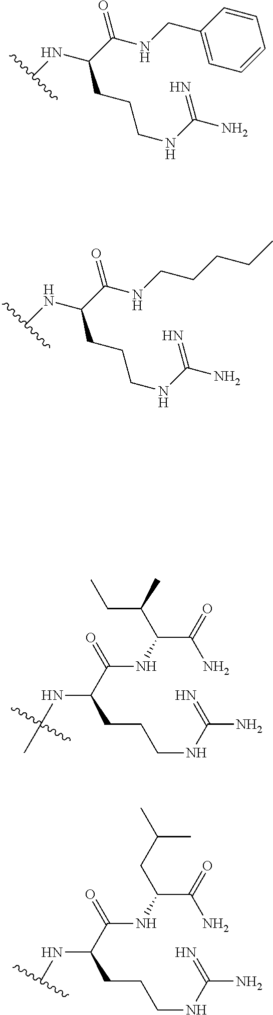 [SMILES: *N[C@H](CCCNC(=N)N)C(=O)NCc1ccccc1 ; *N[C@H](CCCNC(=N)N)C(=O)NCCCCC ; *N[C@H](CCCNC(=N)N)C(=O)N[C@@H](C(N)=O)[C@H](C)CC ; *N[C@H](CCCNC(=N)N)C(=O)N[C@H](CC(C)C)C(N)=O]

wherein each of the above MoTr constitutes an independent embodiment.

In some embodiments, MoTr is selected from

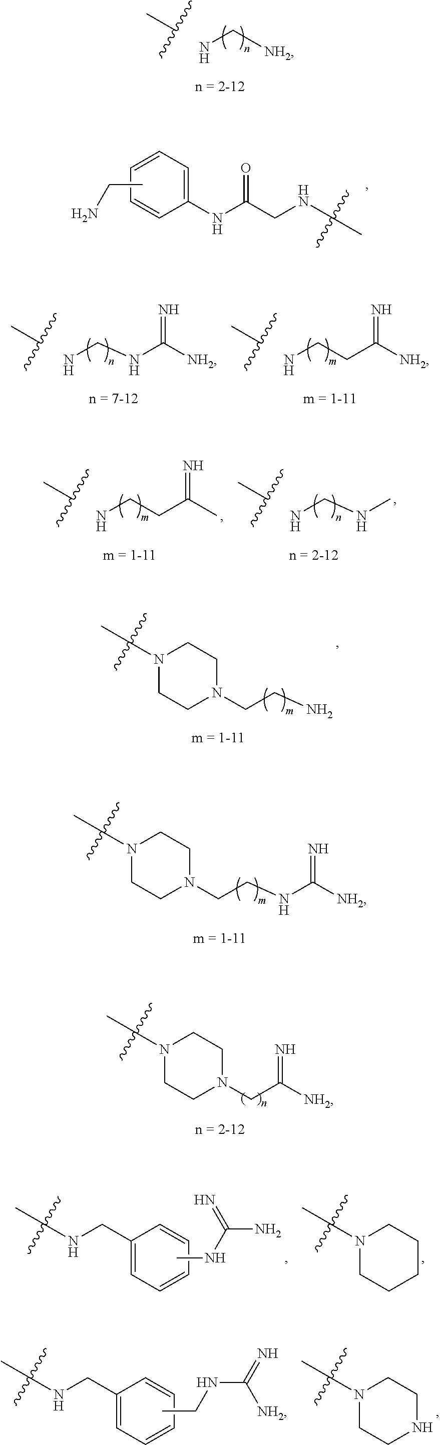

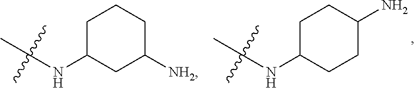 [SMILES: *NC1CCCC(N)C1 ; *NC1CCC(N)CC1]

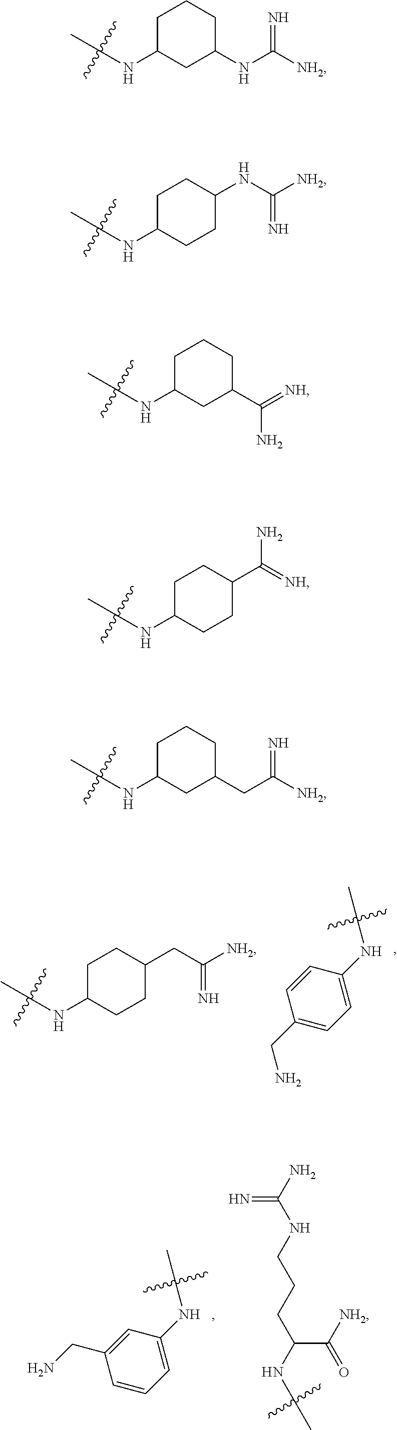

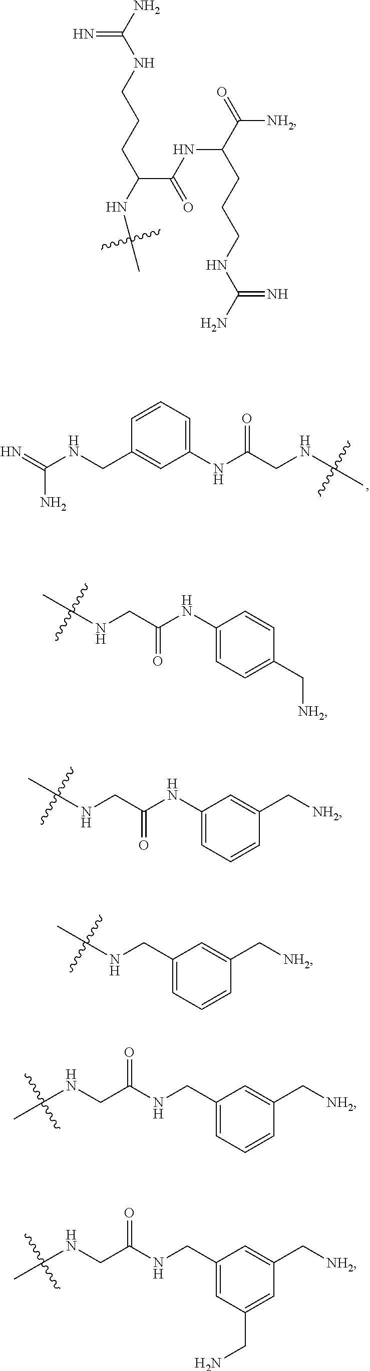

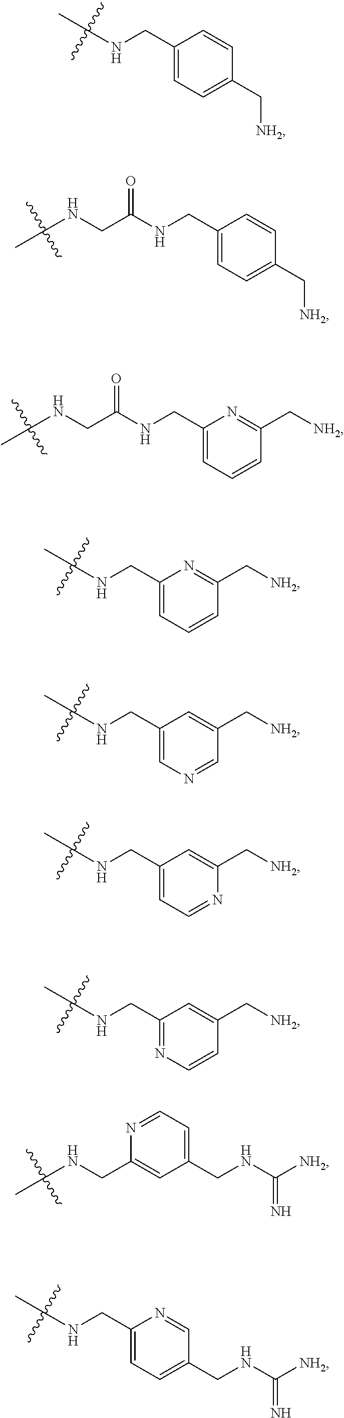

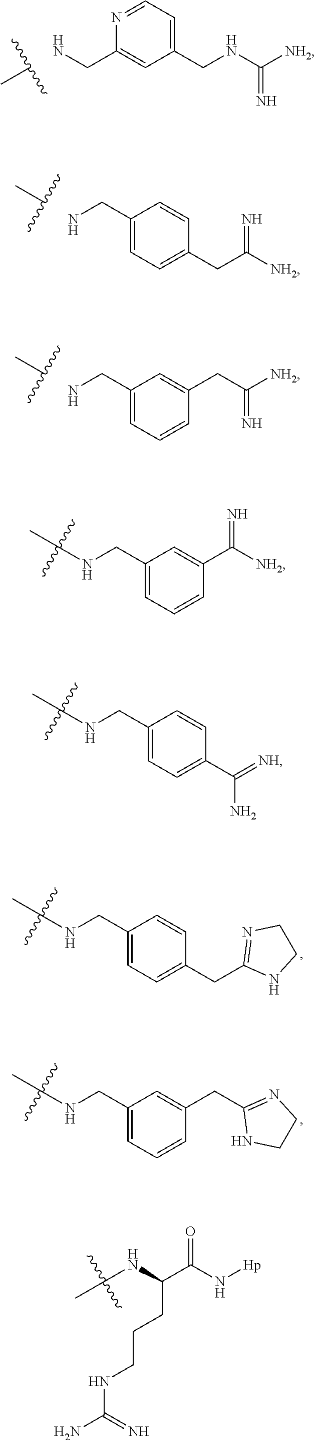

Hp is as defined herein

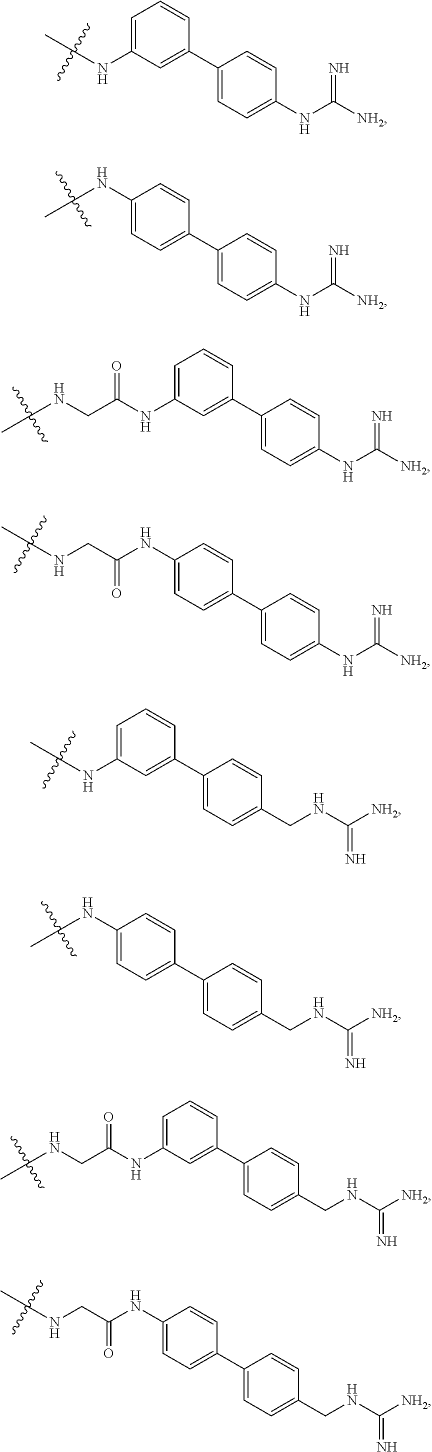

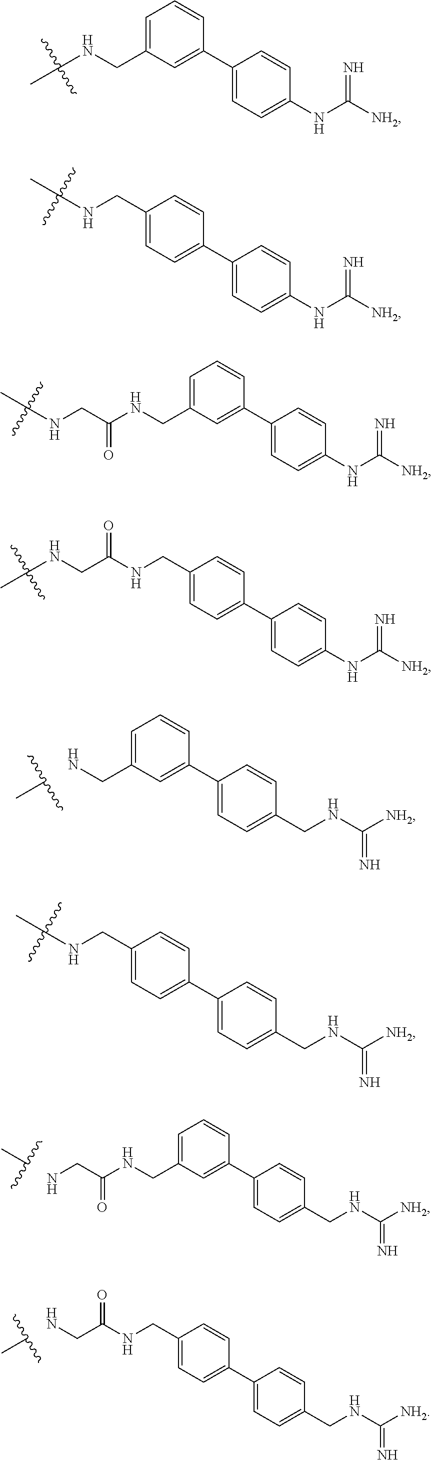

Any MoTr structure provided above constitutes a separate embodiment of the invention.

In some embodiments, MoTr is selected from

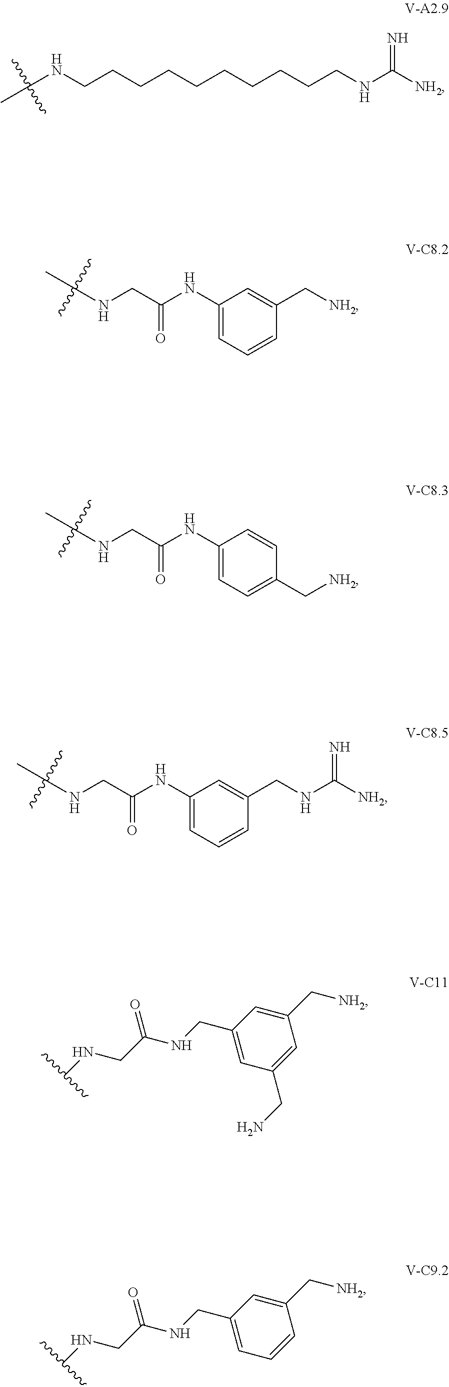 [SMILES: *NCCCCCCCCCCNC(=N)N ; *NCC(=O)Nc1cccc(CN)c1 ; *NCC(=O)Nc1ccc(CN)cc1 ; *NCC(=O)Nc1cccc(CNC(=N)N)c1 ; *NCC(=O)NCc1cc(CN)cc(CN)c1 ; *NCC(=O)NCc1cccc(CN)c1]

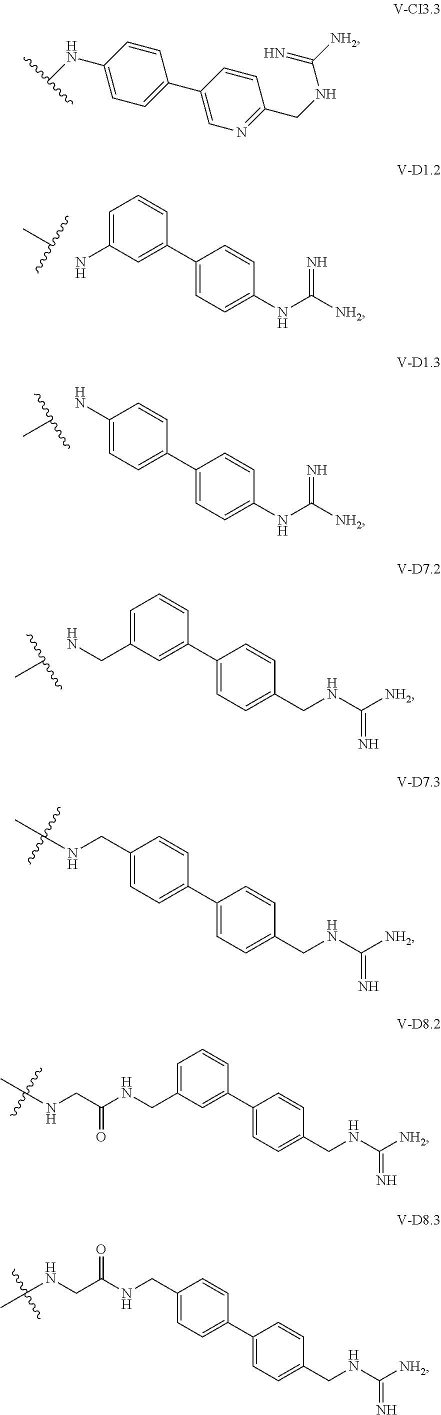

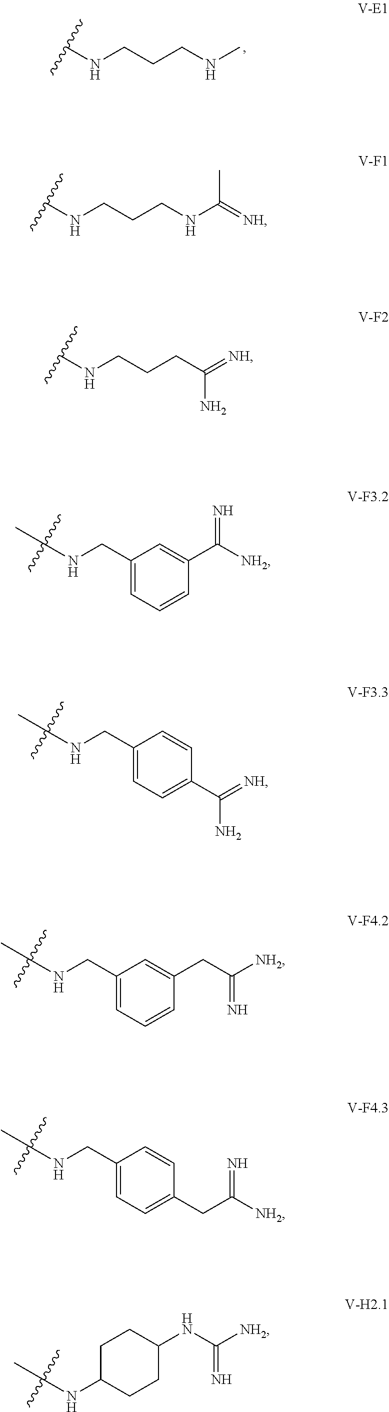

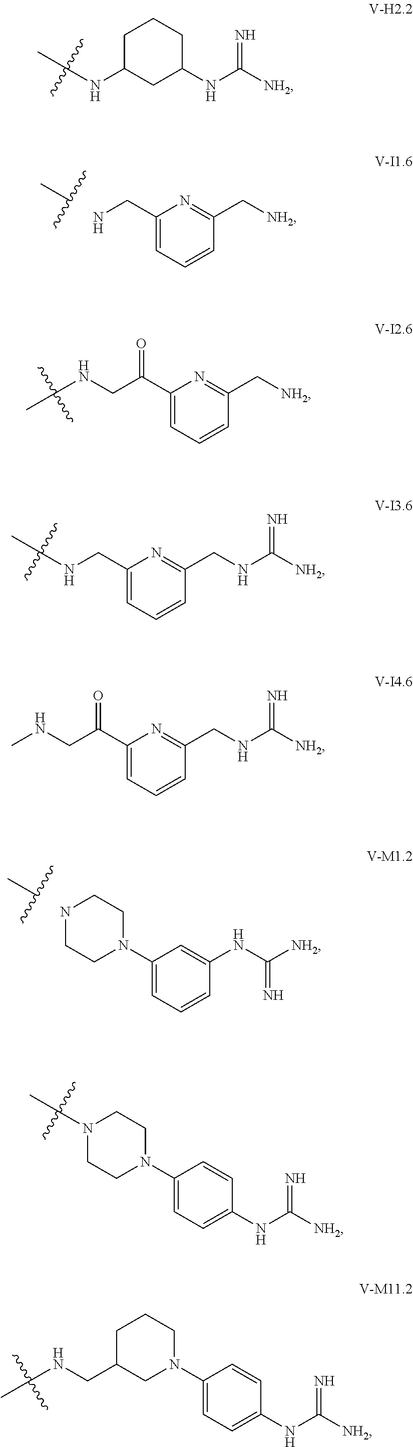

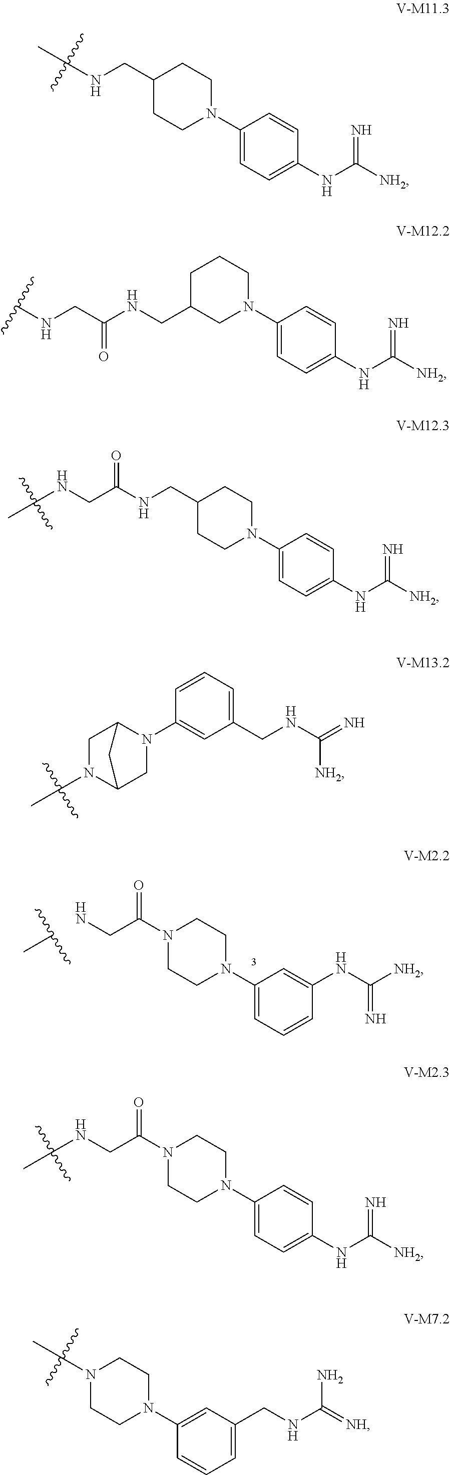

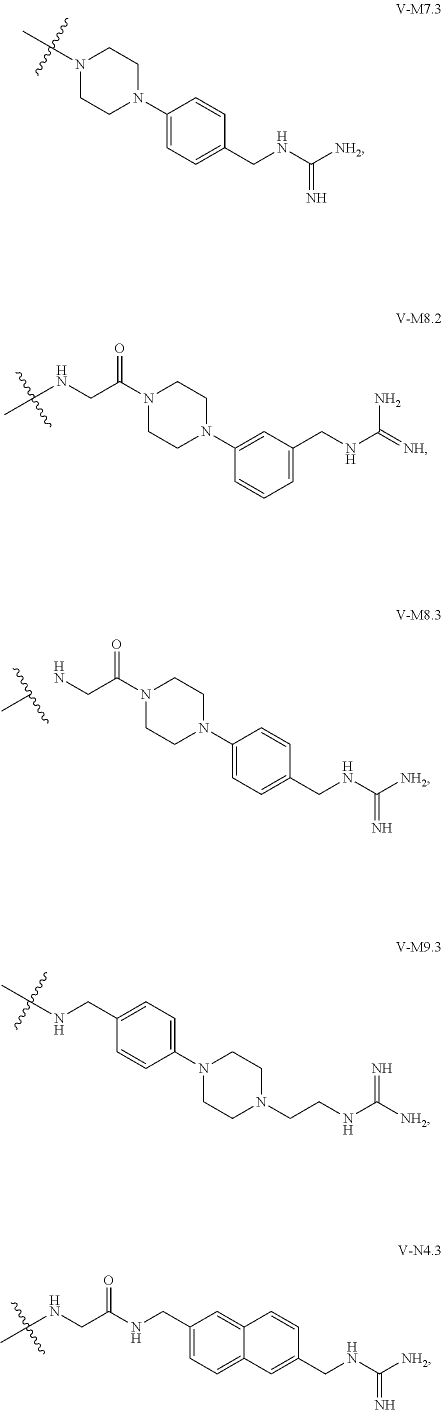

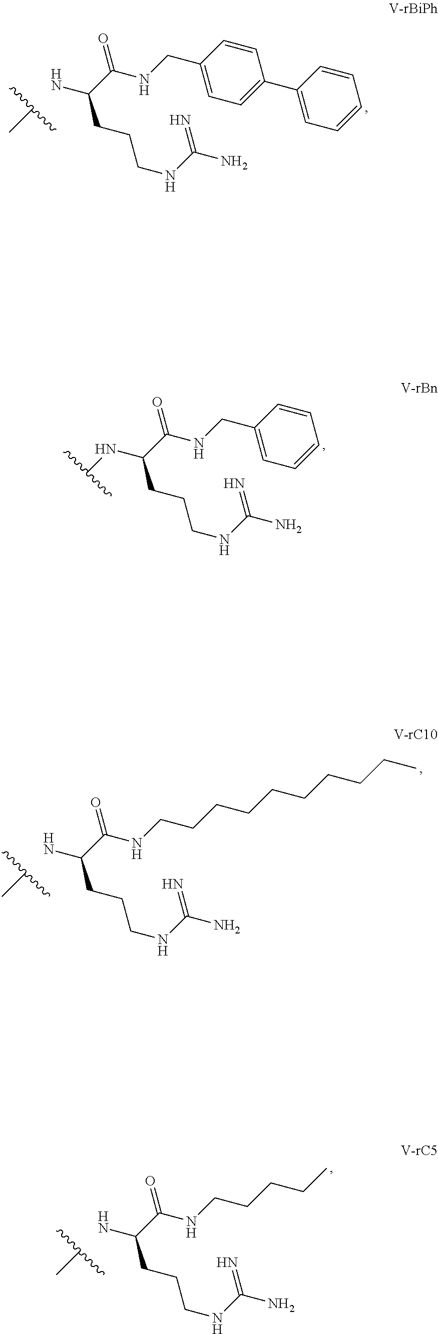

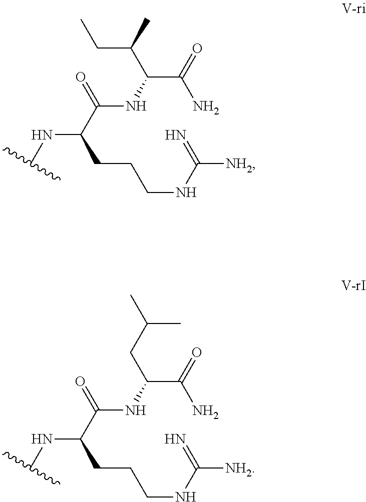 [SMILES: *N[C@H](CCCNC(=N)N)C(=O)N[C@@H](C(N)=O)[C@H](C)CC ; *N[C@H](CCCNC(=N)N)C(=O)N[C@H](CC(C)C)C(N)=O]

Any MoTr structure provided above constitutes a separate embodiment of the invention.

In some embodiments, the MoTr is aryl-based. In some embodiments, the MoTr is alkyl-based. As used herein, “aryl-based” or “alkyl-based” refers to presence or absence of an aromatic or an heteroaromatic ring structure in the MoTr structure. Where such a group is present, the MoTr is regarded as aryl-based, and where absent—it is alkyl-based. Thus, in some embodiments, the aryl-based MoTr are one or more of

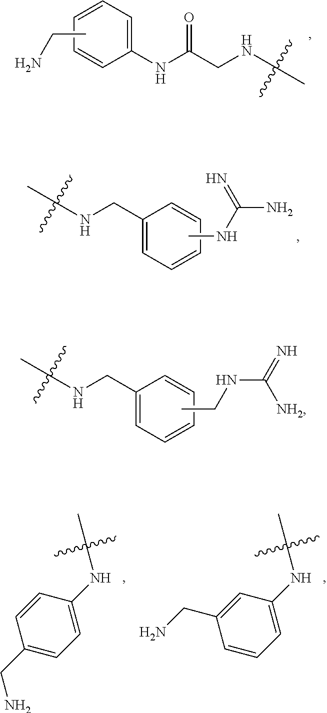

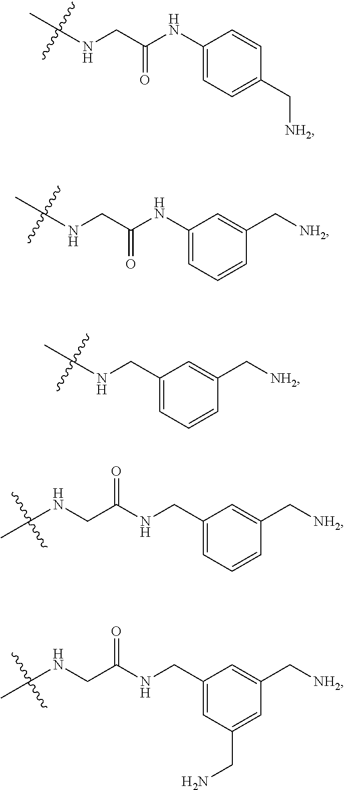 [SMILES: *NCC(=O)Nc1ccc(CN)cc1 ; *NCC(=O)Nc1cccc(CN)c1 ; *NCc1cccc(CN)c1 ; *NCC(=O)NCc1cccc(CN)c1 ; *NCC(=O)NCc1cc(CN)cc(CN)c1]

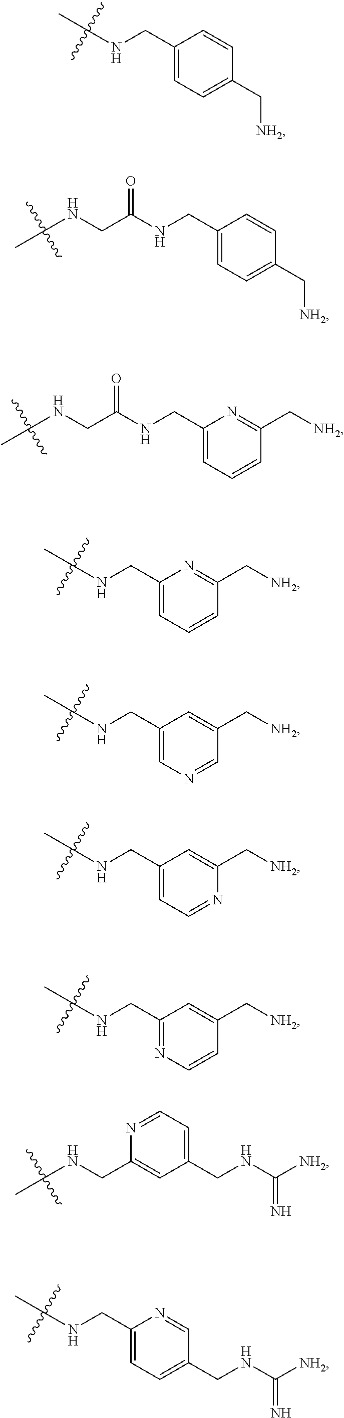

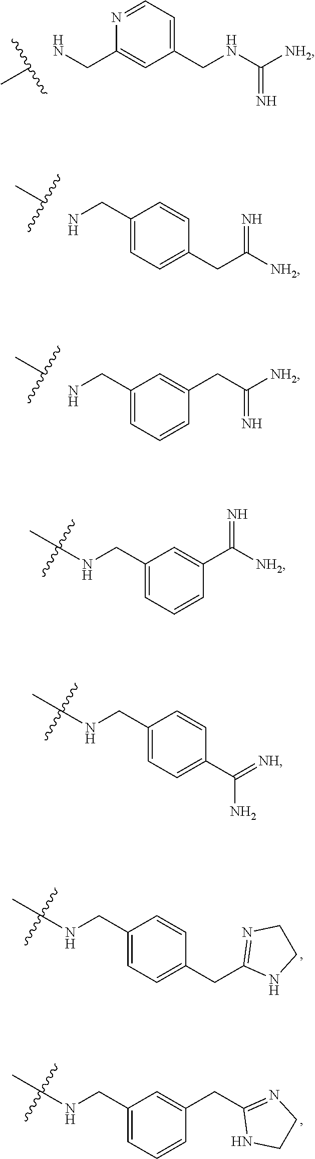

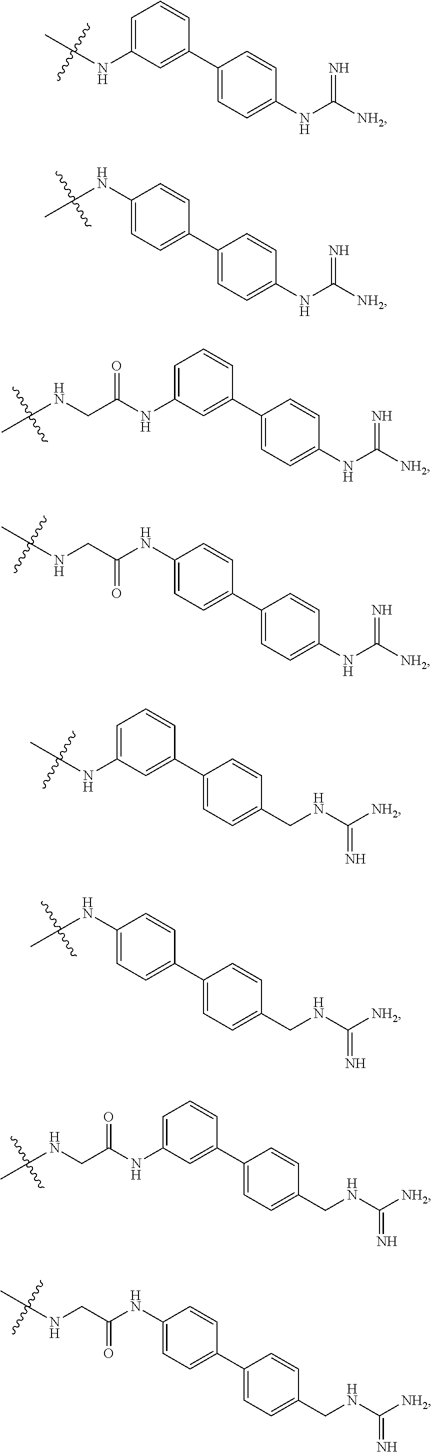

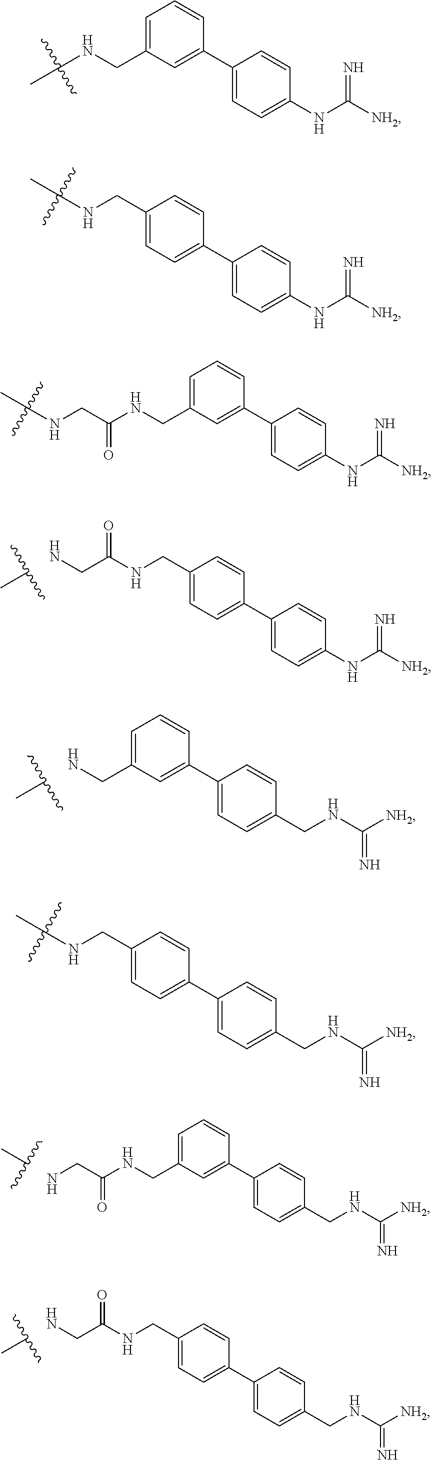

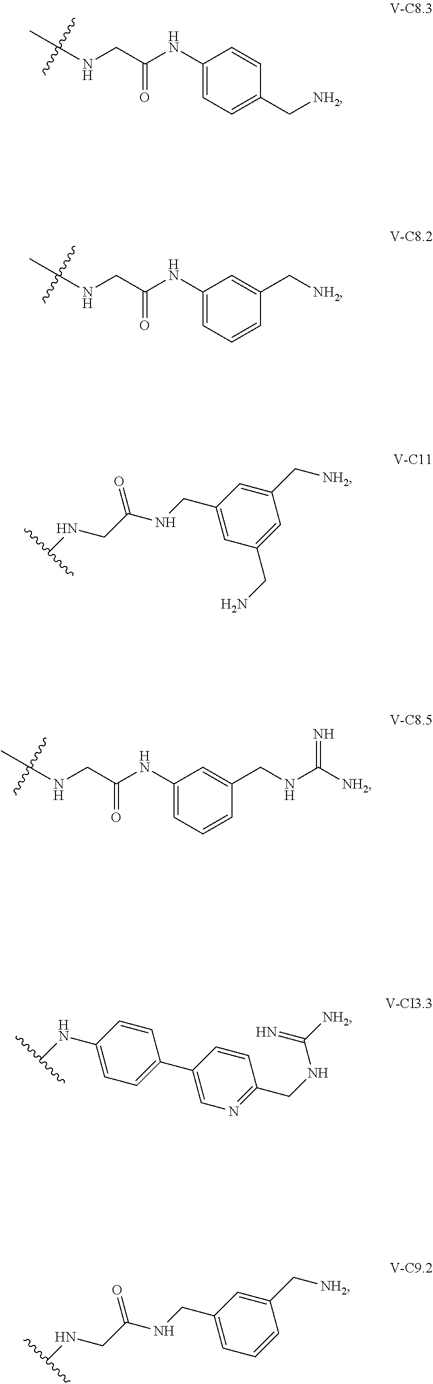 [SMILES: *NCC(=O)Nc1ccc(CN)cc1 ; *NCC(=O)Nc1cccc(CN)c1 ; *NCC(=O)NCc1cc(CN)cc(CN)c1 ; *NCC(=O)Nc1cccc(CNC(=N)N)c1 ; *Nc1ccc(-c2ccc(CNC(=N)N)nc2)cc1 ; *NCC(=O)NCc1cccc(CN)c1]

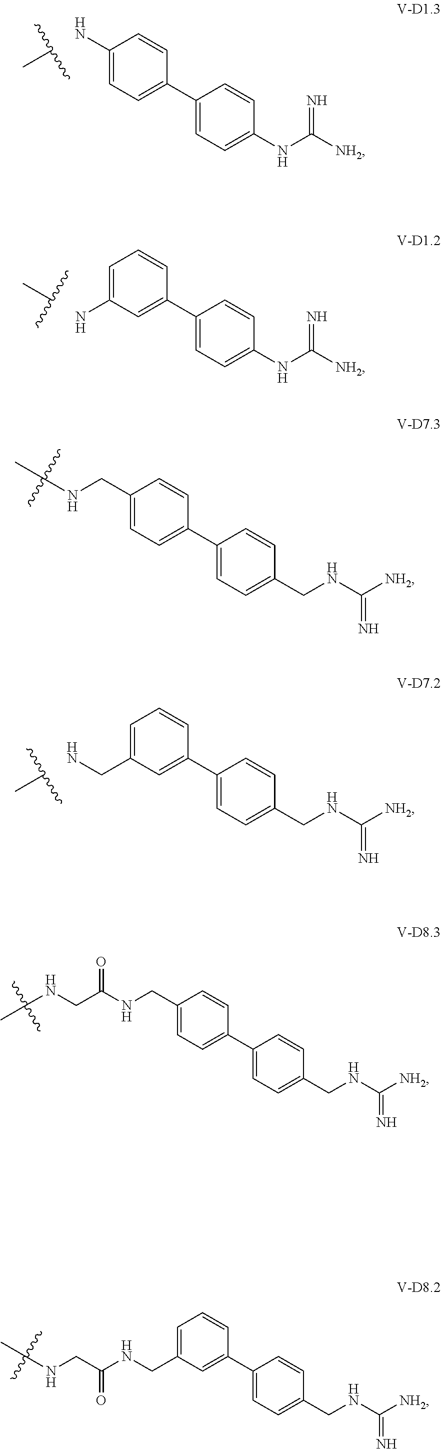

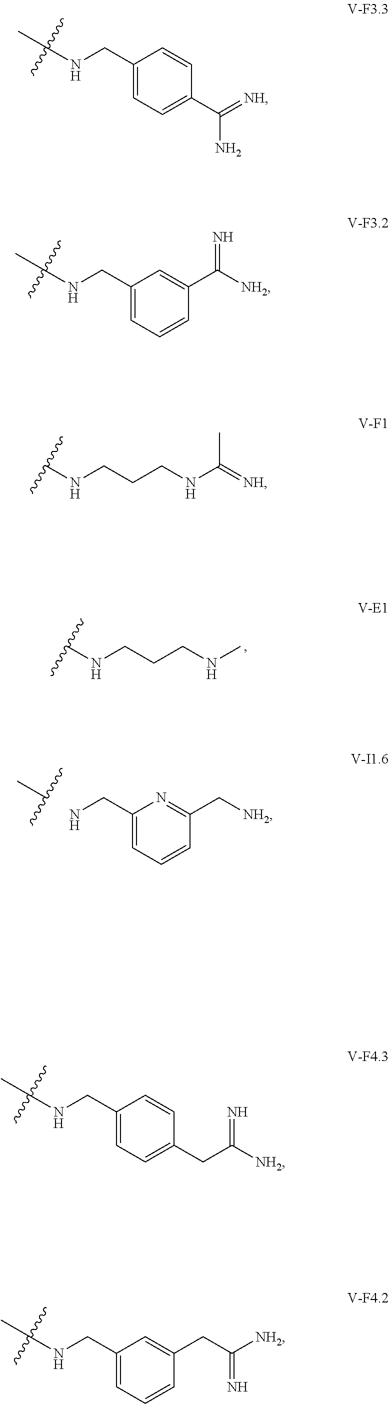

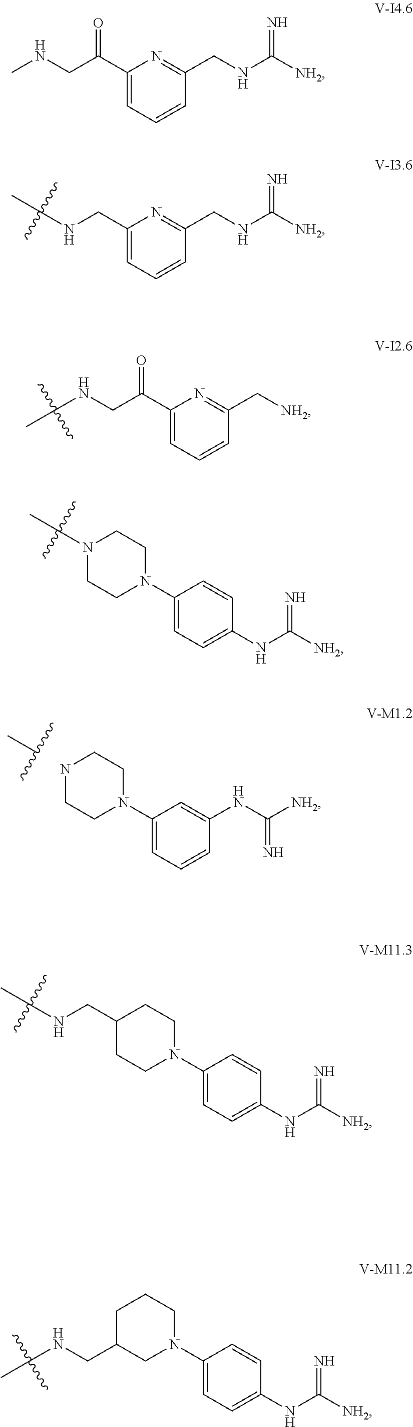

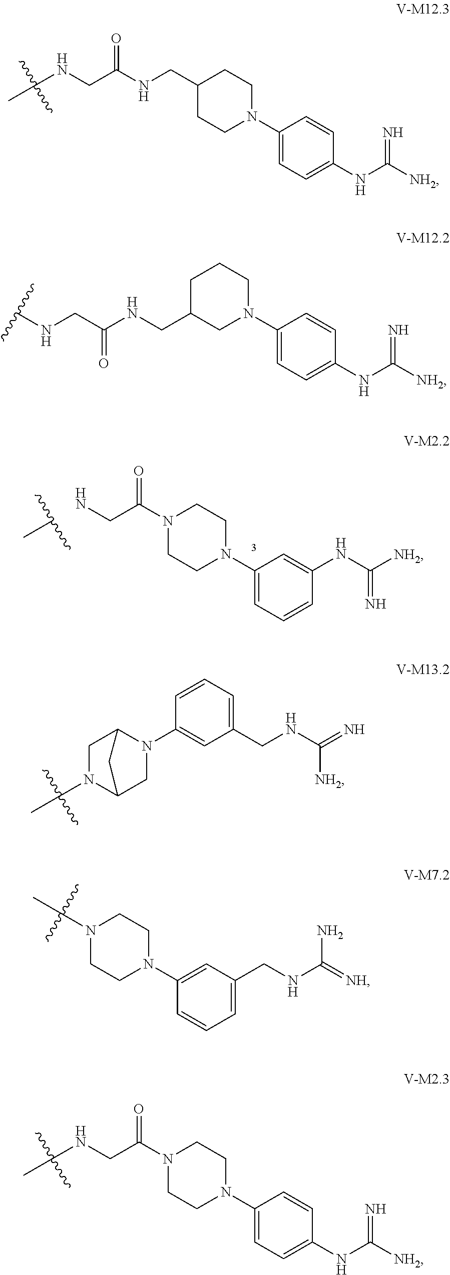

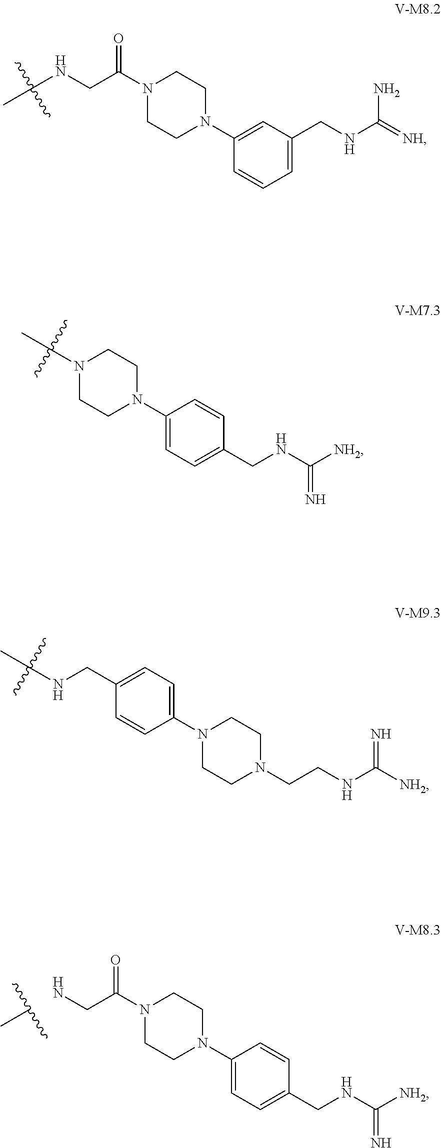

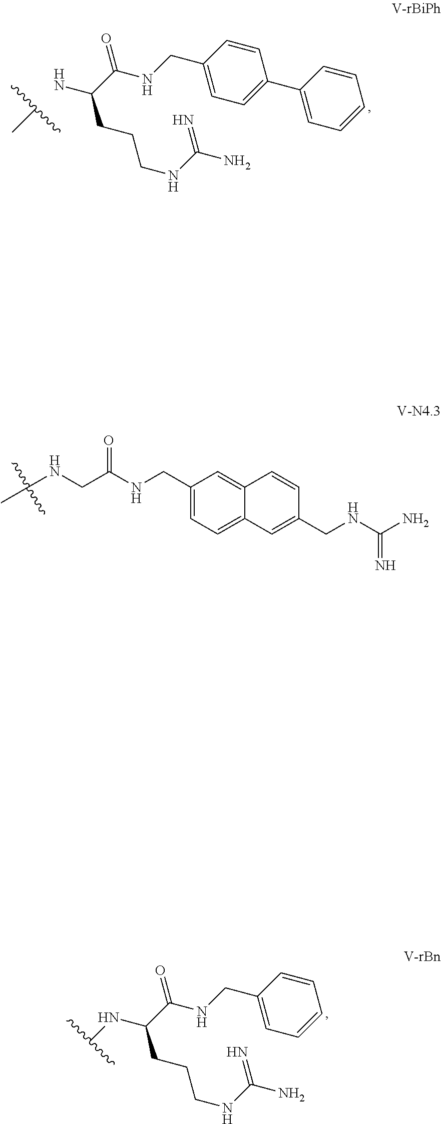

wherein each of the MoTr above constitutes a separate embodiment of the invention.

In some embodiments, the MoTr is alkyl-based and is selected from

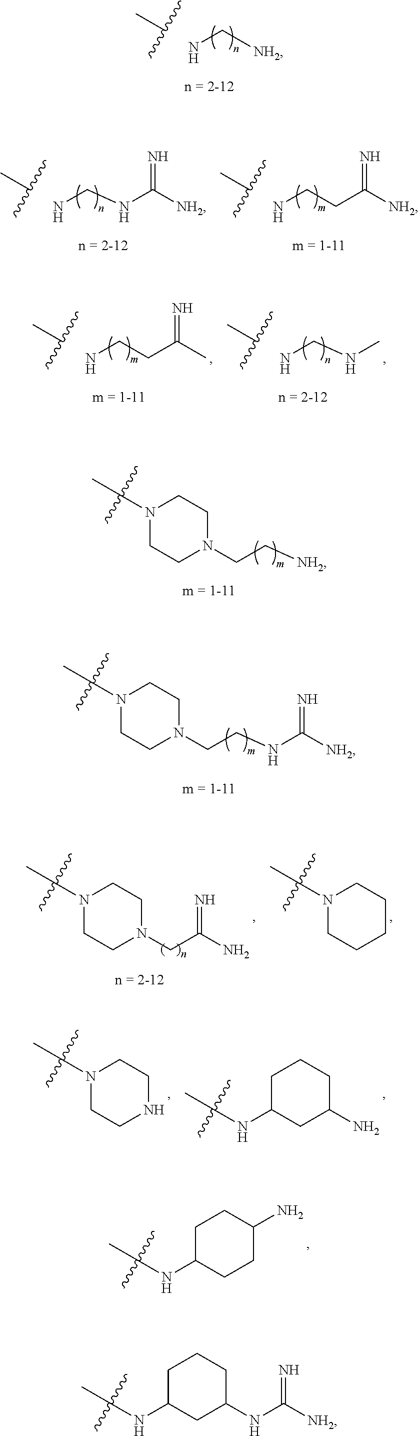

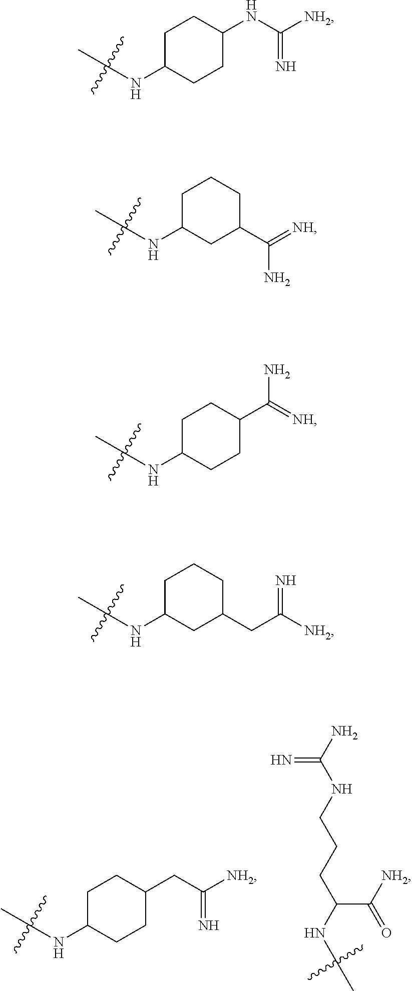 [SMILES: *NC1CCC(NC(=N)N)CC1 ; *NC1CCCC(C(=N)N)C1 ; *NC1CCC(C(=N)N)CC1 ; *NC1CCCC(CC(=N)N)C1 ; *NC1CCC(CC(=N)N)CC1 ; *NC(CCCNC(=N)N)C(N)=O]

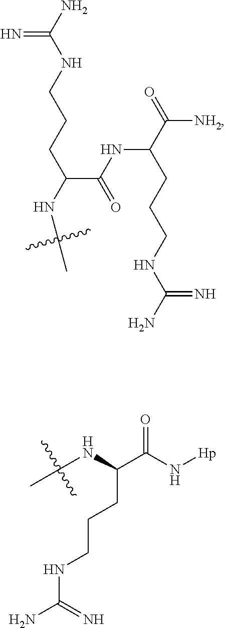

Hp is as defined herein

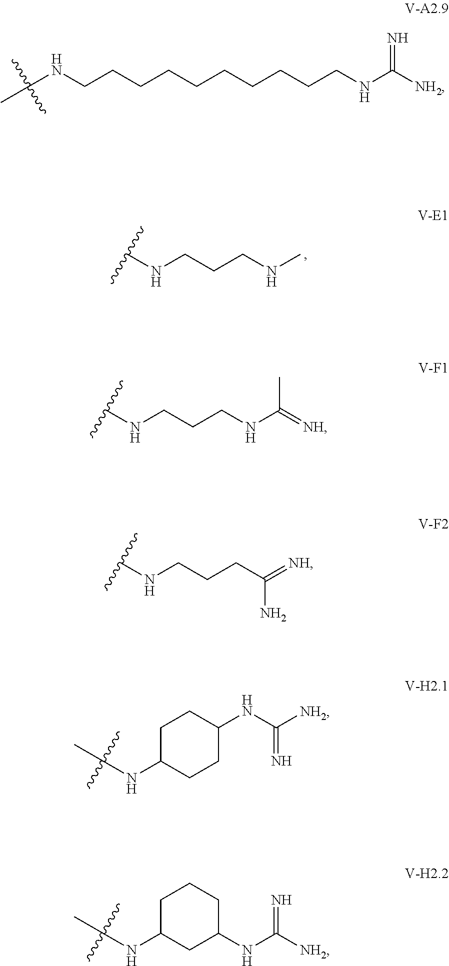

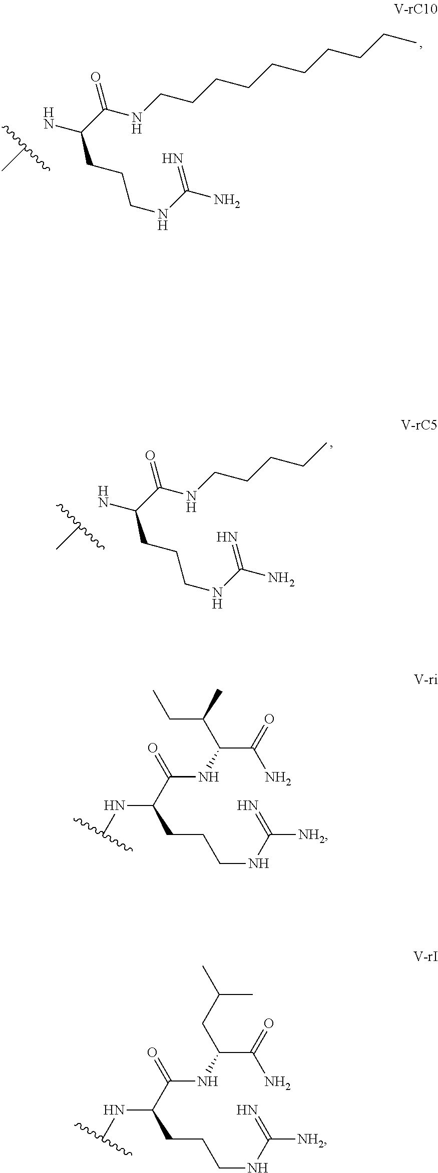

wherein each of the above MoTr structure constitutes a separate embodiment of the invention.

In some embodiments, the MoTr is any one of:

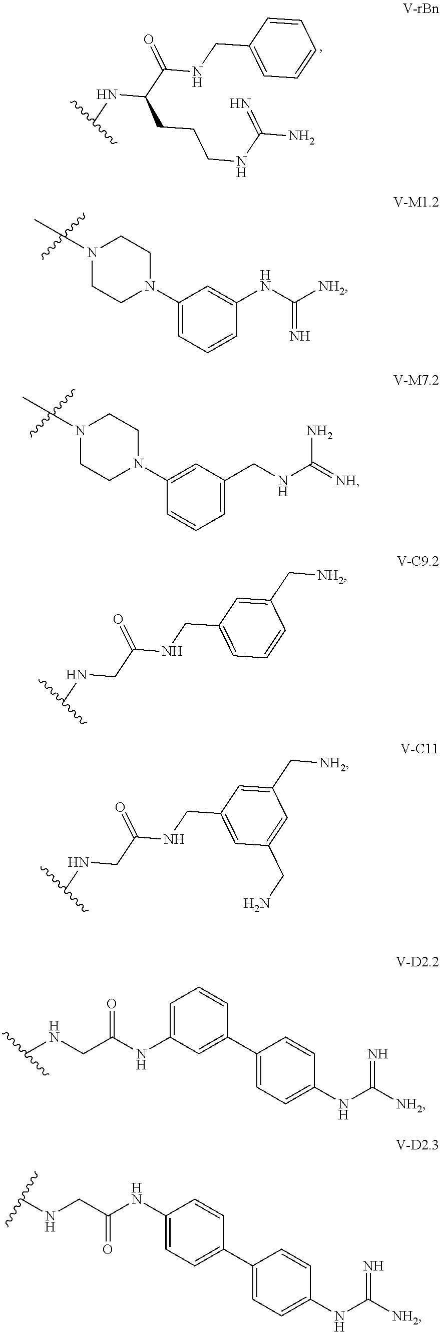

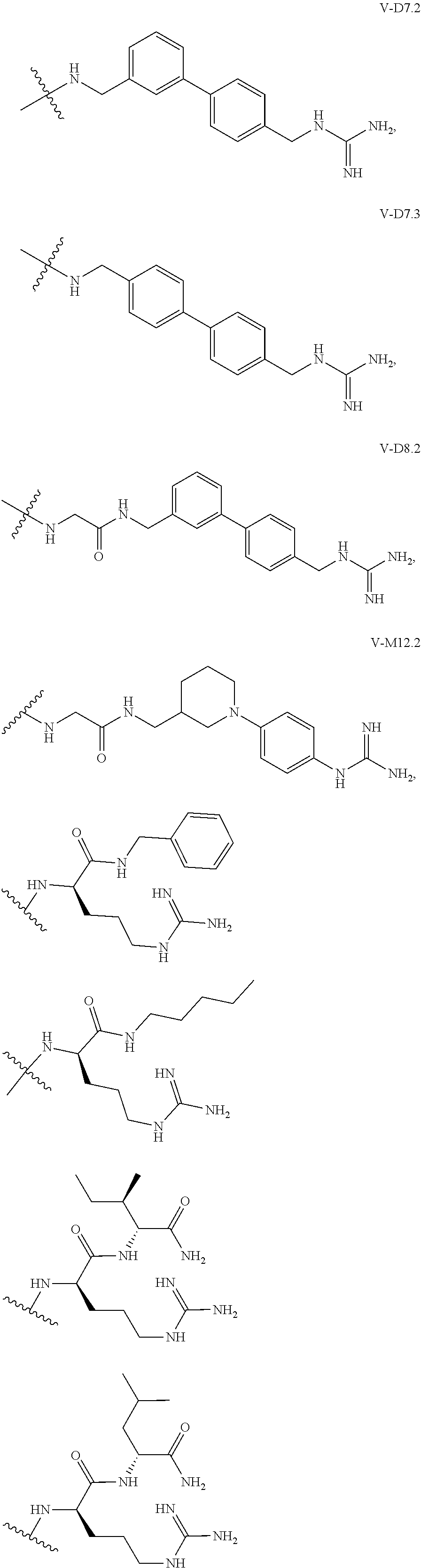

wherein each of the above MoTr constitutes an independent embodiment.

Compounds of the invention may comprise one or more chiral centers. Such chiral centers may be of either the (R) or (S) configuration, or may be a mixture thereof. Thus, compounds provided herein may be enantiomerically pure, or be stereoisomeric or diastereomeric mixtures. It is to be understood that the chiral centers of compounds may undergo epimerization in vivo. As such, one of skill in the art will recognize that administration of a compound in its (R) form is equivalent, for compounds that undergo epimerization in vivo, to administration of the compound in its (S) form. Thus, for example, in a compound of the invention having the Formula A-MoTr, wherein the MoTr is

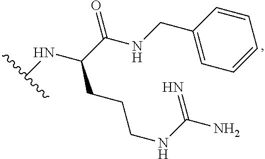 [SMILES: *N[C@H](CCCNC(=N)N)C(=O)NCc1ccccc1]

despite its projected bond configuration, represents both the (S) and (R) configurations as independent embodiments of the invention, in pure enantiomeric or racemic forms.

The invention also provides a compound of the structure A-MoTr, wherein A is an antibiotic, as defined, and MoTr is any one structure of the above aryl-based or alkyl-based structures. In some embodiments, the MoTr is an aryl-based structure.

In some embodiments, the antibiotic is vancomycin and the MoTr is an aryl-based or an alkyl-based MoTr structure.

Any compounds of structure (I) through (VII) can be used for various applications, the scope and extent of which will be detailed further below.

In other aspects, there is provided use of a compound of any of structure (I) through (VII) for use in any one of the following utilities:
1. for inhibiting or eradicating a bacterial infection in vivo and in vitro with a GPB or GNB strains.
2. for inhibiting or eradicating GPB and GNB strains which are antibiotic resistant strains.
4. for inhibiting or eradicating specific GPB strains.
6. for inhibiting or eradicating specific GNB strains.
7. for inhibiting or eradicating antibiotic resistant strains acting multiple drug resistance mechanisms.
8. for inhibiting or eradicating bacteria with lower doses than the antibiotic without the MoTr.
9. for preventing or treating specific infections.
10. for use in combination therapy with other antibiotic and non-antibiotic agents to increase efficacy, reduce toxicity and impart other properties.
11. for use in precision therapy targeting specific pathogens to further improve efficacy and avoid antimicrobial resistance.
12. for use in a method of subcutaneous administration of the compounds and compositions of the invention.
13. for forming a ready to use kit comprising a container (including but not limited to a bag) with the compounds and compositions of the invention at specific concentrations for IV administration.
14. for use as a point of care device permitting to determine the type of bacteria responsible for the infection and administer the compounds and compositions of the invention subcutaneously at predetermined doses.
15. for use as a transdermal patch incorporating additional technologies for increased skin permeability for systemic delivery of the compounds and compositions of the invention.
16. for use as a wearable injector device for convenient and cost-effective self-administration, e.g., Subcutaneous Wearable Bolus Injection (SWBI).
17. for forming nanoparticles encapsulating the compound for controlled drug delivery.

In some embodiments of compounds of the invention, the cargo moiety is an antibiotic moiety. The antibiotic moiety is at least one antibiotic drug that is chemically modified through the association to one or more transporter units of the forms depicted in Formulae (I) through (X). The atom(s) or functional moiety/moieties on the antibiotic drug that enable(s) association may be native atom(s) or moiety/moieties that is/are present on the antibiotic drug or that is/are formed on the drug by chemical modification(s). Such may be an atom or a group of atoms containing nitrogen, oxygen, phosphorus or sulfur atoms. For example, association may be via a hydroxyl group, a thiol group, an amine group, a carboxylic acid group, an aldehyde group, an amide group, a ketone group, or others that are native on the antibiotic drug.

Scheme 1 below depicts only few of the various functionalities on vancomycin that may be used for conjugation, in accordance with the structure of Formula (I):

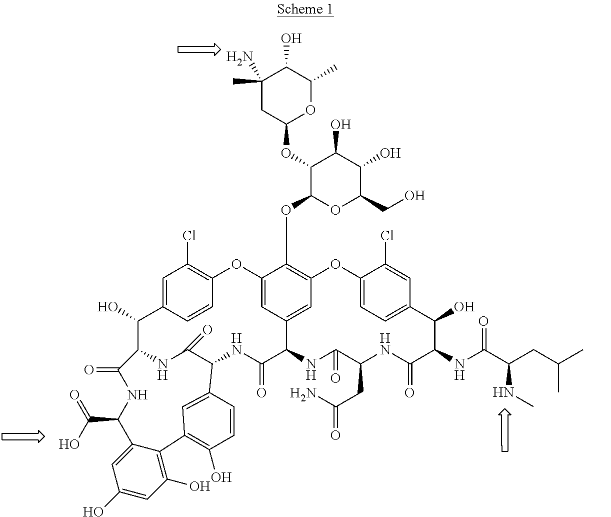 [SMILES: CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O)c3ccc(c(Cl)c3)Oc3cc2cc(c3O[C@@H]2O[C@H](CO)[C@@H](O)[C@H](O)[C@H]2O[C@H]2C[C@](C)(N)[C@H](O)[C@H](C)O2)Oc2ccc(cc2Cl)[C@H]1O]

Any of the other functionalities, as disclosed herein, may also be used to substitute the transport moiety.

Similarly, the antibiotic ampicillin may be substituted at the functionalities marked in Scheme 2:

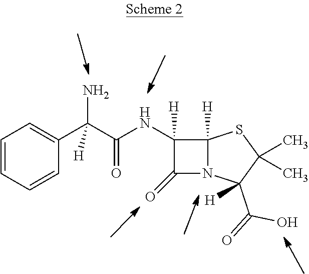 [SMILES: CC1(C)S[C@@H]2[C@H](NC(=O)[C@H](N)c3ccccc3)C(=O)N2[C@H]1C(=O)O]

Similar functionalities are present on other antibiotics, or in general on any cargo moiety which association with an MoTr moiety is desired. As a person of skill would appreciate, the association, e.g., via covalent bonding, between the cargo moiety, e.g., an antibiotic, and the MoTr moiety may proceed by any of the chemical tools available to a chemist. These include acid-base reactions, substitution reactions, nucleophilic or electrophilic reactions, and any of the other specific reactions providing substitution venues for achieving association between the two moieties. A person of skill in the art would also know to choose the particular site of substitution. Typically, the site of substitution may be selected to provide a robust or labile association, as may be the case, as well as to provide a conjugate of superior effectivity. The chemical as well as therapeutic effectivity may be tested utilizing assays and methodologies known to the artisan.

The antibiotic moiety may be any one antibiotic drug known in the art that is suitable for conjugation or that can be modified to be rendered suitable for use in accordance with the invention. The antibiotic is typically one which is approved by the FDA and any other antibiotic drug that is under development, including antibiotics derived from natural sources and synthetic and semisynthetic compounds.

Classes of antibiotics include, for example, penicillins, e.g. penicillin G, penicillin V, methicillin, oxacillin, carbenicillin, nafcillin, ampicillin, etc. penicillins in combination with b-lactamase inhibitors, cephalosporins, e.g. cefaclor, cefazolin, cefuroxime, moxalactam, etc; carbapenems; monobactams; aminoglycosides; tetracyclines; macrolides; lincomycins; oxazolidinones, polymyxins; sulfonamides; quinolones; cloramphenical; metronidazole; spectinomycin; trimethoprim; glycopeptides; and others.

The glycopeptide antibiotics are a class of drugs of microbial origin that are composed of glycosylated cyclic or polycyclic nonribosomal peptides. Significant glycopeptide antibiotics include the anti-infective antibiotics vancomycin, teicoplanin, telavancin, ramoplanin and decaplanin. This class of compounds further encompasses vancomycin analogs and derivatives, for example, oritavancin and dalbavancin (both lipoglycopeptides), and telavancin a semi-synthetic lipoglycopeptide derivative of vancomycin. Other vancomycin analogs are disclosed, for example, in WO 2015022335 and Chen et al. (2003) PNAS 100(10): 5658-5663, each incorporated herein by reference.

In some embodiments, the antibiotic is selected amongst such compounds having a functional group selected from an amine, an alcohol or a carboxylic acid, and others as indicated herein. In some embodiments, the antibiotic may be selected from vancomycin, linezolid, azithromycin, daptomycin, colistin, eperezolid, fusidic acid, rifampicin, tetracyclin, fidaxomicin, clindamycin, lincomycin, rifalazil, and clarithromycin.

In some embodiments, the antibiotic is vancomycin.

The linker moiety L and the linker moiety Lₕ, where present, each independently of the other, may be a short or long linker moiety comprising between 1 and 10 carbon atoms, and optionally one or more heteroatoms selected from N, O, P and S. In some embodiments, the linker is a short linker of between 1 and 5 carbon atoms in a straight or branched carbon chain. In some embodiments, linkers having a number of carbon atoms greater than 5 are excluded from the invention disclosed herein.

The linker may be a bifunctional moiety having one heteroatom capable of covalently associating to a moiety on the antibiotic moiety and another heteroatom capable of associating to the lipophilic moiety, as disclosed herein. Alternatively, the linker moiety L or Lₕ may be a carbon moiety that is chemically associated to the antibiotic and the lipophilic moiety.

In some embodiments, the linker L or Lₕ comprises between 1 and 10 carbon atoms, or between 2 and 10, 3 and 10, 4 and 10, 5 and 10, 6 and 10, 7 and 10, 8 and 10, or 9, or 10 carbon atoms. the linker L or Lₕ comprises between 1 and 5 carbon atoms, or between 2 and 5, 3 and 5, 4 or 5 carbon atoms.

In some embodiments, the number of atoms in the linker L or Lₕ is between 3 and 40 atoms, which may include carbons atoms, hydrogen atoms as well as heteroatoms such as O, N, S, and P.

In some embodiments, the linker is an aliphatic straight or branched carbon chain. In some embodiments, the linker comprises one or more double or triple binds. In some embodiments, the linker comprises one or more aromatic or heteroaromatic ring structure which may or may not be substituted. In some embodiments, the linker comprises at least one heteroatom selected from N, O, P and S.

In some embodiments, L is present and Lₕ is absent.

In some embodiments, L is absent and Lₕ is present.

In some embodiments, where two or more linker moieties L or Lₕ are present, each may be the same of different.

In some embodiments, in compounds of the invention L is absent and Lₕ, where possible, may be present or absent. In some embodiments, both L and Lₕ are absent. In other embodiments, one of L and Lₕ is absent.

In some embodiments, each of L and Lₕ, independently of the other, may be selected from C1-C10alkylene, C2-C10alkenylene, C4-C10carbocyclyl, C4-C10heterocarbocyclyl, C6-C10arylene, C1-C10alkyleneC6-C10arylene, C6-C10aryleneC1-C10alkylene, C1-C10alkyleneC4-C10carbocyclyl, C4-C10carbocyclylC1-C10alkylene, C1-C10alkyleneC4-C10heterocarbocyclyl, C4-C10heterocarbocyclylC1-C10alkylene, C5-C10heteroarylene, C1-C10alkyleneC5-C10heteroarylene, C5-C10heteroaryleneC1-C10alkylene, C1-C10alkylene-C(═O)C1-C10alkylene, C1-C10alkylene-C(═O)O—C1-C10alkylene, C1-C10alkylene-OC(═O)C1-C10alkylene, C1-C10alkylene-C(═O)C6-C10arylene, C1-C10alkylene-C(═O)O—C6-C10arylene, C1-C10alkylene-OC(═O)C6-C10arylene, C1-C10alkylene-C(═O)C5-C10heteroarylene, C1-C10alkylene-C(═O)O—C5-C10heteroarylene, C1-C10alkylene-OC(═O)C5-C10heteroarylene, C1-C10alkylene-O—C1-C10alkylene, C1-C10alkylene-O—C6-C10arylene, C1-C10alkylene-O—C5-C10heteroarylene, oligophosphoester, oligocarbonate, amino acids, short peptides (comprising between 2 and 5 amino acids), and any sulfur-equivalent of the aforementioned oxygen-based linkers.

In some embodiments, the linker L or Lₕ is a C1-C10alkylene, C2-C10alkenylene, C4-C10carbocyclyl, C4-C10heterocarbocyclyl or C6-C10arylene.

In some embodiments, the linker L and Lₕ is C1-C10alkylene.

The lipophilic moiety, Hp, may be regarded as hydrophobic. It may be associated at one end to the linker moiety L and on the other end to a linker moiety Lₕ or directly to an N-containing cationic or non-cationic group, or may be a pendant group extending from the linker moiety L, as depicted above. Alternatively, the lipophilic moiety is substituted on the cargo moiety, e.g., as shown in structure Formula (III).

Notwithstanding the position of the lipophilic moiety in a compound of the invention, the moiety is one which generally comprises lipophilic groups, e.g., groups not capable of hydrogen bonding or ionic association, or groups that generally decrease water solubility of the compound. The lipophilic moiety may therefore be selected from C1-C20alkyl(ene), C2-C20alkenyl(ene), C2-C20alkynyl(ene), C4-C20carbocyclyl(ene), C4-C20heterocarbocyclyl(ene), C6-C12aryl(ene), C1-C20alkyleneC6-C12aryl(ene), C6-C12aryleneC1-C20alkyl(ene), C1-C20alkyleneC4-C20carbocyclyl(ene), C4-C20carbocyclylC1-C20alkyl(ene), C1-C20alkyleneC4-C20heterocarbocyclyl(ene), C4-C10heterocarbocyclylC1-C20alkylene(ene), C5-C12heteroaryl(ene), C1-C20alkyleneC5-C12heteroaryl(ene) and C5-C12heteroaryleneC1-C20alkyl(ene).

As indicated above, any of the MoTr moieties disclosed herein may be associated directly to a cargo moiety to form a compound of Formula (X), or via a linker moiety, L, as defined herein. In such embodiments, the transporter moieties may be associated to A via linker L to form a compound of the formula A-L-MoTr. L is selected as herein.

In some cases, L is an amino acid, herein designated AA, thus forming a compound of the Formula A-AA-MoTr.

The amino acid may be any of the amino acids known in the art. In some embodiments, AA designates a short peptide comprising between 2 and 5 amino acids.

Examples of the amino acids include, but are not limited to, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, selenocysteine, pyrrolysine, pyroglutamate and any derivatives thereof.

It is commonly known that certain amino acids have two stereoisomers designated as L and D amino acids. Amino acids as mentioned herein include L isomer, D isomer, or a mixture thereof Furthermore, any of the L, D, or mixed amino acids may further contain additional steroisomeric center(s) in their structures. The amino and carboxyl groups may be located at alpha, beta, gamma, delta, or other positions. Amino acids suitable for the present disclosure can be naturally occurring amino acid or non-naturally occurring (e.g., synthetic) amino acid.

In some embodiments, AA is glycine.

Moiety X is an N-containing cationic or non-cationic group. The number of X groups that are substituted on a linker moiety, on a lipophilic moiety or directly on the cargo moiety is designated by the integer v, which may be 1 or 2. In other words, the group is a single group or a plurality of such groups that is/are substituted as shown in the various Formulae, and which comprises at least one nitrogen atom. The group may be cationic, namely bearing one or more positive charges; or non-cationic, namely wherein the nitrogen atom(s) is neutral, but which may be converted into a charged form at a given pH.

Moiety X may thus be selected from primary, secondary and tertiary amines, linear or cyclic amines, and ammonium derivatives thereof; imidamides, acetimideamides; amidines, guanidines (e.g., derivable from an amino acid arginine); imidazoline, ureas; indoles and indazoles and others. In some embodiments, moiety X is an amine (primary, secondary, tertiary, linear or cyclic or charged forms thereof) or a guanidine or guanidinium moiety.

As used herein, alkyl, alkenyl and alkynyl carbon chains, or the corresponding alkylene, alkenylene and alkynylene, if not specified, contain from 1 to 20 carbons, or from 2 to 20 carbons, and are straight or branched. Where double or triple bonds are present, the carbon chain may comprise from 2 to 20 carbons.

The expression “C1-C10alkylene” designates an alkylene chain comprising between 1 and 10 carbon atoms in a linear or branched arrangement. The group may comprise 1, 2, 3, 4, 5, 6, 7, 8, 9 or 10 carbon atoms. In some embodiments, the group comprises 1 to 10, 1 to 9, 1 to 8, 1 to 7, 1 to 6, 1 to 5, 1 to 4, 1 to 3, or 1 or 2 carbon atoms. Exemplary alkylene groups herein include, but are not limited to, methylene, ethylene, propylene, isopropylene, isobutylene, n-butylene, sec-butylene, tert-butylene, isohexylene, and others.

The equivalent “alkenylene” or “alkynylene” comprises each carbon chains of from 2 to 20 carbons, in certain embodiments—contain 1 to 8 double bonds or triple bonds; in some embodiments 2 to 16 carbons, with 1 to 5 double bonds or triple bonds.

The expression “alkyl(ene)” or any variation thereof refers to an alkyl group which may be an end-of-chain alkyl or a mid-chain alkylene.

The “carbocyclyl” refers to a saturated mono- or multi-cyclic ring system, in certain embodiments of 4 to 20 carbon atoms, may be composed of one ring or two or more rings which may be joined together in a fused, bridged or spiro-connected fashion. As used herein, “heterocarbocyclyl” refers to a monocyclic or multicyclic non-aromatic ring system, where one or more of the atoms in the ring system is not a carbon atom, namely a heteroatom that is selected from N, O, P and S. The term also encompasses biheterocyclyls and fused heterocycles.

While the “carbocyclyl” and “heterocarbocyclyl” are end-of-chain groups, the corresponding “carbocyclylene” and “heterocarbocyclylene” are each mid-chain moieties.

The “aryl” group is an aromatic monocyclic or multicyclic group containing from 6 to 12 carbon atoms. Aryl groups include but are not limited to groups such as unsubstituted or substituted fluorenyl, unsubstituted or substituted phenyl, and unsubstituted or substituted naphthyl. Other aryls may include biaryls including unsubstituted or substituted biphenyl and aryl-heteroaryl bifunctional compounds. The “arylene” group is similarly a mid-chain aryl.

As used herein, the “heteroaryl” is a monocyclic or multicyclic aromatic ring system, wherein on or more of the atoms in the ring system is a heteroatom, that is, an element other than carbon. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl, bipyridyls and isoquinolinyl. Similarly, “heteroarylene” is a mid-chain heteroaryl.

The group “aralkylene” refers to an alkylene group in which one of the hydrogen atoms of the alkylene is replaced by an aryl or an arylene group. Similarly, the group “heteroaralkylene” refers to an alkylene group in which one of the hydrogen atoms of the alkylene is replaced by a heteroaryl or heteroarylene group.

The expression “C1-C10alkyleneC6-C10arylene” or any other such combined functionality designates a chemical moiety having an alkylene moiety of 1 to 10 carbon atoms that is directly associated to an arylene having 6 to 10 carbon atoms. Similarly, the expression “C1-C10alkylene-C(═O)C1-C10alkylene” refers to a chemical group wherein an alkylene having between 1 and 10 carbon atoms is separated from another alkylene having the same or different number of carbon atoms by a carboxyl moiety —C(═O)—.

The group “—C(═O)—” designates a functionality wherein the carbon atom is associated to two groups via single bonds each and to an oxygen atom via a double bond. This is a carboxyl group.

The group “—C(═O)O—” designates a functionality wherein the carbon atom is associated to a variable group via a single bond, to an oxygen atom via a single bond and to another oxygen atoms via a double bond. This group may designate an ester or an acid group depending on the variable functionalities.

In most general terms, every expression used herein of the form “CY-CZ”, wherein Y and Z are numerals; such as C1-C10, C2-C20, C5-C10, C6-C12, C3-C10 and others, designates the number of carbon atoms in a chemical functionality, where Y designates the minimum number of carbon atoms and Z designates the maximum number of carbon atoms. The expression is inclusive, encompassing the minimum and the maximum value as well as any intermediate integer residing between the minimum and maximum values. For example, a C1-C10alkylene is an alkylene group having 1, 2, 3, 4, 5, 6, 7, 8, 9 or 10 carbon atoms. A C6-C12arylene is an aromatic ring structure having 6, 7, 8, 9, 10, 11 or 12 carbon atoms. Similarly, a C3-C10 heteroarylene is a heteroarylene, as defined, wherein the aromatic heteroring structure comprises 3, 4, 5, 6, 7, 8, 9 or 10 carbon atoms and any number of heteroatoms selected from N, O. P and S. the expression CY-CZ also means that any intermediate range between Y and Z is included. For example, the expression “C1-C10alkylene” encompasses any range such as C1-C10, C1-C9, C1-C8, C1-C7 C1-C6, C1-C5 C1-C4, C1-C3 C2-C10, C2-C9 C2-C8, C2-C7, C2-C6, C2-C5 C2-C4, C3-C10, C3-C9, C3-C8, C3-C7, C3-C6, C3-C5, C4-C10, C4-C9, C4-C8, C4-C7, C4-C6, C5-C10, C5-C9, C5-C8, C5-C7, C6-C10, C6-C9, C6-C8, C7-C10, C7-C9, or C8-C10.

As mentioned herein, some of the functionalities may be substituted. Any functionality that is said to be substituted or optionally substituted, may be substituted with one or more substituents, in certain embodiments one, two, three or four substituents, where the substituents are, for example, H, a halogen (F, Br, Cl or I), —OH, —SH, an amine, —NO₂, an aryl, an alkyl, a double bond or triple bond containing functionality, a carboxylic acid, an ester, an ether, —C(O)NH₂, —S(O)₂O—, —S(O)—, and others.

Any of the transporter moieties meeting the structural definitions provided herein may be associated to any antibiotic moiety, as defined herein. In some embodiments, the antibiotic is vancomycin and the conjugate is of a form selected from:

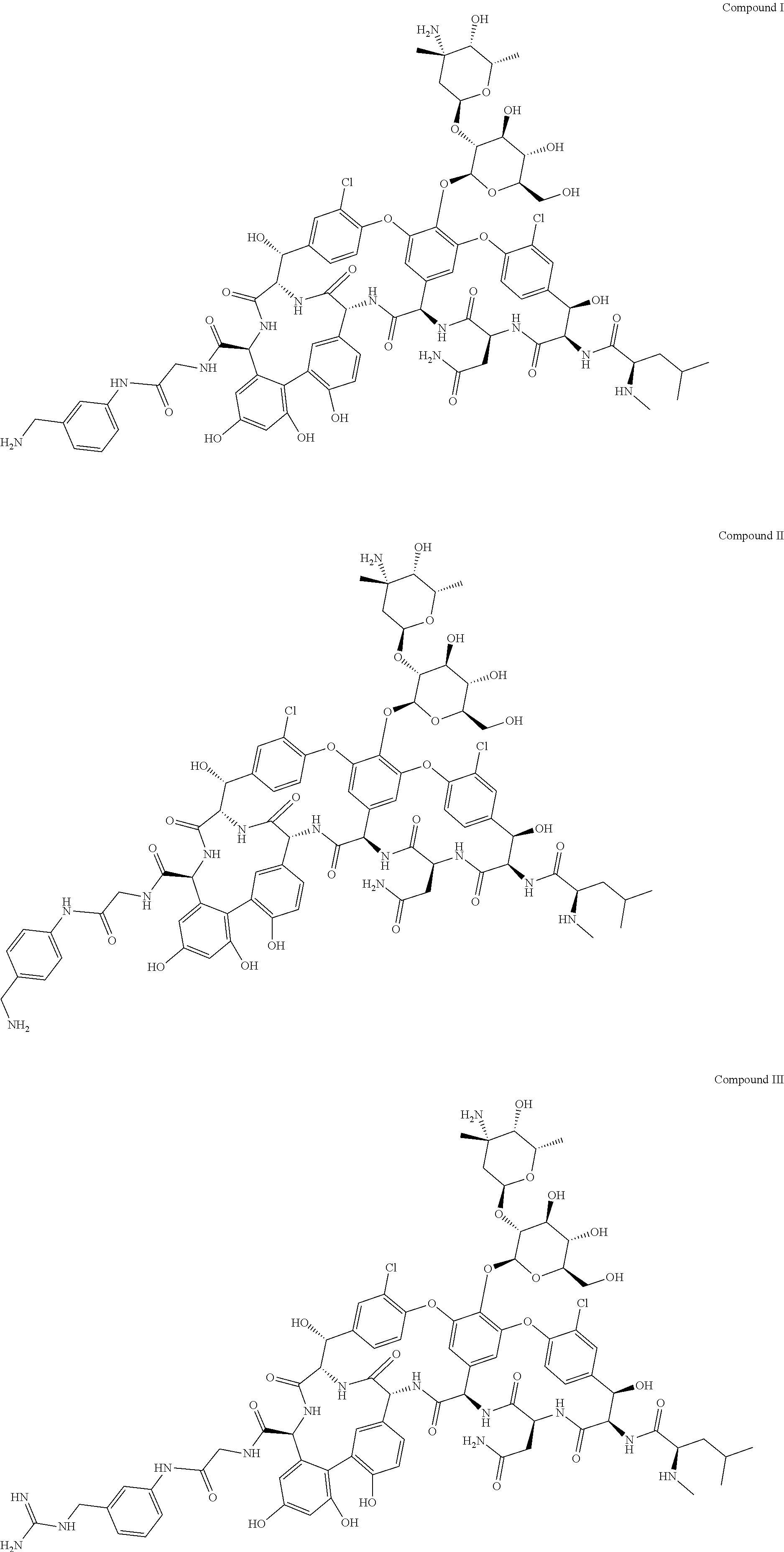

In a similar fashion, other antibiotic-molecular transporter conjugates may be formed.

Also provided are compounds of the general Formula A-MoTr, wherein A is vancomycin or linezolid and MoTr is a transporter moiety comprising a structure as defined hereinabove.

In some embodiments, the compound is of the structure A-L-MoTr, wherein L is a linker moiety selected from C1-C10alkylene, C2-C10alkenylene, C4-C10carbocyclyl, C4-C10heterocarbocyclyl, C6-C10arylene, C1-C10alkyleneC6-C10arylene, C6-C10aryleneC1-C10alkylene, C1-C10alkyleneC4-C10carbocyclyl, C4-C10carbocyclylC1-C10alkylene, C1-C10alkyleneC4-C10heterocarbocyclyl, C4-C10heterocarbocyclylC1-C10alkylene, C5-C10heteroarylene, C1-C10alkyleneC5-C10heteroarylene, C5-C10heteroaryleneC1-C10alkylene, C1-C10alkylene-C(═O)C1-C10alkylene, C1-C10alkylene-C(═O)O—C1-C10alkylene, C1-C10alkylene-OC(═O)C1-C10alkylene, C1-C10alkylene-C(═O)C6-C10arylene, C1-C10alkylene-C(═O)O—C6-C10arylene, C1-C10alkylene-OC(═O)C6-C10arylene, C1-C10alkylene-C(═O)C5-C10heteroarylene, C1-C10alkylene-C(═O)O—C5-C10heteroarylene, C1-C10alkylene-OC(═O)C5-C10heteroarylene, C1-C10alkylene-O—C1-C10alkylene, C1-C10alkylene-O—C6-C10arylene, C1-C10alkylene-O—C5-C10heteroarylene, oligophosphoester, oligocarbonate, amino acids, peptides comprising between 2 and 5 amino acids, and sulfur-equivalent linkers of any of the aforementioned oxygen-based linkers.

In some embodiments, L is an amino acid.

In some embodiments, the compound has a structure as defined in any one of the herein disclosed structural Formulae.

In some embodiments, in compounds of the invention, at least one of X and Hp, separately or in combination, is a structure as defined for MoTr herein.

Also provided is compound as herein designated Compound I-XIII.

Conjugates of the invention may be made into pharmaceutically acceptable salts. Acid addition salts of compounds of the invention include salts derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate, galacruronate (see, for example, Berge S. M., et al., “Pharmaceutical Salts,” J. of Pharmaceutical Science, 66:1-19 (1977)).

The acid addition salts of said basic compounds may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine (see, for example, Berge S. M., et al., “Pharmaceutical Salts,” J. of Pharmaceutical Science, 66:1-19 (1977)).

The base addition salts of acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

As demonstrated herein, compounds of the invention are antibiotic conjugates which may be used in therapeutic and prophylactic methodologies for treating or preventing at least one bacterial infection in a subject and in methods of eradication or prevention of biofilms on surfaces, e.g., medical devices.

Where medical treatment and prevention are concerned, the subject is an animal, including, but not limited to, human and non-human primates, including simians and humans; rodents, including rats and mice; bovines; equines; ovines; felines; canines; and the like.

The conjugates of the invention may be used as such or may be administered or used in a formulation or composition that optionally also comprises at least one carrier. Where pharmaceutical formulations or compositions are concerned, a pharmaceutically acceptable carrier may be used. Such carriers may be used as vehicles, adjuvants, excipients, or diluents in compositions of the invention. It is preferred that the pharmaceutically acceptable carrier be one which is chemically inert to the conjugate and one which has no detrimental side effects or toxicity under the conditions of use.

The choice of carrier will be determined in part by the particular conjugate, as well as by the particular method used to administer a composition comprising same. Accordingly, there is a wide variety of suitable formulations of the pharmaceutical composition of the present invention. These include composition for administration orally, or injected intravenously, subcutaneously, peritoneally, by aerosol, ophthalmically, intra-bladder, topically, vaginally and others.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the conjugate, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia, as well as pastilles comprising the conjugate in an inert base, such as gelatin and glycerin, or sucrose and acacia, emulsions, gels, and the like containing, in addition to the active ingredient, such carriers as are known in the art.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. The conjugates can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in dermal formulations include petroleum, animal, vegetable, or synthetic oils. Specific examples of oils include peanut, soybean, sesame, cottonseed, corn, olive, petrolatum, and mineral. Suitable fatty acids for use in dermal formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxy-ethylenepolypropylene copolymers, (d) amphoteric detergents such as, for example, alkyl-β-aminopriopionates, and 2-alkyl-imidazoline quaternary ammonium salts, and (3) mixtures thereof.

In order to minimize or eliminate irritation at the site of injection, compositions of the invention may contain one or more nonionic surfactants having a hydrophile-lipophile balance (HLB) of from about 12 to about 17. Suitable surfactants include polyethylene sorbitan fatty acid esters, such as sorbitan monooleate and the high molecular weight adducts of ethylene oxide with a lipophilic base, formed by the condensation of propylene oxide with propylene glycol. The parenteral formulations can be presented in unit-dose or multi-dose sealed containers, such as ampules and vials, or in bags ready for infusion and can be stored in a freeze-dried (lyophilized) condition requiring only the addition of the sterile liquid carrier, for example, water saline or glucose solutions, for injections, immediately prior to use. Extemporaneous injection solutions and suspensions can be prepared from sterile powders, granules, and tablets of the kind previously described.

Conjugates of the present invention may be made into injectable formulations. The requirements for effective pharmaceutical carriers for injectable compositions are well known to those of ordinary skill in the art (see Pharmaceutics and Pharmacy Practice, J.B. Lippincott Co., Philadelphia, Pa., Banker and Chalmers, eds., pp. 238-250 (1982), and ASHP Handbook on Injectable Drugs, Toissel, 4^{th} ed., pages 622-630 (1986).

Additionally, conjugates of the invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the conjugate, such carriers as are known in the art to be appropriate.

The compositions of the invention may comprise an ‘effective dose’ of a conjugate of the invention that is selected to achieve a substantial depletion, eradication or reduction to levels below stasis of a bacterial cell population, e.g., at least about 90%, at least about 91%, at least about 92%, at least about 93%, at least about 94%, at least about 95%, at least about 96%, at least about 97%, at least about 98%, at least about 99%, and further, at least about 99.9%, at least about 99.99%, at least about 99.999%, at least about 99.9999%, or more than 99.99999%, compared to an untreated infection. Thus, the compositions may be regarded as bacteriostatic or bactericidal. The effective dose may be based on the MIC, or MBEC, although is typically a higher dose to ensure eradication.

The effective dose of a conjugate is generally at least about from 2-fold to 100-fold less than the effective dose for the corresponding non-conjugated antibiotic, and specifically can be at least about 2-fold, 4-fold, 6-fold, 8-fold and 10-fold less than the dose for the corresponding non-conjugated antibiotic, and further at least about 20-fold, 30-fold, 40-fold, 50-fold, 60-fold, 70-fold, 80-fold, 90-fold and 100-fold less than the dose for the corresponding non-conjugated antibiotic.

The effective dose can be further expressed in terms of time-to-kill (eradication), which is generally shorter than the with the corresponding non-conjugated antibiotic, for example shorter by at least 2-fold, 3-fold, 4-fold, 5-fold, 6-fold, 7-fold, 8-fold, 9-fold and 10-fold, or more than time-to-kill of the corresponding non-conjugated antibiotic.

The effective does may be further articulated in terms of reducing bacterial burden by at least 1 log reduction or more. The reduction may be 2 log reduction, 3 log reduction, 4 log reduction and more, compared to a non-treated infection.

An effective dose of a conjugate may be a dose that achieves a concentration at the target site of at least about 0.0001 μM, at least about 0.001 μM, at least about 0.01 μM, at least about 0.1 μM, at least about 1 μM, at least about 5 μM, at least about 10 μM, at least about 50 μM, at least about 100 μM, at least about 500 μM, at least about 1 mM, at least about 5 mM, at least about 10 mM, at least about 100 mM.

An effective daily dose can be further expressed by way of ranges from about 0.05 mg to about 500 mg per kg human patient, for example at least about 0.05 mg, at least about 0.1 mg, at least about 0.5 mg, at least about 1 mg, at least about 5 mg, at least about 10 mg, at least about 50 mg, at least about 100 mg, at least about 500 mg, per kg human patient.

The composition of the invention may be formulated as a ‘unit dosage form’, comprising physically discrete units that are suitable as unitary dosages for human and animal subjects. Each unit contains a predetermined quantity of conjugates of the invention calculated in an amount sufficient to produce the desired effect in association with a pharmaceutically acceptable diluent, carrier or vehicle. The specifications for the unit dosage forms of the present invention depend on the particular conjugate employed and the effect to be achieved, and the pharmacodynamics associated with the conjugate in the host.

The compositions and pharmaceutical compositions of the invention comprising one or more of the conjugates of the invention can be implemented in a variety of application and methods in vivo and in vitro:

It is one of the important aspects of the invention to provide compositions for use in inhibiting a Gram-positive and/or a Gram-negative bacterial infection in vivo and in vitro

In numerous embodiments the compositions can comprise any compound of Formulae (I) through (X) as detailed above.

In certain embodiments the compositions can comprise a conjugate of vancomycin with MoTr.

The terms ‘Gram positive’ and ‘Gram negative’ bacteria (also GPB and GNB) refer herein to the two large groups of bacteria differentiated by the common technique of Gram staining based on their different cell wall constituents. GPB are usually characterized by a thick peptidoglycan layer and no outer lipid membrane, whilst GNB have a thin peptidoglycan layer and have an outer lipid membrane.

Owing to the structure of the present compounds that permits hydrogen bonding and lipophilic interactions, they are likely to involve one or more of the following mechanisms: a) improved cell surface association with negatively charged groups including but not limited to phosphates; b) effective translocation across the outer membrane leading to enhanced drug uptake, and c) disruption of peptidoglycan synthesis within the periplasmic space.

Irrespective of the exact mechanisms of action, the compounds of the invention can be used for inhibiting a GPB or a GNB infection, and by extension, preventing, alleviating a disease or condition in a subject suffering from or at risk of developing GPB or a GNB infection. Notable examples of GPB pathogenic bacteria are GPB cocci with the primary pathogens such as S. aureus, Strep. pyogenes, and Strep. pneumoniae along with species of lower virulence such as Staph. epidermidis, Staph. saprophyticus and Enterococcus faecalis. Examples of pathogenic GNB are Klebsiella, Acinetobacter, Pseudomonas aeruginosa, and E. coli, part of them constitute the normal flora but may become opportunistic.

In certain embodiments the GPB and GNB infections can comprise a GPB or GNB antibiotic resistant or multi-drug resistant bacterial strain.

In certain embodiments the GPB and GNB infections can consist of a GPB or GNB antibiotic resistant or multi-drug resistant bacterial strain.

In further embodiments the GPB infections can comprise a GPB strain selected from members of the genera Staphylococcus, Streptococcus and Enterococcus. These types are among the most common bacterial causes of clinical infections associated with a diverse spectrum of pathology, ranging from mild skin and soft tissue infections (SSTIs) to life-threatening systemic sepsis and bacterial meningitis.

In certain embodiments the GPB strain can be any one of Staphylococci sp., Streptococci sp., Enterococci sp., C. diptheriae, B. anthracis, C. difficile, methicillin-resistant Staphylococcus aureus (MRSA), glycopeptide-resistant Enterococci (GRE), multidrug resistant (MDR) Streptococcus pneumoniae, MDR Streptococcus agalactiae, Streptococcus pyogenes, Enterococcus faecium, Staphylococcus aureus, and multidrug-resistant Staphylococcus epidermidis (MRSE) and Bacillus anthracis (Anthrax).

The term ‘MRSA’ generally refers herein predominantly to a strain of S. aureus which is resistant to a large group of beta-lactam antibiotics, including penicillins and cephalosporins, and further methicillin, dicloxacillin, nafcillin, and oxacillin, the latter is also referred to as multidrug-resistant S. aureus or oxacillin-resistant S. aureus (ORSA). S. aureus is a cause of a variety of conditions in humans, including skin infections, pneumonia, mastitis, phlebitis, meningitis, scalded skin syndrome, osteomyelitis, urinary tract infections, and food poisoning.

In certain embodiments the GNB strain can be any one of Escherichia coli, Pseudomonas aeruginosa, Neisseria gonorrhoeae, Chlamydia trachomatis, Yersinia pestis, Vibrio cholerae, non-resistant and multidrug-resistant-Acinetobacter, and specifically Acinetobacter baumannii, Bacteroides fragilis, resistant Burkholderia cepacia, Enterobacter cloacae, Klebsiella pneumoniae, Proteus mirabilis, and Staphylococcus saprophyticus, and any drug resistant strain derived therefrom and extended spectrum beta-lactamase (ESBL) and metallo beta-lactamase (MBL) producing strains, including the more recently described NDM-1 type of K. pneumoniae and E. coli. They are an important medical challenge, especially in immunocompromised patients and hospital settings. Some strains such as Neisseria gonorrhoeae and Vibrio Cholerae pose a real challenge in developing countries.

It should be noted that GNB are intrinsically resistant to vancomycin because their outer membranes are impermeable to large glycopeptide molecules, with the exception of some non-gonococcal Neisseria species. Surprisingly, the vancomycin conjugates described herein were effective against a broad range of GNB strains.

This aspect can be further articulated in terms of methods of inhibiting a GPB and/or a GNB infection in vivo and in vitro, with the main step of contacting a biological or a non-biological surface, a cell, a tissue, or an organism with the compositions or formulations comprising the compounds of the invention.

Thus, it is meant that, apart from the aforementioned, compositions of the invention can be further be used to prevent or eradicate the formation of a bacterial biofilm. The term ‘biofilm’ encompasses herein an aggregate of any type of GPB or GNB embedded in a polysaccharide matrix that adheres to solid biological or non-biological surfaces. Biofilms may form on a wide variety of surfaces, including living tissues, medical devices, water system piping, and other surfaces, especially in hospital setting. Bacterial biofilms account for over 80% of hospital-acquired microbial infections. Biofilms play a significant role in the transmission of persistent infectious diseases, including cystic fibrosis pneumonia, infective endocarditis, UTI, periodontitis, chronic infections of the middle ear, and infections through medical devices such as intravenous catheters and artificial joints. Pseudomonas aeruginosa and Proteus mirabilis are common pathogens that form biofilms together in catheter-associated UTI. Currently available antibiotics often fail to eradicate biofilm-associated bacteria, necessitating multiple and intense antibiotic treatment regimens that drive the evolution of resistant pathogens and the exhaustion of last-resort antibiotics. As a consequence, biofilm-associated infections are the cause of significant morbidity and mortality in the clinic.

Further, it is another objective of the invention to provide compositions for use in preventing, alleviating, or treating a disease or a condition comprising a GPB and/or a GNB infection.

In numerous embodiments the GPB and/or the GNB infection can be a systemic infection, or an infection that is present in the blood stream and in other parts and organs.

In numerous embodiments the GPB and/or the GNB infection can be a localized infection, or an infection that is limited to a specific part of the body and has local symptoms.

In certain embodiments the compositions of the invention can be used for inhibiting GPB and GNB infections related to intraabdominal infection (IAI). IAI is a relatively common surgical complication from abdominal surgical procedures, including gastrointestinal dehiscence and fistula. Infections derived from the stomach, duodenum, and proximal small bowel can be caused by GPB and GNB. Colon-derived IAI can be caused by GNB Enterobacteriaceae (E. coli at the first place) and other GNB bacilli and Enterococci (GPB).

In certain embodiments the compositions of the invention can be used for inhibiting GPB and GNB infections related to uncomplicated or complicated urinary tract infections (UTI or cUTI). UTI refers herein to a urinary tract infection occurring in patients who have a normal, unobstructed genitourinary tract, who have no history of recent instrumentation, and whose symptoms are confined to the lower urinary tract. UTIs are most common in young women. Most UTIs are caused by E. coli (GNB). However, GPB have emerged as important causative agents of UTIs, particularly among elderly patients, often associated with co-morbidities, pregnant women and catheterized patients. cUTI refers herein to a urinary tract infection occurring in the setting of a urinary tract that has metabolic, functional, or structural abnormalities. cUTIs may involve both lower and upper tracts, including pyelonephritis. Their primary significance is that they significantly increase the rate of therapy failures. cUTI can be caused by GNB and GPB, with predominance of antibiotic resistant strains because of persistence and chronic manifestation of this condition.

In certain embodiments the compositions of the invention can be used for inhibiting GPB and GNB infections related to a community acquired pneumonia (CAP). CAP refers herein to a pneumonia acquired outside the hospital, most commonly related to Strep. pneumoniae (GPB), Haemophilus influenzae (GNB), atypical bacteria (do not color with Gram staining) such as Chlamydia pneumoniae, Mycoplasma pneumoniae and Legionella species, and viruses.

In certain embodiments the compositions of the invention can be used for inhibiting GPB and GNB infections related to a hospital acquired pneumonia (HAP, also nosocomial pneumonia). HAP refers herein to a pneumonia that occurs 48 h or more after hospital admission and not incubating at the admission time. Ventilator-associated pneumonia (VAP) represents a significant sub-set of HAP. The GNB bacilli are the major pathogens associated with HAP (e.g., P. aeruginosa and A. baumannii) and the pathophysiology is often manifested in the destructive effect on lung tissue.

In further embodiments the compositions of the invention can be used for inhibiting GPB and GNB infections related to a ventilator associated pneumonia (VAP).

In certain embodiments the compositions of the invention can be used for inhibiting GPB and GNB infections related to acute bacterial skin and skin structure infection (ABSSSI). ABSSSIs also referred to skin and skin structure infections (SSSIs) and skin and soft tissue infections (SSTIs), are infections of skin and associated soft tissues (such as loose connective tissue and mucous membranes). ABSSSIs has been related to certain types of bacteria, predominantly methicillin-resistant S. aureus (GPB). However, GNB become increasingly responsible ABSSSIs, accounting about 2% of hospitalizations in the US, for example.

In certain embodiments the compositions of the invention can be used for inhibiting GPB and GNB infections related to sepsis. As used herein, “sepsis” refers to a systemic disease caused by the presence of bacteria (bacteremia) or their products in the blood. Infections that lead to sepsis most often start in the lung, urinary tract, skin, or gastrointestinal tract, predominantly by GNB. GPB can produce specific toxins that can cause defined clinical syndromes in the absence of disseminated sepsis, e.g., botulism, anthrax, and diphtheria.

In certain embodiments the compositions of the invention can be used for inhibiting GPB and GNB infections related to bacterial meningitis, wherein they might have advantages in crossing the blood-brain barrier (BBB). The GPB strains associated with bacterial meningitis have been referred to above. Among GNB strains, the most common GNB cause of this condition is A. baumannii.

This aspect can be further articulated in the form of methods for preventing, alleviating, or treating a disease or a condition comprising a GPB and/or a GNB bacterial a subject who is suffering or at risk of suffering therefrom, with the main step of administering to the subject a composition comprising the compounds of the invention.

In numerous embodiments the composition is being administered via parenteral, enteral, topical, subcutaneous, dermal, vaginal or transdermal administration routes.

In certain embodiments the compositions and methods of the invention can further involve at least one additional therapeutic agent, including but not limited to additional antibiotics permitting to extend the spectrum of antibiotic activities, antibiotic and non-antibiotic agents that may allow synergistic effects to improve efficacy/toxicity balance, and especially beta-lactamase inhibitors to prevent antimicrobial resistance.

In certain embodiments the compositions and methods of the invention can involve additional drugs as per specific conditions, such as anti-inflammatory drugs and others. It is meant that the additional therapeutic agent can be administered by via parenteral, enteral, topical, subcutaneous, dermal, or transdermal administration routes.

In certain embodiment the compositions and methods of the invention can involve other agents meant to enhance their efficacy or mitigate toxicity, such as permeability and solubility enhancers, vitamins, nutrients, and others.

In certain embodiments the present compositions and methods can be implemented in a precision therapy for targeting specific pathogens (as opposed to the previously proposed broad-spectrum therapeutics), to further improve efficacy and avoid antimicrobial resistance.

In certain embodiments the method can further comprise a prior diagnosis of the bacterial strain causing the Gram-positive and/or the Gram-negative bacterial infection in the subject.

The invention further provides a series of products for specific applications.

One of them is a kit comprising the compositions of the invention in predetermined doses/concentrations or dosage forms and instructions for use. A kit further implies distribution of the predetermined doses or dosage forms in ready-to-use packages, containers, ampules, or vials, considering an effective dose the antibiotic conjugate at the target site.

In numerous embodiments an effective dose of the antibiotic conjugate comprised in the kit can be of at least about 0.0001 μM, 0.001 μM, 0.01 μM, 0.1 μM, 1 μM, 5 μM, 10 μM, 50 μM, 100 μM, 500 μM, 1 mM, 5 mM, 10 mM and at least about 100 mM.

In other embodiments an effective dose can be in a range from about 0.05 mg to about 500 mg per kg human patient, and specifically at least about 0.05 mg, 0.1 mg, 0.5 mg, 1 mg, 5 mg, 10 mg, 50 mg, 100 mg and at least about 500 mg, per kg human patient.

One attractive application would be a ready-to-use kit, wherein the predetermined dose of the composition is comprised in a container, or a bag adapted to IV administration.

Another application is a transdermal patch comprising the compounds of the invention. A transdermal patch implies that the compositions of the invention are released from the patch are or delivered in the system through the skin.

Transdermal delivery has a variety of advantages compared with the oral route, and in particular, in avoiding a significant first-pass effect of the liver that can prematurely metabolize drugs. With current delivery methods, successful transdermal drugs have molecular masses that are up to a few hundred Daltons (which is below the range of the present compounds), exhibit octanol-water partition coefficients that heavily favor lipids and require doses of milligrams per day or less. Significant efforts are being made to increase the number of drugs that are amenable to administration by this route. Recognizing the need to increase skin permeability, second-generation delivery strategies offer a series of chemical enhancers based on the traditional pharmaceutical toolbox and new formulations with chemical excipients. Another approach can apply the use of prodrugs through the addition of a cleavable chemical groups that typically increases drug lipophilicity and facilitates the transfer of a drug across the skin. Another approach to achieve an effective skin permeability can be iontophoresis, typically applying a continuous low-voltage current. The strongest asset of iontophoresis is that the rate of drug delivery scales with the electrical current, which can be readily controlled by a microprocessor or, in some cases, the patient. The third generation of transdermal delivery systems targets its effects to the stratum corneum by enabling a stronger disruption of the stratum corneum barrier while still protecting deeper tissues, using novel chemical enhancers, electroporation, cavitational ultrasound and more recently microneedles, thermal ablation and microdermabrasion (Arora, Prausnitz and Mitragotri) to deliver macromolecules, including therapeutic proteins and vaccines.

Another interesting application is a drug delivery system comprising the compositions of the invention. A drug delivery system implies an engineered technology for sustained, targeted delivery and/or controlled release of a therapeutic agent. The performance of a drug delivery system is highly dependent of on the success of the formulation of the desired active ingredient.

In certain embodiments the compositions of the invention can be formulated so that the compound is incorporated, encapsulated or attached to micro- or nanoparticles. Microemulsions and nano-drug delivery systems have been known for their abilities to increase the stability and water solubility of drugs, prolong the cycle time, increase the uptake rate of target cells or tissues, and reduce enzyme degradation, thereby improving the safety and effectiveness of drugs.

Nanoparticles are taken up by cells more efficiently than larger micromolecules and therefore, could be used as effective transport and delivery systems. Nanoparticles are generally <800 nm in at least one dimension and consist of different biodegradable materials such as natural or synthetic polymers, lipids, and surfactants. Polylactic/glycolic acid (PLGA) and PLGA-based nanoparticles are extensively used for the delivery of various macromolecules and nucleic acids. To achieve efficient drug delivery, it is important to understand the interactions of nanomaterials with the specific biological environment, targeting cell-surface receptors, drug release, multiple drug administration, stability of therapeutic agents and molecular mechanisms of cell signaling involved in pathobiology of the disease under consideration. It is also important to understand the barriers to drug such as stability of therapeutic agents in the living cell environment. A drug targeting system should be able to control the fate of a drug entering the organism upon reaching the specific organ or tissue.

Another interesting application is a device configured to provide point of care (POC) diagnostics in vitro of a GPB or a GNB strain in a sample of an individual, coupled to an injection device for subcutaneous administration of a composition of the invention. This application directly stems from the present finding of an advantageous subcutaneous option for the compositions of the invention.

A POC diagnostic using various portable and easy-to-use devices is well known in the art, and so are portable and wearable subcutaneous injection devices for administering compositions and drugs of interest. One example is a wearable injector device for convenient and cost-effective self-administration of large and viscous doses of drugs—Subcutaneous Wearable Bolus Injection (SWBI). The device utilizes forward osmosis technology to generate force for injection of larger pharmaceutical volumes subcutaneously. It is preloaded device with an invisible/automatic needle insertion for injective the drug. This, and similar systems can be easily adapted to include the presently described compositions. Ultimately the invention can be articulated in terms of use of the compositions of the invention for manufacturing of a medicament for preventing, alleviating, or treating a GNP and/or a GNB infection.

### EXAMPLES

In the following, compound numbering or labeling is independent of the labeling of the general formulae shown herein.

### Example 1: Preparation of Antibiotic-Cationic Molecular Transporter Conjugates

Compounds represented by the general formulas I-XIII (encompassed by general formulae I through X) can be prepared by any methodology known in the art. According to one such methodology, the cargo moiety, being, for example, an antibiotic, is modified by substituting at least one native atom thereof, e.g., an oxygen atom of a hydroxyl group or a carboxylic acid group, or a nitrogen atom of an amine group, etc., with the lipophilic and cationic moieties as disclosed herein. The chemical modification is selected to afford a compound of a particular structure according to the structures disclosed herein. In some cases, where the lipophilic and cationic moieties are associated to each other directly or via a linker moiety, as defined, the two moieties may be first associated to each other, and the resulting group is subsequently linked to the cargo moiety. In cases where each moiety is substituted to a different functionality of the cargo moiety, the end-product according to the invention may be formed by sequential or stepwise substitution.

In some cases, as depicted herein, the cationic moiety is covalently bound with the lipophilic moiety and the linker to form the molecular transporter moiety. This is then conjugated to the antibiotic cargo, for example vancomycin on one or several of the conjugation points as illustrated in Scheme 1 above. The conjugation between the molecular transporter and the cargo may be achieved through the formation of an amide bond in a polar solvent using appropriate common coupling reagents such as DCC, EDC HATU, HBTU, DIC TBTU, T3B PyBOP, PyAOP, TFFH, COMU, CDI, IBCF, ECF and (optionally) additives such as HOAt, HOBt in the presence of an appropriate base (e.g., DIPEA or NMM).

### 1.1 Synthesis of Compound I

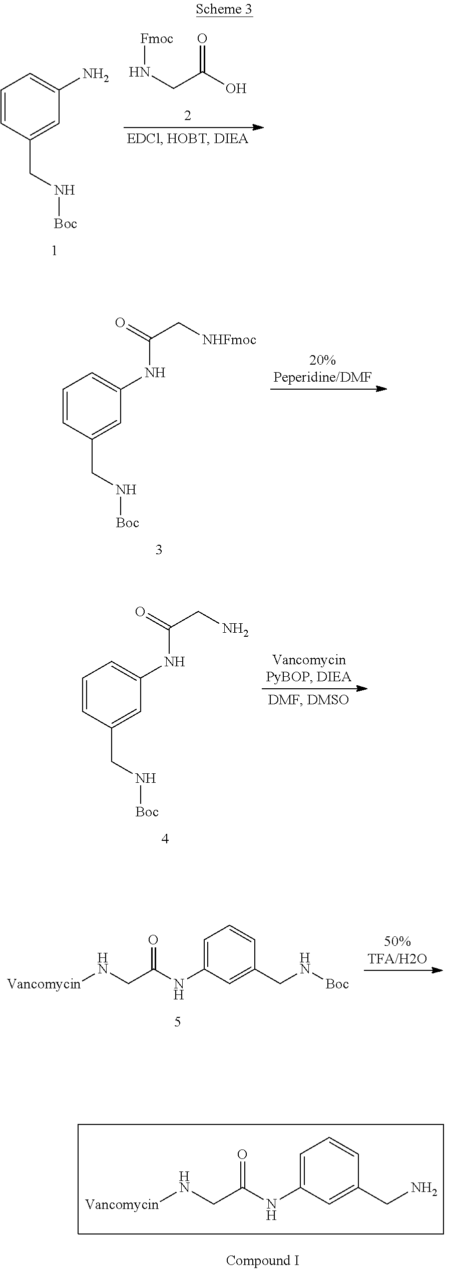

### Synthesis of Intermediate Compound 3

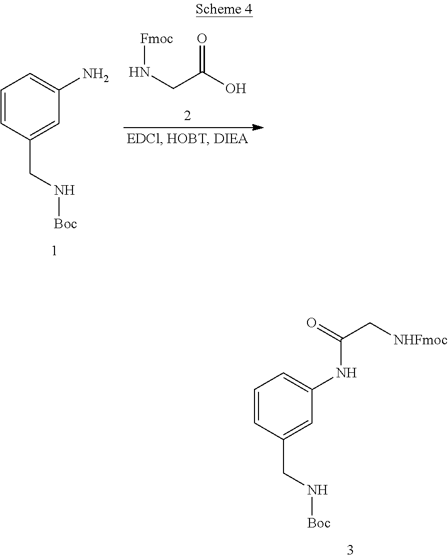

To a solution of 3-[(N-tert-Butoxycarbonylamino)methyl]aniline (compound 1, 200 mg, 900 μmol) and compound 2 (268 mg, 900 umol) in DCM (2.00 mL) was added DIPEA (465 mg, 3.60 mmol, 627 uL), HOBt (146 mg, 1.08 mmol) and EDCI (345 mg, 1.80 mmol). The mixture was stirred at 20° C. for 1 hr. The reaction mixture was diluted with 1 M HCl solution (2.0 mL) and extracted with EtOAc (30 mL), then washed with saturated NaCl (5.0 mL). The resulting organic layer was dried by anhydrous Na₂SO₄ and the solvent was removed by vacuum. Compound 3 (500 mg, crude) was obtained as a white solid. Presence of the desired product was confirmed by LC-MS. The material was carried forward to the next synthetic step without additional purification.

### Synthesis of Intermediate Compound 4

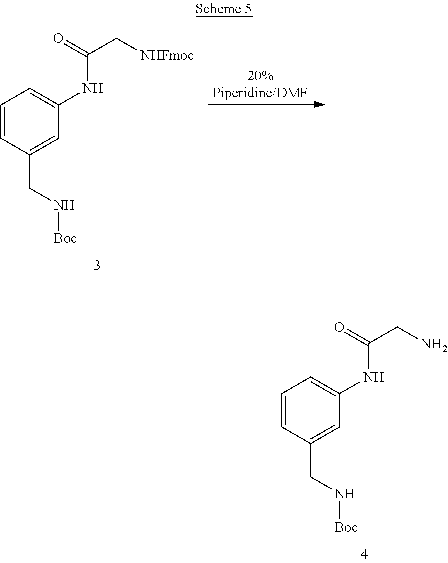

To Compound 3 (200 mg, 399 umol) was added piperidine (0.40 mL) in DMF (1.60 mL). The mixture was stirred at 20° C. for 0.5 hr. LC-MS indicated completed consumption of compound 3, with detection of one main peak having the desired mass. The residue was purified by preparatory HPLC (C18 column, mobile phase: water+0.075% TFA: acetonitrile). Compound 4 (100 mg, 90% yield) was obtained as a white solid, its identity confirmed by LCMS.

### Synthesis of Intermediate Compound 5

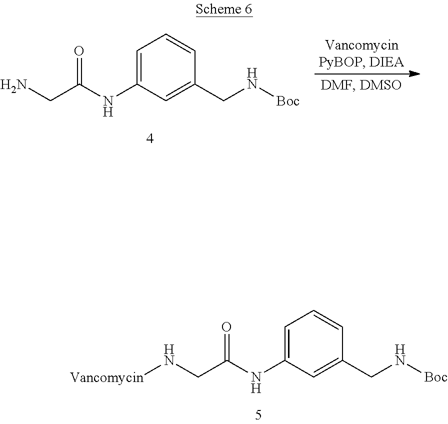

To a solution of compound 4 (100 mg, 0.25 mmol) and Vancomycin (184 mg, 0.13 mmol) in DMF (3.0 mL)/DMSO (3.0 mL) was added PyBOP (100 mg, 0.19 mmol) and DIPEA (82 mg, 0.64 mmol, 0.11 mL). The mixture was stirred at 30° C. for 16 hr. LC-MS indicated complete consumption of Vancomycin and one main peak with the desired product mass. The product was isolated by precipitation with MeCN (50.0 mL) and separated by centrifugation. The isolated material was dried under vacuum to give compound 5 (200 mg) as a white solid. The crude product was used in the next step without further purification.

### Synthesis of Compound I

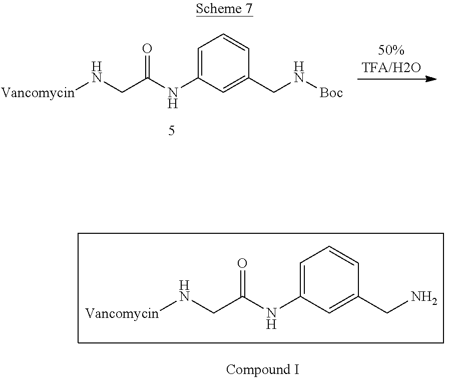

To compound 5 (200 mg, 0.12 mmol) was added an aqueous solution of TFA (50%, 2 mL). The mixture was stirred at 30° C. for 1 hr. LC-MS indicated complete consumption of the starting material (Compound 5) and the formation of one main peak with desired mass. The residue was purified by preparatory HPLC (C18 column, mobile phase: water+0.075% TFA: acetonitrile) to afford the desired product Compound I (40 mg, 21% yield) as a white solid. Identity was confirmed by 1H NMR and TOF-MS.

### 1.2 Synthesis of Compound II

The synthesis of the regioisomer Compound II was carried out according to a similar procedure as outlined above, starting with 4-[(N-tert-Butoxycarbonylamino)methyl]aniline as illustrated by Scheme 8 below:

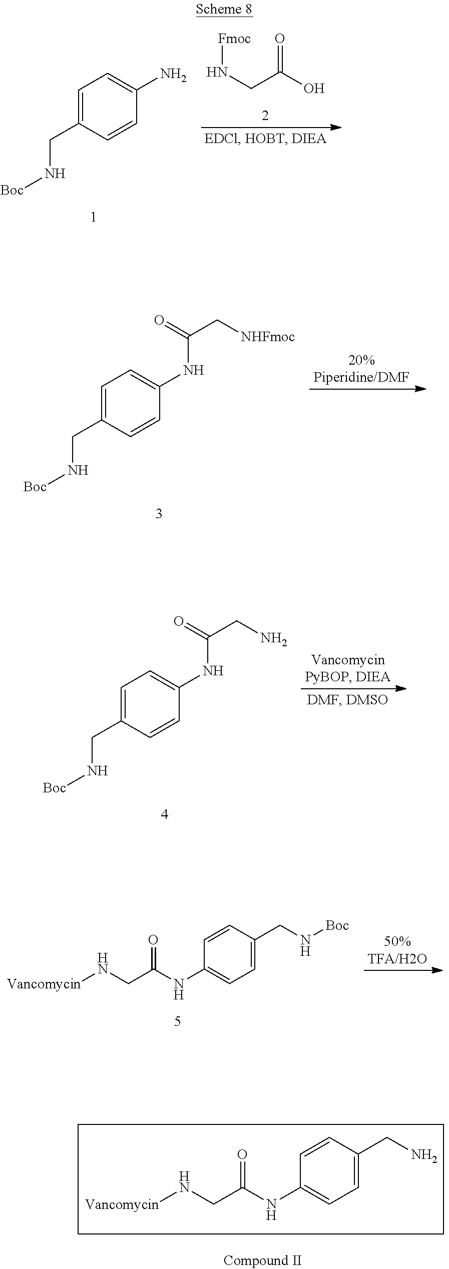

### 1.3 Synthesis of Compound III

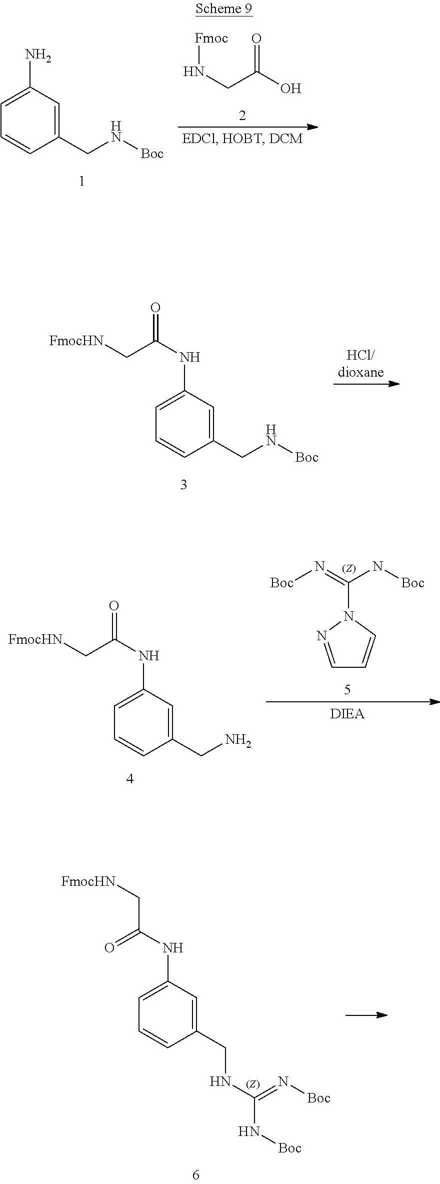

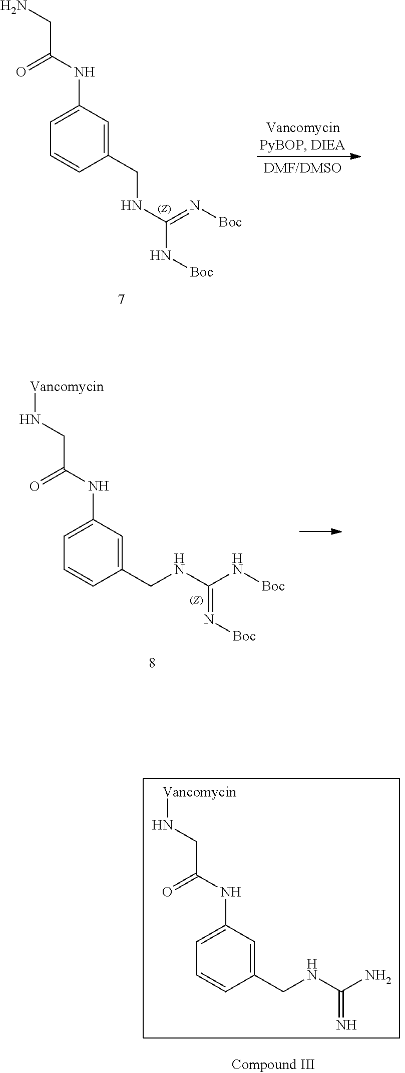

### Preparation of Intermediate Compound 3

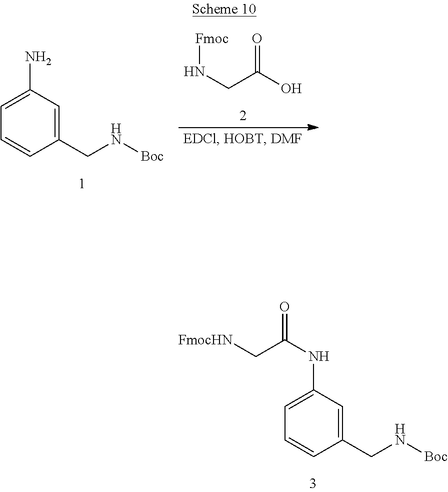

To a solution of compound 1 (400 mg, 900 umol) and compound 2 (268 mg, 900 umol) in DCM (2.00 mL) was added DIPEA (465 mg, 3.60 mmol), HOBt (146 mg, 1.08 mmol) and EDCI (345 mg, 1.80 mmol). The mixture was stirred at ambient temperature for 1 hr. The reaction mixture was diluted with 1 M HCl solution (2.00 mL) and the mixture was extracted with EtOAc (30 mL), then the solution was washed with saturated NaCl (5.0 mL). The resulting organic layer was dried by anhydrous Na₂SO₄ and the solvent was removed by vacuum. Compound 3 (500 mg, crude) was obtained as a white solid.

### Preparation of Intermediate Compound 4

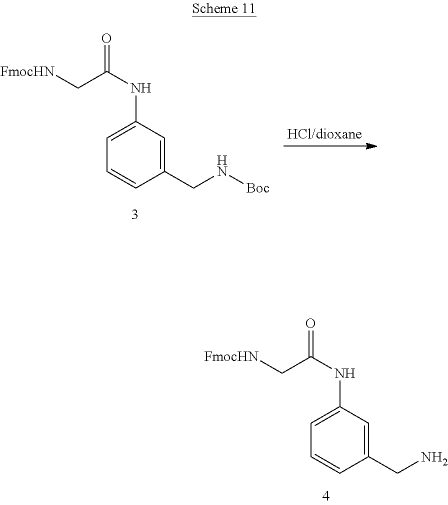

To compound 3 (300 mg, 598 umol) was added HCl/dioxane (6.0 mL, 4M). The mixture was stirred at ambient temperature for 0.5 hr. LC-MS showed Compound 3 was consumed completely and one main peak with desired mass was detected. The residue was purified by preparatory HPLC to get Compound 4 (200 mg, 83% yield) as a white solid.

### Preparation of Intermediate Compound 6

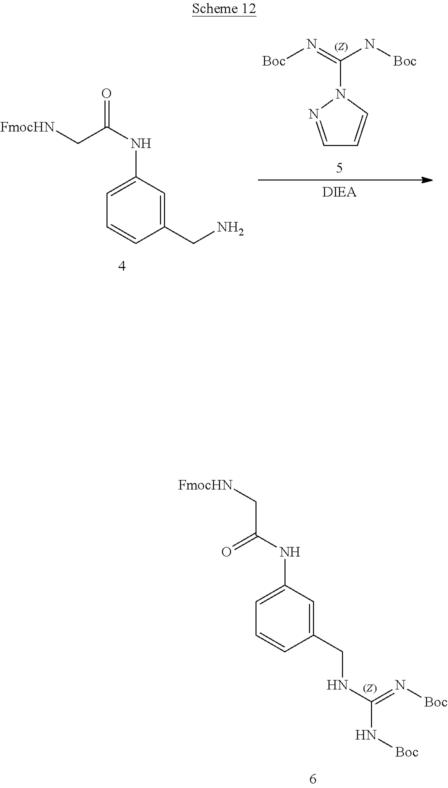

To a solution of compound 4 (180 mg, 448 umol) and compound 5 (139 mg, 448 umol) in MeOH (4.00 mL) was added DIPEA (58.0 mg, 448 umol). The mixture was stirred at ambient temperature for 1 hr. LC-MS showed compound 4 was consumed completely and one main peak with desired mass was detected. The solution was concentrated under reduced pressure to give Compound 6 (250 mg, crude) as a yellow liquid.

### Preparation of Intermediate Compound 7

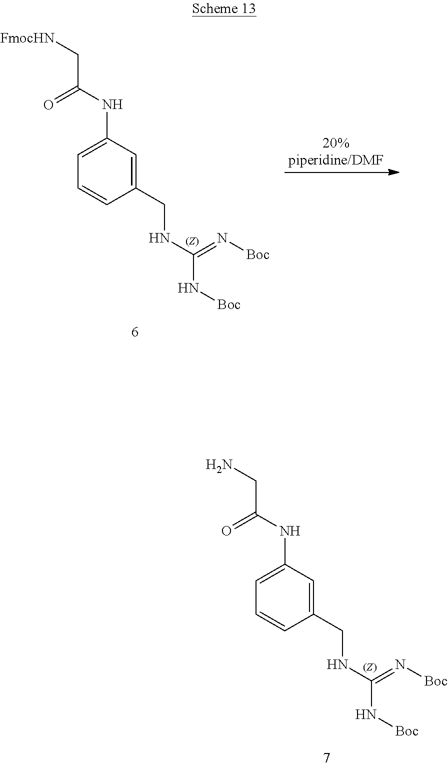

To compound 6 (250 mg, 388 umol) was added 20% piperidine (0.5 mL)/DMF (2 mL). The mixture was stirred at ambient temperature for 0.5 hr. LC-MS showed compound 6 was consumed completely and one main peak with desired mass was detected. The residue was purified by preparatory-HPLC (in presence of TFA) to give Compound 7 (110 mg, 67% yield) as a white solid.

### Preparation of Intermediate Compound 8

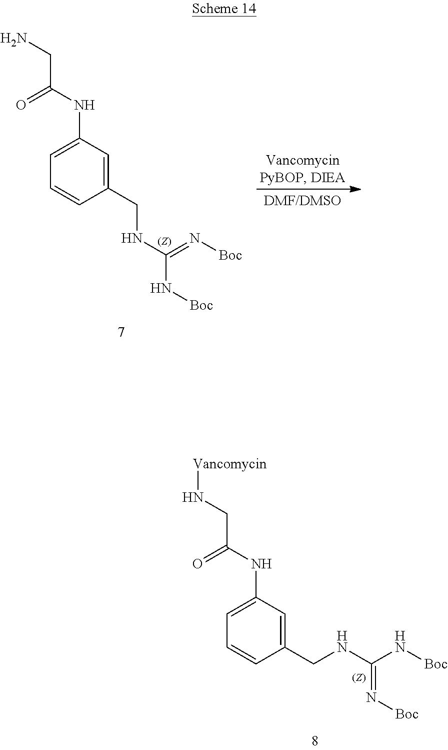

To a solution of compound 7 (100 mg, 187 umol) and Vancomycin (135 mg, 93.4 umol) in DMF (3 mL)/DMSO (3 mL) was added PyBOP (73 mg, 140 umol) and DIPEA (60.3 mg, 467 umol, 81.3 uL). The mixture was stirred at ambient temperature overnight. LC-MS showed complete consumption of vancomycin and one main peak with desired mass was detected. The product was isolated by precipitation with MeCN (50.0 mL) and centrifugation. The crude peptide was dried under vacuum to give compound 8 (150 mg, crude) as a white solid.

### Preparation of Compound III

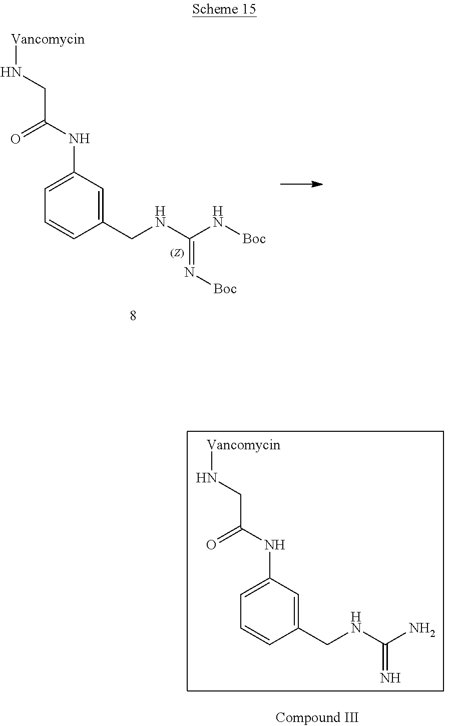

To compound 8 (150 mg, 81.0 umol) was added 50% water (2.00 mL)/TFA (2.00 mL). The mixture was stirred at 25° C. for 1 hr. LC-MS indicated that compound 8 had been fully consumed and one main peak with desired mass was detected. The residue was purified by preparatory HPLC to give Compound III (24 mg 18% yield) as a white solid. Identity was confirmed by 1H NMR and TOF-MS.

### 1.4 Synthesis of Compound IV (V-C9.2)

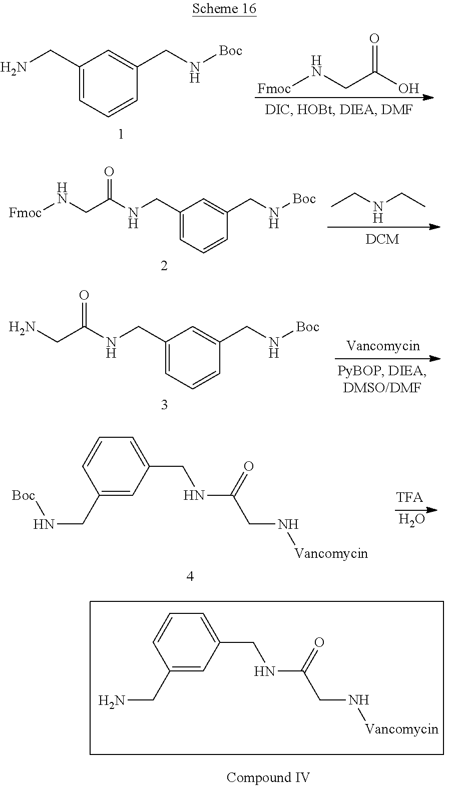

### Preparation of Intermediate Compound 2

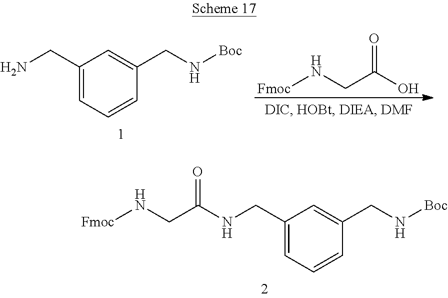

A mixture of compound 1 (500 mg, 2.12 mmol), 2-(9H-fluoren-9-ylmethoxycarbonylamino) acetic acid (629 mg, 2.12 mmol), HOBt (343 mg, 2.54 mmol), DIC (320 mg, 2.54 mmol) and DIEA (547 mg, 4.23 mmol) in DMF (5.0 mL) was stirred at 25° C. for 12 hrs. The reaction was diluted with 0.3 M HCl (200 mL) and extracted with DCM, dried over Na₂SO₄, filtered and concentrated under reduced pressure. The resulting residue was purified by column chromatography (SiO₂, EtOAc/DCM), affording compound 2 (466 mg, 42.7% yield) as a colourless oil. Identity of the title compound was confirmed by ¹H NMR and LCMS.

### Preparation of Intermediate Compound 3

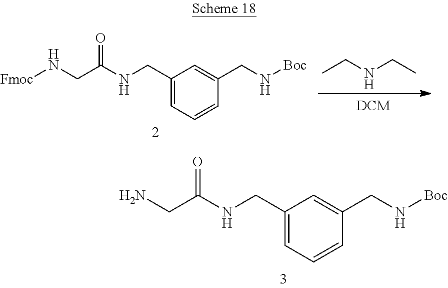

A mixture of compound 2 (460 mg, 892 umol), N-ethylethanamine (1.31 g, 17.8 mmol) in DCM (20.0 mL) was stirred at room temperature for 12 hrs. The reaction mixture was concentrated under reduced pressure and the resulting residue was purified by prep-HPLC (TFA condition), affording Compound 3 (120 mg, 45.9% yield) as a yellow oil. Identity of the title compound was confirmed by ¹H NMR and LCMS.

### Preparation of Intermediate Compound 4

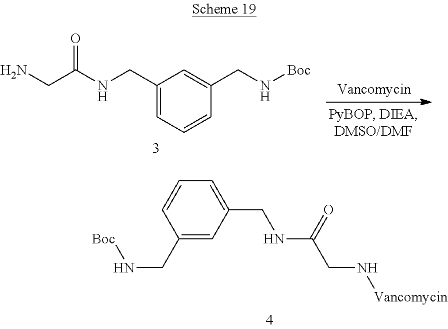

A mixture of Vancomycin (412 mg, 284 umol) in DMSO (0.50 mL) and DMF (0.50 mL) was added compound 3 (100 mg, 341 umol), PyBOP (222 mg, 426 umol), DIEA (184 mg, 1.42 mmol), then degassed and purged with nitrogen. The resulting mixture was stirred at ambient temperature under inert atmosphere for 8 hrs. The solution was triturated with MeCN and centrifuged. The resulting suspension was filtered, and the isolated product was dried under vacuum to afford compound 4 (611 mg, crude) as a white solid.

### Preparation of Compound IV (V-C9.2)

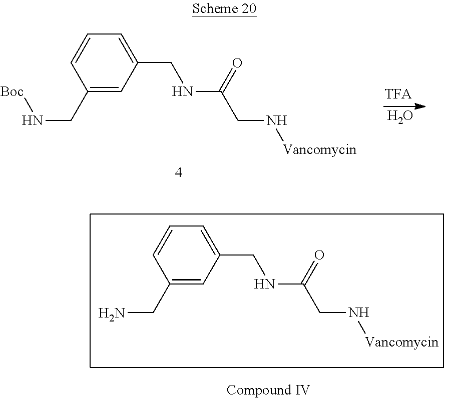

A mixture of compound 4 (200 mg, 116 umol) in TFA (2.50 mL) and water (2.50 mL) was stirred under inert atmosphere at room temperature until the reaction was deemed complete by LCMS. The reaction mixture was filtered, the filtrate concentrated under reduced pressure and the resulting residue was purified by prep-HPLC (TFA condition). The target Compound IV (93 mg, 49.2% yield, 93.4% purity) was isolated as white solid. Identity was verified by LC-MS.

### 1.5 Synthesis of Compound V (V-C11)

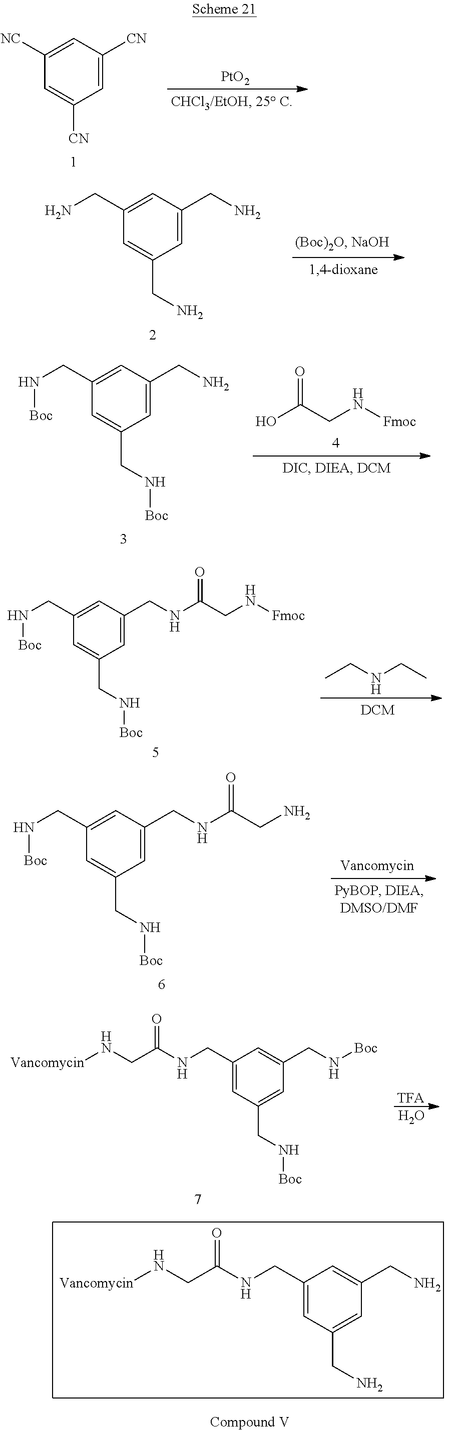

### Preparation of Intermediate Compound 2

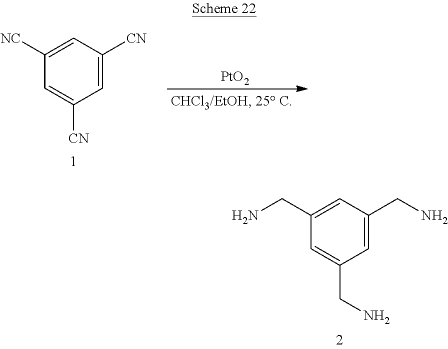 [SMILES: N#Cc1cc(C#N)cc(C#N)c1 ; "PtO2" ; NCc1cc(CN)cc(CN)c1]

A 100 mL reactor was charged with a mixture of compound 1 (500 mg, 3.26 mmol) and PtO₂ (111 mg, 490 umol) in CHCl₃ (1.0 mL) and EtOH (16.6 mL). The mixture was degassed and purged with nitrogen and then charged with hydrogen, while maintaining a constant system pressure at 71 psi during the reaction. The reaction was stirred at room temperature for 2 days, then purged with nitrogen, filtered and the filtrate was concentrated under reduced pressure. The crude compound 2 (750 mg) was obtained as a yellow solid, its identity verified by ¹H NMR and LCMS.

### Preparation of Intermediate Compound 3

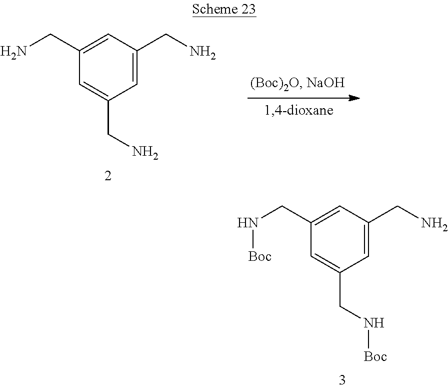 [SMILES: NCc1cc(CN)cc(CN)c1 ; CC(C)(C)OC(=O)NCc1cc(CN)cc(CNC(=O)OC(C)(C)C)c1]

To a solution of compound 2 (500 mg, 1.82 mmol, 3HCl) in 1,4-dioxane (10 mL) was added (Boc)₂O (238 mg, 1.09 mmol) and NaOH (0.2 M, 22.8 mL). The mixture was stirred at ambient temperature until deemed complete by LCMS. The reaction mixture was concentrated under reduced pressure, the resulting residue was diluted with water and extracted with DCM. The combined organic layers were washed with brine, dried over Na₂SO₄, filtered and concentrated under reduced pressure. The obtained residue was purified by prep-HPLC (TFA condition) to afford Compound 3 (129 mg, 14.8% yield, TFA salt) as a white solid. Identity was confirmed by LCMS.

### Preparation of Intermediate Compound 5

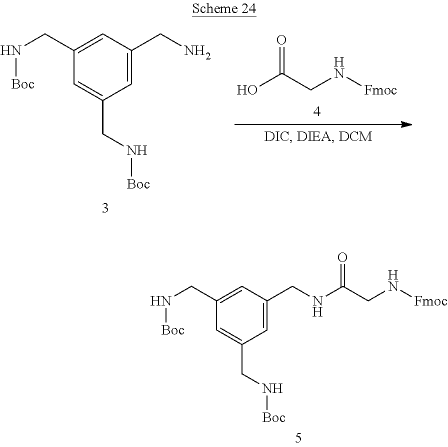

To a solution of compound 3 (124 mg, 259 umol, TFA) in DCM (5 mL) was added 2-(9H-fluoren-9-ylmethoxycarbonylamino) acetic acid (92 mg, 310 umol), DIC (39 mg, 310 umol), HOBt (42 mg, 310 umol) and DIEA (100 mg, 776 umol). The mixture was stirred at room temperature overnight. The reaction mixture was concentrated under reduced pressure and the resulting residue was purified by column chromatography (SiO₂, Petroleum ether/Ethyl acetate=5/1 to 1/1). The isolated product compound 5 (98.0 mg, 58.8% yield) was obtained as a light-yellow oil. Identity of the title compound was verified by ¹H NMR.

### Preparation of Intermediate Compound 6

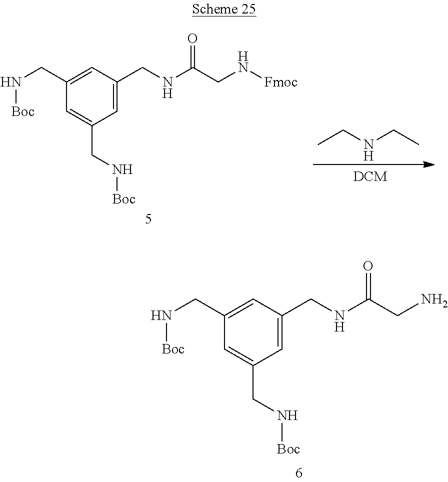

A mixture of compound 5 (110 mg, 171 umol), N-ethylethanamine (187 mg, 2.56 mmol) in DCM (2 mL) was stirred at ambient temperature until the reaction was deemed complete. The reaction mixture was concentrated under reduced pressure and the resulting residue was purified by prep-HPLC (TFA condition) to afford compound 6 (33 mg, 45.8% yield) as a light-yellow oil.

### Preparation of Intermediate Compound 7

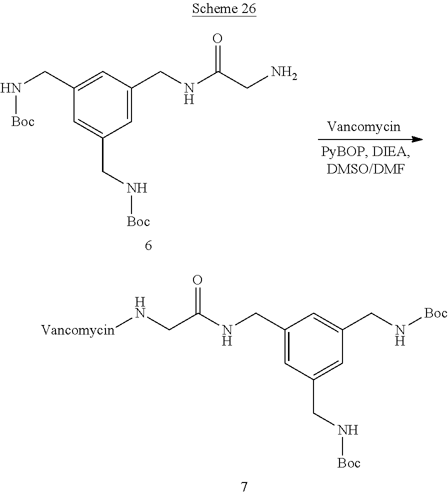

To a solution of Vancomycin (110 mg, 75.9 umol) in 50:50 DMSO:DMF (2 mL) was added compound 6 (32 mg, 76 umol), PyBOP (59 mg, 114 umol) and DIEA (29 mg, 228 umol), The reaction mixture was purged with nitrogen and stirred at ambient temperature under inert atmosphere for 8 h. The mixture was filtered, the filtrate concentrated under reduced pressure and the residue was purified by prep-HPLC (TFA condition). The title compound 7 (158 mg, crude) was obtained as white solid, as verified by LCMS.

### Preparation of Compound V (V-C11)

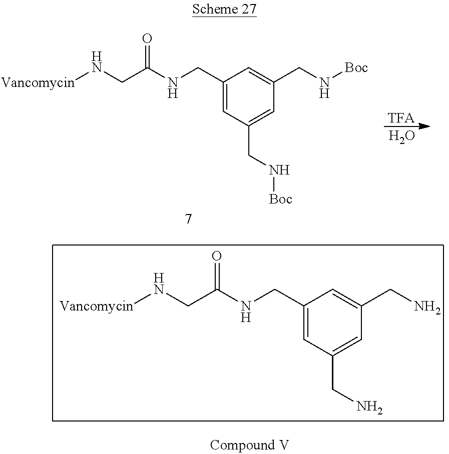

A mixture of compound 7 (100 mg, 54 umol), TFA (1.54 g, 13.5 mmol), and water (1 mL) was purged with nitrogen, and the mixture was stirred at ambient temperature for 1 h. The mixture was combined with another batch of crude product (50 mg). The resulting mixture was filtered, the filtrated was concentrated and the resulting residue was purified by prep-HPLC (TFA condition). The compound V-C11 (24 mg, 17.9% yield, 95.8% purity) was obtained as white solid.

### 1.6 Synthesis of Compound VI (V-D2.2)

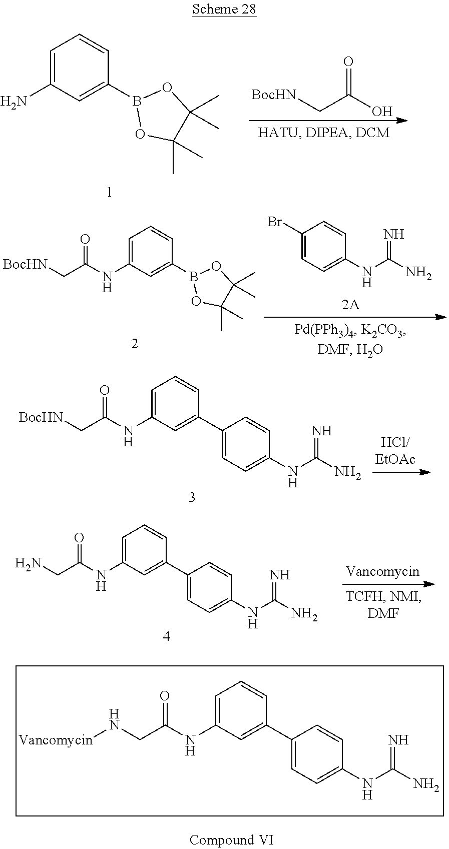

### Preparation of Intermediate Compound 2

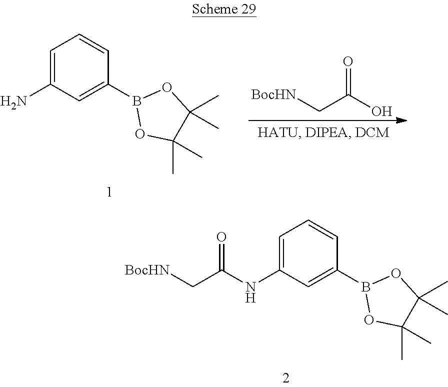 [SMILES: CC1(C)OB(c2cccc(N)c2)OC1(C)C ; CC(C)(C)OC(=O)NCC(=O)O ; CC(C)(C)OC(=O)NCC(=O)Nc1cccc(B2OC(C)(C)C(C)(C)O2)c1]

To a mixture of 2-(tert-butoxycarbonylamino)acetic acid (1.20 g, 6.85 mmol), HATU (2.60 g, 6.85 mmol) and DIPEA (1.18 g, 9.13 mmol) in DCM (20 mL) was added Compound 1 (1.00 g, 4.56 mmol) at ambient temperature. The reaction was stirred for 12 h until deemed complete by LCMS. The reaction mixture was concentrated under reduced pressure and the resulting residue was purified by column chromatography (SiO₂, Petroleum ether/Ethyl acetate=1/0 to 3/1) to give Compound 2 (2.10 g, crude) as a light-yellow oil.

### Preparation of Intermediate Compound 2A

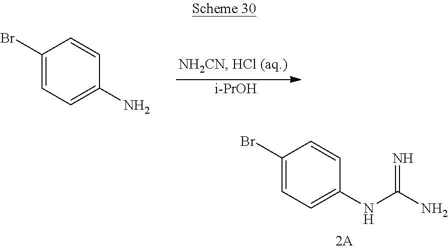 [SMILES: "i-PrOH" ; Nc1ccc(Br)cc1 ; N=C(N)Nc1ccc(Br)cc1]

To a mixture of p-bromoaniline (10.0 g, 58.1 mmol) and cyanamide (4.88 g, 116 mmol) in IPA (100 mL) was added HCl (12 M, 5.91 mL) at ambient temperature. The reaction was stirred at 80° C. for 12 h. The reaction mixture was quenched by saturated sodium carbonate solution (60 mL) and filtered. The solid was washed with H₂O (60 mL) to afford Compound 2A (9.00 g, crude) as a brown solid.

### Preparation of Intermediate Compound 3

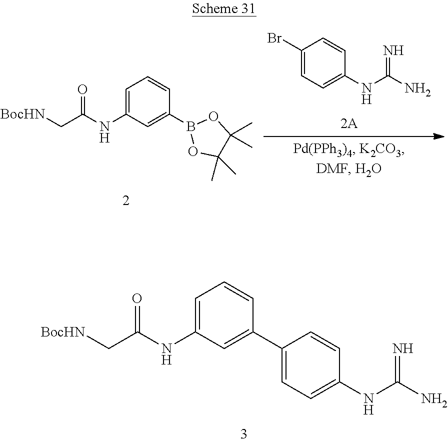 [SMILES: CC(C)(C)OC(=O)NCC(=O)Nc1cccc(B2OC(C)(C)C(C)(C)O2)c1 ; N=C(N)Nc1ccc(Br)cc1 ; CC(C)(C)OC(=O)NCC(=O)Nc1cccc(-c2ccc(NC(=N)N)cc2)c1]

To a mixture of Compound 2A (950 mg, 4.44 mmol) and Compound 2 (2.00 g, 5.33 mmol,) in dioxane (10 mL) and H₂O (1 mL) were added K₂CO₃ (1.23 g, 8.88 mmol) and Pd(PPh₃)₄ (257 mg, 222 umol) at ambient temperature. The mixture was stirred at 80° C. for 12 h under N₂ atmosphere until deemed complete by LCMS. The reaction mixture was filtered, and the filtrate was concentrated. The resulting residue was purified by prep-HPLC (column: Phenomenex luna C18 250 mm*100 mm*15 um; mobile phase: [water (0.05% HCl)-ACN]; B %: 15%-45%, 20 min) to give Compound 3 (550 mg, 29.5% yield, HCl) as a yellow solid.

### Preparation of Intermediate Compound 4

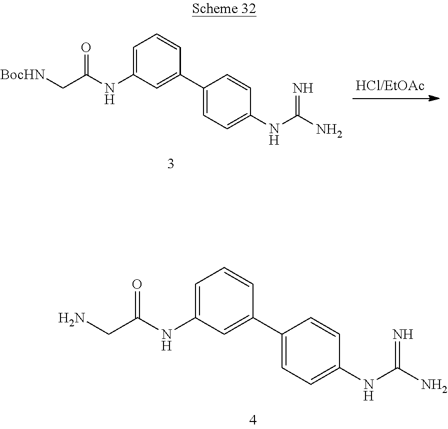 [SMILES: CC(C)(C)OC(=O)NCC(=O)Nc1cccc(-c2ccc(NC(=N)N)cc2)c1 ; N=C(N)Nc1ccc(-c2cccc(NC(=O)CN)c2)cc1]

A mixture of Compound 3 (550 mg, 1.31 mmol) in HCl/EtOAc (4 M, 10 mL) was stirred at ambient temperature for 0.5 h. The reaction mixture was concentrated under reduced pressure to afford Compound 4 (500 mg, crude) as a yellow solid.

### Preparation of Compound VI (V-D2.2)

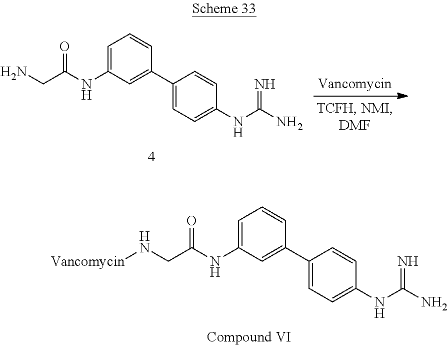

To a solution of Vancomycin (600 mg, 404 umol) and Compound 4 (194 mg, 606 umol) in DMF (5 mL) were added TCFH (227 mg, 808 umol) and NMI (166 mg, 2.02 mmol, 161 uL) at ambient temperature. The reaction was stirred for 12 h until deemed complete by LCMS. The residue was purified by prep-HPLC (column: Phenomenex Gemini-NX 150*30 mm*5 um; mobile phase: [water (0.1% TFA)-ACN]; B %: 1%-20%, 32 min) to give V-D1.2 (53.8 mg, 7.7% yield, 99.0% purity) as a white solid. Identity was confirmed by 1H-NMR and TOF-MS.

### 1.7 Synthesis of Compound VII (V-D7.2)

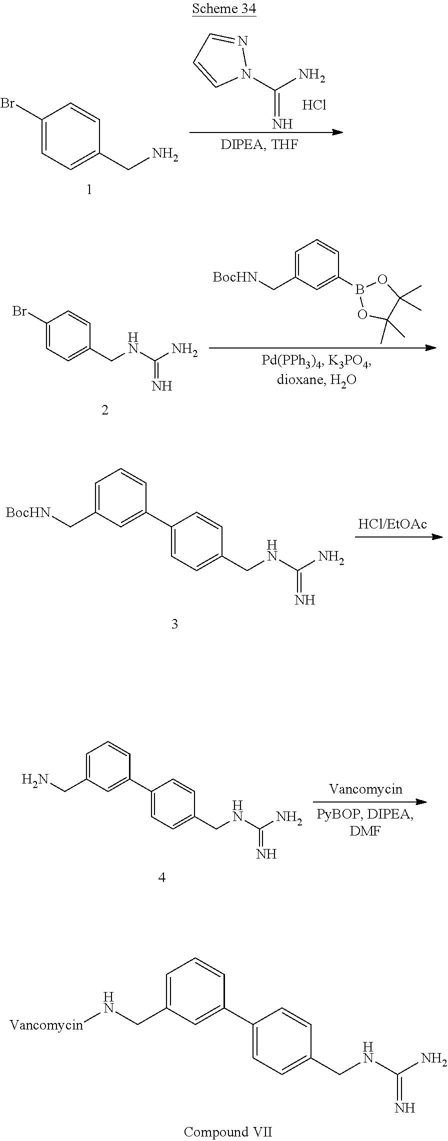

### Preparation of Intermediate Compound 2

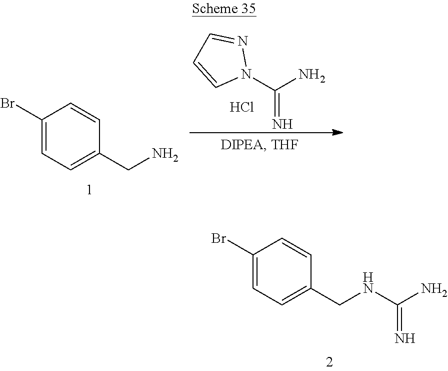 [SMILES: NCc1ccc(Br)cc1 ; N=C(N)n1cccn1 ; Cl ; N=C(N)NCc1ccc(Br)cc1]

A mixture of Compound 1 (5.00 g, 26.9 mmol), pyrazole-1-carboxamidine; hydrochloride (3.94 g, 26.9 mmol) and DIPEA (3.48 g, 26.9 mmol) in THF (75 mL) was stirred at ambient temperature under inert atmosphere until the reaction was deemed complete by LCMS. The reaction mixture was concentrated under reduced pressure and the resulting residue was purified by reversed-phase MPLC (TFA condition) to give Compound 2 (9.00 g, crude, TFA) as a light-yellow oil.

### Preparation of Intermediate Compound 3

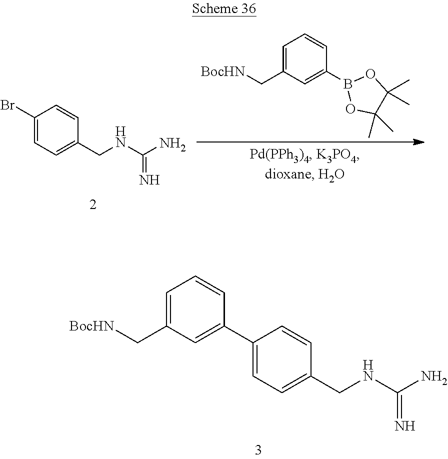 [SMILES: N=C(N)NCc1ccc(Br)cc1 ; CC(C)(C)OC(=O)NCc1cccc(B2OC(C)(C)C(C)(C)O2)c1 ; CC(C)(C)OC(=O)NCc1cccc(-c2ccc(CNC(=N)N)cc2)c1]

A mixture of Compound 2 (650 mg, 1.9 mmol), tert-butyl N-[[3-(4,4,5,5-tetramethyl-1,3,2-dioxaborolan-2-yl)phenyl]methyl]carbamate (540 mg, 1.6 mmol), K₃PO₄ (807 mg, 3.8 mmol) and Pd(PPh₃)₄ (220 mg, 190 umol) in dioxane (15 mL) and H₂O (1 mL) was purged with nitrogen at ambient temperature, and then the mixture was stirred at 80° C. for 12 h under inert atmosphere. The reaction mixture was concentrated under reduced pressure and the residue was purified by prep-HPLC (column: Phenomenex luna C18 (250*70 mm, 15 um)) to yield Compound 3 (600 mg, 80.8% yield) as a yellow solid. Identity of the intermediate was confirmed by 1H-NMR.

### Preparation of Intermediate Compound 4

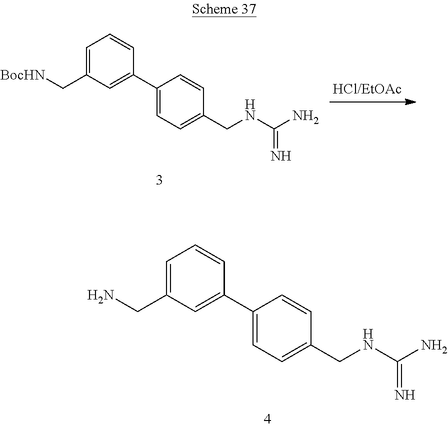 [SMILES: CC(C)(C)OC(=O)NCc1cccc(-c2ccc(CNC(=N)N)cc2)c1 ; N=C(N)NCc1ccc(-c2cccc(CN)c2)cc1]

A mixture of Compound 3 (600 mg, 1.69 mmol) in HCl/EtOAc (4 M, 10 mL) was stirred at 25° C. for 0.5 h. The reaction mixture was concentrated under reduced pressure to give Compound 4 (510 mg, crude) as a light-yellow solid.

### Preparation of Compound VII (V-D7.2)

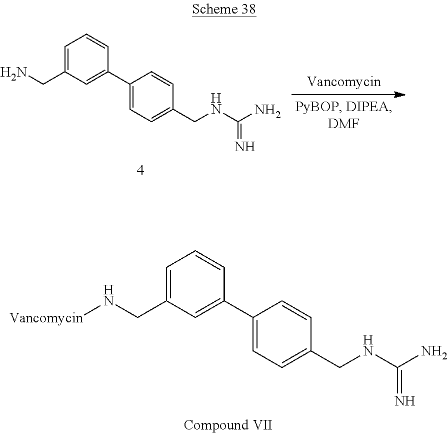

To a mixture of Vancomycin (600 mg, 404 umol, 1.00 eq) and Compound 4 (235 mg, 808 umol, 2.00 eq, HCl) in DMF (10 mL) were added DIPEA (261 mg, 2.02 mmol, 352 uL, 5.00 eq) and PyBOP (420 mg, 808 umol, 2.00 eq) at 25° C. The reaction was stirred ambient temperature for 12 h until deemed complete by LC-MS. The residue was purified by prep-HPLC (column: Phenomenex Luna C18 100*30 mm*5 um) to give Compound VII (239 mg, 34.5% yield, 98.4% purity) as a white solid. The title compound was characterised by mass spectrometry.

### 1.8 Synthesis of Compound VIII (V-D8.2)

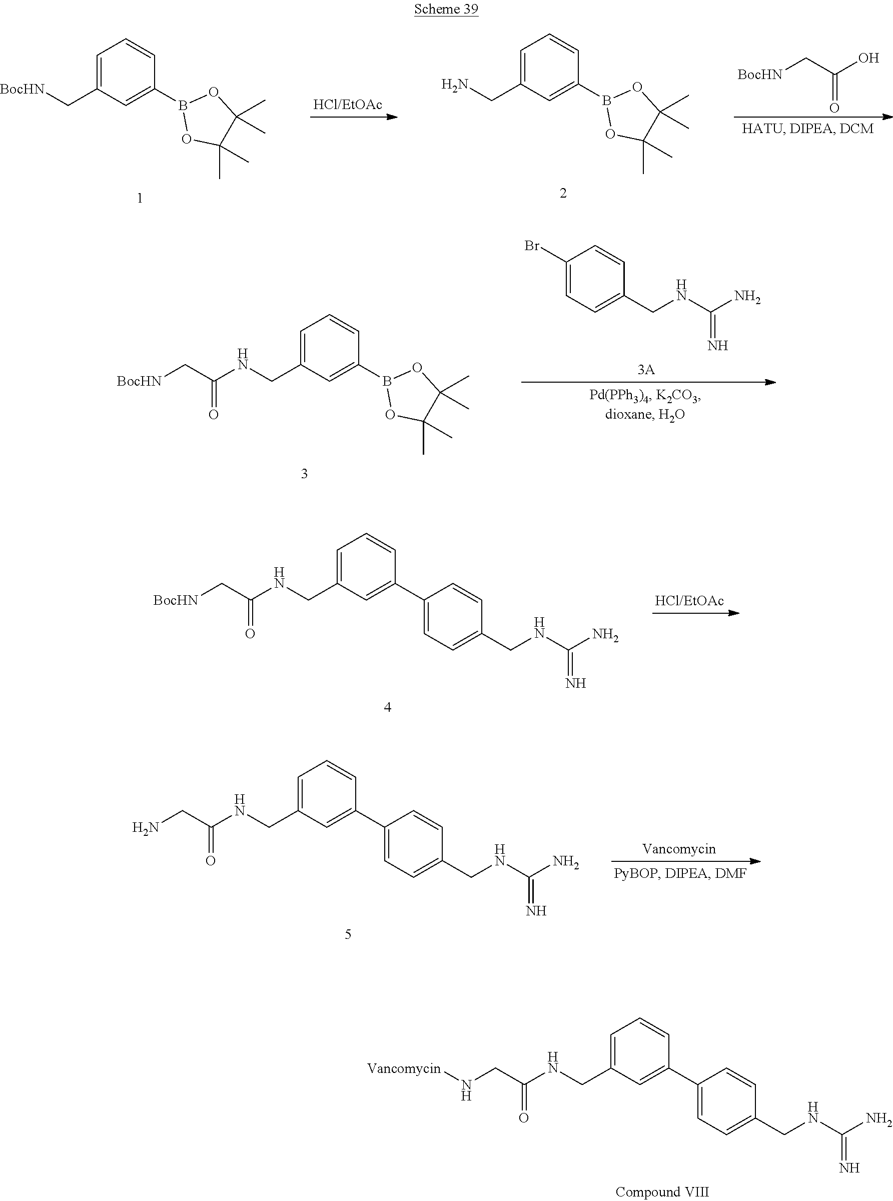

### Preparation of Intermediate Compound 2

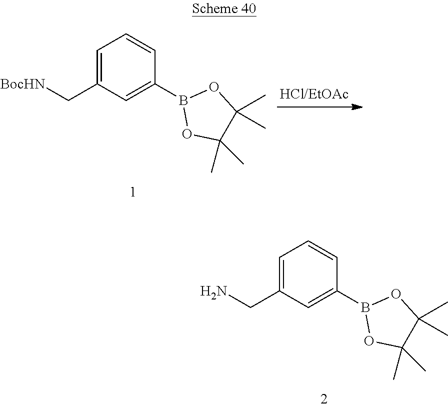 [SMILES: CC(C)(C)OC(=O)NCc1cccc(B2OC(C)(C)C(C)(C)O2)c1 ; CC1(C)OB(c2cccc(CN)c2)OC1(C)C]

A mixture of Compound 1 (750 mg, 2.25 mmol) in HCl/EtOAc (4 M, 7.48 mL) was stirred at ambient temperature for 1 h. The reaction mixture was concentrated under reduced pressure to afford Compound 2 (620 mg, crude) as a white solid.

### Preparation of Intermediate Compound 3

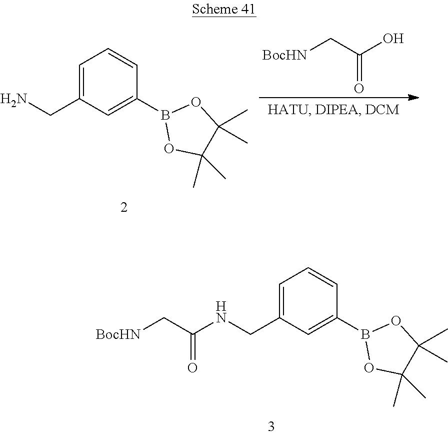 [SMILES: CC1(C)OB(c2cccc(CN)c2)OC1(C)C ; CC(C)(C)OC(=O)NCC(=O)O ; CC(C)(C)OC(=O)NCC(=O)NCc1cccc(B2OC(C)(C)C(C)(C)O2)c1]

To a mixture of 2-(tert-butoxycarbonylamino)acetic acid (606 mg, 3.46 mmol) in DCM (5 mL) were added HATU (1.31 g, 3.45 mmol), DIPEA (595 mg, 4.60 mmol) and Compound 2 (620 mg, 2.30 mmol) at room temperature. The mixture was stirred until deemed complete by LC-MS (˜12 h). The reaction mixture was concentrated under reduced pressure and the resulting residue was purified by column chromatography (SiO₂, Petroleum ether/Ethyl acetate=1/0 to 3/1) to afford Compound 3 (922 mg, crude) as a colourless oil.

### Preparation of Intermediate Compound 3A

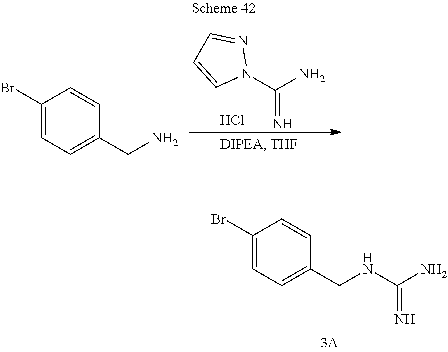 [SMILES: NCc1ccc(Br)cc1 ; N=C(N)n1cccn1 ; Cl ; N=C(N)NCc1ccc(Br)cc1]

A mixture of 4-bromophenyl methylamine (5.00 g, 26.9 mmol), pyrazole-1-carboxamidine; hydrochloride (3.94 g, 26.9 mmol) and DIPEA (3.48 g, 26.9 mmol) in THF (75 mL) was stirred at ambient temperature under N₂ atmosphere for 12 h. The reaction mixture was concentrated under reduced pressure and the residue was purified by RP-MPLC (TFA condition) to give Compound 3A (9.00 g, crude, TFA) as a light-yellow oil.

### Preparation of Intermediate Compound 4

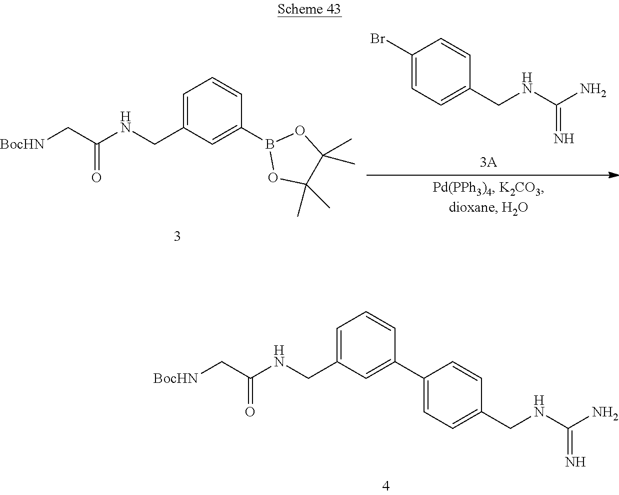 [SMILES: CC(C)(C)OC(=O)NCC(=O)NCc1cccc(B2OC(C)(C)C(C)(C)O2)c1 ; N=C(N)NCc1ccc(Br)cc1 ; CC(C)(C)OC(=O)NCC(=O)NCc1cccc(-c2ccc(CNC(=N)N)cc2)c1]

To a mixture of Compound 3 (870 mg, 2.23 mmol) in dioxane (5 mL) and H₂O (0.3 mL) were added Compound 3A (763 mg, 2.23 mmol), K₂CO₃ (618 mg, 4.47 mmol) and Pd(PPh₃)₄ (129 mg, 112 umol) at room temperature, and then the mixture was heated and stirred at 85° C. for 12 h under N₂ atmosphere. The reaction mixture was purified by prep-HPLC (column: Phenomenex luna C18 (250*70 mm, 15 um)) to afford Compound 4 (581 mg, 63.3% yield) as a white solid. Identity of the product was verified by 1H NMR.

### Preparation of Intermediate Compound 5

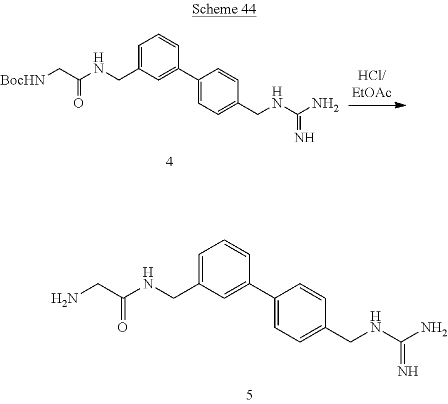 [SMILES: CC(C)(C)OC(=O)NCC(=O)NCc1cccc(-c2ccc(CNC(=N)N)cc2)c1 ; N=C(N)NCc1ccc(-c2cccc(CNC(=O)CN)c2)cc1]

A mixture of Compound 4 (581 mg, 1.41 mmol) in HCl/EtOAc (4 M, 15 mL) was stirred at ambient temperature for 0.5 h until the reaction was deemed complete. The reaction mixture was concentrated under reduced pressure to give Compound 5 (586 mg, crude) as a white solid.

### Preparation of Compound VIII (V-D8.2)

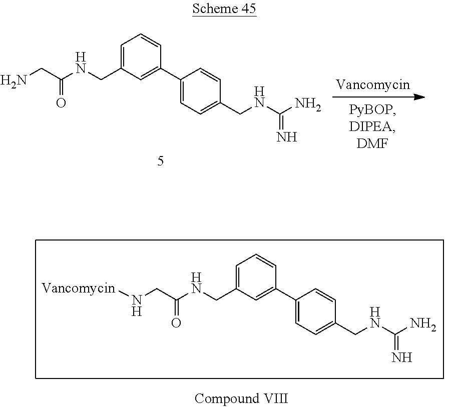

To a mixture of Vancomycin (600 mg, 404 umol, 1.00 eq) and Compound 5 (211 mg, 607 umol) in DMF (10 mL) were added PyBOP (231 mg, 444 umol) and DIPEA (261 mg, 2.02 mmol,) at room temperature. The mixture was stirred for 12 h and then purified by prep-HPLC (column: Phenomenex Luna C18 100*30 mm, 5 um) to afford Compound VIII (89 mg, 11.8% yield, 99.6% purity, TFA) as a white solid.

### 1.9 Synthesis of Compound IX (V-rBn)

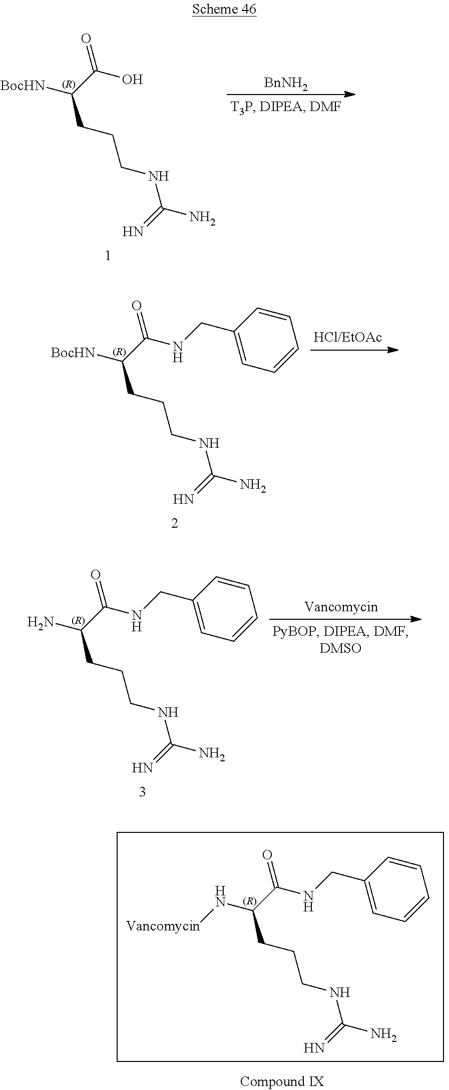

### Preparation of Intermediate Compound 2

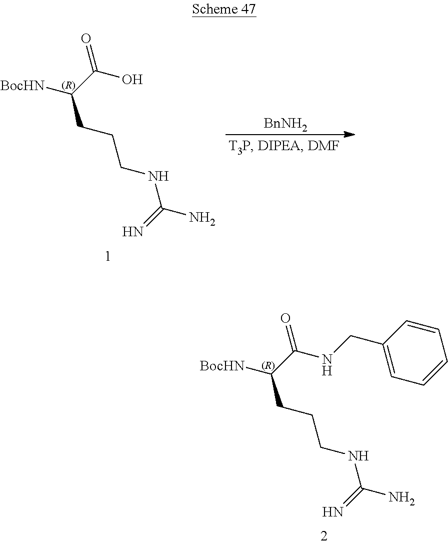

To a solution of Compound 1 (2.00 g, 6.44 mmol) in DMF (20 mL) were added T₃P (6.14 g, 9.65 mmol, 50% in EtOAc), DIPEA (3.33 g, 25.7 mmol) and BnNH₂ (828 mg, 7.72 mmol) at 15° C. The mixture was stirred at 15° C. for 12 h. The reaction mixture was concentrated under reduced pressure and the resulting residue was purified by prep-HPLC (Agela DuraShell C18 250*70 mm*10 um) to afford Compound 2 (1.30 g, 50.5% yield) as a white solid. The structure was verified by 1H-NMR.

### Preparation of Intermediate Compound 3

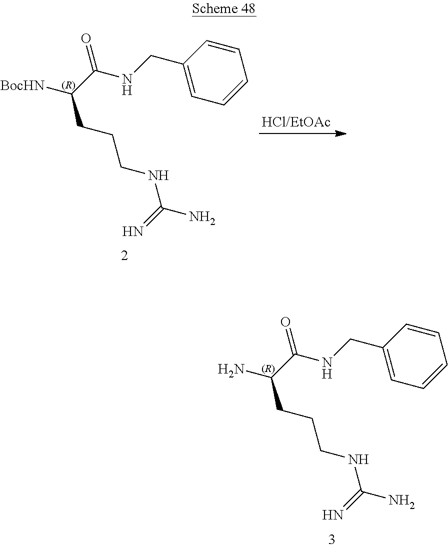

A mixture of Compound 2 (1.30 g, 3.25 mmol) in HCV/EtOAc (4 M, 20 mL) was stirred at 15° C. for 2 h. The reaction mixture was concentrated under reduced pressure to afford Compound 3 (1.00 g, crude, HCl) as a white solid.

### Preparation of Compound IX (V-rBn)

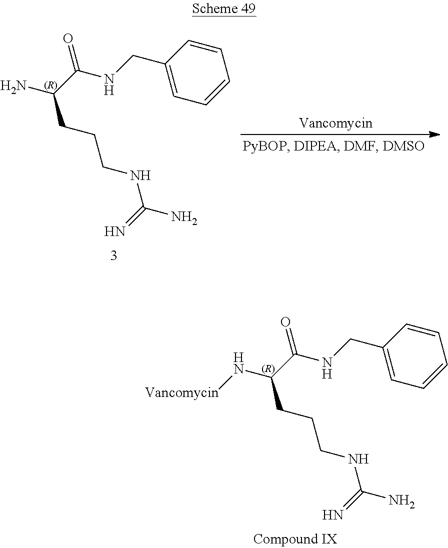

To a solution of Compound 3 (96.9 mg, 323 umol,) and Vancomycin (400 mg, 269 umol) in DMF (3 mL) and DMSO (3 mL) were added PyBOP (210 mg, 404 umol) and DIPEA (174 mg, 1.35 mmol) at 15° C. Stirring was continued at the same temperature for 12 h until starting material was consumed completely. The residue was purified by prep-HPLC (column: Luna Omega 5u Polar C18 100A;) to afford the title compound (166 mg, 34.0% yield, 99.8% purity) as a white solid. The structure was confirmed by TOF-MS.

### 1.10 Synthesis of Compound X (V-M1.2)

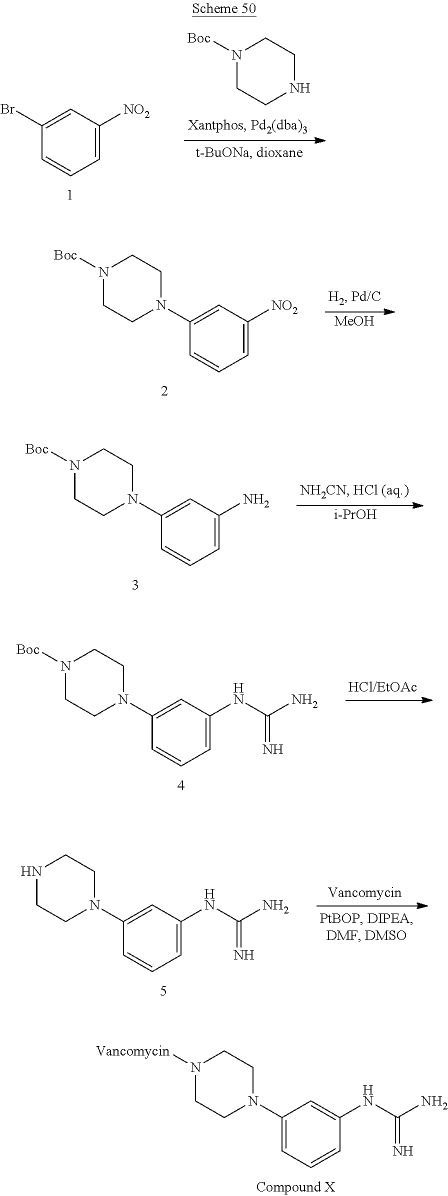

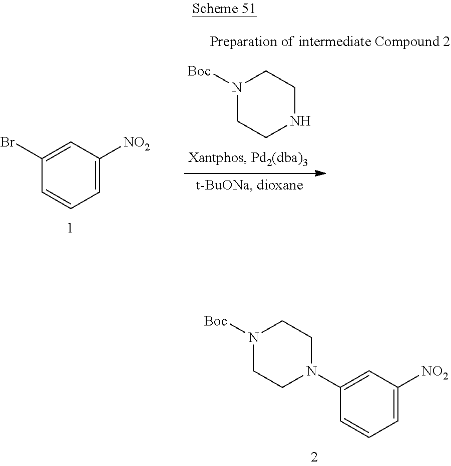 [SMILES: CC(C)(C)OC(=O)N1CCNCC1 ; O=[N+]([O-])c1cccc(Br)c1 ; CC(C)(C)OC(=O)N1CCN(c2cccc([N+](=O)[O-])c2)CC1]

To a mixture of Compound 1 (3.00 g, 14.9 mmol) in dioxane (40 mL) were added tert-butyl piperazine-1-carboxylate (3.32 g, 17.8 mmol), Pd₂(dba)₃ (1.36 g, 1.49 mmol), Xantphos (1.72 g, 2.97 mmol) and t-BuONa (2.85 g, 29.7 mmol) at 15° C. The resulting stirred mixture was heated at 90° C. for 12 h under N₂. Water (30 mL) was added, the mixture was extracted with EtOAc (10 mL*3), then the combined organic layers were dried over Na₂SO₄, filtered, and concentrated under reduced pressure. The residue was purified by column chromatography (SiO₂, Petroleum ether/Ethyl acetate=1/0 to 2/1) to afford Compound 2 (1.90 g, 41.6% yield) as a yellow solid.

### Preparation of Intermediate Compound 3

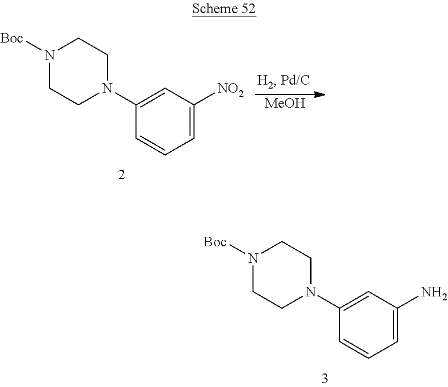 [SMILES: "MeOH" ; CC(C)(C)OC(=O)N1CCN(c2cccc([N+](=O)[O-])c2)CC1 ; CC(C)(C)OC(=O)N1CCN(c2cccc(N)c2)CC1]

To a mixture of 10% Pd/C (200 mg, 50% purity) in MeOH (10 mL) was added Compound 2 (1.80 g, 5.86 mmol) at 15° C. The resulting mixture was stirred and heated at 30° C. for 12 h under H₂ (50 psi). The reaction mixture was filtered, and the filtrate was concentrated under reduced pressure to afford Compound 3 (1.64 g, crude) as a light-yellow oil.

### Preparation of Intermediate Compound 4

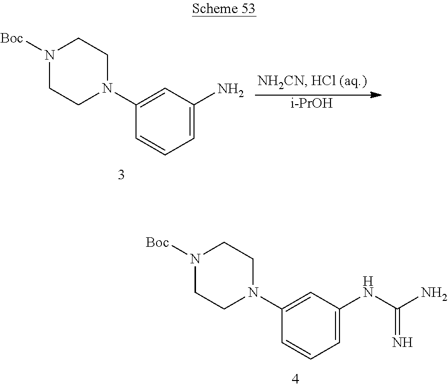 [SMILES: "i-PrOH" ; CC(C)(C)OC(=O)N1CCN(c2cccc(N)c2)CC1 ; CC(C)(C)OC(=O)N1CCN(c2cccc(NC(=N)N)c2)CC1]

To a mixture of Compound 3 (1.64 g, 5.91 mmol) in IPA (25 mL) were added NH₂CN (994 mg, 11.8 mmol, 50% purity) and HCl (12.0 M, 601 uL) at 15° C., then the mixture was stirred at 80° C. for 12 h. The reaction mixture was concentrated under reduced pressure and the resulting residue was purified by prep-HPLC (column: Agela DuraShell C18 250*70 mm*10 um) to afford Compound 4 (592 mg, 28.2% yield, HCl salt) as a white solid.

### Preparation of Intermediate Compound 5

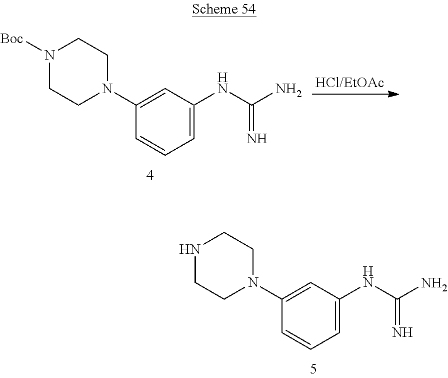 [SMILES: CC(C)(C)OC(=O)N1CCN(c2cccc(NC(=N)N)c2)CC1 ; N=C(N)Nc1cccc(N2CCNCC2)c1]

A mixture of Compound 4 (592 mg, 1.66 mmol) in HCl/EtOAc (4 M, 10 mL) was stirred at 15° C. for 2 h. The reaction mixture was concentrated under reduced pressure to afford Compound 5 (450 mg, crude) as a white solid.

### Preparation of Compound X (V-M1.2)

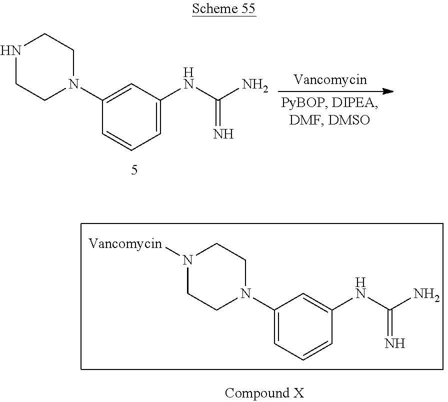

To a mixture of Vancomycin (200 mg, 135 umol, 1.00 eq) in DMF (2 mL) and DMSO (2 mL) were added Compound 5 (68.9 mg, 269 umol), PyBOP (105 mg, 202 umol) and DIPEA (87.0 mg, 673 umol) at 15° C. The resulting mixture was stirred at 15° C. for 12 h. The residue was purified by prep-HPLC (column: Luna Omega 5u Polar C18 100A) to afford Compound X (101 mg, 42.4% yield, 100% purity, TFA) as a white solid.

### 1.11 Synthesis of Compound XI (V-M2.2)

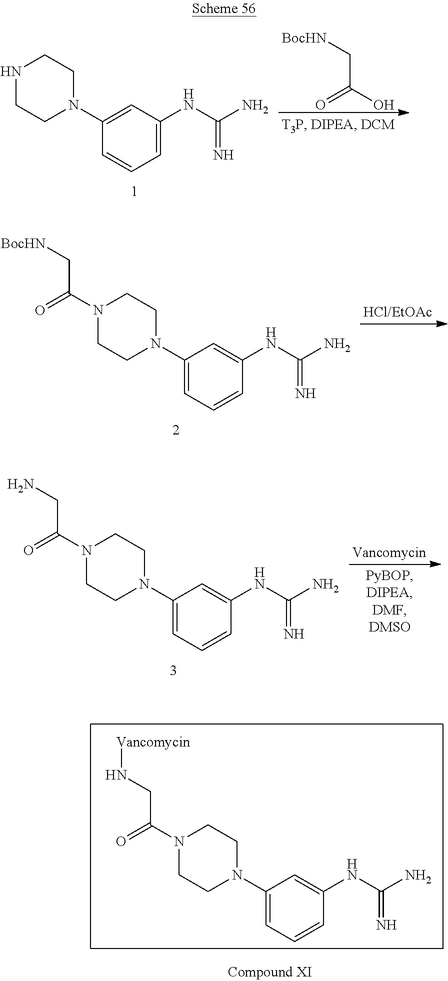

### Preparation of intermediate Compound 1

For detailed experimental procedure for intermediate Compound 1 (precursor to Compound XI), see ‘Preparation of intermediate Compound 5’ in section 1.10 (relating to the Synthesis of Compound X) above.

### Preparation of Intermediate Compound 2

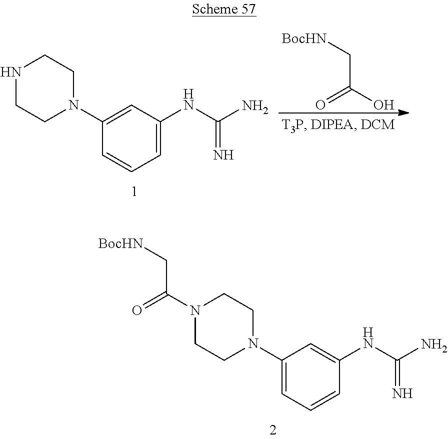 [SMILES: N=C(N)Nc1cccc(N2CCNCC2)c1 ; CC(C)(C)OC(=O)NCC(=O)O ; CC(C)(C)OC(=O)NCC(=O)N1CCN(c2cccc(NC(=N)N)c2)CC1]

To a mixture of Compound 1 (300 mg, 1.17 mmol, HCl salt) in DCM (4 mL) were added 2-(tert-butoxycarbonylamino)acetic acid (247 mg, 1.41 mmol), T₃P (1.12 g, 1.76 mmol, 50% in EtOAc) and DIPEA (455 mg, 3.52 mmol) at 15° C., with continued stirring for 12 h. The reaction mixture was concentrated under reduced pressure and the resulting residue was purified by prep-HPLC (HCl condition) to afford Compound 2 (300 mg, 61.9% yield, HCl salt) as an orange oil.

### Preparation of Intermediate Compound 3

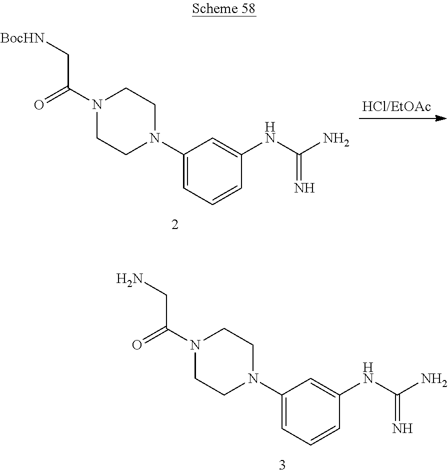 [SMILES: CC(C)(C)OC(=O)NCC(=O)N1CCN(c2cccc(NC(=N)N)c2)CC1 ; N=C(N)Nc1cccc(N2CCN(C(=O)CN)CC2)c1]

A mixture of Compound 2 (300 mg, 727 umol, HCl salt) in HCl/EtOAc (4 M, 10 mL) was stirred at 15° C. for 2 h (until deemed complete by LCMS). The reaction mixture was concentrated under reduced pressure to afford Compound 3 (227 mg, 726 umol, 99.9% yield, HCl salt) as a brown solid.

### Preparation of Compound XI (V-M2.2)

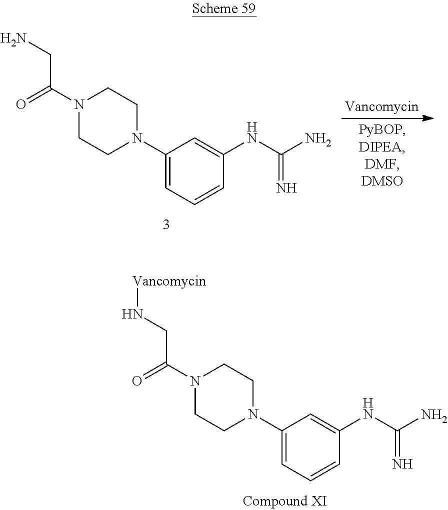

To a mixture of Vancomycin (200 mg, 135 umol, 1.00 eq) in DMF (1.5 mL) and DMSO (1.5 mL) were added Compound 3 (63.2 mg, 202 umol, HCl salt), PyBOP (105 mg, 202 umol) and DIPEA (87.0 mg, 673 umol) at 15° C., and the contents of the vessel were stirred at 15° C. for 12 h. The isolated residue was purified twice by prep-HPLC (column: Luna Omega 5u Polar C18 100A), to afford Compound XI (32 mg, 13.5% yield, 99.4% purity, FA salt) as a white solid. Identity was confirmed by TOF-HRMS.

### 1.12 Synthesis of Compound XII (V-M7.2)

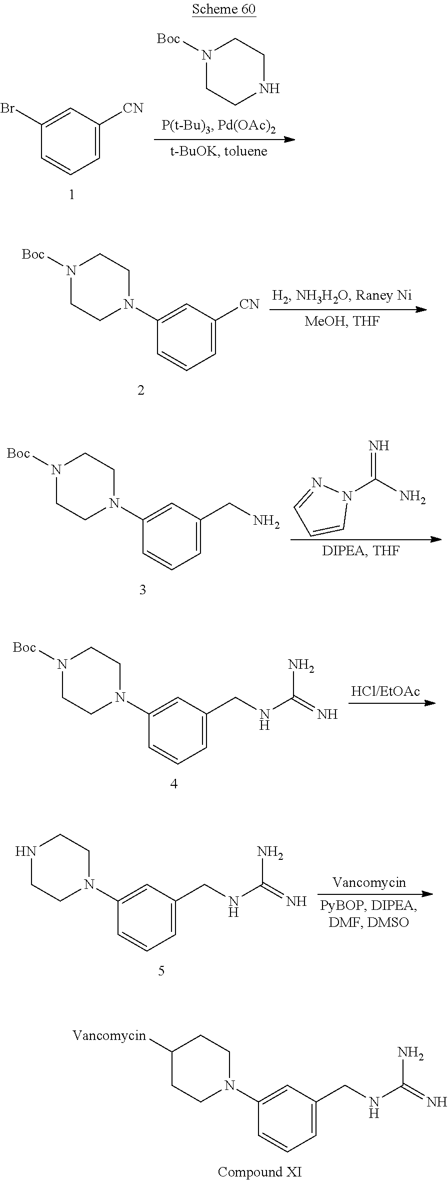

### Preparation of Intermediate Compound 2

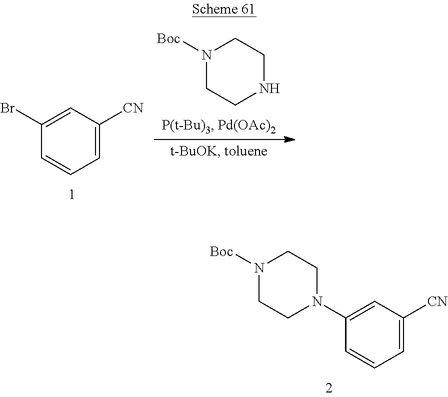 [SMILES: CC(C)(C)OC(=O)N1CCNCC1 ; N#Cc1cccc(Br)c1 ; CC(C)(C)OC(=O)N1CCN(c2cccc(C#N)c2)CC1]

To a mixture of Compound 1 (12.7 g, 69.8 mmol) in toluene (250 mL) were added tert-butyl piperazine-1-carboxylate (19.5 g, 105 mmol), P(t-Bu)₃ (2.82 g, 1.40 mmol 10% purity), Pd(OAc)₂ (157 mg, 698 umol) and t-BuOK (9.40 g, 83.8 mmol) at 15° C. The resulting mixture was stirred and heated at 50° C. for 12 h under N₂. After cooling to room temperature the mixture was concentrated under reduced pressure and the resulting residue was purified by column chromatography (SiO₂, Petroleum ether/Ethyl acetate=1/0 to 3/1) to afford Compound 2 (1.10 g, 3.83 mmol) as a white solid.

### Preparation of Intermediate Compound 3

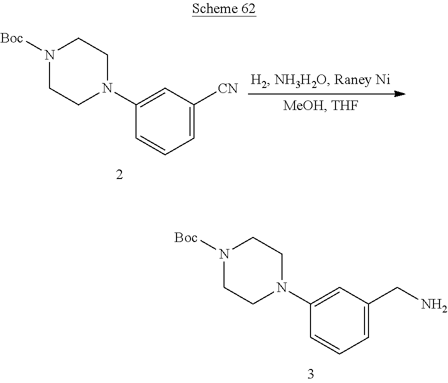 [SMILES: CC(C)(C)OC(=O)N1CCN(c2cccc(C#N)c2)CC1 ; CC(C)(C)OC(=O)N1CCN(c2cccc(CN)c2)CC1]

To a suspension of Raney-Ni (1.00 g, 11.7 mmol) in THF (10 mL) and MeOH (10 mL) were added Compound 2 (1.00 g, 3.48 mmol) and NH₃—H₂O (9.10 g, 64.9 mmol, 10.0 mL, 25% purity) at 15° C., the mixture was degassed under vacuum and purged with H₂ several times. The reaction mixture was stirred under H₂ (15 psi) at 15° C. for 4 h, after which it was purged with nitrogen. The mixture was then filtered, and the filtrate was concentrated under reduced pressure to afford Compound 3 (810 mg, crude) as a light green oil.

### Preparation of Intermediate Compound 4

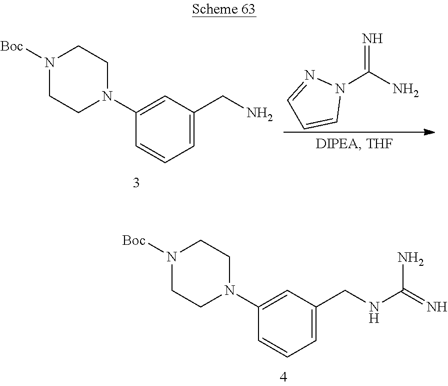

To a mixture of Compound 3 (750 mg, 2.57 mmol) and pyrazole-1-carboxamidine (283 mg, 2.57 mmol) in THF (10 mL) was added DIPEA (336 mg, 2.57 mmol) at 15° C. The mixture was stirred at 15° C. for 6 h and then concentrated under reduced pressure. The resulting residue was purified by prep-HPLC (column: Welch Xtimate C18 100*25 mm*3 um) to afford Compound 4 (570 mg, 59.9% yield, HCl) as a light-yellow oil.

### Preparation of Intermediate Compound 5

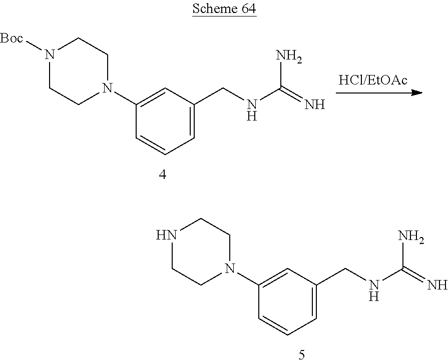 [SMILES: CC(C)(C)OC(=O)N1CCN(c2cccc(CNC(=N)N)c2)CC1 ; N=C(N)NCc1cccc(N2CCNCC2)c1]

To a mixture of Compound 4 (550 mg, 1.49 mmol, HCl salt) in EtOAc (5 mL) was added HCl/EtOAc (4 M, 5 mL) at 15° C., and the mixture was stirred at 15° C. until the reaction was deemed complete by LC-MS. The mixture was concentrated under reduced pressure to afford Compound 5 (380 mg, 94.7% yield, HCl) as a white solid.

### Preparation of Compound XII (V-M7.2)

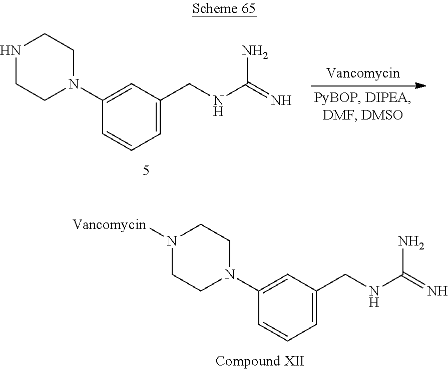

To a mixture of Compound 5 (79.9 mg, 296 umol, HCl salt) in DMF (2 mL) and DMSO (2 mL) was added Vancomycin (400 mg, 269 umol), PyBOP (210 mg, 404 umol) and DIPEA (174 mg, 1.35 mmol) at 15° C. The resulting mixture was stirred at 15° C. for 12 h (until deemed complete). The mixture was then purified by prep-HPLC (column: Luna Omega 5u Polar C18 100A) to afford the TFA salt of the title compound (132 mg, 27.1% yield, 98.5% purity) as a white solid. Identity was verified by TOF-HRMS.

### 1.13 Synthesis of Compound XIII (V-M12.2)

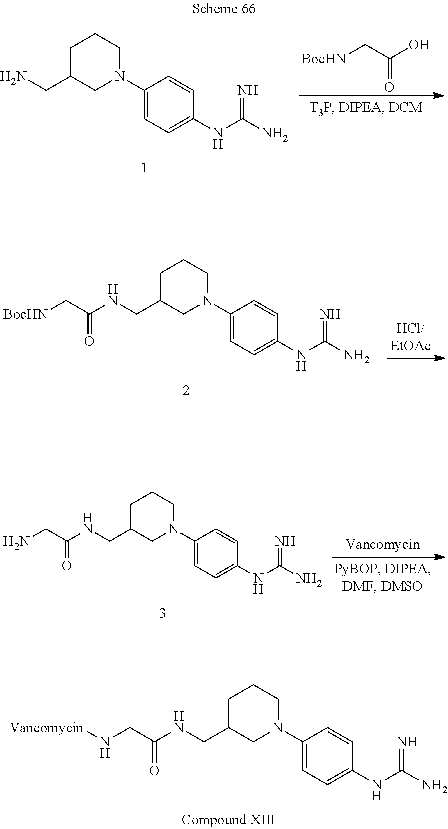

### Route of Synthesis for Intermediate Compound 1

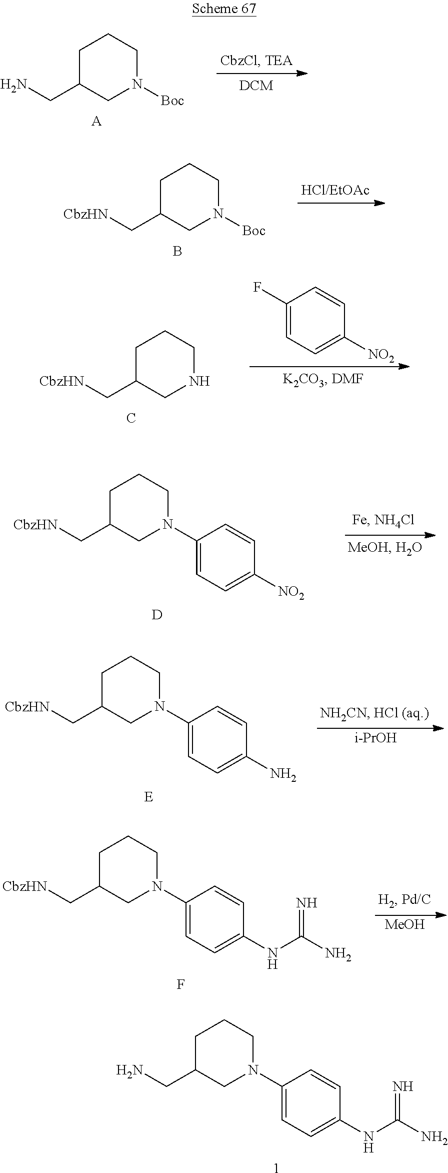

### Preparation of Intermediate Compound B

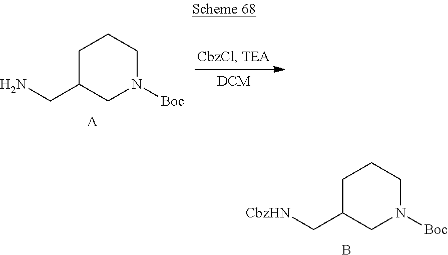

To a mixture of Compound A (25.0 g, 117 mmol) in DCM (100 mL) was added TEA (15.4 g, 152 mmol) at 15° C., followed by further cooling to 0° C. and addition of CbzCl (21.9 g, 128 mmol) in DCM (60 mL). The resulting mixture was stirred at 15° C. for 5 h. The reaction mixture was washed with aq. HCl (1.0 M), and brine, dried over Na₂SO₄, filtered and concentrated under reduced pressure. The resulting residue was purified by column chromatography (SiO₂, Petroleum ether/Ethyl acetate=1/0 to 4/1) to afford Compound B (34.0 g, 83.7% yield) as a colorless oil.

### Preparation of Intermediate Compound C

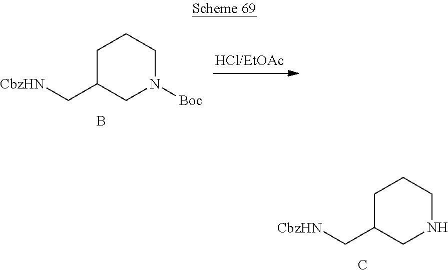

A solution of Compound B (34.0 g, 97.6 mmol) in HCl/EtOAc (4.0 M, 100 mL) was stirred at 15° C. for 12 h. The mixture was concentrated under reduced pressure to afford Compound C (25.0 g, crude, HCl salt) as a colourless oil.

### Preparation of Intermediate Compound D

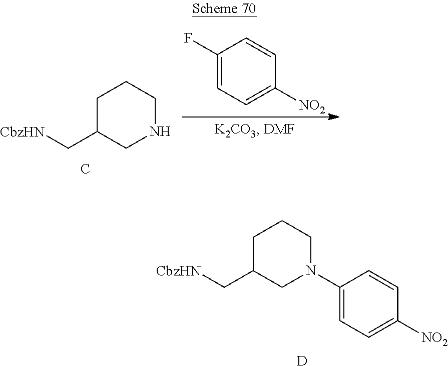

To a solution of Compound C (17.0 g, 59.7 mmol, HCl salt) in DMF (100 mL) were added 1-fluoro-4-nitro-benzene (9.27 g, 65.7 mmol) and K₂CO₃ (16.5 g, 119 mmol) at 15° C. The resulting mixture was stirred at 90° C. for 4 h. Water (50 mL) was added, the mixture was extracted with EtOAc, the combined organic layers were dried over Na₂SO₄, filtered, and concentrated under reduced pressure. The residue was purified by column chromatography (SiO₂, Petroleum ether/Ethyl acetate=1/0 to 1/1) to afford Compound D (21.0 g, 95.2% yield) as a yellow solid.

### Preparation of Intermediate Compound E

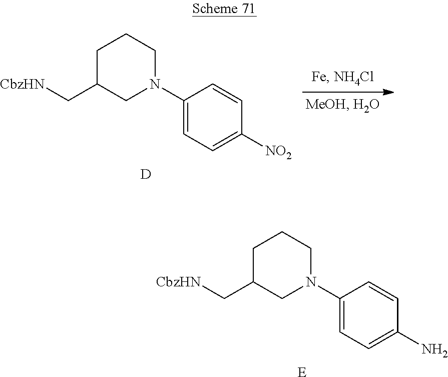

To a solution of Compound D (15.0 g, 40.6 mmol) in MeOH (120 mL) and H₂O (60 mL) were added Fe (11.3 g, 203 mmol) and NH₄Cl (6.52 g, 122 mmol) at 15° C. The resulting mixture was heated and stirred at 80° C. overnight. The mixture was cooled and filtered and the filtrate was concentrated under reduced pressure to afford Compound E (13.0 g, crude) as a black solid.

### Preparation of Intermediate Compound F

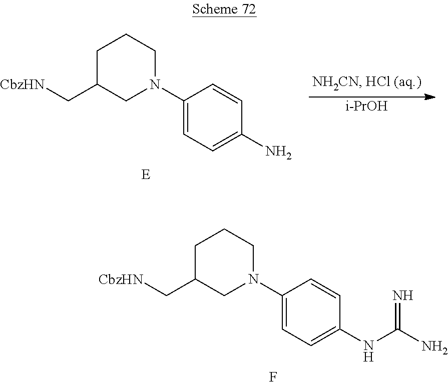

To a mixture of Compound E (5.00 g, 14.7 mmol) and NH₂CN (2.48 g, 29.5 mmol, 50% purity,) in IPA (150 mL) was added HCl (12.0 M, 1.50 mL) at 15° C. The resulting mixture was heated and stirred at 80° C. overnight. The mixture was cooled to ambient temperature and concentrated under reduced pressure. The residue was purified by prep-HPLC (column: Agela DuraShell C18 250*70 mm*10 um) to afford Compound F (2.30 g, 37.4% yield, HCl salt) as a black oil.

### Preparation of Intermediate Compound 1

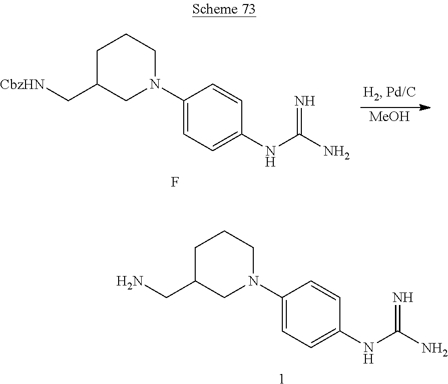

To a mixture of 10% Pd/C (300 mg, 50% in water) in MeOH (40 mL) was added Compound F (2.20 g, 5.26 mmol, HCl) at 15° C. The resulting mixture was degassed under vacuum and purged with H₂ several times. The mixture was stirred under H₂ (50 psi) at 30° C. until the reaction was deemed complete. The mixture was cooled to room temperature, purged with nitrogen and filtered. The filtrate was concentrated under reduced pressure to afford Compound 1 (1.30 g, crude, HCl) as brown oil.

### Preparation of Intermediate Compound 2

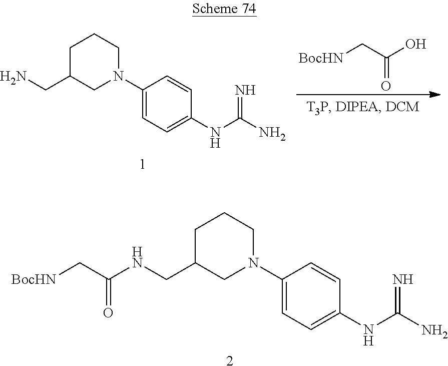

To a mixture of Compound 1 (500 mg, 1.76 mmol) in DCM (10 mL) were added 2-(tert-butoxycarbonylamino)acetic acid (340 mg, 1.94 mmol), DIPEA (455 mg, 3.52 mmol) and T₃P (1.57 mL, 2.64 mmol, 50% in EtOAc) at 15° C. Stirring of the resulting mixture was continued at 15° C. for 12 h. The mixture was concentrated under reduced pressure and the residue was purified by prep-HPLC (column: Welch Xtimate C18 100*25 mm*3 um;) to afford Compound 2 (380 mg, 48.9% yield, HCl salt) as a light yellow oil.

### Preparation of Intermediate Compound 3

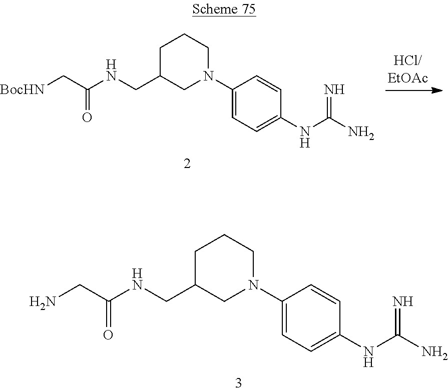 [SMILES: CC(C)(C)OC(=O)NCC(=O)NCC1CCCN(c2ccc(NC(=N)N)cc2)C1 ; "2" ; N=C(N)Nc1ccc(N2CCCC(CNC(=O)CN)C2)cc1 ; "3"]

A solution of Compound 2 (380 mg, 862 umol) in HCl/EtOAc (4.0 M, 5 mL) was stirred at 15° C. for 1 h. The mixture was concentrated under reduced pressure to afford Compound 3 (290 mg, 98.7% yield, HCl) as a white solid.

### Preparation of Compound XIII (V-M12.2)

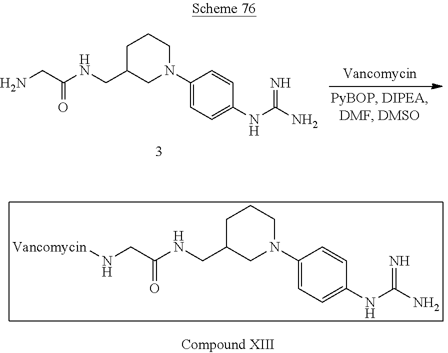

To a mixture of Vancomycin (200 mg, 135 umol) in DMF (1 mL) and DMSO (1 mL) were added Compound 3 (59.7 mg, 175 umol), PyBOP (105 mg, 202 umol) and DIPEA (117 uL, 673 umol) at 15° C. The resulting mixture was stirred at 15° C. for 4 h and then purified by prep-HPLC (column: Luna Omega 5u Polar C18 100A) to afford Compound XIII (16.5 mg, 6.1% yield, 88.3% purity, FA salt) as a white solid. The identity of the title compound was identified by TOF-HRMS.

### Example 2: General Procedure for the Synthesis of Linezolid MoTr Conjugates

The general synthetic procedure for the preparation of linezolid MoTr analogues from the precursor Des-Ac-LIN is outlined in Scheme 77 below:

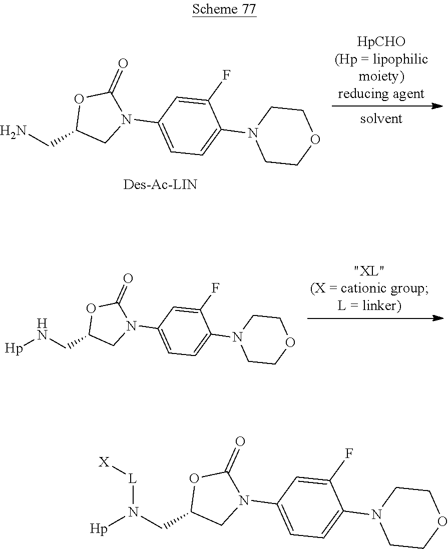

### Synthesis of (R)-2-amino-N-benzyl-N—(((S)-3-(3-fluoro-4-morpholinophenyl)-2-oxooxazolidin-5-yl)methyl)-5-guanidinopentanamide

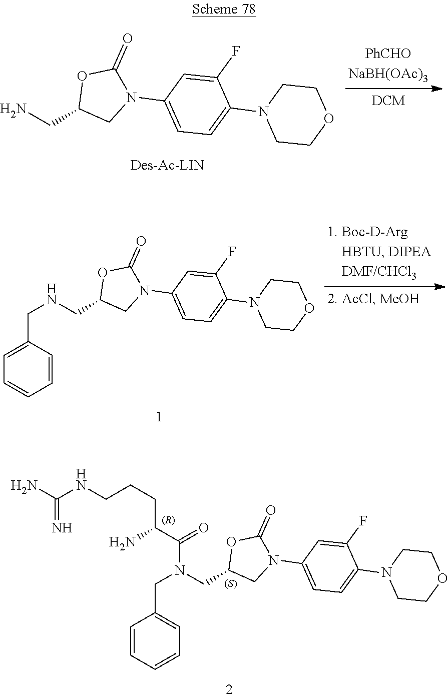

Benzaldehyde (1.1 equiv) was added into a solution of deacetyl linezolid (1 equiv., 300 mg) and NaBH(OAc)₃ (1.3 equiv.) in dichloromethane (DCM, 10 mL) The resulting mixture was stirred under an inert atmosphere at ambient temperature overnight, after which the reaction was quenched by the addition of saturated sodium hydrogen carbonate The solution was then extracted with DCM. The organic layer was subsequently washed with water then brine. Subsequently, the organic layer was dried over anhydrous MgSO₄, filtrated, and concentrated under reduced pressure, and the resulting residue was purified using column chromatography (hexane:ethyl acetate=5:1 to 1:1 v/v and with 0.5% triethylamine) to obtain the intermediate compound 1.

To a stirred and cooled (0° C.) solution of intermediate 1 in DMF/CHCl₃ (2.5:1, 10 mL) was added DIPEA (3 equiv.). To this solution was then added HBTU (1.25 equiv.). Stirring of the resulting mixture was continued for 5 min at 0° C. and was followed by addition of Boc-protected D-arginine (1.25 equiv.). The mixture was stirred at 0° C. for 30 min and subsequently at ambient temperature overnight. The chlorinated solvent was evaporated under reduced pressure and the resulting solution was diluted by addition of EtOAc. This mixture was subsequently washed with 0.5 M KHSO₄, water, and brine. The combined organic layers were dried over anhydrous Mg₂SO₄, filtered, and the filtrate was concentrated under reduced pressure. The resulting residue was purified by column chromatography on silica gel (hexane:ethyl acetate=5:1 v/v and with 5% triethylamine) to obtain the intermediate Boc-protected peptide.

This intermediate compound was then resuspended in methanol and the resulting solution stirred at 0° C., Acetyl chloride (6 equiv.) was added carefully to the cold solution under strict temperature control in order to regulate the exotherm and increase the solubility of the HCl. The solution was allowed to gradually warm to ambient temperature and the reaction was stirred until deemed complete. The title compound (2) was characterized by 1H NMR, 13C NMR, and mass spectrometry.

### Example 3: Microbial Susceptibility (Minimum Inhibitory Concentration, MIC) Testing

Susceptibility testing was performed based on Clinical and Laboratory Standards Institute (CLSI) guidelines M07-A11 and M100-S29. All bacterial strains were recovered from long-term storage (−80° C.) and cultured on nutrient agar (NA) at 37° C. under aerobic conditions for approximately 20 h, except E. coli ATCC BAA-2469 that was recovered from long-term storage on nutrient agar before further passaging onto nutrient agar+25 μg/mL imipenem (according to ATCC guidelines). Minimum inhibitory concentration (MIC) assays were performed in BD non cation adjusted Mueller-Hinton broth (MHB) in 96-well flat-bottomed polystyrene plates (Corning #3370). Test articles and comparator antibiotics were prepared in sterile water to a concentration of 5.12 mg/mL. For each antibiotic stock, 2× top concentrations of test compounds and comparators were prepared in the appropriate growth medium and added to well 1 of a 96-well plate. Serial 1:1 dilutions were performed from columns 1-11 (changing tips at each dilution step); column 12 served as a positive (growth) control. A negative (sterility) control was set up in available spare wells. A suspension of each strain was prepared in sterile phosphate-buffered saline (PBS) to a density equivalent to a 0.5 McFarland standard. Bacterial suspensions were then diluted 1:150 in the appropriate medium and used to inoculate assay plates (columns 1 through 11) to provide a starting inoculum of ˜2 to 8×105 CFU/mL. Assay plates were incubated at 35±2° C. for 18 h under aerobic conditions (24 h for S. aureus strains against vancomycin). The MIC was defined as the lowest concentration of test articles resulting in complete inhibition of visible growth.

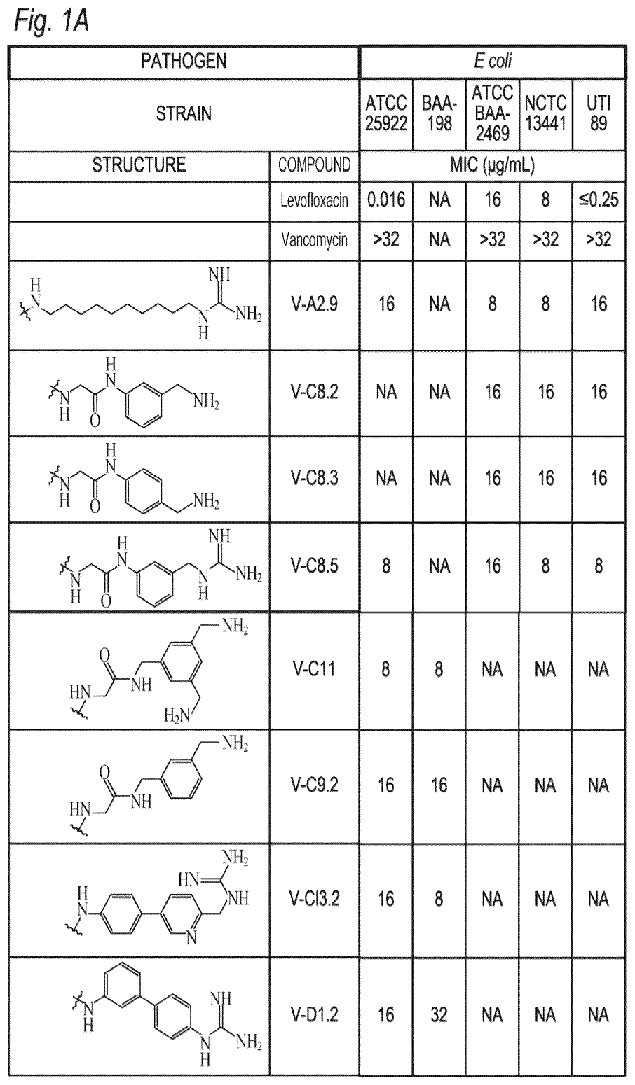

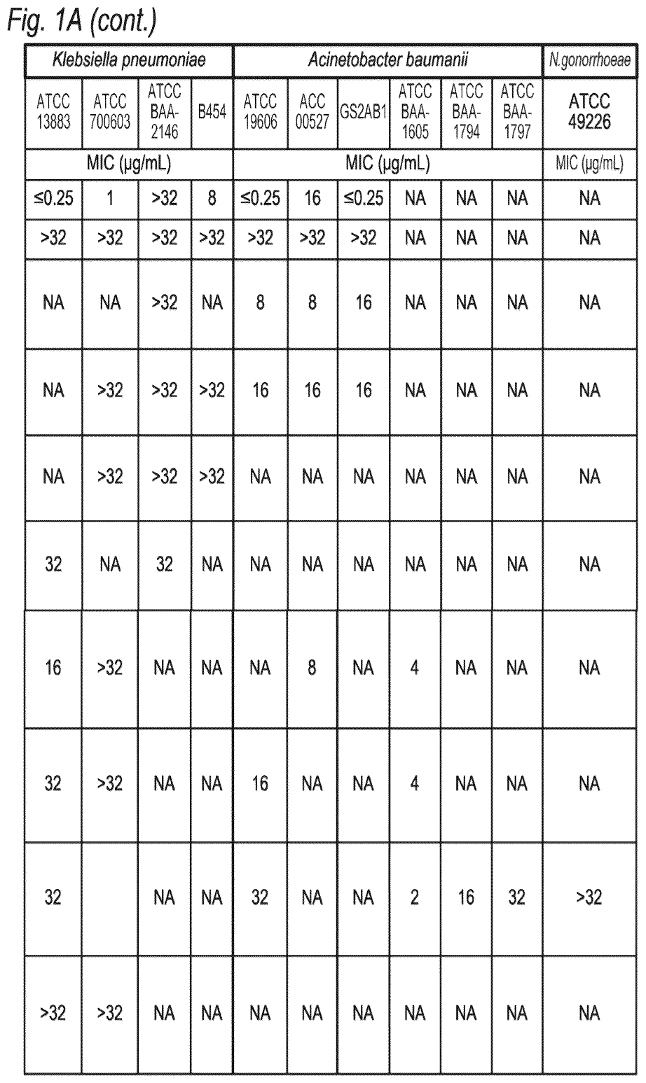

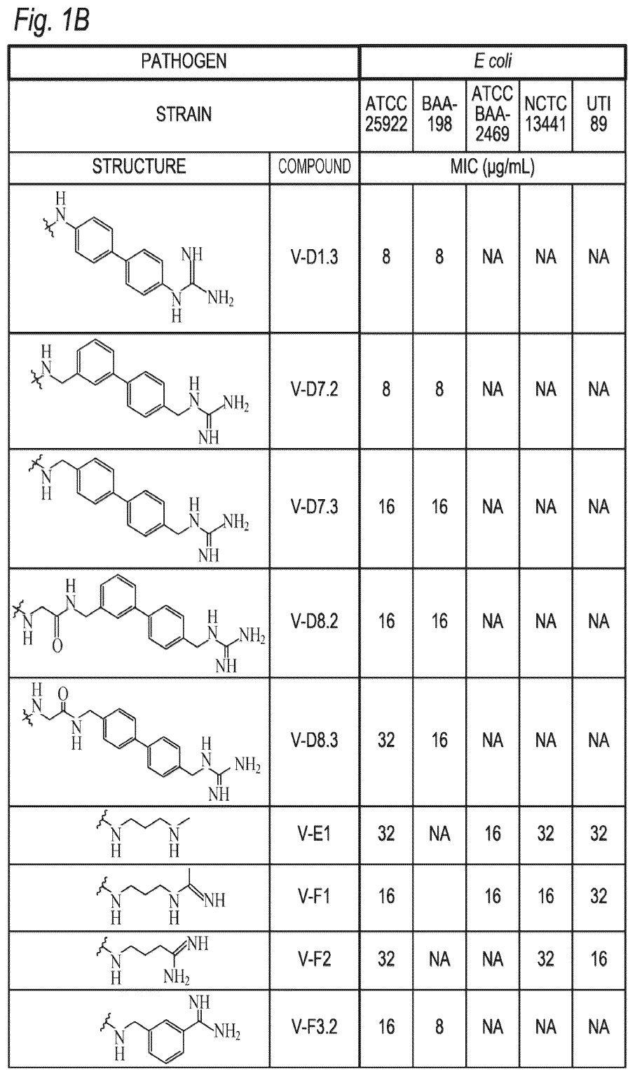

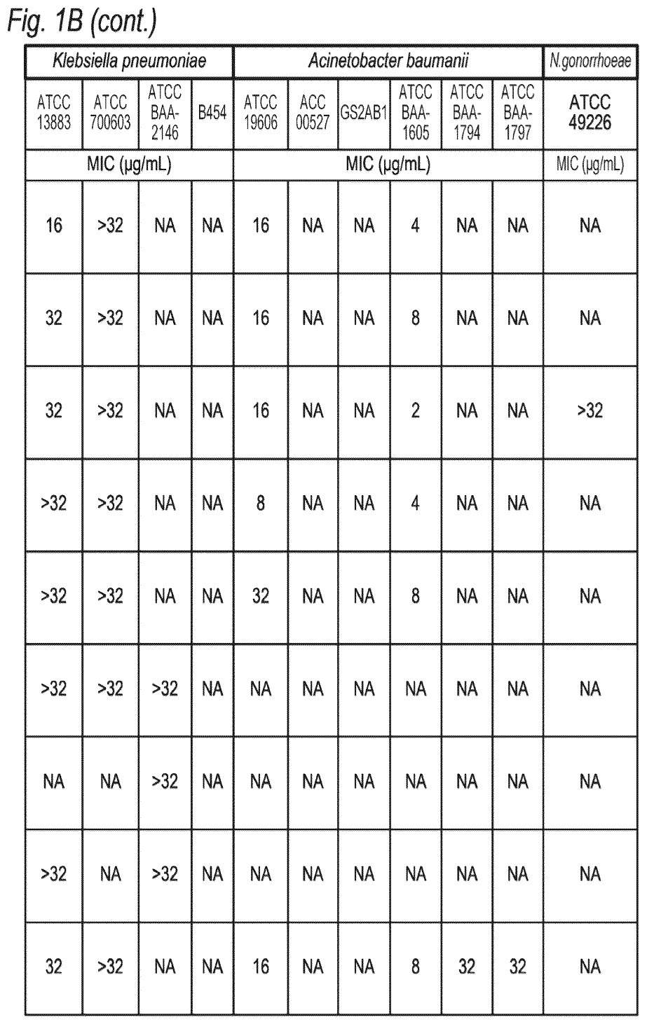

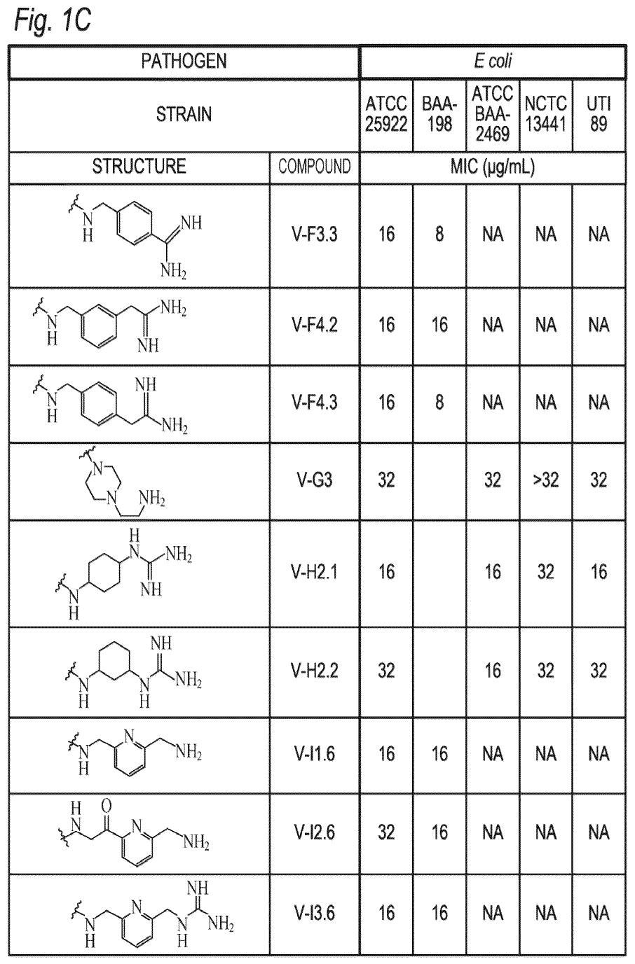

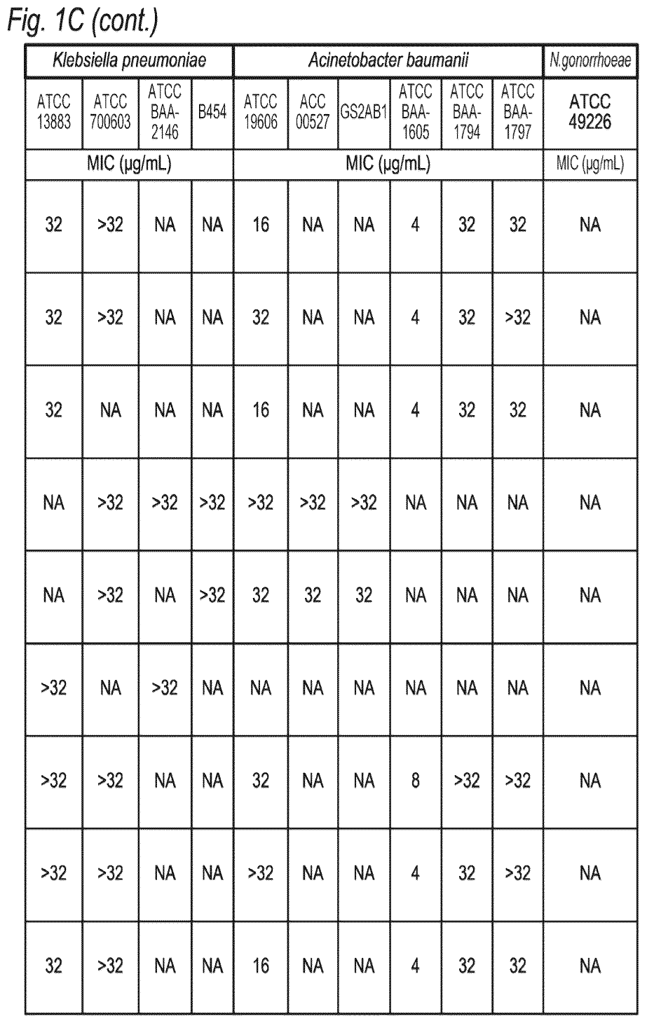

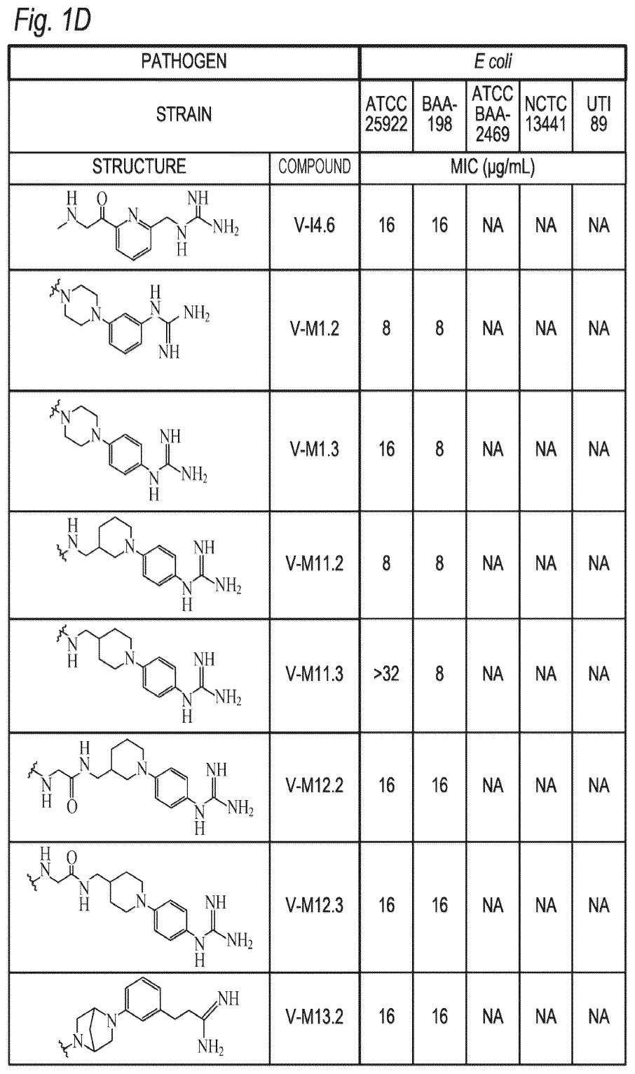

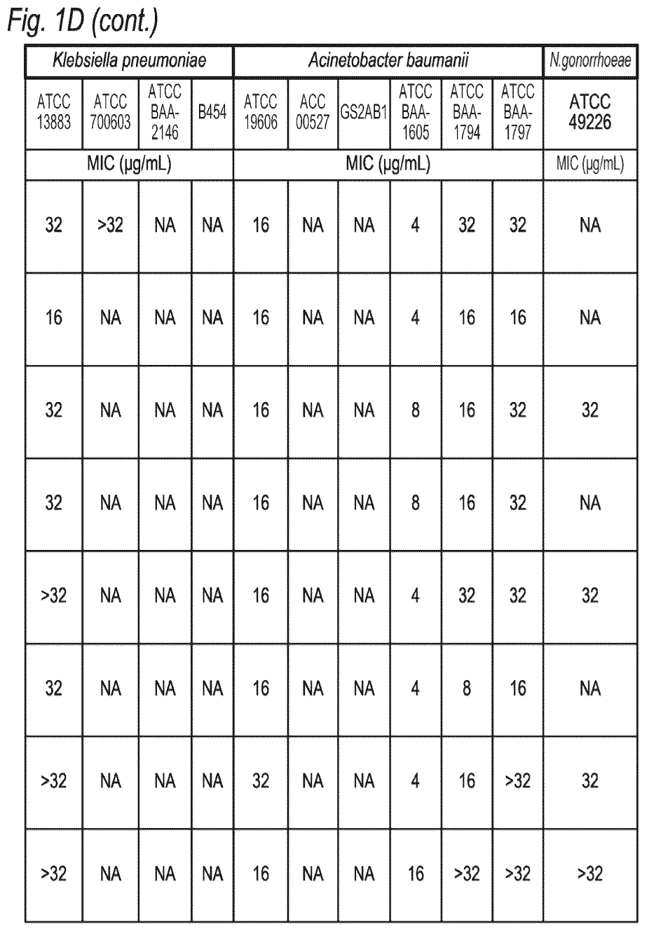

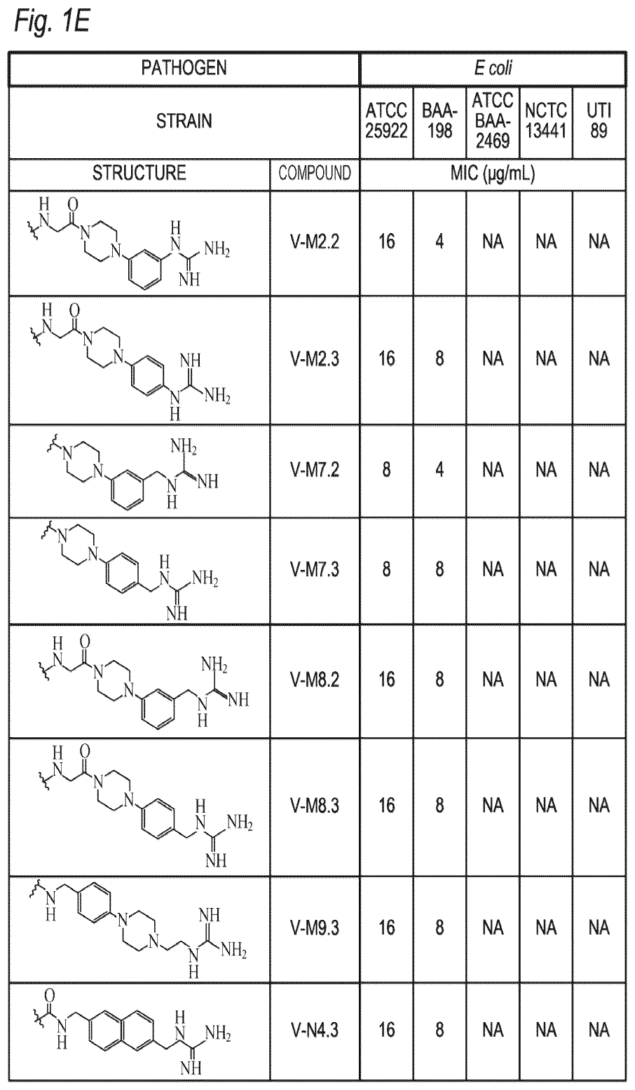

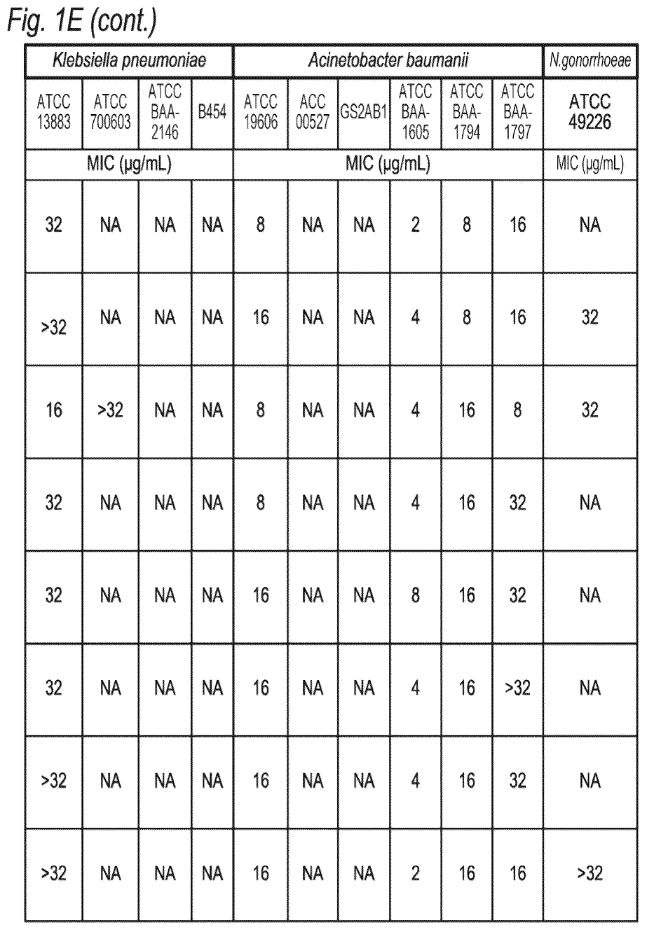

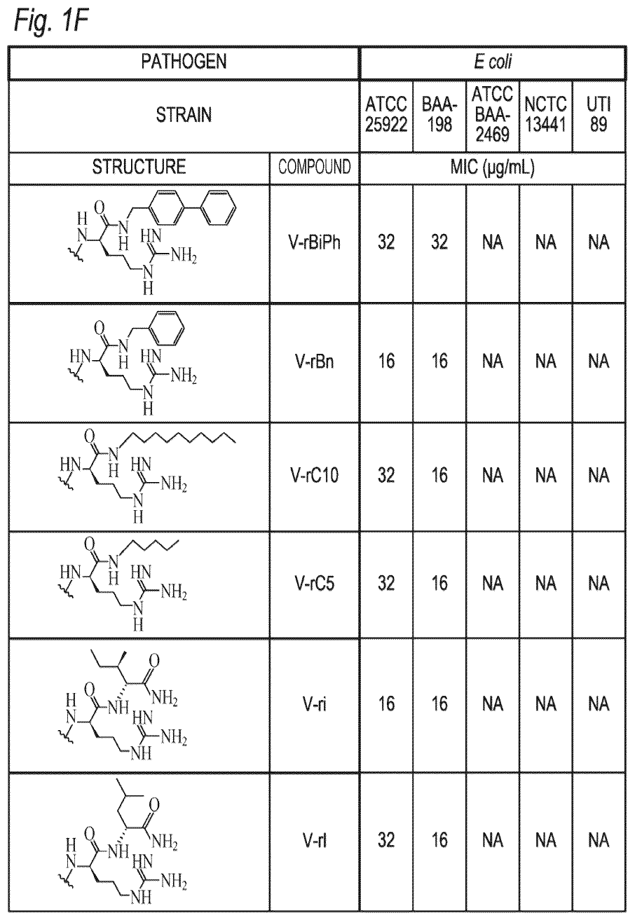

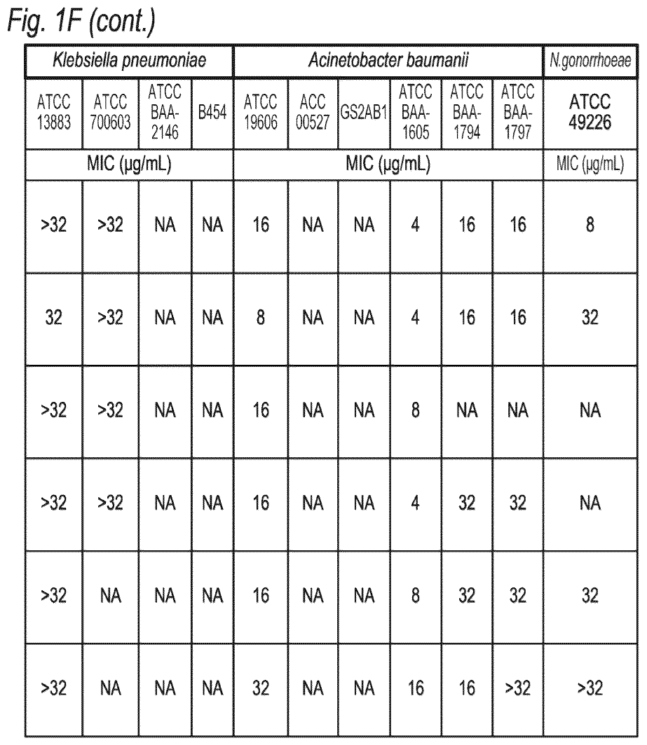

Selected vancomycin conjugates (see EXAMPLE 1) were tested against a series of GPB and GNB pathogens, including multi-drug resistant strains of significant clinical importance. A summary of antimicrobial susceptibility data (Minimum Inhibitory Concentration, MIC) against a selection of GNB pathogens is provided (see FIGS. 1A-1F). This list served as the basis for selection of candidate compounds with the best antimicrobial properties, i.e., affording MIC of <16 ug/mL in at least two Acinetobacter and E. coli strains and <32 ug/mL in K. Pneumoniae. In vitro toxicity and in vivo tolerability profiles of selected candidate compounds was evaluated.

In addition to superior/improved antimicrobial activity against a selection of E coli, Acinetobacter and K pneumonia strains, specific compounds demonstrated enhanced in vitro toxicity and tolerability profiles compared to the previously known vancomycin conjugates, including compound QC14 and others by Haldar et al (J. Med. Chem. 2014, 57, 11, 4558-4568 ACS Infect. Dis. 2016, 2, 2, 132-139) and vancomycin conjugates reported by Boger et al (ACS Infect Dis 2020).

Maximum tolerated dose in mice estimated to 50-75 mg/kg, several fold lower than the tolerable dose observed with Compounds of present invention. (see Table 1).

TABLE 1
Toxicity/ Tolerability of the compounds with the best
antimicrobial properties

| | In vitro tox | | In vivo (mouse) |
|---|---|---|---|
| | IC₅₀ (ug/mL) | | tolerability |
| Compound ID | RBC | HepG2 | at 200 mg/kg |
| V-C9.2 | >400 | >512 | — |
| V-C11 | >400 | >512 | — |
| V-D2.2 | >400 | 212 | — |
| V-D7.2 | >400 | 189 | — |
| V-D8.2 | >400 | NA | — |
| V-rBn | >512 | >256 | Well tolerated |
| V-M1.2 | >512 | >256 | Well tolerated |
| V-M2.2 | >512 | 140 | Tolerated |
| V-M7.2 | >512 | >256 | Well tolerated |
| V-M12.2 | >512 | >256 | — |

### Example 4: In Vivo Targeting of E coli with Vancomycin-Arginine (V-r)

Previous studies suggested that conjugation of L-arginine to vancomycin, vancomycin-L-arginine (V-R), can provide promising Gram-negative properties via a cell wall mode-of-action. This prompted the inventors to investigate the corresponding diastereomer using the D-isomer of arginine, vancomycin-D-arginine (V-r), to reduce the risk of conjugate proteolysis. The structures of vancomycin and V-r are shown below.

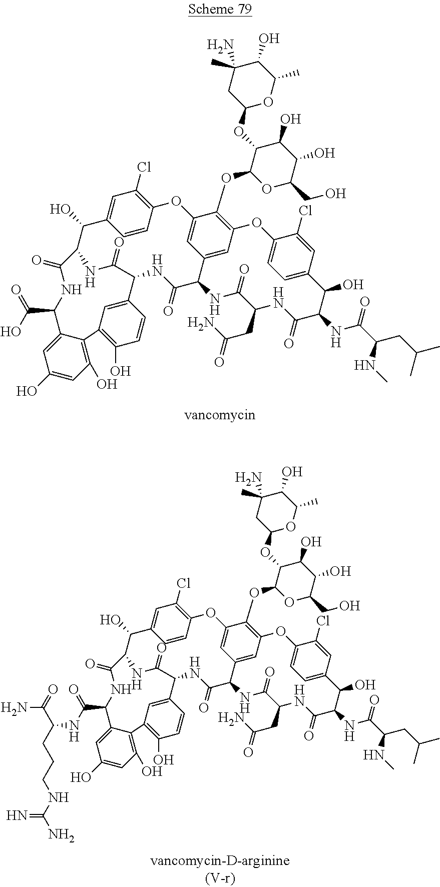

V-r was synthesized in a single chemical step from commercially available vancomycin-HCl (StruChem, China) and D-arginine amide di-hydrochloride (Aladdin Chemical Co., China). The crude compound was purified and isolated as the corresponding HCl salt at 95% purity by HPLC based on previously described procedure. Identity was confirmed by 1H-NMR and TOF-MS, and HCl content was quantified by ion exchange chromatography.

In a variety of physicochemical screens, V-r behaved similarly to vancomycin, including no observed cellular cytotoxicity at concentrations ranging from 100 to 750 μM in human erythrocytes, HepG2, and chronic kidney tubular cell screens employing fetal bovine serum (FBS)-deficient media to negate compound quenching (see Table 2).

TABLE 2
Physicochemical properties of vancomycin-arginine
(V-r) and vancomycin

| Physicochemical properties | V-r | Vancomycin |
|---|---|---|
| Molecular weight (free base) | 1604.5 | 1449 |
| LogD (Octanol/buffer) | <−4.01 | −5.14^{a} |
| TD solubility in saline (mg/ml) | 373 | >50 |
| PPB, % bound, mouse/human | 65/76 | 50/50 |
| Red blood cell lysis (CC₅₀, μM) | >750 | >750 |
| HepG2 cell cytotoxicity (CC₅₀, μM) | >750 | >750 |
| hRPTEC biomarkers^{b} (CC₅₀, μM) | >100 | >100 |
| FOR (at 8 × MIC) | <2.32 × 10⁻¹⁰ | n.d. |

^{a}LogD vancomycin reported according to Dave and Morris (29).
^{b}Includes cell count, nuclear size, DNA structure, Mitochondrial mass, Mitochondrial membrane potential, Phospholipidosis and Glutathione content.
TD, Thermodynamic solubility;
PPB, Plasma Protein Binding;
hRPTEC, human renal proximal tubular epithelial cells;
CC₅₀, the concentration at which 50% cytotoxicity is observed;
FoR, Frequency of Resistance.
n.d. = not determined.

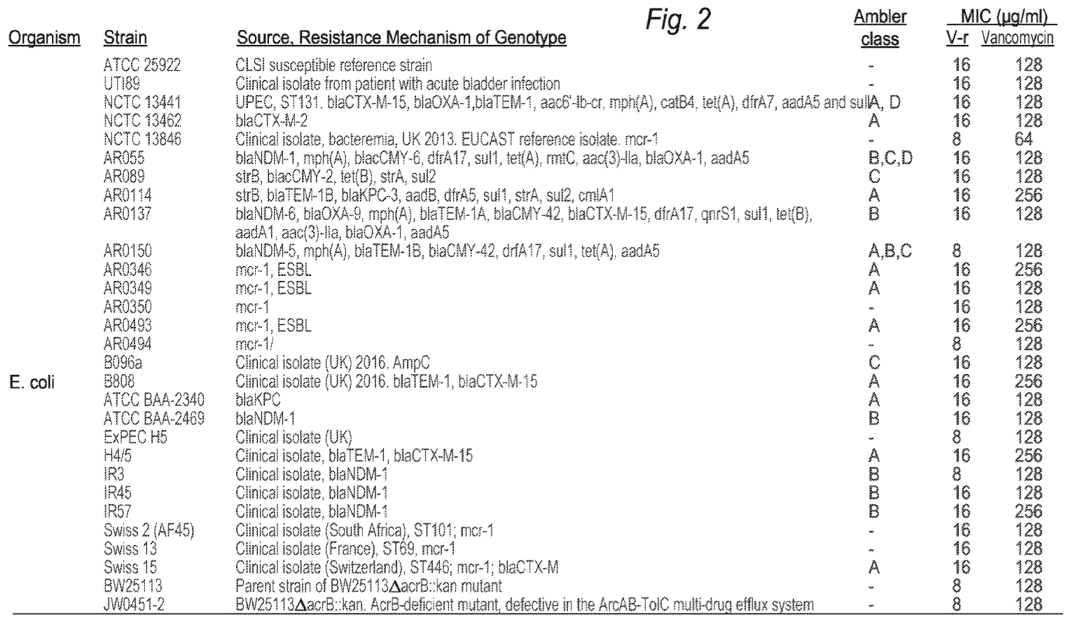

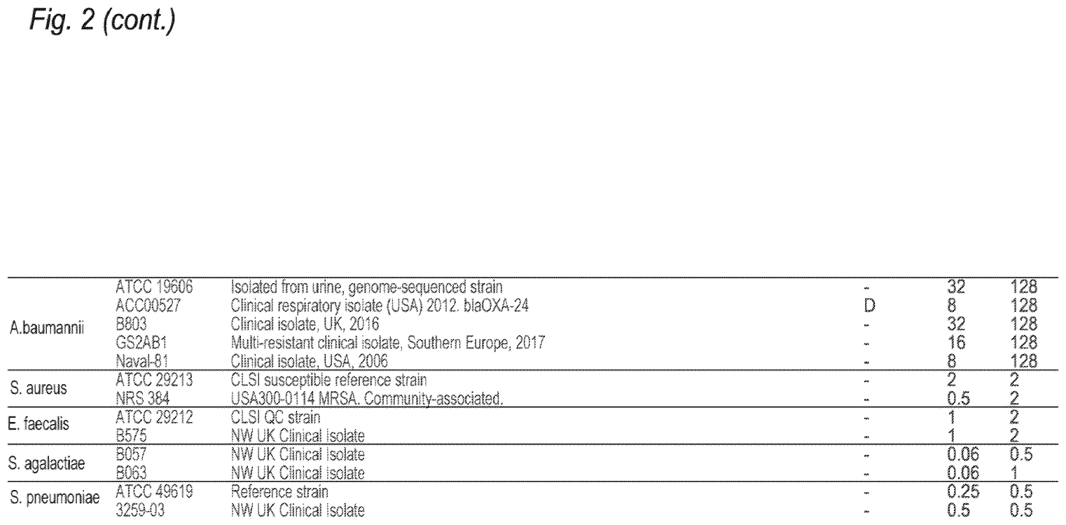

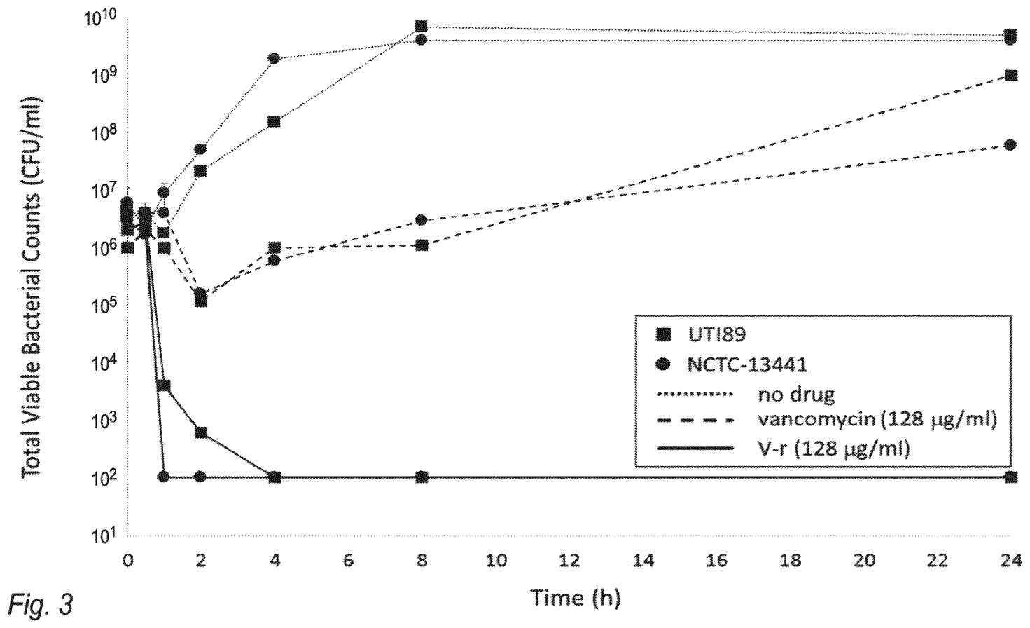

MICs were determined using standard methodologies as previously described. The MIC range of V-r against 29 different E. coli strains was 8-16 μg/ml (MIC₉₀=16 μg/ml), including those with multiple resistance mechanisms. The MIC of V-r against the efflux pump mutant strain JW0451-2 was 8 μg/ml, suggesting that V-r is unlikely to be a substrate for efflux in this pathogen. Notably, the MIC of V-r was also 8 μg/ml against 2/5 A. baumannii strains tested. In comparison, the MICs of vancomycin were significantly higher, at 64-256 μg/ml against all E. coli and A. baumannii strains tested. Importantly, the antimicrobial potency of V-r towards a number of GPB remained intact (see FIG. 2). In frequency-of-resistance (FoR) assays at 8×MIC V-r (128 mg/ml), E. coli ATCC 25922 demonstrated an extremely low FoR at <2.3×10¹⁰, which is similar, or lower than standard-of-care therapies, such as ciprofloxacin. Time-kill assays were performed against uropathogenic E. coli strains, including the sequence type 131 (ST131) NCTC-13341 isolate. V-r, but not vancomycin, demonstrated rapid bactericidal activity to limits of detection (LOD, 100 CFU/ml) within 1 or 4 h of exposure and maintained up to 24 h (see FIG. 3).

Plasma pharmacokinetics (PK) of V-r following subcutaneous (SC) administration (20 mg/kg and 121 mg/kg) was determined in naïve male CD-1 mice (n=3 per group, see Table 3) using LC-MS/MS for analysis with a lower limit of quantitation (LLOQ) of 5 ng/ml. V-r displayed first-order elimination, similar to vancomycin following SC administration. Prior to efficacy studies, a single SC administration of V-r was shown to be well tolerated in male CD-1 mice (n=3) at the highest dose tested (800 mg/kg).

TABLE 3
PK parameters of V-r in CD-1 mice following SC administration

| Pharmacokinetic | | |
|---|---|---|
| parameters | V-r | |
| Dose (mg/kg) | 20 | 121 |
| t_{1/2} (h) | 0.87 | 1.29 |
| Cₘₐₓ (mg/ml) | 20.4 | 98.4 |
| Cl (ml/min/kg) | 7.8 | 5.4 |
| AUC (mg.h/l) | 42.7 | 366.1 |
| Vd (l/kg) | 0.59 | 0.60 |

Using a screening-based strategy, preliminary proof-of-concept studies with V-r employed an abbreviated 9 h thigh muscle infection model in male CD-1 mice rendered neutropenic. To that end, E. coli ATCC 25922 was inoculated at 9.7×10⁴ CFUs into both thigh muscles per mouse (n=5 per experimental group). V-r was administered q2h SC (110-880 mg/kg total dose) starting 1 h post-infection. At 9 h, thigh homogenates were prepared and CFUs were enumerated following culture on CLED agar. Compared to pre-treatment and vehicle burdens of 5.1±0.18 and 7.1±0.1 log₁₀ CFU/g tissue respectively, V-r exhibited a dose-dependent reduction in bacterial burden of 1.2 to 3.4 log₁₀ as compared to vehicle (see Table 4; Kruskal-Wallis one-way analysis of variance). V-r doses at 440 and 880 mg/kg afforded 1.0 and 1.3 log₁₀ reduction below stasis respectively, with an extrapolated static dose of 215 mg/kg.

TABLE 4
Efficacy of V-r in E. coli thigh muscle infection model in CD-1 mice

| | Log₁₀ Group | Logio change | Statistical |
|---|---|---|---|
| Group, total dose | Geometric mean | from vehicle | difference |
| over 9 h (mg/kg) | (CFU/g) ± SD | (CFU/g) | (versus vehicle) |
| Pre-treatment | 5.1 ± 0.18 | −2.01 | p = 0.0045 |
| Vehicle | 7.11 ± 0.12 | 0 | 0 |
| V-r, 110 | 5.87 ± 0.60 | −1.24 | p = 0.0415 |
| V-r, 440 | 4.14 ± 0.63 | −2.97 | p < 0.0001 |
| V-r, 880 | 3.76 ± 0.40 | −3.35 | p < 0.0001 |
| vancomycin, 800 | 6.60 ± 0.66 | −0.51 | non-significant |

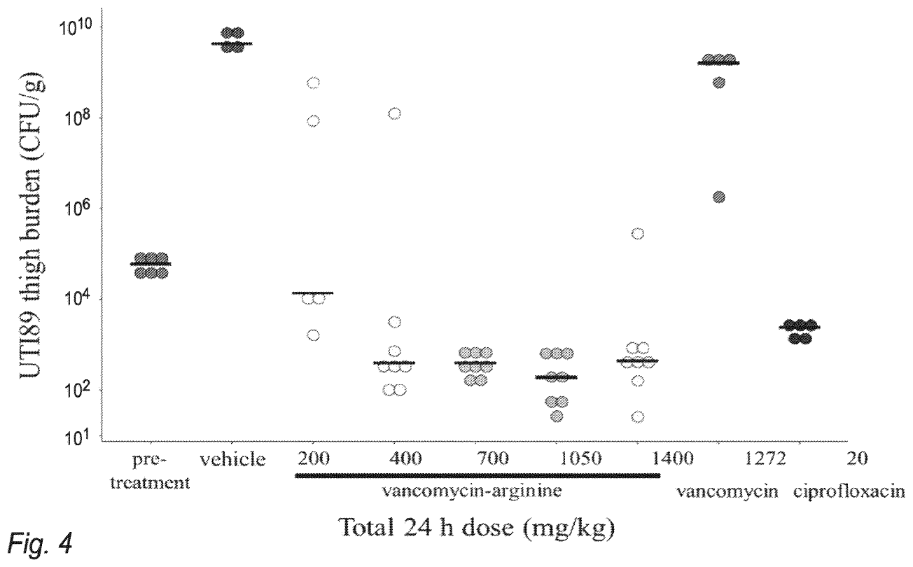

Further, as anticipated, vancomycin failed to significantly impact E. coli burden at a dose equivalent to the highest dose of V-r. In a 24 h thigh muscle infection model, E. coli UTI89 was inoculated at 7.8×10⁴ CFU into one thigh muscle per mouse (n=5-8/group) and treated with V-r (total dose of 200-1400 mg) using a q6h dosing regimen from 1 h post-infection. All doses higher than 200 mg/kg significantly reduced burden below stasis by up to 2.7 log₁₀ CFU/g. These bactericidal effects of V-r were statistically superior to ciprofloxacin, which induced a log₁₀ 1.4 reduction from stasis (see FIG. 4 and Table 5). Overall, V-r caused a ˜4 to 7.5 log₁₀ reduction in bacterial burden, as compared to vehicle control, over the entire dose range.

TABLE 5
Efficacy of V-r in reducing E. coli burden in CD-1 mice

| | Logio Group | Logio change | Statistical |
|---|---|---|---|
| Group, total dose | Geometric mean | from vehicle | difference |
| over 24 h (mg/kg) | (CFU/g) ± SD | (CFU/g) | (versus vehicle) |
| Pre-treatment | 4.76 ± 0.18 | −4.95 | p = 0.0248 |
| Vehicle | 9.71 ± 0.17 | 0 | 0 |
| V-r, 200 | 5.60 ± 2.28 | −4.11 | p = 0.0217 |
| V-r, 400 | 3.27 ± 1.88 | −6.43 | p < 0.0001 |
| V-r, 700 | 2.58 ± 0.25 | −7.13 | p < 0.0001 |
| V-r, 1050 | 2.08 ± 0.89 | −7.63 | p < 0.0001 |
| V-r, 1400 | 2.68 ± 1.38 | −7.03 | p < 0.0001 |
| vancomycin, 1272 | 8.48 ± 1.31 | −1.23 | non-significant |
| ciprofloxacin, 20 | 3.32 ± 0.14 | −6.39 | p < 0.0007 |

In summary, the MIC data confirm that the coupling of arginine to vancomycin confers significant antimicrobial activity of the V-r conjugate against E. coli, while remaining effective against MRSA. The in vitro findings were further effectively translated into thigh muscle infection models, where a total 24 h dose of 250 mg/kg V-r reduced E. coli burden to pre-treatment (stasis) levels. Since AUC/MIC is the primary PK/PD predictor of vancomycin, this static dose corresponds with a tAUC₀₋₂₄ₕ/MIC=47.3. Based on a free fraction of 35%, as determined in plasma protein binding studies (Table 2), the fAUC₀₋₂₄ₕ/MIC of V-r=16.5. As an approximation of exposure using allometric scaling (FDA's Guidance for Industry, ‘Estimating the maximum safe starting dose in initial clinical trials for therapeutics in adult healthy volunteers’ 2005, see also Table 6), this would be equivalent to a human dose of about 20 mg/kg, with a dose of 28 mg/kg required to elicit an additional 1 log₁₀ kill. Such allometric doses of V-r are in line with the daily and loading doses of vancomycin in humans.

The positive efficacy data support the notion that the cationic feature of arginine within V-r allows breaching of the stubborn outer membrane of E. coli and possibly other GNB. The sequelae of events leading to V-r mediated E. coli eradication is likely involve: a) improved cell surface association with negatively charged groups; b) effective translocation across the outer membrane leading to enhanced drug uptake, and c) disruption of peptidoglycan synthesis within the periplasmic space.

Overall, the current findings provide strong evidence that a minimally modified vancomycin-cationic transporter conjugate causes a marked abrogation of E. coli burden in vivo. Since V-r was highly effective in time-kill assays against E. coli NCTC-13441, a pandemic uropathogenic clone, a logical next step would be to evaluate the conjugate in a model of urinary tract infection. Based on the high renal elimination of vancomycin in humans in a non-metabolized form, it is reasonable to hypothesize that V-r could drive a highly targeted therapeutic intervention to combat E. coli-associated UTIs.

### Example 5: Efficacy of V-r in the Treatment of Urinary Tract Infections In Vivo

Female C3H/Hej mice were placed on a 5% glucose water starting 6 days prior to infection and then for the remainder of the study. The complicated urinary tract infection (cUTI) model was established via trans-urethral injection of the bacterial inoculum. Mice were anesthetized with Ketamine HCL at 40 mg/kg body weight and Xylazine at 6 mg/kg body weight in 0.15 ml PBS injected intra-peritoneally. Using a dissection scope (10×), the urethral orifice was located and a tapered PE10 catheter (attached to MRE40 tubing and a 23 G blunt hub syringe) was inserted and 0.05 ml of the prepared inoculum slowly injected. Consequently, the bacteria ascend through the urinary tract and localize in the kidney, establishing an infection site. E. coli UNT057-1 (CTX-M-15) strain was used for the cUTI model and inoculum grown in Tryptic soy broth (TSB) to 1.0×10¹⁰ CFU/ml suspension. Four (4) days post-infection, mice were treated IV with V-r (1-50 mg/kg) given q12h for 3 days. A positive control of meropenem was given at 300 mg/kg q12h by sub-cutaneous administration for a total of 3 days. All treatment groups consisted of 10 animals with CFU (kidneys, bladder and urine) bacterial counts at 7 days post-infection used as the endpoint for efficacy. At 7 days post-infection, urine was collected from mice into 1.5 ml sterile tubes and then mice were euthanized. Kidneys and bladders were homogenized, and the homogenates and urine were serially diluted in 1×PBS and plated onto Brain heart infusion and 0.5% charcoal agar. Agar plates were incubated at 37° C., and colony counts were recorded the following day.

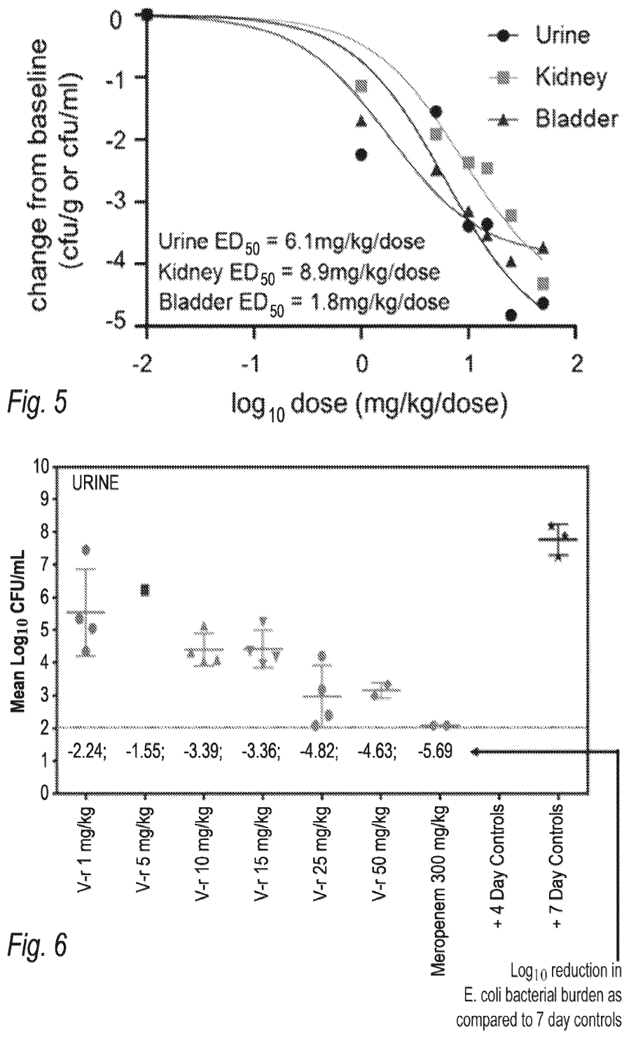

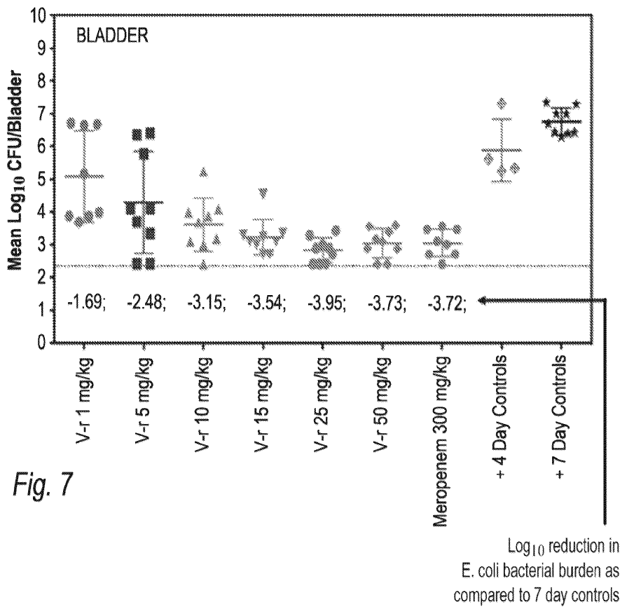

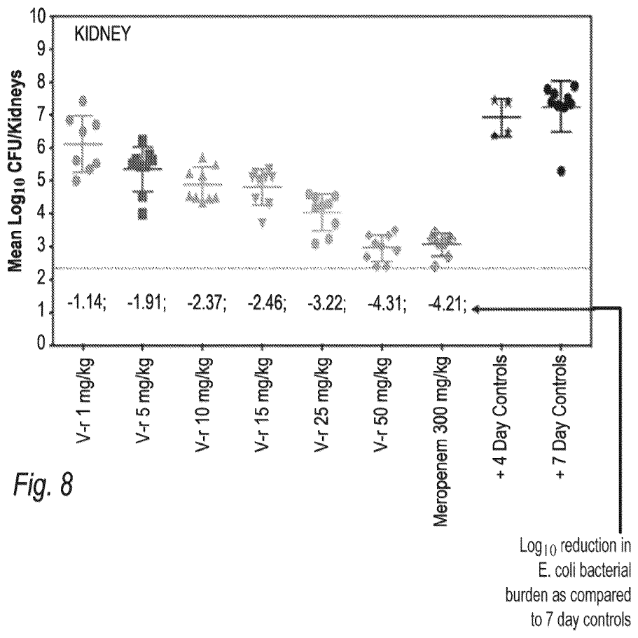

The ED₅₀ (mg/kg) of V-r in abrogating bacterial burden in the mouse cUTI E. coli model was calculated by regression analysis and determined to be 1.8-8.9 mg/kg for bladder, urine and kidney (see FIG. 5). Maximal abrogation of bacterial burden was observed following a q12h V-r dose of 25 mg/kg in urine, (see FIG. 6), bladder (FIG. 7) and 50 mg/kg for kidney (FIG. 8) compartments.

The daily 24 h dose of vancomycin in humans to treat MRSA infections is 2 g, given as 15 mg/kg q12h, with an initial loading dose often given as 4 g (25-30 mg/kg q12h) to cover the first 24 h of therapy. In the current E. coli cUTI model, the required daily 24 h doses of V-r to cause maximal reductions in bacterial burden are 50 mg/kg (urine and bladder) and 100 mg/kg (kidney). Based on allometric dosing using surface area algorithms, the theoretical V-r therapeutic doses to combat E. coli-associated cUTIs in rats, dogs and humans can be calculated (see Table 6). The rat equivalent dose is 0.504 multiplied by the mouse doses, the dog equivalent dose is 0.146 multiplied by the mouse doses and the human equivalent dose is 0.081 multiplied by the mouse doses. Based on these equivalent allometric doses, the effective human dose of V-r to combat a cUTI caused by E. coli in humans can be about 0.13-0.28 of the vancomycin dose required to treat MRSA infections in humans (see Table 6). Thus, the addition of arginine to vancomycin allows a much lower effective dose of the vancomycin conjugate in the treatment of cUTIs, thereby significantly mitigating potential toxicities.

TABLE 6
Equivalent effective according to FDA's Guidance for Industry

| Effective dose (mg/kg) of V-r that causes | Equivalent effective V-r doses In rats, dogs and humans based on allometric dosing | | |
|---|---|---|---|
| maximum reduction of E. coli bacterial | Equivalent rat | Equivalent dog | Equivalent human |
| burden in a mouse cUTI model | effective dose (mg/kg) | effective dose (mg/kg) | effective dose (mg/kg) |
| 25 | 12.62 | 3.66 | 2.03 |
| 50 | 25.25 | 7.44 | 4.13 |

Equivalent effective doses in rats, dogs and humans according to the FDA's “Guidance for Industry” - Estimating the maximum safe starting dose in initial clinical trials for therapeutics in adult healthy volunteers(https://www.fda.gov/media/72309/download).

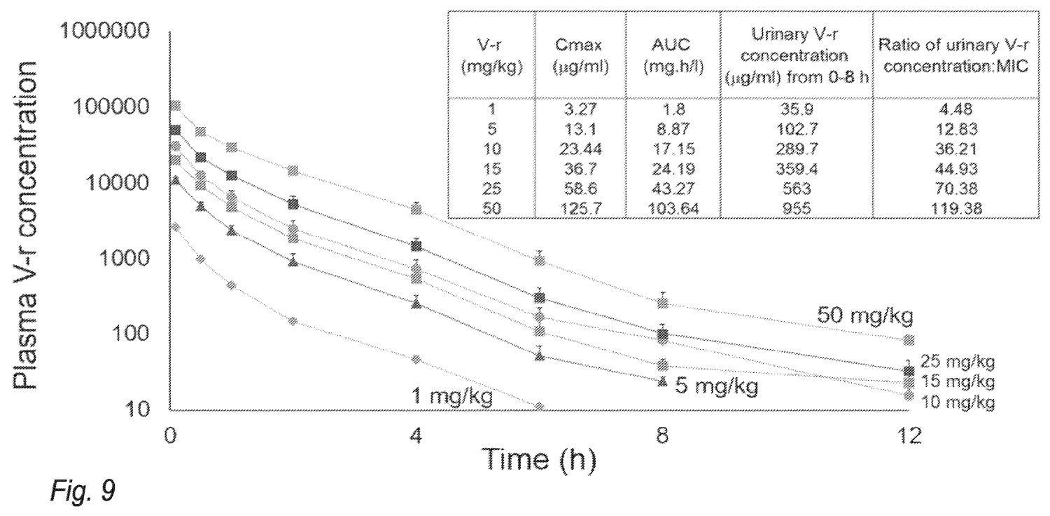

An alternative way to evaluate if a low dose therapy with V-r in humans might be feasible is to determine the required target exposure, i.e., Area Under the Curve (AUC). To that end, the PK characteristics of V-r were determined in mice following a single IV administration at identical doses (1-50 mg/kg) to those used in the cUTI model (see FIG. 9). In addition, the concentration of V-r was determined in urine during a period of 8 h following drug administration to determine the ratio of V-r concentration:MIC. V-r's PK profile was found to be linear, with target AUCs at the effective 25 and 50 mg/kg doses being 43.27 and 103.64 mg·h/l respectively. Furthermore, the ratio of V-r:MIC was 70.38 and 119.38, thus well in excess to drive the desired antibacterial effect.

According to previous PK/PD studies in rats, dogs and humans, the target AUC of vancomycin in the treatment of MRSA infections is approximately 400 mg·h/l, achieved by dosing at 150 mg/kg in rats, 100 mg/kg in dogs and 15 mg/kg in humans. Since the PK characteristics of vancomycin are linear and that V-r and vancomycin profiles are extremely similar in mice, it is possible to calculate the purported therapeutic doses of V-r in different species that generate target AUCs of 43.27 and 103.64 mg·h/l.

TABLE 7
Theoretical effective doses of V-r in rats, dogs and humans

| | theoretical V-r doses that drive target AUC in rats, dogs and | | |
|---|---|---|---|
| Target V-r AUC (mg.h/l) in mice that causes | humans based on published findings | | |
| maximum reduction of E. coli bacterial | Equivalent rat | Equivalent dog | Equivalent human |
| burden in a mouse cUTI model | effective dose (mg/kg) | effective dose (mg/kg) | effective dose (mg/kg) |
| 43.27 (at a q12h dose of 25 mg/kg) | 16.23 | 10.82 | 1.62 |
| 103.64 (at a q12h dose of 50 mg/kg) | 38.87 | 25.91 | 3.88 |

Equivalent effective doses in rats, dogs and humans according to the FDA's “Guidance for Industry” -Estimating the maximum safe starting dose in initial clinical trials for therapeutics in adult healthy volunteers(https://www.fda.gov/media/72309/download).

The theoretical effective doses of V-r in rats, dogs and humans were calculated based on therapeutic AUCs (see Table 7). To generate an AUC of 43.27 mg·h/l, the equivalent doses can be calculated as follows: rats=16.23 mg/kg; dogs=10.82 mg/kg; and humans=1.62 mg/kg. Similarly, for the AUC of 103.64, the equivalent doses are: rats=38.87 mg/kg; dogs=25.91 mg/kg; and humans=3.88 mg/kg. As a result, the effective q12h human dose of V-r to combat a cUTI in humans caused by E. coli can be ˜0.11-0.26 of the human q12h dose of vancomycin (15 mg/kg). Thus, either by allometric dosing (Table 6) or AUC targeting (Table 7), an unexpected low dose therapy of V-r to treat E. coli-associated cUTIs in humans seems highly feasible.

Experimental evidence for a low dose V-r therapy was further supported by a PK study in a beagle dog following the infusion of a single dose of V-r (25 mg/kg) administered as a 60 min IV infusion. This dose was chosen since it represents an effective split dose therapy for vancomycin treatment of GPB infections in dogs (DeStefano et al, 2019; J Vet Int Med 33: 200-207) as well as being an approximate canine equivalent allometric dose according to the dosage of vancomycin in the treatment of such infections in humans.

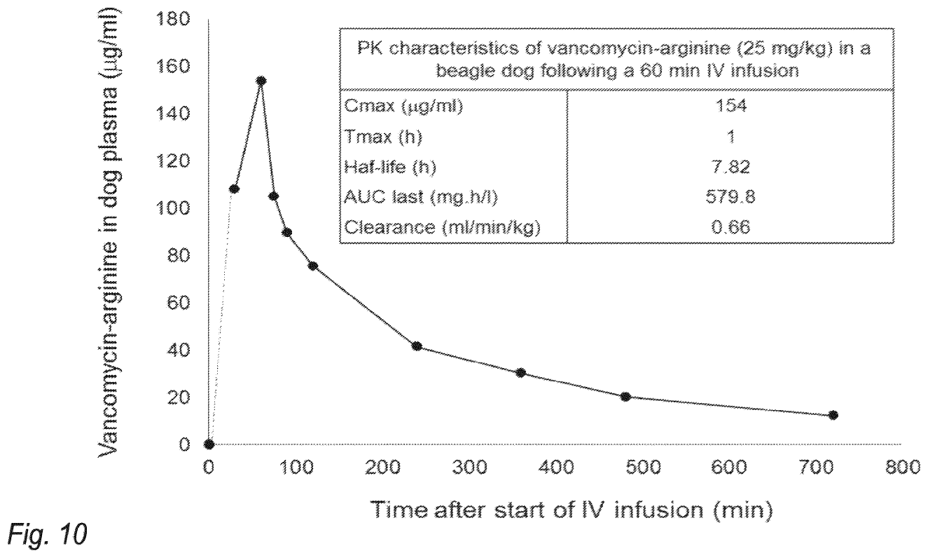

Accordingly, a single dose of 25 mg/kg V-r in the dog generated an AUC of 580 mg·h/l (see FIG. 10). The equivalent dose in humans to generate an identical AUC would be expected to be 14 mg/kg. With V-r's target AUCs ranging from approximately 40-100 mg·h/l (FIG. 9) and assuming a linear PK relationship in humans, the required allometric human q12h doses of V-r to elicit such AUCs might be expected to be 0.94 mg/kg to 2.35 mg/kg for patients suffering from E. coli-associated cUTIs. Assuming a 10-day therapy for a 70 kg patient, this would equate to a total dose of 1.31 g to 3.29 g. Such amounts are significantly lower than the vancomycin dose to treat GPB infections (15 mg/kg q12h for 10 days), which totals 20 g. Furthermore, as compared to currently used IV antibiotics to treat cUTIs driven by GNB pathogens (e.g., Piperacillin-Tazobactam, Ceftazidime/Avibactam, Ertapenem, Cefiderocol), the total dose of V-r is expected to be substantially less, ranging from approximately 0.96-13% of the total doses of comparator drugs (see Table 10).

TABLE 10
Anticipated V-r dosing to treat E. coli-associated cUTIs in humans

| | IV dosing | | Total dose (g) |
|---|---|---|---|
| | regimen | Total daily | based on a 10 |
| Drug | per day | dose (g) | day therapy |
| Vancomycin- | 0.065-0.165 g BID | 0.13-0.33 | 1.3-3.3 |
| arginine | | | |
| Piperacillin- | 4.5 g TID | 13.5 | 135 |
| Tazobactam | | | |
| Ceftazidime/ | 2.5 g TID | 7.5 | 75 |
| Avibactam | | | |
| Ertapenem | 1 g QD | 1 | 10 |
| Cefiderocol | 2 g TID | 6 | 60 |
| Vancomycin | 1 g BID | 2 | 20 |
| to treat | | | |
| MRSA | | | |

Calculations of the anticipated V-r dosing are based on allometric AUC equivalent dosing, comparing the total 10 day amounts of V-r as with other IV injectables.

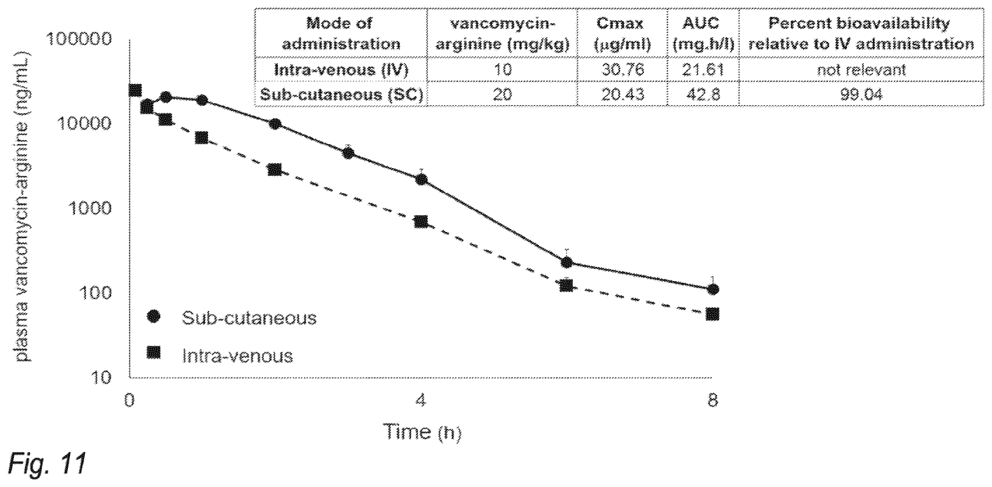

Adaptation of V-r as a low-dose therapy to treat E. coli-associated cUTIs via sub-cutaneous administration was further investigated. Vancomycin can only be given by IV infusion therapy to treat systemic infections. Due to the very low dose therapy of V-r, additional modes of administration of the drug can be pursued besides IV administration pending retention of high bioavailability. In mice, the exposure of V-r (measured by the AUC) following sub-cutaneous administration was essentially 100% of that afforded by IV administration (see FIG. 11). These data suggest that V-r is amenable for administration via both IV and SC routes. Such a finding is critically important for hospitalized cUTIs (and other infections) whereby the SC route might offer a “go-home sooner” of patients and thus early discharge from the hospital for continued low dose V-r therapy from outpatient departments or within the clinical community settings. Such an approach facilitates the practice of prudent antimicrobial stewardship.

1-80. (canceled)

81. A compound of the general Formula A-MoTr, wherein A is a cargo moiety and MoTr is a transporter moiety comprising a lipophilic moiety, a cationic moiety and optionally a linker moiety.

82. The compound according to claim 81, wherein the cargo moiety is an antibiotic.

83. The compound according to claim 81, wherein MoTr is any one of

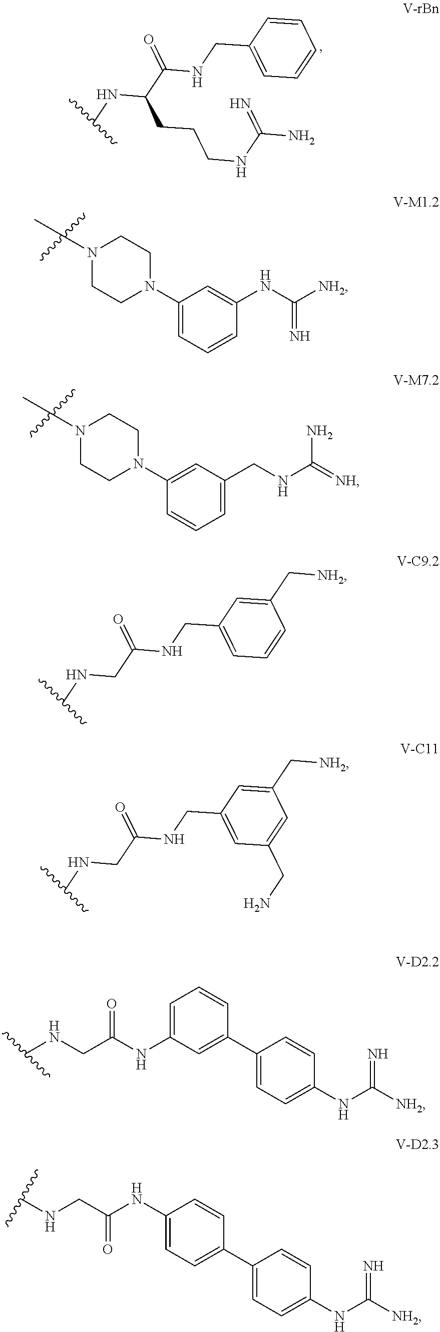

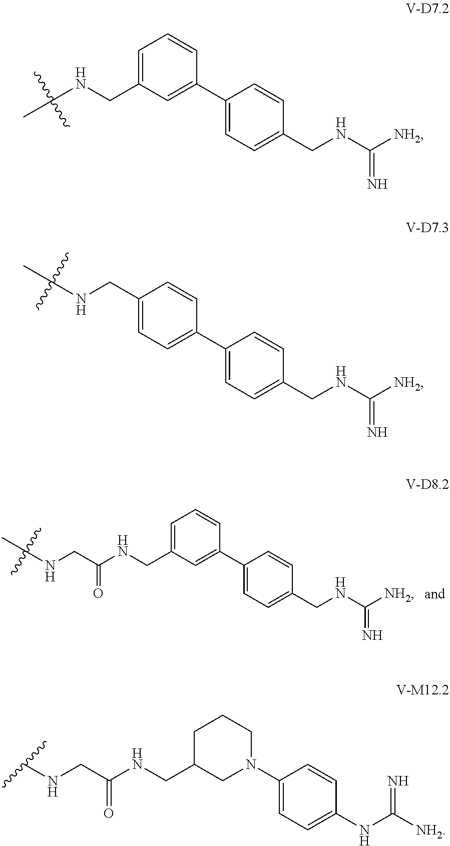 [SMILES: *NCc1cccc(-c2ccc(CNC(=N)N)cc2)c1 ; *NCc1ccc(-c2ccc(CNC(=N)N)cc2)cc1 ; *NCC(=O)NCc1cccc(-c2ccc(CNC(=N)N)cc2)c1 ; "and" ; *NCC(=O)NCC1CCCN(c2ccc(NC(=N)N)cc2)C1]

84. The compound according to claim 81, wherein MoTr is any one of

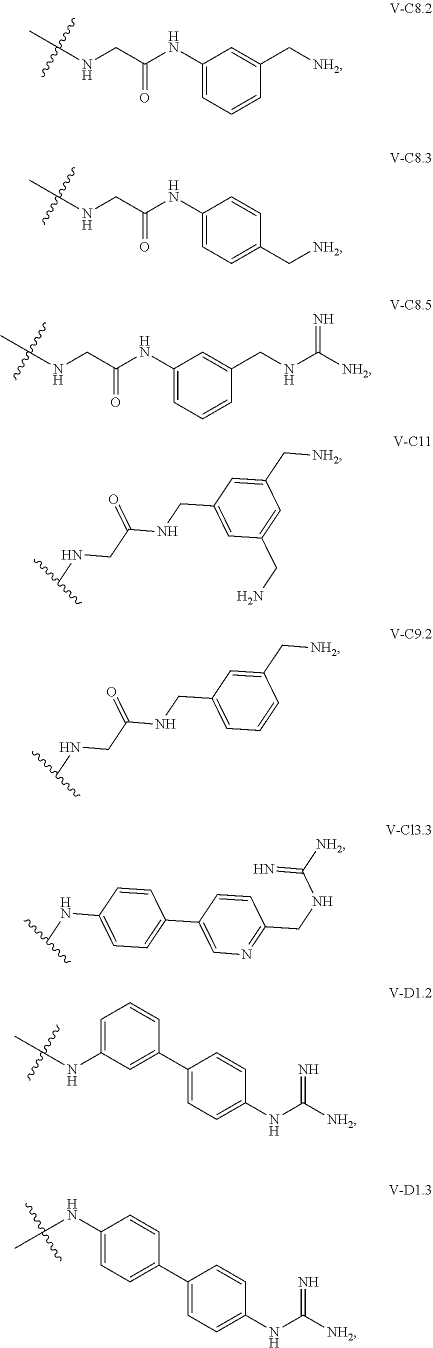

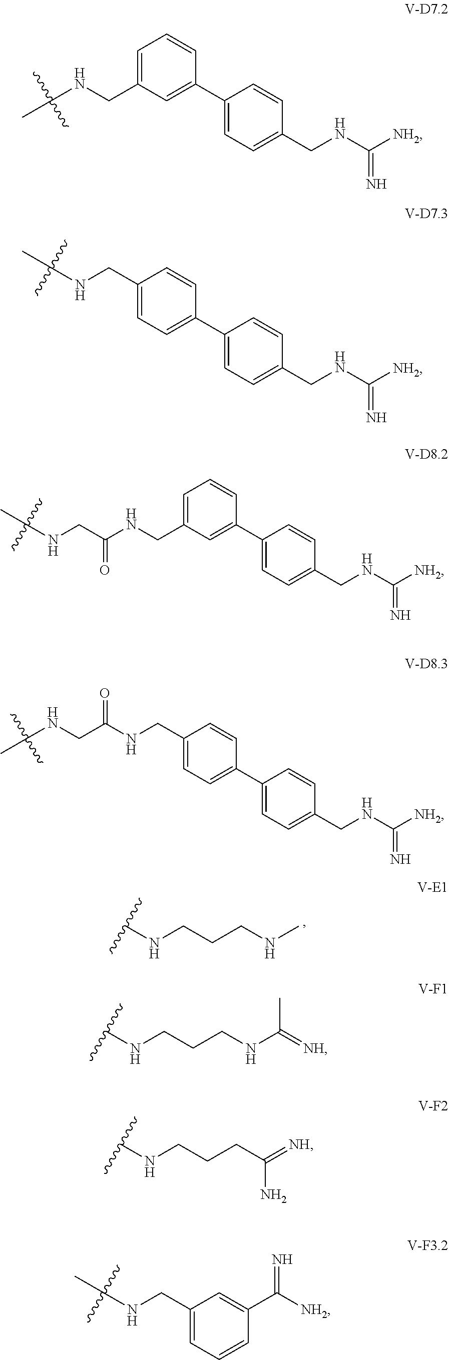

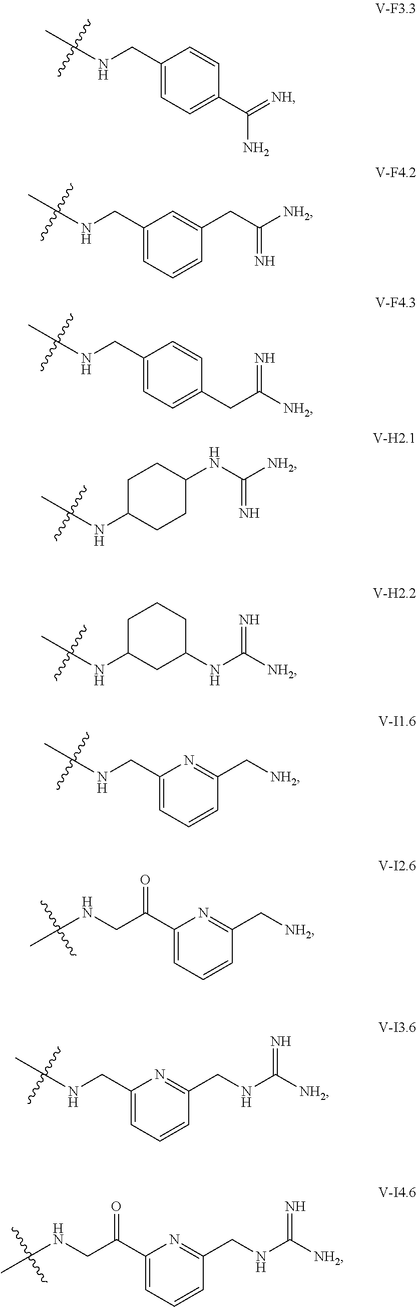

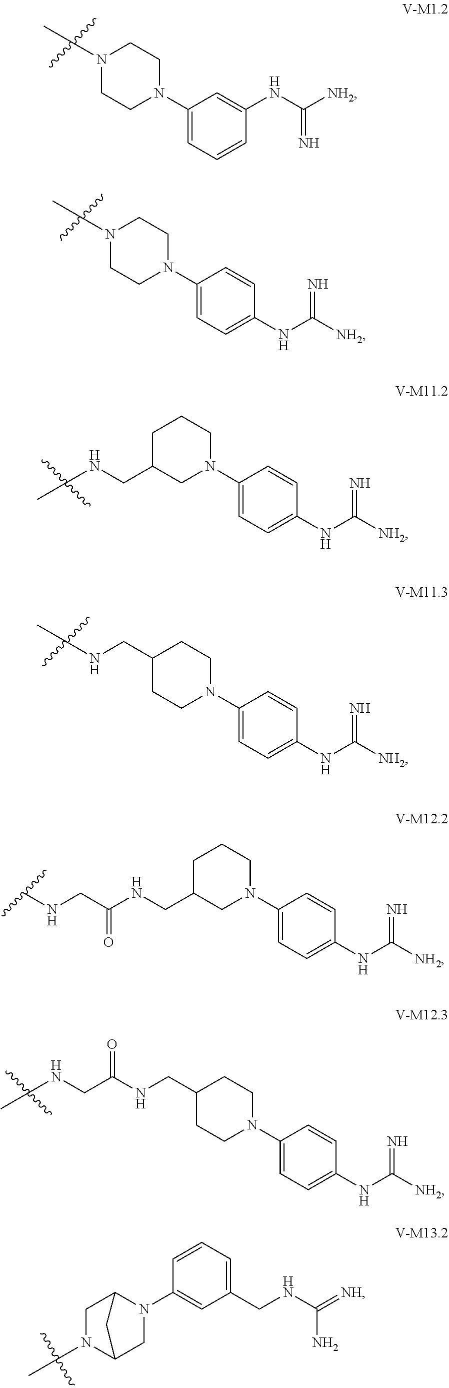

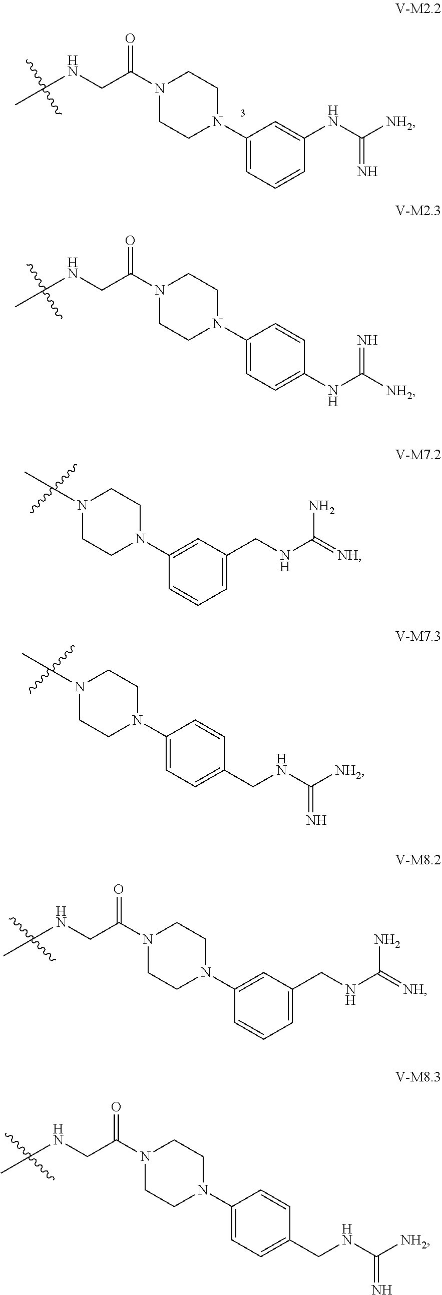 [SMILES: *NCC(=O)N1CCN(c2cccc(NC(=N)N)c2)CC1 ; *NCC(=O)N1CCN(c2ccc(NC(=N)N)cc2)CC1 ; *N1CCN(c2cccc(CNC(=N)N)c2)CC1 ; *N1CCN(c2ccc(CNC(=N)N)cc2)CC1 ; *NCC(=O)N1CCN(c2cccc(CNC(=N)N)c2)CC1 ; *NCC(=O)N1CCN(c2ccc(CNC(=N)N)cc2)CC1]

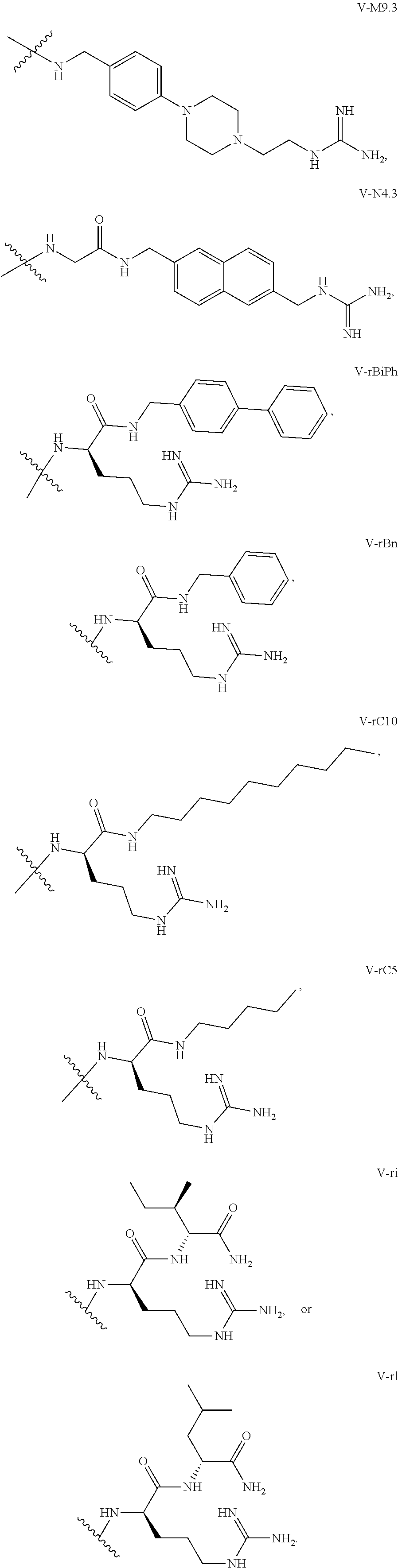

85. The compound according to claim 81, wherein MoTr is any one of 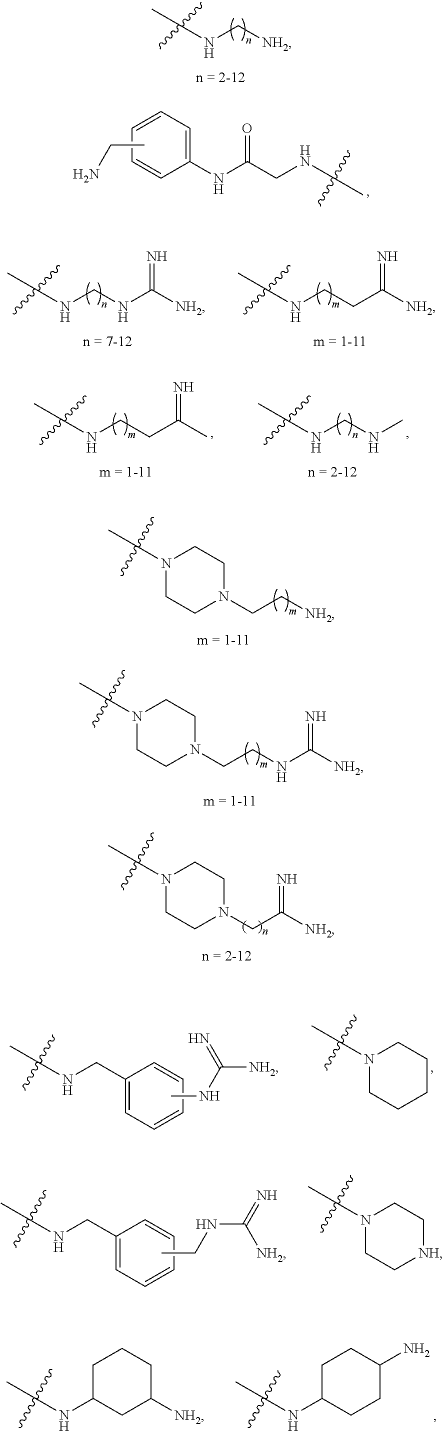 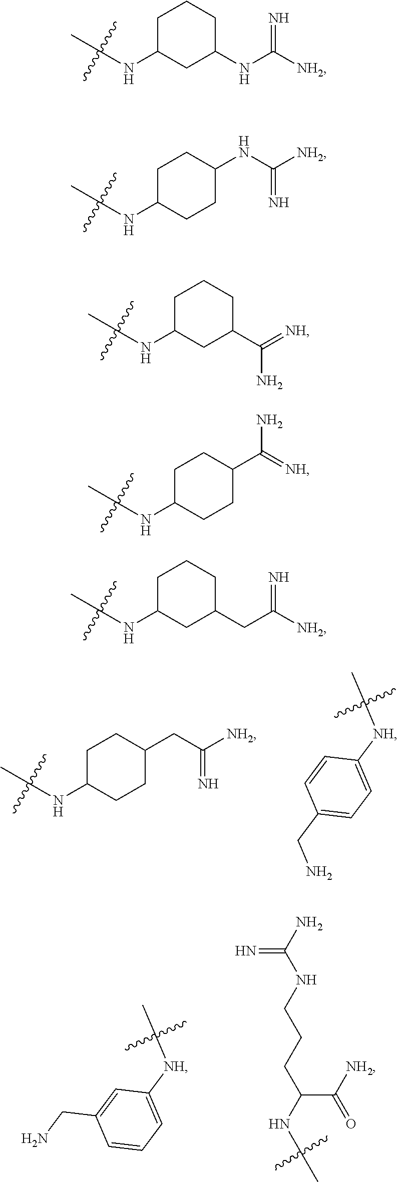 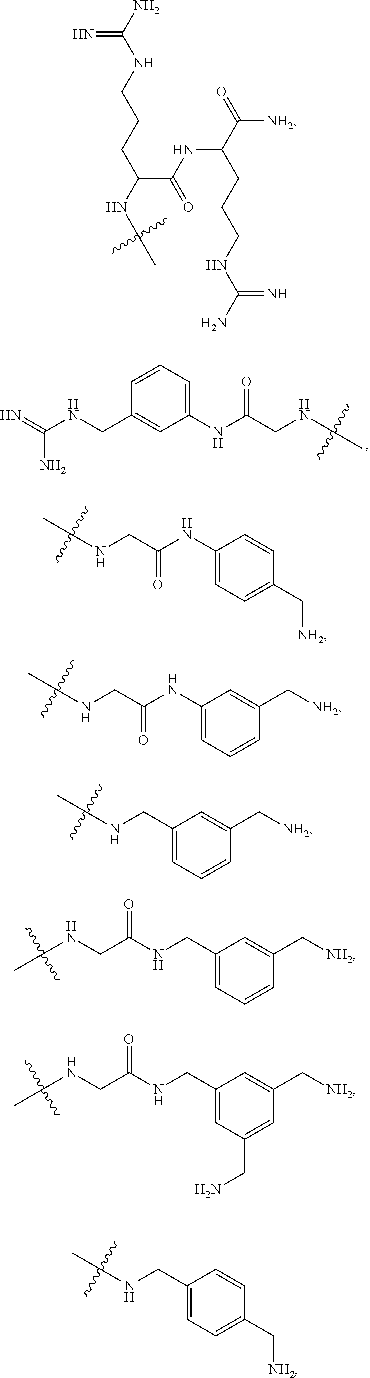 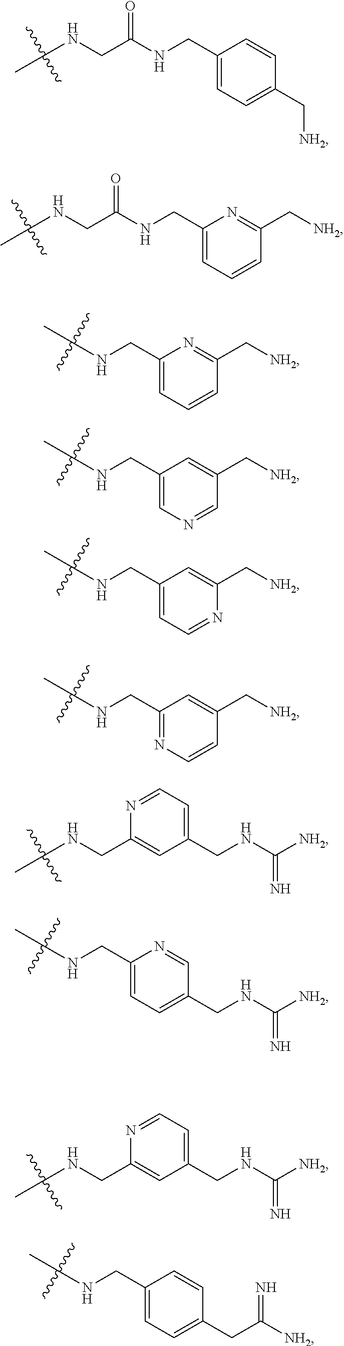 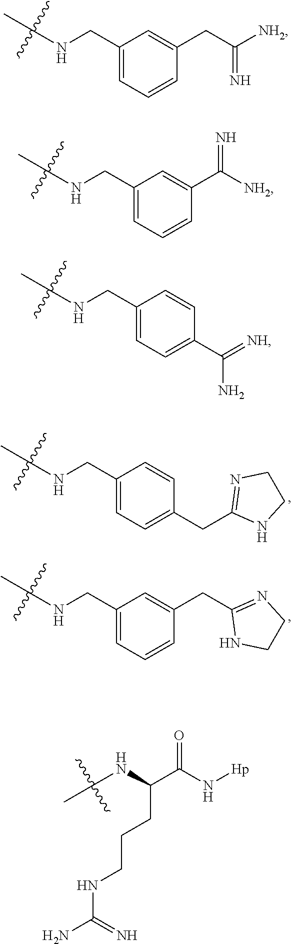
Hp is as defined herein

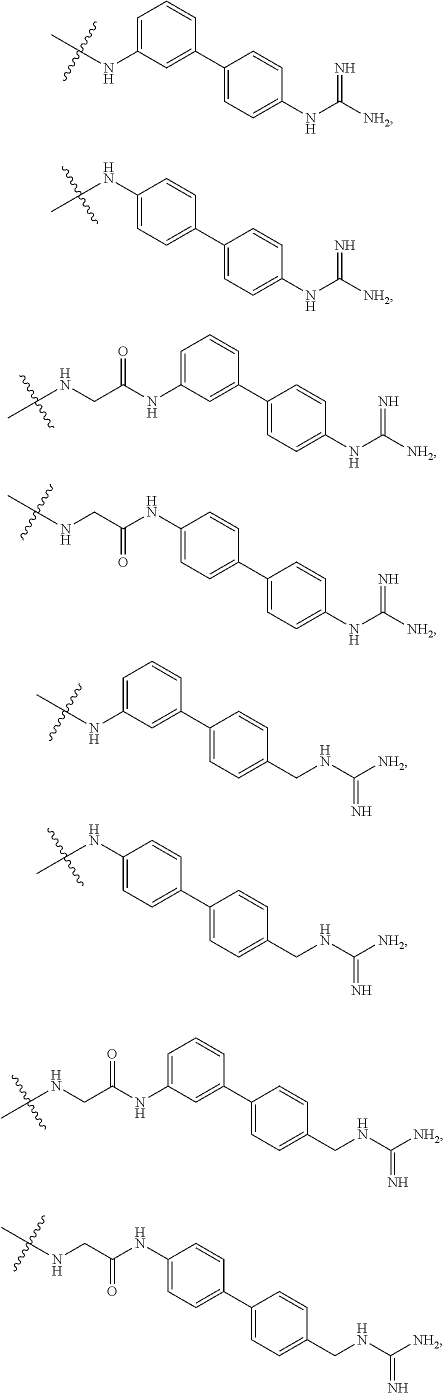

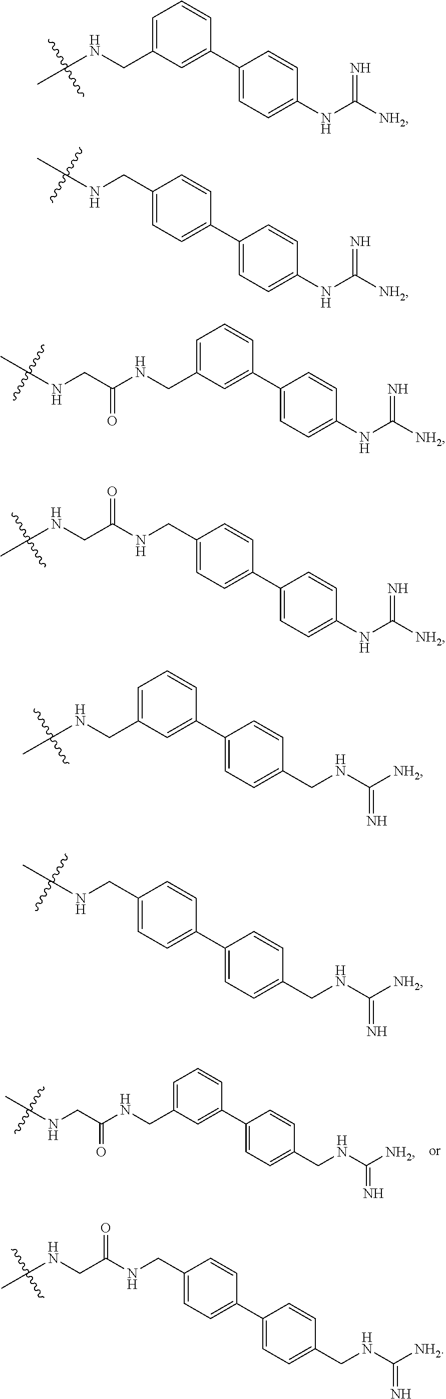

86. The compound according to claim 81, being a compound of Formula (I): 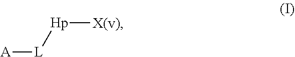
wherein
A is a cargo moiety,
L is a linker moiety, which is optionally absent,
Hp is a lipophilic moiety directly associated with X or via linker moiety -Lₕ-
X is an N-containing cationic group, and
v is 1 or 2.

87. The compound according to claim 86 being of Formula (Ia) or (Ib): 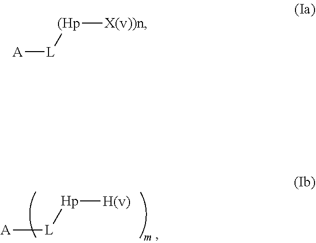
wherein A, L, Hp, X, v, n and m are each as defined in claim 85.

88. The compound according to claim 86, represented by Formula (II): 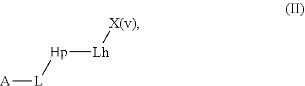
wherein A, L, Hp, X, v, n and m are each as defined in claim 86.

89. The compound according to claim 86, having a structure selected from Formulae (III), (IV), (V), (VI) and (VII): 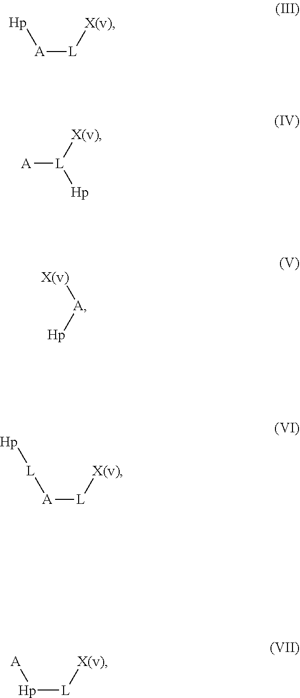
wherein A, L, Hp, X, v, n and m are each as defined in claim 86.

90. The compound according to claim 89, wherein a compound of Formula (III) is a compound of Formula (IIIa): 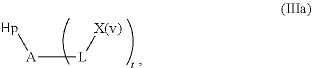
wherein A, L, Hp, X, v, n and m are each as defined in claim 86 and wherein t is an integer between 1 and 10.

91. The compound according to claim 86, represented by Formula (VIII) or (IX): 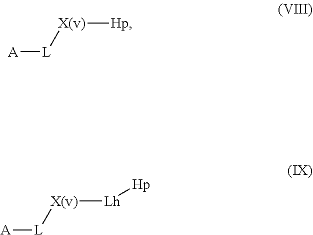
wherein A, L, Hp, X, v, n and m are each as defined in claim 86.

92. The compound according to claim 91, represented by Formula (VIIIa) or (VIIIb): 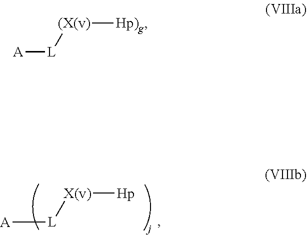
wherein A, L, Hp, X, v, n and m are each as defined in claim 88 and each of g and j, independently of the other, is an integer between 1 and 10.

93. The compound according to claim 86, wherein the cargo moiety is an antibiotic.

94. The compound according to claim 93, wherein the antibiotic is linezolid or vancomycin.

95. A compound of the general Formula A-MoTr, wherein A is vancomycin or linezolid and MoTr is a transporter moiety comprising a structure as defined in claim 83.

96. The compound according to claim 85, wherein MoTr is any one of

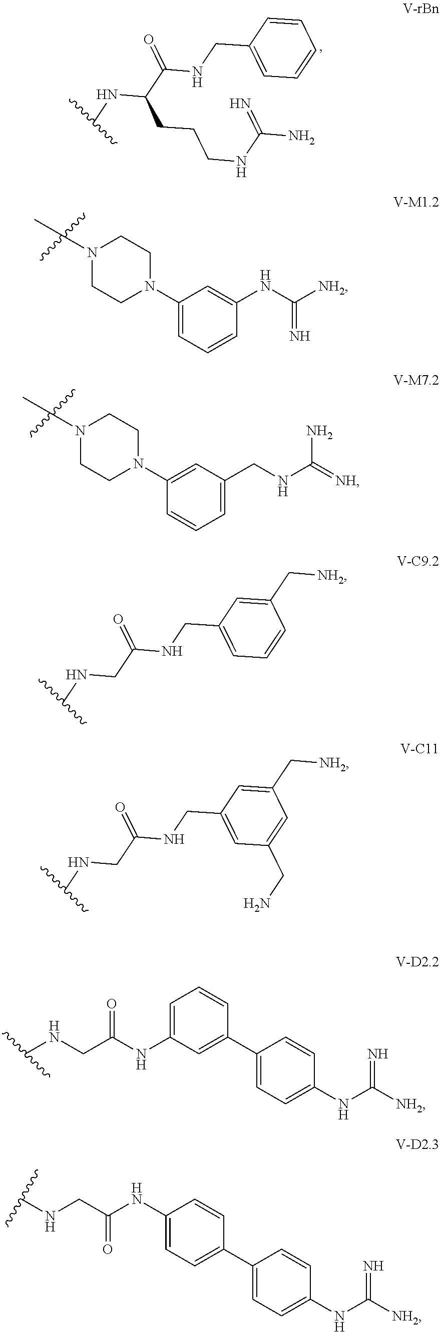

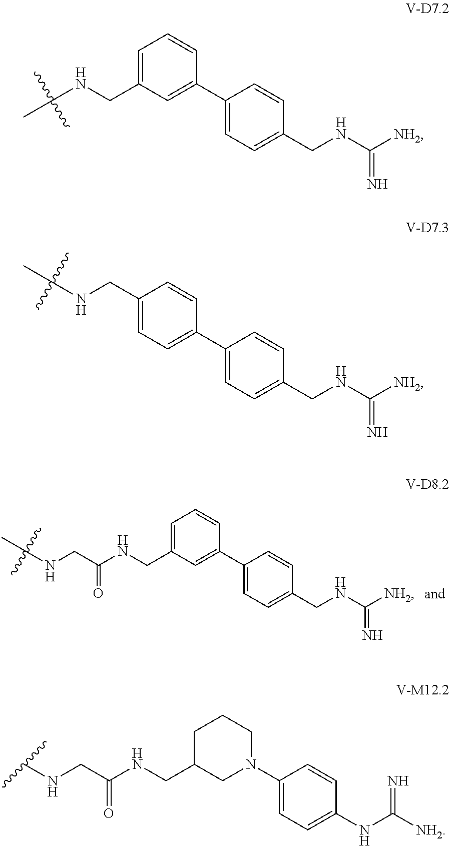 [SMILES: *NCc1cccc(-c2ccc(CNC(=N)N)cc2)c1 ; *NCc1ccc(-c2ccc(CNC(=N)N)cc2)cc1 ; *NCC(=O)NCc1cccc(-c2ccc(CNC(=N)N)cc2)c1 ; "and" ; *NCC(=O)NCC1CCCN(c2ccc(NC(=N)N)cc2)C1]

97. The compound according to claim 95, wherein MoTr is

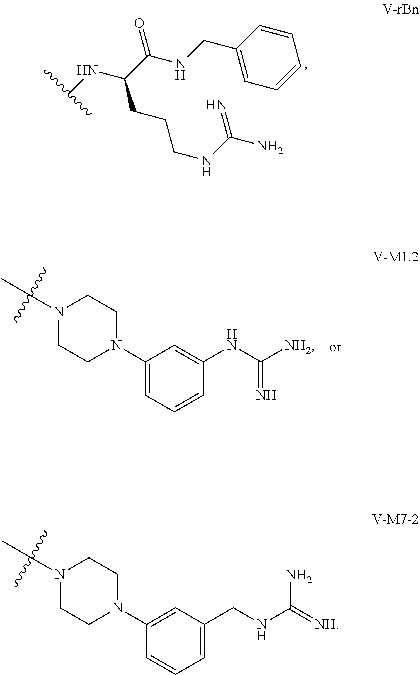 [SMILES: *N[C@H](CCCNC(=N)N)C(=O)NCc1ccccc1 ; *N1CCN(c2cccc(NC(=N)N)c2)CC1 ; *N1CCN(c2cccc(CNC(=N)N)c2)CC1]

98. A pharmaceutical composition comprising a compound according to claim 81 and a pharmaceutically acceptable buffer, carrier and/or excipient.

99. A method of inhibiting a Gram-positive or a Gram-negative bacterial infection in vivo and in vitro, the method comprises contacting a biological or a non-biological surface, a cell, a tissue, or an organism with a composition comprising a compound according to claim 81.

100. A method for preventing, alleviating, or treating a disease or a condition comprising a Gram-positive and/or a Gram-negative bacterial infection in a subject who is suffering or at risk of suffering therefrom, the method comprises administering to the subject a composition comprising a compound according to claim 81.
